(19) United States
(12) Patent Application Publication
(10) Pub. No.: US 2025/0297185 A1
(43) Pub. Date: Sep. 25, 2025

(54) FLUORINE-CONTAINING ETHER COMPOUND, LUBRICANT FOR MAGNETIC RECORDING MEDIUM, AND MAGNETIC RECORDING MEDIUM

(71) Applicant: Resonac Corporation, Tokyo (JP)

(72) Inventors: Takuya UNO, Tokyo (JP); Tsuyoshi KATO, Tokyo (JP); Yutaka TANJI, Tokyo (JP)

(73) Assignee: Resonac Corporation, Tokyo (JP)

(21) Appl. No.: 18/860,993

(22) PCT Filed: Dec. 6, 2023
(86) PCT No.: PCT/JP2023/043611

§ 371 Date: Oct. 28, 2024

(30) Foreign Application Priority Data
Dec. 9, 2022 (JP) 2022-197136

(51) Int. Cl. C10M 107/38; C08G 65/331; C10N 20/04; C10N 40/18; G11B 5/725

(52) U.S. Cl. CPC C10M 107/38; C08G 65/3312; G11B 5/7253; C10M 2213/0606; C10N 2020/04; C10N 2040/18

(57) ABSTRACT

This fluorine-containing ether compound is a fluorine-containing ether compound represented by the following formula:

R¹—CH₂—R²—(CH₂—OCH₂—CR³R⁴—CH₂O—CH₂—R²)_{z}—CH₂—R⁵

(z represents 1 or 2. R² is a perfluoropolyether chain. R¹ and R⁵ are terminal groups having 1 to 50 carbon atoms and having at least one polar group. R³ and R⁴ may be the same as or different from each other, and are organic groups which do not include a secondary hydroxy group and a tertiary hydroxy group, include one primary hydroxy group, and have 1 to 5 carbon atoms bonded to a tetrasubstituted carbon atom. The organic group may include an ether oxygen atom between the carbon atoms, and a bonding terminal to the tetrasubstituted carbon atom may be an ether oxygen atom.)

### TECHNICAL FIELD

The present invention relates to a fluorine-containing ether compound, a lubricant for a magnetic recording medium, and a magnetic recording medium. Priority is claimed on Japanese Patent Application No. 2022-197136, filed Dec. 9, 2022, the content of which is incorporated herein by reference.

### BACKGROUND ART

In order to increase the recording density in magnetic recording and reproducing devices, development of a magnetic recording medium suitable for a high recording density has been in progress.

In the related art, as a magnetic recording medium, there is a magnetic recording medium in which a recording layer is formed on a substrate and a protective layer made of carbon or the like is formed on the recording layer. The protective layer protects information recorded in the recording layer and improves the sliding property of a magnetic head. In addition, the protective layer coats the recording layer and prevents a metal contained in the recording layer from being corroded by an environmental substance.

However, the durability of the magnetic recording medium is not sufficiently obtained only by providing the protective layer on the recording layer. Therefore, a lubricant is applied to the surface of the protective layer to form a lubricating layer. The lubricating layer improves the durability and protective power of the protective layer and prevents the infiltration of a contaminant into the magnetic recording medium.

As a lubricant used in the case of forming the lubricating layer of the magnetic recording medium, a lubricant containing a compound having a polar group such as a hydroxy group at a terminal of a fluorine-based polymer having a repeating structure including —CF₂— has been proposed.

For example, Patent Document 1, Patent Document 2, and Patent Document 3 disclose fluorine-containing ether compounds including two perfluoropolyether chains in a molecule, in which a linking group having a secondary hydroxy group is disposed between two perfluoroether chains.

Patent Document 4 discloses a fluorine-containing ether compound including two perfluoropolyether chains in a molecule, in which a linking group having a primary hydroxy group and a secondary hydroxy group is disposed between two perfluoroether chains.

Patent Document 5, Patent Document 6, and Patent Document 7 disclose fluorine-containing ether compounds having a skeleton in which three perfluoropolyether chains are bonded through a linking group having a secondary hydroxy group, in which terminal groups having a polar group are each bonded to either side thereof through methylene groups (—CH₂—).

### CITATION LIST

### Patent Documents

Patent Document 1: PCT International Publication No. WO2021/251335
Patent Document 2: United States Patent Application, Publication No. 2020/0002640
Patent Document 3: PCT International Publication No. WO 2016/084781
Patent Document 4: PCT International Publication No. WO 2021/019998
Patent Document 5: United States Patent Application, Publication No. 2016/0260452
Patent Document 6: PCT International Publication No. WO 2018/116742
Patent Document 7: PCT International Publication No. WO 2017/145995

### SUMMARY OF INVENTION

### Technical Problem

In magnetic recording and reproducing devices, it is required to further reduce the floating height of the magnetic head. Therefore, it is required to further reduce the thickness of the protective layer and/or the lubricating layer in the magnetic recording medium.

However, in general, in a case where the thickness of the lubricating layer is reduced, the chemical substance resistance of the magnetic recording medium tends to deteriorate. In addition, in a case where the floating height of the magnetic head is reduced, there is a case where pickup in which the fluorine-containing ether compound in the lubricating layer adheres to the magnetic head occurs.

The present invention has been made in view of the above circumstances, and an object of the present invention is to provide a fluorine-containing ether compound which has excellent chemical substance resistance, can form a lubricating layer capable of suppressing pickup, and can be suitably used as a material for a lubricant for a magnetic recording medium.

In addition, another object of the present invention is to provide a lubricant for a magnetic recording medium which contains the fluorine-containing ether compound of the present invention, has good chemical substance resistance, and can form a lubricating layer capable of suppressing pickup.

In addition, still another object of the present invention is to provide a magnetic recording medium which has a lubricating layer containing the fluorine-containing ether compound of the present invention and has good chemical substance resistance and in which pickup is suppressed.

### Solution to Problem

The present inventors have conducted intensive studies to achieve the above objects.

As a result, it was found that the object can be achieved by a fluorine-containing ether compound having two or three perfluoropolyether chains, in which a specific divalent linking group including a tetrasubstituted carbon atom to which two organic groups having a primary hydroxy group are bonded is disposed between adjacent perfluoropolyether chains through methylene groups, and a methylene group and a specific terminal group are each disposed in this order at both terminals of a skeleton including a perfluoropolyether chain, and the present invention was completed. That is, the present invention relates to the following matters.
[1]A fluorine-containing ether compound represented by Formula (1),

R¹—CH₂—R²—(CH₂—OCH₂—CR³R⁴—CH₂O—CH₂—R²)_{z}—CH₂—R⁵ (1)
(In Formula (1), z is 1 or 2. R² is a perfluoropolyether chain. (z+1) R²'s may be the same in part or in whole, or may be different from each other. R¹ and R⁵ may be the same as or different from each other and are terminal groups having 1 to 50 carbon atoms and having at least one polar group. R³ and R⁴ may be the same as or different from each other, and are organic groups which do not include a secondary hydroxy group and a tertiary hydroxy group, include one primary hydroxy group, and have 1 to 5 carbon atoms bonded to a tetrasubstituted carbon atom. The organic group may include an ether oxygen atom between the carbon atoms, and a bonding terminal to the tetrasubstituted carbon atom may be the ether oxygen atom. In a case where z is 2, two —CR³R⁴-'s may be the same as or different from each other.)
[2] The fluorine-containing ether compound according to [1], in which R³ and R⁴ in Formula (1) are each independently any one of groups represented by Formulae (2-1) to (2-3),

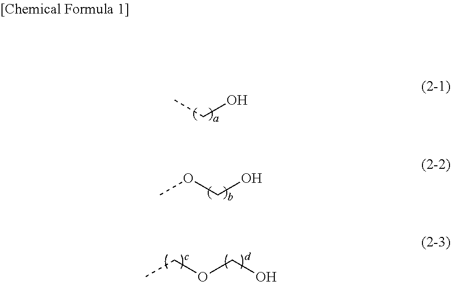

(In Formula (2-1), a is an integer of 1 to 5.)
(In Formula (2-2), b is an integer of 1 to 5.)
(In Formula (2-3), c and d are each independently an integer of 1 to 4, and c+d<5.)
[3] The fluorine-containing ether compound according to [1] or [2], in which the number of the polar groups contained in each of R¹ and R⁵ in Formula (1) is any one of 1 to 3.
[4] The fluorine-containing ether compound according to any one of [1] to [3], in which the polar groups contained in R¹ and R⁵ in Formula (1) are at least one selected from the group consisting of a hydroxy group, an amino group, a carboxy group, a formyl group, a carbonyl group, a sulfo group, a cyano group, and a group having an amide bond.
[5] The fluorine-containing ether compound according to any one of [1] to [4], in which R¹ and R⁵ in Formula (1) each independently have at least one hydroxy group as the polar group.
[6] The fluorine-containing ether compound according to any one of [1] to [5], in which R¹ and R⁵ in Formula (1) are each independently any of terminal groups represented by Formulae (4-1) to (4-3).

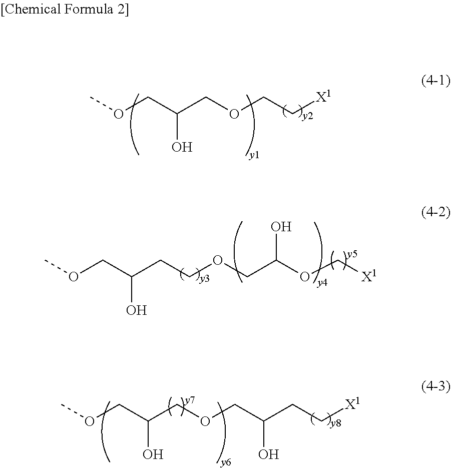

(In Formula (4-1), y1 is 1 or 2, and y2 is an integer of 0 to 3. X¹ is an aromatic hydrocarbon group, an unsaturated heterocyclic group, an alkenyl group, an alkynyl group, or a polar group. In a case where y1 is 1, X¹ is a polar group. In a case where X¹ is an aromatic hydrocarbon group or an unsaturated heterocyclic group, an atom constituting a ring structure in X¹ is bonded to a methylene group adjacent to X¹. In a case where X¹ is an alkenyl group or an alkynyl group, a carbon atom constituting an unsaturated bond in X¹ is bonded to a methylene group adjacent to X¹).
(In Formula (4-2), y3 is an integer of 1 to 3, y4 is 0 or 1, and y5 is an integer of 0 to 3. X¹ is an aromatic hydrocarbon group, an unsaturated heterocyclic group, an alkenyl group, an alkynyl group, or a polar group. In a case where y4 is 0, X¹ is a polar group. In a case where X¹ is an aromatic hydrocarbon group or an unsaturated heterocyclic group, an atom constituting a ring structure in X¹ is bonded to a methylene group adjacent to X¹. In a case where X¹ is an alkenyl group or an alkynyl group, a carbon atom constituting an unsaturated bond in X¹ is bonded to a methylene group adjacent to X¹).
(In Formula (4-3), y6 is 0 or 1, y7 is an integer of 1 to 3, and y8 is an integer of 1 to 3. X¹ is an aromatic hydrocarbon group, an unsaturated heterocyclic group, an alkenyl group, an alkynyl group, or a polar group. In a case where y6 is 0, X¹ is a polar group. In a case where X¹ is an aromatic hydrocarbon group or an unsaturated heterocyclic group, an atom constituting a ring structure in X¹ is bonded to a methylene group adjacent to X¹. In a case where X¹ is an alkenyl group or an alkynyl group, a carbon atom constituting an unsaturated bond in X¹ is bonded to a methylene group adjacent to X¹).
[7] The fluorine-containing ether compound according to any one of [1] to [6], in which (z+1) R²'s in Formula (1) are each independently a perfluoropolyether chain represented by Formula (3).

—(CF₂)_{w1}—O—(CF₂O)_{w2}—(CF₂CF₂O)_{w3}—(CF₂CF₂CF₂O)_{w4}—(CF₂CF₂CF₂CF₂O)_{w5}—(CF₂)_{w6}— (3)
(In Formula (3), w2, w3, w4, and w5 represent average degrees of polymerization and each independently represent 0 to 20. Here, there are no cases where all of w2, w3, w4, and w5 become 0 at the same time. w1 and w6 are average values representing the number of CF₂'s and each independently represent 1 to 3. An order of arrangement of (CF₂O), (CF₂CF₂O), (CF₂CF₂CF₂O), and (CF₂CF₂CF₂CF₂O), which are repeating units in Formula (3), is not particularly limited.
[8] The fluorine-containing ether compound according to any one of [1] to [7], in which (z+1) R²'s in Formula (1) are each independently any one selected from perfluoropolyether chains represented by Formulae (3-1) to (3-4).

—CF₂—(OCF₂CF₂)ₙ—(OCF₂)ₒ—OCF₂— (3-1)
(In Formula (3-1), n and o represent average degrees of polymerization, n represents 1 to 20, and o represents 0 to 20.)

—CF₂CF₂—(OCF₂CF₂CF₂)ₚ—OCF₂CF₂— (3-2)
(In Formula (3-2), p represents an average degree of polymerization and represents 1 to 15.)

—CF₂CF₂CF₂—(OCF₂CF₂CF₂CF₂)_{q}—OCF₂CF₂CF₂— (3-3)
(In Formula (3-3), q represents an average degree of polymerization and represents 1 to 10.)

—(CF₂)_{w7}—O—(CF₂CF₂CF₂O)_{w8}—(CF₂CF₂O)_{w9}—(CF₂)_{w10}— (3-4)
(In Formula (3-4), w8 and w9 represent average degrees of polymerization and each independently represent 1 to 20. w7 and w10 are average values representing the number of CF₂'s and each independently representing 1 to 2.)
[9] The fluorine-containing ether compound according to any one of [1] to [8], in which in Formula (1), R³'s and R⁴'s in z —CR³R⁴'s— are all the same as each other.
[10] The fluorine-containing ether compound according to any one of [1] to [9], in which all (z+1) R²'s in Formula (1) are all the same as each other.
[11] The fluorine-containing ether compound according to any one of [1] to [10], in which R¹ and R⁵ in Formula (1) are the same as each other.
[12] The fluorine-containing ether compound according to any one of [1] to [11], in which a number-average molecular weight is within a range of 500 to 10000.
[13]A lubricant for a magnetic recording medium, including the fluorine-containing ether compound according to any one of [1] to [12].
[14]A magnetic recording medium including at least in order, on a substrate, a magnetic layer, a protective layer, and a lubricating layer,
in which the lubricating layer contains the fluorine-containing ether compound according to any one of [1] to [12].
[15] The magnetic recording medium according to [14], in which an average film thickness of the lubricating layer is 0.5 nm to 2.0 nm.

### Advantageous Effects of Invention

The fluorine-containing ether compound of the present invention is a compound represented by Formula (1) and is suitable as a material for a lubricant for a magnetic recording medium.

The lubricant for a magnetic recording medium of the present invention contains the fluorine-containing ether compound of the present invention, and thus has good chemical substance resistance and can form a lubricating layer having a high pickup suppression effect.

The magnetic recording medium of the present invention has a lubricating layer containing the fluorine-containing ether compound of the present invention. Therefore, the magnetic recording medium of the present invention has good chemical substance resistance, has a high pickup suppression effect, and has excellent reliability and durability. In addition, since the magnetic recording medium of the present invention has a lubricating layer that has good chemical substance resistance and can suppress pickup, the thickness of the lubricating layer can be reduced, and the floating height of a magnetic head can be reduced.

### BRIEF DESCRIPTION OF DRAWINGS

FIG. 1 is a schematic cross-sectional view showing one embodiment of a magnetic recording medium of the present invention.

### DESCRIPTION OF EMBODIMENTS

In order to achieve the above objects, the present inventors have conducted intensive studies as described below.

In the related art, as a material for a lubricant for a magnetic recording medium (hereinafter, may be abbreviated as “lubricant”) to be applied to a surface of a protective layer, a fluorine-containing ether compound having a polar group such as a hydroxy group is preferably used. The polar group contained in the fluorine-containing ether compound is bonded to an active point on the protective layer to improve the adhesion of a lubricating layer to the protective layer. In the fluorine-containing ether compound of the related art, the polar group is disposed at a terminal in a chain-like structure. In addition, in a case where the fluorine-containing ether compound has a plurality of perfluoropolyether chains, the polar group is also disposed between adjacent perfluoropolyether chains.

However, in a case where a thin lubricating layer is formed on a protective layer using a lubricant in the related art, it is difficult to realize a lubricating layer having good chemical substance resistance and a high pickup suppression effect.

The reason for this is that, in the fluorine-containing ether compound contained in the lubricating layer, there is a polar group that is not adsorbed to a large number of active points present on the protective layer.

In a case where there is a polar group that is not adsorbed to the active points on the protective layer in the fluorine-containing ether compound contained in the lubricating layer, the state of the lubricant in the lubricating layer becomes bulky, and the coating state of the lubricating layer with respect to the protective layer becomes non-uniform. In addition, the surface energy of all fluorine-containing ether compound molecules increases, and a chemical substance is likely to adhere to the lubricating layer including the fluorine-containing ether compound. From these, in a case where there are many polar groups that are not adsorbed to the active points on the protective layer in the fluorine-containing ether compound contained in the lubricating layer, the chemical substance resistance and pickup suppression effect of the lubricating layer are likely to become insufficient.

Therefore, the present inventors focused on the bonding between the polar group contained in the fluorine-containing ether compound and the active point on the protective layer, and conducted intensive studies in order to realize a fluorine-containing ether compound in which a polar group that does not participate in the bonding with the active point on the protective layer is less likely to be generated. As a result, the present inventors have found that, among the polar groups included in the fluorine-containing ether compound, a secondary hydroxy group included in a divalent linking group disposed between adjacent perfluoropolyether chains is particularly less likely to participate in the bonding with the active point on the protective layer. That is, fluorine-containing ether compounds having a divalent linking group containing a secondary hydroxy group between adjacent perfluoropolyether chains still have room for improvement from the viewpoint of the adsorptivity to the protective layer. Therefore, the present inventors further conducted studies to develop a fluorine-containing ether compound having a divalent linking group capable of further improving the bonding with a protective layer between adjacent perfluoropolyether chains.

Specifically, the present inventors converted the secondary hydroxy group included in the divalent linking group disposed between adjacent perfluoropolyether chains in the fluorine-containing ether compound into a primary hydroxy group having high mobility and adsorptivity, and set the number of the primary hydroxy groups included in the divalent linking group to two so that the bonding point with the protective layer can be sufficiently secured. In addition, a lubricating layer was formed using this fluorine-containing ether compound. As a result, it was found that a lubricating layer having good chemical substance resistance and a high pickup suppression effect can be obtained.

Furthermore, the present inventors have conducted intensive studies and found that a fluorine-containing ether compound having two or three perfluoropolyether chains, in which a specific divalent linking group including a tetrasubstituted carbon atom to which two organic groups having one primary hydroxy group are bonded is disposed between adjacent perfluoropolyether chains through methylene groups (—CH₂—), and a methylene group and a specific terminal group having at least one polar group are disposed in this order at each of both terminals of a skeleton including the perfluoropolyether chains. The specific divalent linking group in the fluorine-containing ether compound has two organic side chains branched from the tetrasubstituted carbon atom. Two organic side chains may be the same as or different from each other, and are organic groups which do not include a secondary hydroxy group and a tertiary hydroxy group, include one primary hydroxy group, and have 1 to 5 carbon atoms. The organic group may include an ether oxygen atom between the carbon atoms, and a bonding terminal to the tetrasubstituted carbon atom may be the ether oxygen atom.

In such a fluorine-containing ether compound, due to the reasons <1> to <5> described below, a polar group that does not participate in the bonding with the active point present on the protective layer is less likely to be generated. Therefore, in the fluorine-containing ether compound, both end parts of two or three perfluoropolyether chains are each caused to closely adhere to the protective layer by two primary hydroxy groups included in the divalent linking group (or two primary hydroxy groups included in the divalent linking group and the polar group in the terminal group). Therefore, the fluorine-containing ether compound of the present embodiment is less likely to become bulky when having been applied onto the protective layer, easily wets and spreads on the protective layer, and makes it easy for a lubricating layer having a uniform coating state to be obtained. As a result, the fluorine-containing ether compound has good adhesion to the protective layer, has a high coating rate, and is less likely to incorporate a contaminant. Therefore, it is presumed that the fluorine-containing ether compound can form a lubricating layer having excellent chemical substance resistance and a good pickup suppression effect.
<1> In the fluorine-containing ether compound, the above-described divalent linking group has two organic side chains branched from the tetrasubstituted carbon atom. Each of two organic side chains is an organic group which has one primary hydroxy group and has 1 to 5 carbon atoms bonded to the tetrasubstituted carbon atom. Therefore, each of two primary hydroxy groups included in the above-described divalent linking group has an appropriate distance from the tetrasubstituted carbon atom in the divalent linking group. From this, both of two primary hydroxy groups included in the above-described divalent linking group are less likely to be inhibited from bonding to the active points on the protective layer by a bulky portion in the fluorine-containing ether compound, such as a perfluoropolyether chain in proximity or the tetrasubstituted carbon atom in the divalent linking group.
<2> Two primary hydroxy groups in the above-described divalent linking group are each disposed in the organic group having 1 to 5 carbon atoms bonded to the tetrasubstituted carbon atom. Therefore, the distance between two primary hydroxy groups included in the divalent linking group is appropriate. Therefore, aggregation between the primary hydroxy groups due to the distance between two primary hydroxy groups being too close is less likely to occur. In addition, since the distance between two primary hydroxy groups is not far, in a case where one of the primary hydroxy groups included in the above-described divalent linking group is bonded to the protective layer, the distance between the other primary hydroxy group and the protective layer is also close. As a result, the other primary hydroxy group can have an orientation that is likely to induce adsorption to the protective layer. Therefore, two primary hydroxy groups included in the divalent linking group do not inhibit each other from bonding to the active point on the protective layer, and two primary hydroxy groups are likely to bond to the active points on the protective layer at the same time.
<3> Two organic side chains are organic groups which do not include a secondary hydroxy group and a tertiary hydroxy group, include one primary hydroxy group, and have 1 to 5 carbon atoms bonded to the tetrasubstituted carbon atom. Therefore, the vicinity of the primary hydroxy group disposed in each organic group is three-dimensionally empty. In addition, the primary hydroxy group generally has a high degree of freedom and can move freely as compared with the secondary hydroxy group. Therefore, two primary hydroxy groups included in the above-described divalent linking group can each voluntarily move to the active points on the protective layer. Therefore, both of two primary hydroxy groups included in the above-described divalent linking group can easily form a bond with the active point on the protective layer.
<4> In a case where the fluorine-containing ether compound has three perfluoropolyether chains, the number of the above-described divalent linking groups present in the molecule is two. In this case, a perfluoropolyether chain is disposed between two adjacent divalent linking groups. Therefore, the distance between two primary hydroxy groups included in two adjacent divalent linking groups does not become too close. Therefore, both of two primary hydroxy groups included in the above-described two divalent linking groups are less likely to be inhibited from bonding to the active points on the protective layer by the primary hydroxy groups included in the adjacent divalent linking groups. In addition, aggregation with the primary hydroxy groups included in two adjacent divalent linking groups is also less likely to occur.
<5> In the fluorine-containing ether compound, a perfluoropolyether chain is disposed between the divalent linking group and each of both terminal groups. Therefore, the distance between two primary hydroxy groups included in the above-described divalent linking group and the polar group included in each terminal group does not become too close. As a result, two primary hydroxy groups included in the above-described divalent linking group and the polar group included in each terminal group are also less likely to be inhibited from bonding to the active points on the protective layer by the adjacent polar groups. In addition, since the distance between the above-described divalent linking group and each terminal group is appropriate, the primary hydroxy group included in the above-described divalent linking group and the polar group in each terminal group are less likely to aggregate. Therefore, two primary hydroxy groups included in the above-described divalent linking group and the polar group included in each terminal group are also likely to bond to the active points on the protective layer.

Moreover, the fluorine-containing ether compound has two or three perfluoropolyether chains. Two or three perfluoropolyether chains included in the lubricating layer coat the surface of the protective layer and impart chemical substance resistance to the lubricating layer due to the low surface energy.

Furthermore, the present inventors confirmed that a lubricating layer having good chemical substance resistance and a good pickup suppression effect can be formed using a lubricant containing the fluorine-containing ether compound, and completed the present invention.

Hereinafter, a fluorine-containing ether compound, a lubricant for a magnetic recording medium, and a magnetic recording medium of the present invention will be described in detail. The present invention is not limited only to embodiments shown below.

### [Fluorine-Containing Ether Compound]

A fluorine-containing ether compound of the present embodiment is represented by Formula (1).

R¹—CH₂—R²—(CH₂—OCH₂—CR³R⁴—CH₂O—CH₂—R²)_{z}—CH₂—R⁵ (1)

(In Formula (1), z is 1 or 2. R² is a perfluoropolyether chain. (z+1) R²'s may be the same in part or in whole, or may be different from each other. R¹ and R⁵ may be the same as or different from each other and are terminal groups having 1 to 50 carbon atoms and having at least one polar group. R³ and R⁴ may be the same as or different from each other, and are organic groups which do not include a secondary hydroxy group and a tertiary hydroxy group, include one primary hydroxy group, and have 1 to 5 carbon atoms bonded to a tetrasubstituted carbon atom. The organic group may include an ether oxygen atom between the carbon atoms, and a bonding terminal to the tetrasubstituted carbon atom may be the ether oxygen atom. In a case where z is 2, two —CR³R⁴-'s may be the same as or different from each other.)

In the fluorine-containing ether compound represented by Formula (1), z is 1 or 2. Since z is 2 or less, in the fluorine-containing ether compound represented by Formula (1), the molecule does not become too large. Therefore, a fluorine-containing ether compound, which can freely move on the protective layer, easily wets and spreads on the protective layer, and makes it easy for a lubricating layer having a uniform film thickness to be obtained, is obtained. In addition, since z is 1 or more, a fluorine-containing ether compound that can form a lubricating layer having good adhesion to the protective layer is obtained, as compared with a case where z is 0.

(Divalent linking group represented by —OCH₂—CR³R⁴—CH₂O—)

The divalent linking group represented by —OCH₂—CR³R⁴—CH₂O has two organic side chains (R³ and R⁴) branched from the tetrasubstituted carbon atom. Two organic groups represented by R³ and R⁴ are each an organic group which does not include a secondary hydroxy group and a tertiary hydroxy group, includes one primary hydroxy group, and has 1 to 5 carbon atoms bonded to the tetrasubstituted carbon atom. The organic group may include an ether oxygen atom between the carbon atoms, and a bonding terminal to the tetrasubstituted carbon atom may be the ether oxygen atom.

The organic groups represented by R³ and R⁴ in the fluorine-containing ether compound represented by Formula (1) may be the same as or different from each other. In a case where the organic groups represented by R³ and R⁴ are the same as each other, the adsorptivity and the mobility with respect to the protective layer of two primary hydroxy groups included in the divalent linking group positioned between the perfluoropolyether chains represented by R² (hereinafter, may be referred to as “PFPE chains”) are the same as each other, and there is no significant difference in the adsorption process of two primary hydroxy groups to the protective layer. Therefore, it is possible to form a lubricating layer having better adhesion to the protective layer, which is preferable.

In a case where z in Formula (1) is 2, two —CR³R⁴-'s in the fluorine-containing ether compound may be the same as or different from each other. In a case where two —CR³R⁴-'s are the same as each other, the compound is a fluorine-containing ether compound which is easy to produce, which is preferable. In a case where z in Formula (1) is 2, it is more preferable that all of R³'s and R⁴'s in two —CR³R⁴-'s are the same as each other.

In the lubricating layer containing the fluorine-containing ether compound represented by Formula (1), the PFPE chain represented by R² contained in the fluorine-containing ether compound does not have an electrostatic interaction with the protective layer. Therefore, the PFPE chain included in the fluorine-containing ether compound in the lubricating layer is present at a position separated from the protective layer by 3 A or more. On the other hand, the organic groups represented by R³ and R⁴ in the fluorine-containing ether compound each include one primary hydroxy group, which is a polar group. The polar group contained in the fluorine-containing ether compound in the lubricating layer can have an electrostatic interaction with the protective layer. However, in order for the polar group contained in the fluorine-containing ether compound in the lubricating layer to have an electrostatic interaction with the protective layer, it is necessary to bring the distance between the polar group and the protective layer close to about 2 A.

Therefore, in a case where the distance between two primary hydroxy groups included in the divalent linking group positioned between two or three PFPE chains contained in the fluorine-containing ether compound and the PFPE chain is too short, it becomes difficult for two primary hydroxy groups included in the divalent linking group to come close to the protective layer due to the PFPE chain present at the position separated from the protective layer by 3 A or more. That is, two primary hydroxy groups included in the divalent linking group positioned between the PFPE chains are inhibited from having an electrostatic interaction with the protective layer. As a result, it becomes difficult to obtain an electrostatic interaction between two hydroxy groups included in the divalent linking group and the protective layer, and the adhesion between the lubricating layer and the protective layer becomes insufficient. As a result, the chemical substance resistance of a magnetic recording medium provided with the lubricating layer deteriorates, and the fluorine-containing ether compound is likely to adhere to the magnetic head.

Specifically, for example, in a case where the divalent linking group positioned between two or three PFPE chains in the fluorine-containing ether compound represented by Formula (1) includes a secondary hydroxy group (for example, in a case where —CH(OH)— is disposed instead of —CR³R⁴— in Formula (1) or in a case where the divalent linking group is —OCH₂—CH(OH)—CH₂O—CH₂—CR³R⁴—CH₂—OCH₂—CH(OH)—CH₂O—), the distance between the hydroxy group included in the divalent linking group and the PFPE chain is likely to become close as compared with the fluorine-containing ether compound represented by Formula (1). Moreover, since the carbon atom to which the hydroxy group is bonded has two substituents, the degree of freedom of movement in the molecule of the secondary hydroxy group is low as compared with the primary hydroxy group. Therefore, the secondary hydroxy group included in the divalent linking group is likely to be inhibited from having an electrostatic interaction with the protective layer. As a result, in a case where the fluorine-containing ether compound in the lubricating layer has a divalent linking group containing a secondary hydroxy group, the divalent linking group is less likely to sufficiently develop a function as an adsorption unit to the protective layer.

In addition, in a case where a plurality of hydroxy groups with different steroelectronic environments coexist in the divalent linking group positioned between the PFPE chains of the fluorine-containing ether compound, a hydroxy group with a high degree of freedom of movement is preferentially bonded to the protective layer and occupies the active point on the protective layer. Therefore, an active point to which a hydroxy group with low mobility can be bonded is lost, and a hydroxy group that is not adsorbed to the protective layer is likely to be generated.

Specifically, for example, in a case where a primary hydroxy group and a secondary hydroxy group with different steroelectronic environments coexist in the divalent linking group positioned between two or three PFPE chains of the fluorine-containing ether compound represented by Formula (1) (for example, in a case where —OCH₂CH(OH)CH₂OH is disposed instead of R³ and/or R⁴ in Formula (1) or in a case where the divalent linking group is —O—CH₂—CH(OH)—CH₂—O—CH₂—C(CH₂OH)₂—CH₂—O—CH₂—CH(OH)—CH₂—O—), the primary hydroxy group having higher mobility and higher adsorptivity than the secondary hydroxy group is preferentially bonded to the active point on the protective layer. Therefore, the active point on the protective layer to which the secondary hydroxy group can be bonded is lost, and the secondary hydroxy group is likely to be in a state of being liberated from the protective layer without being capable of electrostatically interacting with the protective layer. As a result, the surface energy of all of the fluorine-containing ether compound molecules increases, and a chemical substance is likely to adhere to the lubricating layer including the fluorine-containing ether compound.

In addition, even when the divalent linking group includes a primary hydroxy group, for example, in a case where one of the organic groups represented by R³ and R⁴ in the fluorine-containing ether compound represented by Formula (1) is a hydrogen atom, the function of the divalent linking group positioned between two or three PFPE chains as an adsorption unit to the protective layer cannot be sufficiently obtained.

The organic groups represented by R³ and R⁴ in the fluorine-containing ether compound represented by Formula (1) are organic groups having 1 to 5 carbon atoms, and are preferably organic groups having 1 to 3 carbon atoms. Since the numbers of carbon atoms in the organic groups represented by R³ and R⁴ are 1 or more, the distance between the tetrasubstituted carbon atom and the primary hydroxy group in the divalent linking group and the distance between two primary hydroxy groups in the divalent linking group become appropriate.

In addition, since the numbers of carbon atoms of the organic groups represented by R³ and R⁴ are 5 or less, the flexibility of the divalent linking group positioned between the PFPE chains is improved, the state of the fluorine-containing ether compound applied onto the protective layer is less likely to become bulky, and a lubricating layer having a uniform film thickness can be formed. In addition, since the numbers of carbon atoms of the organic groups represented by R³ and R⁴ are 5 or less, the mobility of two primary hydroxy groups included in the divalent linking group are not too high. Therefore, both of two primary hydroxy groups are less likely to have an orientation that is liberated from the protective layer. As a result, two primary hydroxy groups have an orientation liberated from the protective layer, whereby the surface energy of all of the fluorine-containing ether compound molecules increases, and it is possible to prevent a chemical substance from adhering to the lubricating layer containing the fluorine-containing ether compound. Furthermore, since the numbers of carbon atoms of the organic groups represented by R³ and R⁴ are 5 or less, an interaction between two primary hydroxy groups included in the divalent linking group and an interaction between the primary hydroxy group in the divalent linking group and the polar groups in the terminal groups represented by R¹ and R⁵ are less likely to occur. Therefore, two primary hydroxy groups included in the divalent linking group are less likely to be inhibited from bonding to the active point on the protective layer, and are likely to bond to the active point on the protective layer.

The organic group represented by R³ and R⁴ may include an ether oxygen atom between the carbon atoms, and a bonding terminal to the tetrasubstituted carbon atom may be an ether oxygen atom. In a case where the organic group represented by R³ and/or R⁴ includes an ether oxygen atom, the flexibility of R³ and/or R⁴ including an ether oxygen atom is improved, and the primary hydroxy group in R³ and/or R⁴ including an ether oxygen atom is more likely to closely adhere to the protective layer, which is preferable.

The number of the ether oxygen atoms which may be included in the organic groups represented by R³ and R⁴ may be one or may be plural, but is preferably only one since the fluorine-containing ether compound applied onto the protective layer is less likely to become bulky.

In a case where the organic groups represented by R³ and R⁴ do not include an ether oxygen atom, R³ and R⁴ are each independently preferably a group represented by Formula (2-1). In a case where the organic groups represented by R³ and R⁴ include an ether oxygen atom, R³ and R⁴ are each independently preferably a group represented by Formula (2-2) or Formula (2-3).

The group represented by Formula (2-2) has a linear alkylene chain having 1 to 5 carbon atoms to which a primary hydroxy group is bonded, and a bonding terminal to the tetrasubstituted carbon atom is an ether oxygen atom. In addition, the group represented by Formula (2-3) includes one ether oxygen atom between methylene groups forming a linear alkylene chain having 1 to 5 carbon atoms to which a primary hydroxy group is bonded.

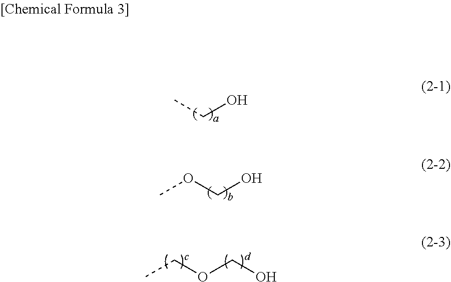

(In Formula (2-1), a is an integer of 1 to 5.)
(In Formula (2-2), b is an integer of 1 to 5.)
(In Formula (2-3), c and d are each independently an integer of 1 to 4, and c+d <5.)

In a case where R³ and R⁴ are each independently any group represented by any of Formulae (2-1) to (2-3), since a in Formula (2-1), b in Formula (2-2), and c and d in Formula (2-3) are each 1 or more, the primary hydroxy groups included in the organic groups represented by R³ and R⁴ can be at an appropriate distance from the tetrasubstituted carbon atom in the divalent linking group. Therefore, it becomes easy for the primary hydroxy groups in R³ and R⁴ to move freely without receiving any influences derived from the bulkiness of the tetrasubstituted carbon atom in the divalent linking group. As a result, the primary hydroxy groups in R³ and R⁴ are likely to closely adhere to the protective layer, and the lubricating layer containing the fluorine-containing ether compound represented by Formula (1) is less likely to float from the protective layer. In addition, since a, b, c, and d are each 1 or more, the distance between two primary hydroxy groups included in the divalent linking group becomes appropriate, and the primary hydroxy groups are less likely to aggregate.

Since a in Formula (2-1) and b in Formula (2-2) are 5 or less, c and d in Formula (2-3) are each independently 4 or less, and c+d is 5 or less, the mobility of the organic groups represented by R³ and R⁴ does not become excessively high, and the flexibility of the organic groups represented by R³ and R⁴ becomes appropriate. Therefore, the interaction between two primary hydroxy groups included in the divalent linking group and the interaction between the primary hydroxy group in the divalent linking group and the terminal group represented by R¹ or R⁵ are suppressed, and two primary hydroxy groups can be effectively bonded to the active points on the protective layer.

In order to easily obtain these effects, it is preferable that a in Formula (2-1) is 1 to 3. b in Formula (2-2) is preferably 1 to 3 and more preferably 1 or 2. In addition, c and d in Formula (2-3) are each independently preferably an integer of 1 to 3, and c and d are each more preferably 1 or 2. In addition, c+d in Formula (2-3) is preferably 2 to 4 and more preferably 2 or 3.

### (Perfluoropolyether Chain Represented by R²)

In the fluorine-containing ether compound represented by Formula (1), (z+1) R²'s are each independently a PFPE chain. In a case where a lubricant containing the fluorine-containing ether compound of the present embodiment is applied onto the protective layer to form a lubricating layer, the PFPE chain represented by R² coats the surface of the protective layer and imparts lubricity to the lubricating layer to reduce the frictional force between the magnetic head and the protective layer. The PFPE chain represented by R² is appropriately selected depending on the performance or the like required for the lubricant including the fluorine-containing ether compound.

In the fluorine-containing ether compound represented by Formula (1), (z+1) R²'s may be the same as each other in part or in whole, or may be different from each other. It is preferable that all of (z+1) R²'s are the same as each other. This is because the coating state of the fluorine-containing ether compound with respect to the protective layer becomes uniform, and the lubricating layer has better adhesion. Two or more R²'s among (z+1) R²'s being the same means that two or more R²'s having the same structure of the repeating unit of the PFPE chain are included among (z+1) R²'s. The same R²'s also includes R²'s having the same structure of the repeating unit but different average degrees of polymerization.

Examples of the PFPE chain represented by R² include chains composed of a polymer or copolymer of a perfluoroalkylene oxide. Examples of the perfluoroalkylene oxide include perfluoromethylene oxide, perfluoroethylene oxide, perfluoro-n-propylene oxide, perfluoroisopropylene oxide, and perfluorobutylene oxide.

It is preferable that (z+1) R²'s in Formula (1) are each independently, for example, a PFPE chain represented by Formula (3) derived from a polymer or copolymer of a perfluoroalkylene oxide.

—(CF₂)_{w1}—O—(CF₂O)₂—(CF₂CF₂O)_{w3}—(CF₂CF₂CF₂O)_{w4}—(CF₂CF₂CF₂CF₂O),s—(CF₂)_{w6}—(3)

(In Formula (3), w2, w3, w4, and w5 represent average degrees of polymerization and each independently represent 0 to 20. Here, there are no cases where all of w2, w3, w4, and w5 become 0 at the same time. w1 and w6 are average values representing the number of CF₂'s and each independently represent 1 to 3. An order of arrangement of (CF₂O), (CF₂CF₂O), (CF₂CF₂CF₂O), and (CF₂CF₂CF₂CF₂O), which are repeating units in Formula (3), is not particularly limited.

In Formula (3), w2, w3, w4, and w5 represent the average degrees of polymerization, each independently represent 0 to 20, and are preferably 0 to 15 and more preferably 0 to 10.

In Formula (3), w1 and w6 are the average values indicating the number of CF₂'s, and each independently represent 1 to 3. w1 and w6 are determined depending on the structure of the repeating unit disposed at the end part of the chain-like structure in the PFPE chain represented by Formula (3), and the like.

(CF₂O), (CF₂CF₂O), (CF₂CF₂CF₂O), and (CF₂CF₂CF₂CF₂O) in Formula (3) are repeating units. The order of arrangement of the repeating units in Formula (3) is not particularly limited. In addition, the number of the kinds of repeating units in Formula (3) is not particularly limited.

It is preferable that (z+1) R²'s in Formula (1) are each independently any one selected from PFPE chains represented by Formulae (3-1) to (3-4).

In a case where (z+1) R²'s are each independently any one selected from the PFPE chains represented by Formulae (3-1) to (3-4), a fluorine-containing ether compound from which a lubricating layer having good lubricity can be obtained is obtained. In addition, in a case where (z+1) R²'s are each independently any one selected from the PFPE chains represented by Formulae (3-1) to (3-4), the proportion of the number of oxygen atoms (the number of ether bonds (—O—)) in the number of carbon atoms in the PFPE chain is appropriate. Therefore, the fluorine-containing ether compound has appropriate hardness. Therefore, the fluorine-containing ether compound applied onto the protective layer is less likely to aggregate on the protective layer, and a lubricating layer having a thinner thickness can be formed with a sufficient coating rate. In addition, the fluorine-containing ether compound has appropriate flexibility, whereby a lubricating layer having better chemical substance resistance can be formed.

—CF₂—(OCF₂CF₂)ₙ—(OCF₂)ₒ—OCF₂— (3-1)
(In Formula (3-1), n and o represent average degrees of polymerization, n represents 1 to 20, and o represents 0 to 20.)

—CF₂CF₂—(OCF₂CF₂CF₂)ₚ—OCF₂CF₂— (3-2)
(In Formula (3-2), p represents an average degree of polymerization and represents 1 to 15.)

—CF₂CF₂CF₂—(OCF₂CF₂CF₂CF₂)_{q}—OCF₂CF₂CF₂— (3-3)
(In Formula (3-3), q represents an average degree of polymerization and represents 1 to 10.)

—(CF₂)_{w7}—O—(CF₂CF₂CF₂O)_{w8}—(CF₂CF₂O)_{w9}—(CF₂)_{w10}— (3-4)
(In Formula (3-4), w8 and w9 represent average degrees of polymerization and each independently represent 1 to 20. w7 and w10 are average values representing the number of CF₂'s and each independently representing 1 to 2.)

In Formula (3-1), the order of arrangement of (OCF₂CF₂) and (OCF₂), which are repeating units, is not particularly limited. In Formula (3-1), the number n of (OCF₂CF₂)'s and the number o of (OCF₂)'s may be the same as or different from each other. The PFPE chain represented by Formula (3-1) may be a polymer of (OCF₂CF₂). In addition, the PFPE chain represented by Formula (3-1) may be any one of a random copolymer, block copolymer, or alternating copolymer composed of (OCF₂CF₂) and (OCF₂).

In Formulae (3-1) to (3-3), since n indicating the average degree of polymerization is 1 to 20, o is 0 to 20, p is 1 to 15, and q is 1 to 10, a fluorine-containing ether compound from which a lubricating layer having good lubricity can be obtained is obtained. In addition, in Formulae (3-1) to (3-3), since n and o indicating the average degrees of polymerization are 20 or less, p is 15 or less, and q is 10 or less, the viscosity of the fluorine-containing ether compound does not become excessively high, and it is easy to apply a lubricant including this fluorine-containing ether compound, which is preferable. Since a fluorine-containing ether compound, which easily wets and spreads on the protective layer and makes it easy for a lubricating layer having a uniform film thickness to be obtained, is obtained, n, o, p, and q representing the average degrees of polymerization are preferably 1 to 10, more preferably 1.5 to 8, and still more preferably 2 to 7.

In Formula (3-4), the order of arrangement of (CF₂CF₂CF₂O) and (CF₂CF₂O), which are repeating units, is not particularly limited. In Formula (3-4), the number w8 of (CF₂CF₂CF₂O)'s and the number w9 of (CF₂CF₂O)'s may be the same as or different from each other. Formula (3-4) may a PFPE chain containing any one of a random copolymer, block copolymer, or alternating copolymer composed of monomer units (CF₂CF₂CF₂O) and (CF₂CF₂O).

In Formula (3-4), w8 and w9 representing the average degrees of polymerization are each independently 1 to 20, preferably 1 to 15, and more preferably 1 to 10. w7 and w10 in Formula (3-4) are the average values indicating the number of CF₂'s, and each independently represent 1 to 2. w7 and w10 are determined depending on the structure of the repeating unit disposed at the end part of the chain-like structure in the perfluoropolyether chain represented by Formula (3-4).

(Terminal groups represented by R¹ and R⁵) In the fluorine-containing ether compound represented by Formula (1), R¹ and R⁵ may be the same as or different from each other, and are terminal groups having 1 to 50 carbon atoms and having at least one polar group. The terminal groups represented by R¹ and R⁵ are each independently preferably organic groups having 1 to 20 carbon atoms, and more preferably organic groups having 2 to 12 carbon atoms. In a case where the number of carbon atoms is within the above-described range, the proportion of the number of carbon atoms in the number of the polar groups becomes appropriate, and a fluorine-containing ether compound having an appropriate molecular polarity.

In the terminal groups represented by R¹ and R⁵, it is preferable that an end part bonding to the adjacent methylene groups is an oxygen atom. In this case, R¹ and R⁵ are each bonded to the adjacent methylene groups through an ether bond, thereby forming a fluorine-containing ether compound having appropriate hardness. Therefore, the fluorine-containing ether compound applied onto the protective layer is less likely to aggregate on the protective layer, and a lubricating layer having a thinner thickness can be formed with a sufficient coating rate.

Examples of the polar group contained in the terminal group represented by R¹ and/or R⁵ include a hydroxy group (—OH), an amino group (—NR¹⁰R¹¹; R¹⁰ and R¹¹ are each independently a hydrogen atom or an organic group), a carboxy group (—COOH), a formyl group (—(C═O)H), a carbonyl group (—CO—), a sulfo group (—SO₃H), a cyano group (—CN), and a group having an amide bond (—NR⁶COR⁷ or —CONR⁸R⁹; R⁶, R⁷, R⁸, and R⁹ are each independently a hydrogen atom or an organic group). The “group having an amide bond” includes both a group bonding to a carbon atom constituting the amide bond (for example, a carboxamide group (—C(═O)NH₂)) and a group bonding to a nitrogen atom constituting the amide bond (for example, an acetamide group (—NHC(═O)CH₃)), as shown in the above formulae. In the group having an amide bond, R⁶ and R⁷ may be bonded to each other to form a ring, or R⁵ and R⁹ may be bonded to each other to form a ring. It is preferable that R⁶, R⁷, R⁸, and R⁹ in the group having an amide bond are each independently selected from the group consisting of a hydrogen atom, a methyl group, an ethyl group, a propyl group, and a butyl group.

In order to further improve the chemical substance resistance, at least one of the terminal groups represented by R¹ and R⁵ preferably has at least one hydroxy group as a polar group. As a result, a fluorine-containing ether compound that can form a lubricating layer having high adhesion to the protective layer and having more excellent chemical resistance and pickup suppression effect can be obtained. Since the fluorine-containing ether compound can form a lubricating layer having further improved adhesion to the protective layer, it is more preferable that R¹ and R⁵ each independently have at least one hydroxy group as a polar group.

Since the fluorine-containing ether compound can form a lubricating layer having further improved adhesion to the protective layer, the numbers of polar groups contained in the terminal groups represented by R¹ and R⁵ are preferably any one of 1 to 3 and more preferably 2 or 3. In the fluorine-containing ether compound represented by Formula (1), in order to improve the adhesion to the protective layer and to realize the thinning of the lubricating layer, the total number of the polar groups contained in R¹ and the polar groups contained in R⁵ is preferably 3 to 6. In a case where the total number is 3 or more, the lubricating layer containing the fluorine-containing ether compound has higher adhesion (adhesion) to the protective layer. In addition, in a case where the total number is 6 or less, in the magnetic recording medium having the lubricating layer containing the fluorine-containing ether compound, it is possible to prevent the occurrence of pickup in which the fluorine-containing ether compound adheres to the magnetic head as a foreign matter (smear) due to the excessively high polarity.

The number of the polar groups included in R¹ and the number of the polar groups included in R⁵ may be the same as or different from each other, and are preferably the same as each other. For example, it is preferable that R¹ and R⁵ each include two polar groups, or R¹ and R⁵ each include three polar groups. In this case, the lubricant containing the fluorine-containing ether compound closely adheres to the protective layer in a well-balanced manner. Therefore, it is easy to obtain a lubricating layer having a high coating rate and having more excellent chemical substance resistance and pickup suppression effect.

In a case where R¹ and/or R⁵ includes two or more polar groups, the kinds of the polar groups included in R¹ and/or R⁵ may be the same in part or in while or may be different in whole.

In a case where R¹ and/or R⁵ includes two or more polar groups, it is preferable that two or more polar groups are each bonded to different carbon atoms and one or more carbon atoms are included between carbon atoms to which the adjacent polar groups are bonded. In this case, the adjacent polar groups are bonded to each other at an appropriate interatomic distance, as compared with a case where the carbon atoms to which the adjacent polar groups are bonded are directly bonded to each other. Therefore, a plurality of the polar groups contained in R¹ and/or R⁵ are oriented such that all of the polar groups can closely adhere to the protective layer. As a result, the plurality of polar groups contained in R¹ and/or R⁵ are less likely to aggregate and can easily form a bond with the active point on the protective layer.

The terminal group represented by R¹ and/or R⁵ may be an organic group having at least one polar group and further having a carbon-carbon unsaturated bond site. In a case where the terminal group represented by R¹ and/or R⁵ has a carbon-carbon unsaturated bond site, the terminal group is preferably an organic group having at least one selected from the group consisting of an aromatic hydrocarbon group, an unsaturated heterocyclic group, an alkenyl group, and an alkynyl group.

Examples of the aromatic hydrocarbon group include a phenyl group, a methoxyphenyl group, a fluorophenyl group, a naphthyl group, and a methoxynaphthyl group. As described above, the aromatic hydrocarbon group also includes a group in which a substituent (a methoxy group, a fluoro group, or the like) is bonded to an aromatic hydrocarbon.

Examples of the unsaturated heterocyclic group include a pyrrolyl group, a pyrazolyl group, a methylpyrazolyl group, an imidazolyl group, a furyl group, a furfuryl group, an oxazolyl group, an isoxazolyl group, a thienyl group, a thiazolyl group, an isothiazolyl group, a pyridyl group, a pyrimidinyl group, a pyridazinyl group, a pyrazinyl group, an indolinyl group, a benzofuranyl group, a benzothienyl group, a benzimidazolyl group, a benzoxazolyl group, a benzothiazolyl group, a benzopyrazolyl group, a benzisoxazolyl group, a benzisothiazolyl group, a quinolyl group, an isoquinolyl group, a quinazolinyl group, a quinoxalinyl group, a phthalazinyl group, and a cinnolinyl group. The unsaturated heterocyclic group also includes a group in which a substituent (a methyl group or the like) is bonded to an unsaturated heterocyclic ring, as described above.

Examples of the alkenyl group include a vinyl group, an allyl group, a butenyl group, a pentenyl group, and a hexenyl group.

Examples of the alkynyl group include a 1-propynyl group, a propargyl group, a butynyl group, a pentynyl group, and a hexynyl group.

In a case where the terminal group represented by R¹ and/or R⁵ has a carbon-carbon unsaturated bond site, the lubricating layer containing the fluorine-containing ether compound is preferable since it has excellent adhesion to the protective layer and can be made thinner. The reason will be described below.

A large number of active points present on the protective layer include a locally charged portion and a portion where the charge distribution is spread. The hydroxy group contained in R³ and R⁴ in Formula (1) (and the hydroxy group in a case where R¹ and/or R⁵ has a hydroxy group) and the carbon-carbon unsaturated bond site contained in the terminal group represented by R¹ and/or R⁵ are adsorbed to different sites on the protective layer.

Specifically, the hydroxy groups contained in R³ and R⁴ in Formula (1) (and the hydroxy group in a case where R¹ and/or R⁵ has a hydroxy group) exhibit an adsorption ability by a hydrogen atom interacting with the locally charged portion on the protective layer through a hydrogen bond. On the other hand, since the carbon-carbon unsaturated bond site included in the terminal group represented by R¹ and/or R⁵ has a non-local charge, the carbon-carbon unsaturated bond site exhibits an adsorption ability by an interaction with the portion where the charge distribution is spread on the protective layer.

Therefore, the hydroxy groups contained in R³ and R⁴ in Formula (1) (and the hydroxy group in a case where R¹ and/or R⁵ has a hydroxy group) and the carbon-carbon unsaturated bond site contained in the terminal group represented by R¹ and/or R⁵ can each independently interact with the active points on the protective layer. As a result, the lubricating layer containing the fluorine-containing ether compound in which the terminal group represented by R¹ and/or R⁵ has the carbon-carbon unsaturated bond site is more excellent in adhesion to the protective layer, exhibits good chemical substance resistance even in a case where the thickness is small, and has a high pickup suppression effect.

It is preferable that the terminal groups represented by R¹ and R⁵ are each a terminal group containing two or three polar groups, in which at least one of the polar groups is a hydroxy group. Specifically, the terminal groups represented by R¹ and R⁵ are each independently preferably any terminal group represented by any of Formulae (4-1) to (4-3).

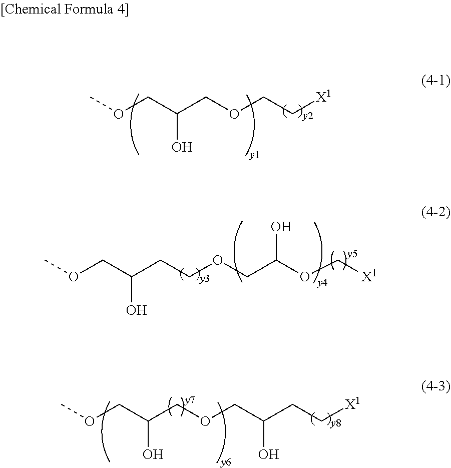

(In Formula (4-1), y1 is 1 or 2, and y2 is an integer of 0 to 3. X¹ is an aromatic hydrocarbon group, an unsaturated heterocyclic group, an alkenyl group, an alkynyl group, or a polar group. In a case where y1 is 1, X¹ is a polar group. In a case where X¹ is an aromatic hydrocarbon group or an unsaturated heterocyclic group, an atom constituting a ring structure in X¹ is bonded to a methylene group adjacent to X¹. In a case where X¹ is an alkenyl group or an alkynyl group, a carbon atom constituting an unsaturated bond in X¹ is bonded to a methylene group adjacent to X¹).

(In Formula (4-2), y3 is an integer of 1 to 3, y4 is 0 or 1, and y5 is an integer of 0 to 3. X¹ is an aromatic hydrocarbon group, an unsaturated heterocyclic group, an alkenyl group, an alkynyl group, or a polar group. In a case where y4 is 0, X¹ is a polar group. In a case where X¹ is an aromatic hydrocarbon group or an unsaturated heterocyclic group, an atom constituting a ring structure in X¹ is bonded to a methylene group adjacent to X¹. In a case where X¹ is an alkenyl group or an alkynyl group, a carbon atom constituting an unsaturated bond in X¹ is bonded to a methylene group adjacent to X¹).

(In Formula (4-3), y6 is 0 or 1, y7 is an integer of 1 to 3, and y8 is an integer of 1 to 3. X¹ is an aromatic hydrocarbon group, an unsaturated heterocyclic group, an alkenyl group, an alkynyl group, or a polar group. In a case where y6 is 0, X¹ is a polar group. In a case where X¹ is an aromatic hydrocarbon group or an unsaturated heterocyclic group, an atom constituting a ring structure in X¹ is bonded to a methylene group adjacent to X¹. In a case where X¹ is an alkenyl group or an alkynyl group, a carbon atom constituting an unsaturated bond in X¹ is bonded to a methylene group adjacent to X¹).

In Formulae (4-1) to (4-3), in a case where X¹ is an aromatic hydrocarbon group, an atom constituting a ring structure in X¹ is bonded to a methylene group adjacent to X¹. In a case where X¹ is an aromatic hydrocarbon group, the aromatic hydrocarbon group exemplified above can be used as X¹.

In Formulae (4-1) to (4-3), in a case where X¹ is an unsaturated heterocyclic group, an atom constituting a ring structure in X¹ is bonded to a methylene group adjacent to X¹. In a case where X¹ is an unsaturated heterocyclic group, the unsaturated heterocyclic group exemplified above can be used as X¹.

In Formulae (4-1) to (4-3), in a case where X¹ is an alkenyl group, a carbon atom constituting an unsaturated bond in X¹ is bonded to a methylene group adjacent to X¹. In a case where X¹ is an alkenyl group, examples of X¹ include —CH═CH₂, —CH═CHR¹² (R¹² is an organic group), —CR¹³═CHR¹⁴ (R¹³ and R¹⁴ are organic groups), and —CR¹⁵═CR¹⁶R¹⁷ (R¹⁵, R¹⁶, and R¹⁷ are organic groups). The organic groups represented by R¹² to R¹⁷ are each preferably a hydrocarbon group having 1 to 3 carbon atoms. In a case where X¹ in Formulae (4-1) to (4-3) is an alkenyl group, X¹ is preferably —CH═CH₂. —CH═CH₂ has an appropriate bulkiness. Therefore, the lubricating layer containing the fluorine-containing ether compound having a terminal group in which X¹ is —CH═CH₂ is likely to be in a state where the bulkiness of the fluorine-containing ether compound on the protective layer is low and has good pickup characteristics.

In Formulae (4-1) to (4-3), in a case where X¹ is an alkynyl group, a carbon atom constituting an unsaturated bond in X¹ is bonded to a methylene group adjacent to X¹. In a case where X¹ is an alkynyl group, examples of X¹ include —C≡CH and —C≡C¹⁸ (R¹⁸ is an organic group). The organic group represented by R¹⁸ is preferably a hydrocarbon group having 1 to 3 carbon atoms. In a case where X¹ in Formulae (4-1) to (4-3) is an alkynyl group, X¹ is preferably —C═CH since X¹ becomes a terminal group having an appropriate bulkiness.

In Formulae (4-1) to (4-3), in a case where X¹ is a polar group, the polar group exemplified above can be used as X¹. Among these polar groups, X¹ is preferably a hydroxy group, a group having an amide bond, or a cyano group. In a case where X¹ is a hydroxy group, a group having an amide bond, or a cyano group, a more suitable interaction occurs between the lubricating layer and the protective layer in a case where the lubricating layer is formed on the protective layer using a lubricant containing X¹.

In a case where X¹ in Formulae (4-1) to (4-3) is a polar group, the lubricating layer containing the fluorine-containing ether compound is more excellent in adhesion to the protective layer and can be made thinner, which is preferable. The reason will be described below.

In Formulae (4-1) to (4-3), the secondary hydroxy group in Formulae (4-1) to (4-3) and X¹ are bonded to each other through a divalent organic group which may include an ether bond. Therefore, even in a case where X¹ is a polar group, the distance between the secondary hydroxy group in Formulae (4-1) to (4-3) and the polar group represented by X¹ becomes appropriate. As a result, the secondary hydroxy group in Formulae (4-1) to (4-3) and the polar group represented by X¹ are less likely to be inhibited from bonding to the active point on the protective layer by another polar group. In addition, the secondary hydroxy group in Formulae (4-1) to (4-3) and the polar group represented by X¹ are less likely to aggregate.

Therefore, the secondary hydroxy group in each of Formulae (4-1) to (4-3) and the polar group represented by X¹ can be each independently adsorbed to the active point on the protective layer. As a result, the lubricating layer containing the fluorine-containing ether compound having a terminal group in which X¹ in Formulae (4-1) to (4-3) is a polar group is more excellent in adhesion to the protective layer, exhibits good chemical substance resistance even in a case where the thickness is small, and has a high pickup suppression effect.

Among the above, X¹ in Formulae (4-1) to (4-3) is preferably any one of a hydroxy group, a group having an amide bond, a cyano group, or —CH═CH₂. This is because a fluorine-containing ether compound capable of forming a lubricating layer having a higher coating rate and more excellent chemical substance resistance and pickup suppression effect can be obtained.

In the terminal group represented by Formula (4-1), y1 is 1 or 2, and y2 is an integer of 0 to 3. In a case where y1 is 1, X¹ is a polar group, and Formula (4-1) has two polar groups. In this case, since Formula (4-1) has two polar groups, a lubricating layer having good adhesion to the protective layer can be formed. In a case where y1 is 2, X¹ may be any of an aromatic hydrocarbon group, an unsaturated heterocyclic group, an alkenyl group, an alkynyl group, or a polar group. Even in a case where y1 is 2 and X¹ is any one of an aromatic hydrocarbon group, an unsaturated heterocyclic group, an alkenyl group, or an alkynyl group, Formula (4-1) has two polar groups. Therefore, a lubricating layer having good adhesion to the protective layer can be formed. In addition, since X¹ is an aromatic hydrocarbon group, an unsaturated heterocyclic group, an alkenyl group, or an alkynyl group, a lubricating layer having excellent chemical substance resistance and pickup suppression effect can be formed by the 7n-7n interaction between the carbon-carbon unsaturated bond site of X¹ and the protective layer without impairing the adhesion of two hydroxy groups included in Formula (4-1) to the protective layer. In addition, in a case where y1 is 2 and X¹ is a polar group, Formula (4-1) has three polar groups. Therefore, it is possible to form a lubricating layer that exhibits more excellent adhesion to the protective layer.

In the terminal group represented by Formula (4-1), y2 is an integer of 0 to 3. In the terminal group represented by Formula (4-1), even in a case where X¹ in Formula (4-1) is a polar group, the distance between X¹ and the secondary hydroxy group in Formula (4-1) is not too close, and thus the polar group in Formula (4-1) is less likely to aggregate. In a case where X¹ in Formula (4-1) is a polar group, the distance between X¹ and the secondary hydroxy group in Formula (4-1) becomes more appropriate, and thus y2 is preferably 1 or more. In the terminal group represented by Formula (4-1), since y2 is 3 or less, the mobility of X¹ in Formula (4-1) does not become excessively high, and each polar group contained in the terminal group can sufficiently closely adhere to the protective layer. y2 is more preferably 2 or less.

In the terminal group represented by Formula (4-2), y3 is an integer of 1 to 3. In a case where y4 is 0, X¹ is a polar group. Since y3 is an integer of 1 or more, in a case where y4 is 0, the distance between X¹ and the secondary hydroxy group in Formula (4-2) becomes appropriate, and even in a case where X¹ is a polar group, the polar group in Formula (4-2) is less likely to aggregate. In addition, since y3 is an integer of 1 or more, in a case where y4 is 1, the distance between the secondary hydroxy groups in Formula (4-2) does not become too close, and the secondary hydroxy groups in Formula (4-2) are less likely to aggregate. In the terminal group represented by Formula (4-2), since y3 is 3 or less, the mobility of the terminal group represented by Formula (4-2) does not become excessively high, and each polar group contained in the terminal group can sufficiently closely adhere to the protective layer. y3 is preferably 2 or less.

In the terminal group represented by Formula (4-2), y4 is 0 or 1. In a case where y4 is 0, X¹ is a polar group, and Formula (4-2) has two polar groups. In this case, since Formula (4-2) has two polar groups, a lubricating layer having good adhesion to the protective layer can be formed. In a case where y4 is 1, X¹ may be any of an aromatic hydrocarbon group, an unsaturated heterocyclic group, an alkenyl group, an alkynyl group, or a polar group. Even in a case where y4 is 1 and X¹ is any one of an aromatic hydrocarbon group, an unsaturated heterocyclic group, an alkenyl group, or an alkynyl group, Formula (4-2) has two polar groups. Therefore, a lubricating layer having good adhesion to the protective layer can be formed. In addition, since X¹ is an aromatic hydrocarbon group, an unsaturated heterocyclic group, an alkenyl group, or an alkynyl group, a lubricating layer having excellent chemical substance resistance and pickup suppression effect can be formed by the 7t-n interaction between the carbon-carbon unsaturated bond site of X¹ and the protective layer without impairing the adhesion of two hydroxy groups included in Formula (4-2) to the protective layer. In addition, in a case where y4 is 1 and X¹ is a polar group, Formula (4-2) has three polar groups. Therefore, it is possible to form a lubricating layer that exhibits excellent adhesion to the protective layer.

In the terminal group represented by Formula (4-2), y5 is an integer of 0 to 3. In the terminal group represented by Formula (4-2), even in a case where X¹ in Formula (4-2) is a polar group, the distance between X¹ and the secondary hydroxy group in Formula (4-2) is not too close, and thus the polar group in Formula (4-2) is less likely to aggregate. In a case where X¹ in Formula (4-2) is a polar group, the distance between X¹ and the secondary hydroxy group in Formula (4-2) becomes more appropriate, and thus y5 is preferably 1 or more. In addition, in a case where y4 is 0, the distance between X¹, which is a polar group, and the secondary hydroxy group in Formula (4-2) becomes appropriate due to y3 methylene groups even in a case where y5 is 0. In a case where y4 is 0, the distance between X¹, which is a polar group, and the secondary hydroxy group in Formula (4-2) becomes more appropriate due to y3+y5 methylene groups even in a case where y5 is 1. In the terminal group represented by Formula (4-2), since y5 is 3 or less, the mobility of X¹ in Formula (4-2) does not become excessively high, and each polar group contained in the terminal group can sufficiently closely adhere to the protective layer. y5 is preferably 2 or less.

In the terminal group represented by Formula (4-3), y6 is 0 or 1. In a case where y6 is 0, X¹ is a polar group, and Formula (4-3) has two polar groups. In this case, since Formula (4-3) has two polar groups, a lubricating layer having good adhesion to the protective layer can be formed. In a case where y6 is 1, X¹ may be any of an aromatic hydrocarbon group, an unsaturated heterocyclic group, an alkenyl group, an alkynyl group, or a polar group. Even in a case where y6 is 1 and X¹ is an aromatic hydrocarbon group, an unsaturated heterocyclic group, an alkenyl group, or an alkynyl group, Formula (4-3) has two polar groups. Therefore, a lubricating layer having good adhesion to the protective layer can be formed. In addition, since X¹ is an aromatic hydrocarbon group, an unsaturated heterocyclic group, an alkenyl group, or an alkynyl group, a lubricating layer having excellent chemical substance resistance and pickup suppression effect can be formed by the 7r-7r interaction between the carbon-carbon unsaturated bond site of X¹ and the protective layer without impairing the adhesion of two hydroxy groups contained in Formula (4-3) to the protective layer. In addition, in a case where y6 is 1 and X¹ is a polar group, Formula (4-3) has three polar groups. Therefore, it is possible to form a lubricating layer that exhibits excellent adhesion to the protective layer.

In the terminal group represented by Formula (4-3), v7 is an integer of 1 to 3. Since y7 is 1 or more, in a case where y6 is 1, the distance between the secondary hydroxy groups in Formula (4-3) does not become too close. Therefore, the secondary hydroxy groups in Formula (4-3) are less likely to aggregate. In the terminal group represented by Formula (4-3), since y7 is 3 or less, the mobility of the terminal group represented by Formula (4-3) does not become excessively high, and each polar group contained in the terminal group can sufficiently closely adhere to the protective layer. y7 is preferably 2 or less.

In the terminal group represented by Formula (4-3), y8 is an integer of 1 to 3. In the terminal group represented by Formula (4-3), since y8 is 1 or more, even in a case where X¹ in Formula (4-3) is a polar group, the distance between X¹ and the secondary hydroxy group in Formula (4-3) does not become too close. Therefore, the polar group in Formula (4-3) is less likely to aggregate. In a case where X¹ in Formula (4-3) is a polar group, the distance between X¹ and the secondary hydroxy group in Formula (4-3) becomes more appropriate, and thus y8 is preferably 2 or more. In the terminal group represented by Formula (4-3), since y8 is 3 or less, the mobility of X¹ in Formula (4-3) does not become excessively high, and each polar group contained in the terminal group can sufficiently closely adhere to the protective layer.

In the fluorine-containing ether compound represented by Formula (1), R¹ and R⁵ may be the same as or different from each other. In a case where R¹ and R⁵ are the same as each other, the coating state of the fluorine-containing ether compound with respect to the protective layer is more uniform, and a lubricating layer having better adhesion can be formed.

In the fluorine-containing ether compound represented by Formula (1), the kinds of the terminal groups represented by R¹ and R⁵ can be appropriately selected depending on the performance required for the lubricant containing the fluorine-containing ether compound and the like.

In the fluorine-containing ether compound represented by Formula (1), it is preferable that z is 1, R¹ and R⁵ are the same as each other, and two R²'s are the same as each other. This is because the fluorine-containing ether compound becomes easy to synthesize.

In the fluorine-containing ether compound represented by Formula (1), it is preferable that z is 2, R¹ and R⁵ are the same as each other, and three R²'s are the same as one another. This is because the fluorine-containing ether compound becomes easy to synthesize. Furthermore, in a case where z is 2, it is preferable that two —CR³R⁴-'s are the same as each other. This is because the fluorine-containing ether compound becomes easier to synthesize.

Specifically, the fluorine-containing ether compound represented by (1) is preferably any of compounds represented by Formulae (1A) to (1T) and (2A) to (2T) below. In a case where the compound represented by Formula (1) is a compound represented by any of Formulae (1A) to (1T) and (2A) to (2T) below, a raw material is easily procured, and moreover, a lubricating layer having excellent adhesion even in a case where the thickness is small and having more excellent chemical substance resistance and more excellent chemical pickup suppression effect can be formed.

In all of the compounds represented by Formulae (1A) to (1T) and (2A) to (2T), (z+1) the PFPE chains represented by R² in Formula (1) are all the same as each other.

In the compounds represented by Formulae (TA) to (1T) and (2A) to (2T), Rf₁ and Rf₂ representing the PFPE chain are each the following structure. That is, in the compounds represented by Formulae (1A) to (1F), (1J), (1K), (1N), (1P) to (1T), (2A) to (2F), (2J), (2K), (2N), and (2P) to (2T), Rf₂ is the PFPE chain represented by Formula (3-2). In the compounds represented by Formulae (1G) to (11), (IL), (IM), (10), (2G) to (21), (2L), (2M), and (20), Rf₁ is the PFPE chain represented by Formula (3-1). j and k in Rfⱼ and 1 in Rf₂, which represent the PFPE chain, in Formulae (TA) to (IT) and (2A) to (2T) are values indicating the average degrees of polymerization and are thus not necessarily limited to integers.

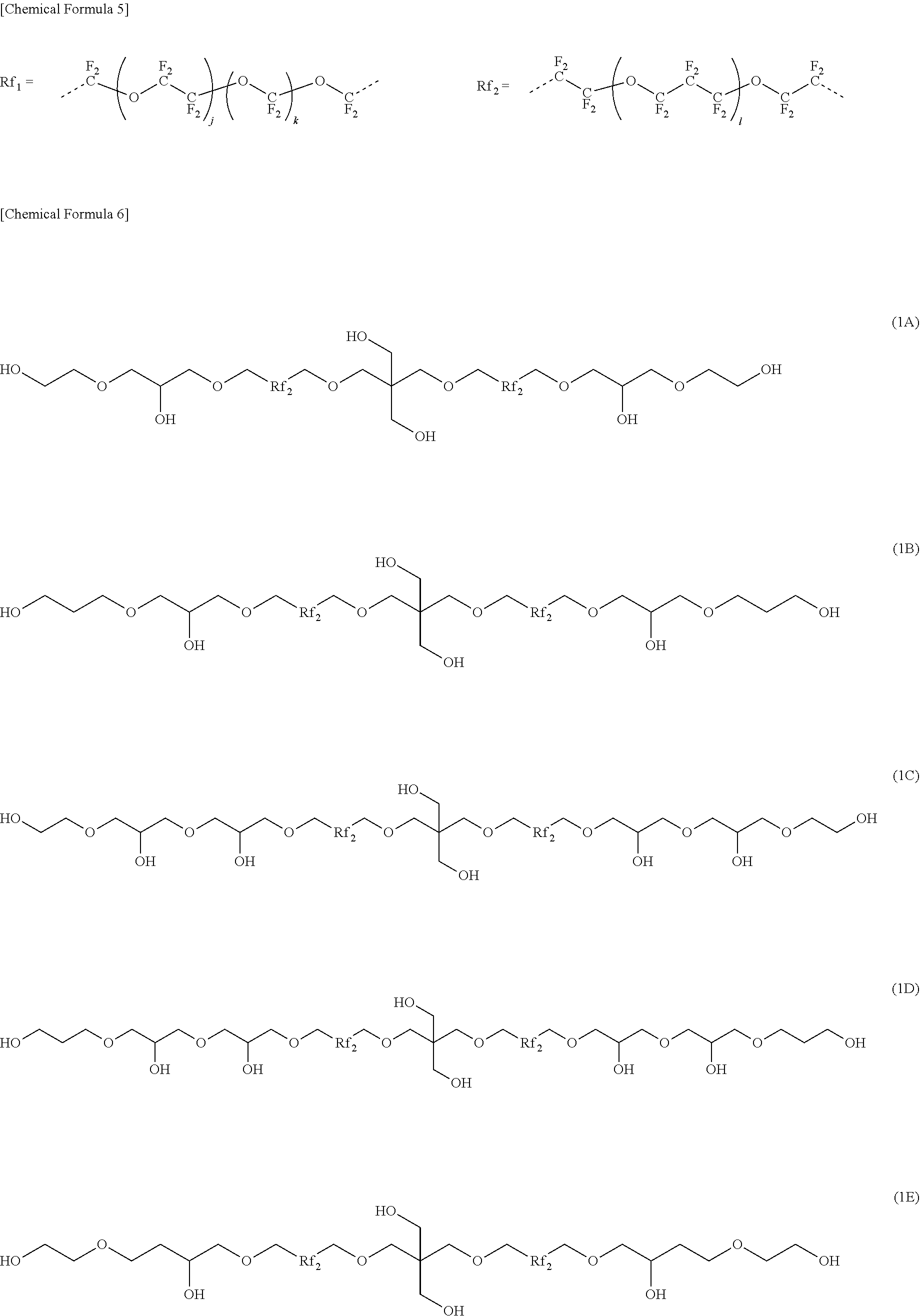

(In two Rf₂'s in Formula (1A), 1 represents the average degree of polymerization and represents 1 to 15. 1's in two Rf₂'s may be the same as or different from each other.)
(In two Rf₂'s in Formula (1B), 1 represents the average degree of polymerization and represents 1 to 15. 1's in two Rf₂'s may be the same as or different from each other.)
(In two Rf₂'s in Formula (1C), 1 represents the average degree of polymerization and represents 1 to 15. 1's in two Rf₂'s may be the same as or different from each other.)
(In two Rf₂'s in Formula (1D), 1 represents the average degree of polymerization and represents 1 to 15. 1's in two Rf₂'s may be the same as or different from each other.)
(In two Rf₂'s in Formula (1E), 1 represents the average degree of polymerization and represents 1 to 15. 1's in two Rf₂'s may be the same as or different from each other.)

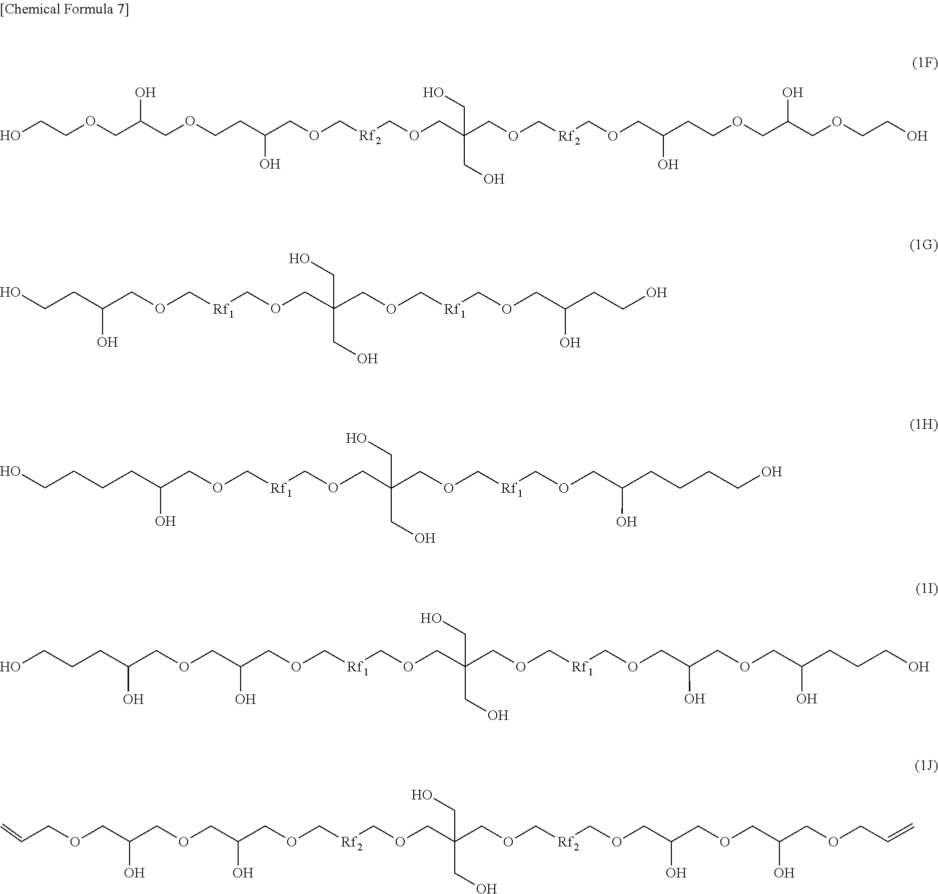

(In two Rf₂'s in Formula (1F), 1 represents the average degree of polymerization and represents 1 to 15. 1's in two Rf₂'s may be the same as or different from each other.)
(In two Rf₁'s in Formula (1G), j and k represent the average degrees of polymerization, j represents 1 to 20, and k represents 0 to 20. j and k in two Rf₁'s may be the same as or different from each other.)
(In two Rf₁'s in Formula (1H), j and k represent the average degrees of polymerization, j represents 1 to 20, and k represents 0 to 20. j and k in two Rf₁'s may be the same as or different from each other.)
(In two Rf₁'s in Formula (11), j and k represent the average degrees of polymerization, j represents 1 to 20, and k represents 0 to 20. j and k in two Rf₁'s may be the same as or different from each other.)
(In two Rf₂'s in Formula (1 J), 1 represents the average degree of polymerization and represents 1 to 15. 1's in two Rf₂'s may be the same as or different from each other.)

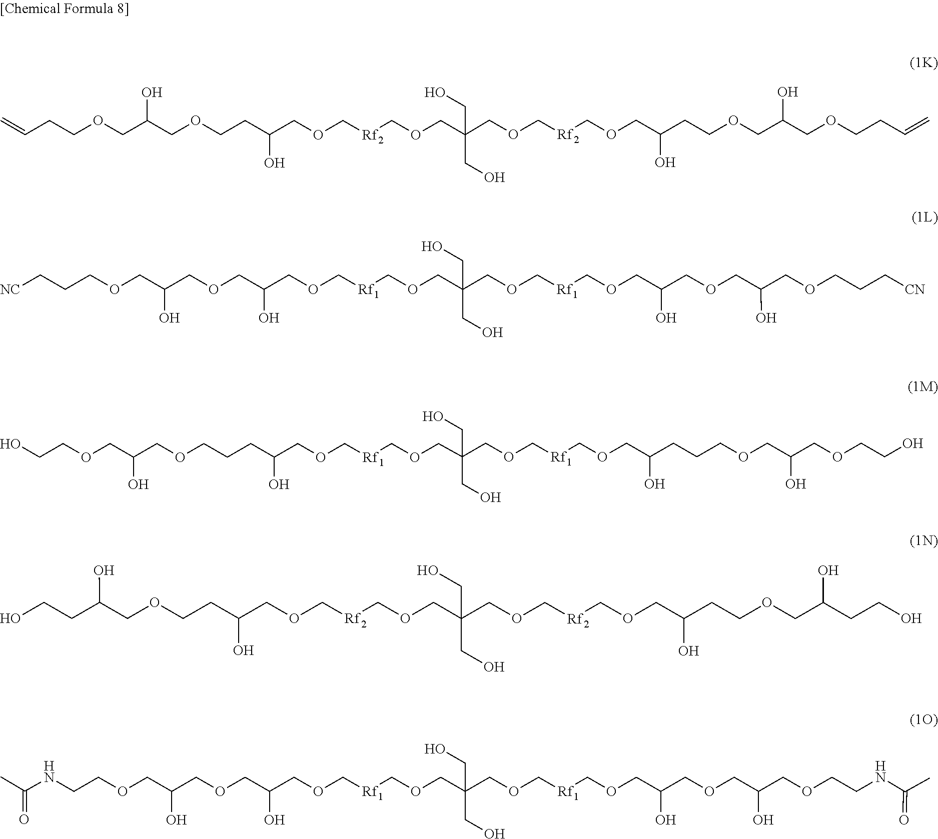

(In two Rf₂'s in Formula (1K), l represents the average degree of polymerization and represents 1 to 15. 1's in two Rf₂'s may be the same as or different from each other.)
(In two Rf₁'s in Formula (1L), j and k represent the average degrees of polymerization, j represents 1 to 20, and k represents 0 to 20. j and k in two Rf₁'s may be the same as or different from each other.)
(In two Rf₁'s in Formula (1M), j and k represent the average degrees of polymerization, j represents 1 to 20, and k represents 0 to 20. j and k in two Rf₁'s may be the same as or different from each other.)
(In two Rf₂'s in Formula (1N), l represents the average degree of polymerization and represents 1 to 15. 1's in two Rf₂'s may be the same as or different from each other.)
(In two Rf₁'s in Formula (10), j and k represent the average degrees of polymerization, j represents 1 to 20, and k represents 0 to 20. j and k in two Rf₁'s may be the same as or different from each other.)

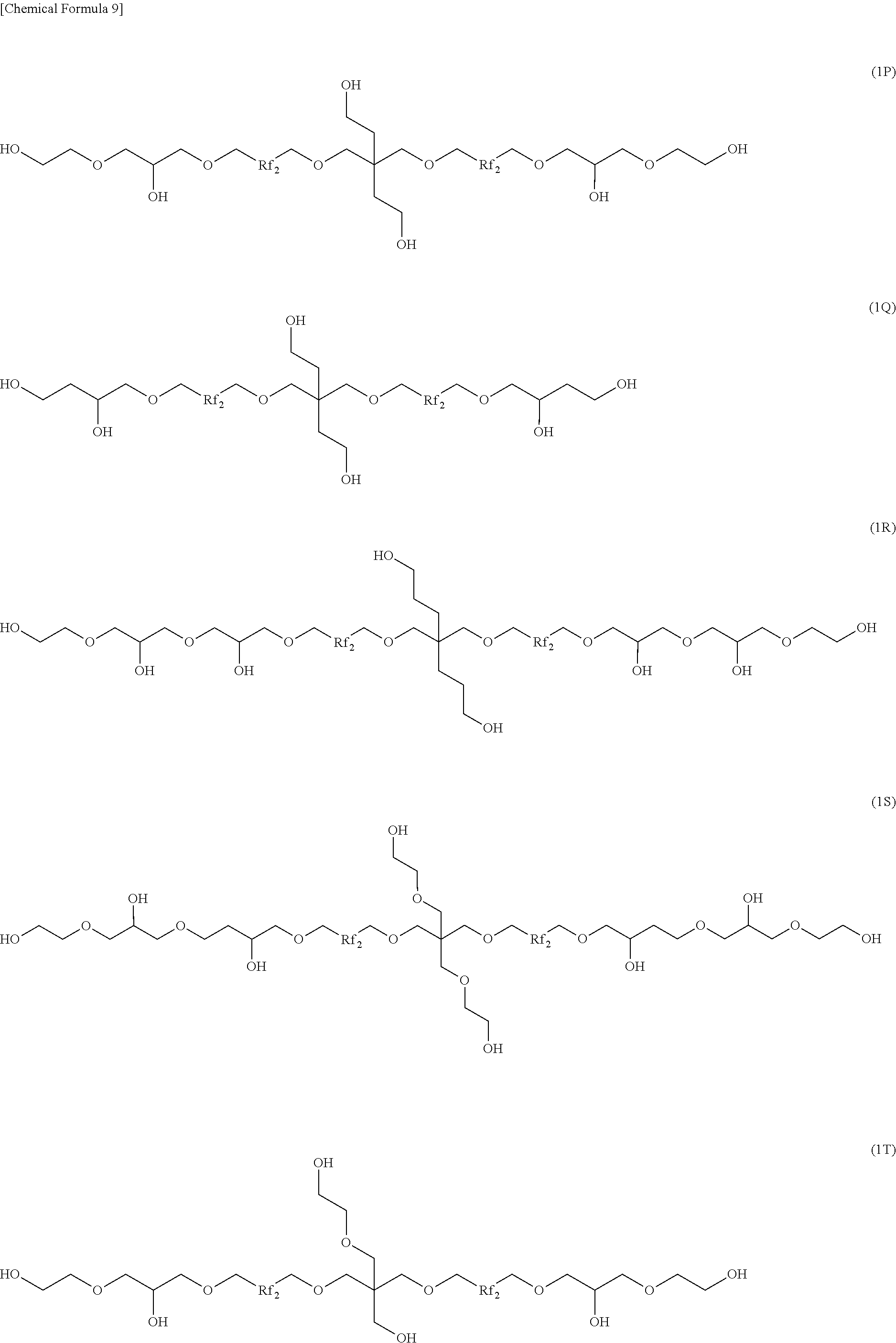

(In two Rf₂'s in Formula (1P), l represents the average degree of polymerization and represents 1 to 15. 1's in two Rf₂'s may be the same as or different from each other.)
(In two Rf₂'s in Formula (1Q), l represents the average degree of polymerization and represents 1 to 15. 1's in two Rf₂'s may be the same as or different from each other.)
(In two Rf₂'s in Formula (1R), l represents the average degree of polymerization and represents 1 to 15. 1's in two Rf₂'s may be the same as or different from each other.)
(In two Rf₂'s in Formula (1S), l represents the average degree of polymerization and represents 1 to 15. 1's in two Rf₂'s may be the same as or different from each other.)
(In two Rf₂'s in Formula (1T), l represents the average degree of polymerization and represents 1 to 15. 1's in two Rf₂'s may be the same as or different from each other.)

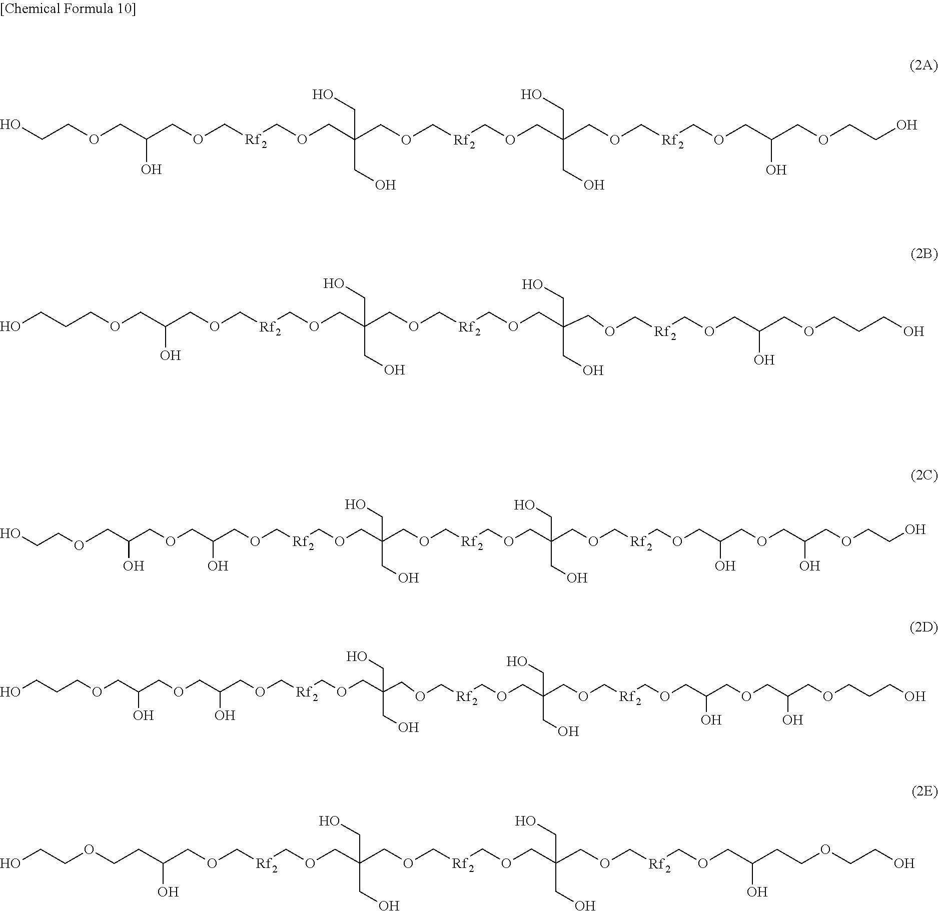

(In three Rf₂'s in Formula (2A), l represents the average degree of polymerization and represents 1 to 15. 1's in three Rf₂'s may be different from each other, or may be the same in part or in whole.)
(In three Rf₂'s in Formula (2B), l represents the average degree of polymerization and represents 1 to 15. 1's in three Rf₂'s may be different from each other, or may be the same in part or in whole.)
(In three Rf₂'s in Formula (2C), l represents the average degree of polymerization and represents 1 to 15. 1's in three Rf₂'s may be different from each other, or may be the same in part or in whole.)
(In three Rf₂'s in Formula (2D), l represents the average degree of polymerization and represents 1 to 15. 1's in three Rf₂'s may be different from each other, or may be the same in part or in whole.)
(In three Rf₂'s in Formula (2E), l represents the average degree of polymerization and represents 1 to 15. 1's in three Rf₂'s may be different from each other, or may be the same in part or in whole.)

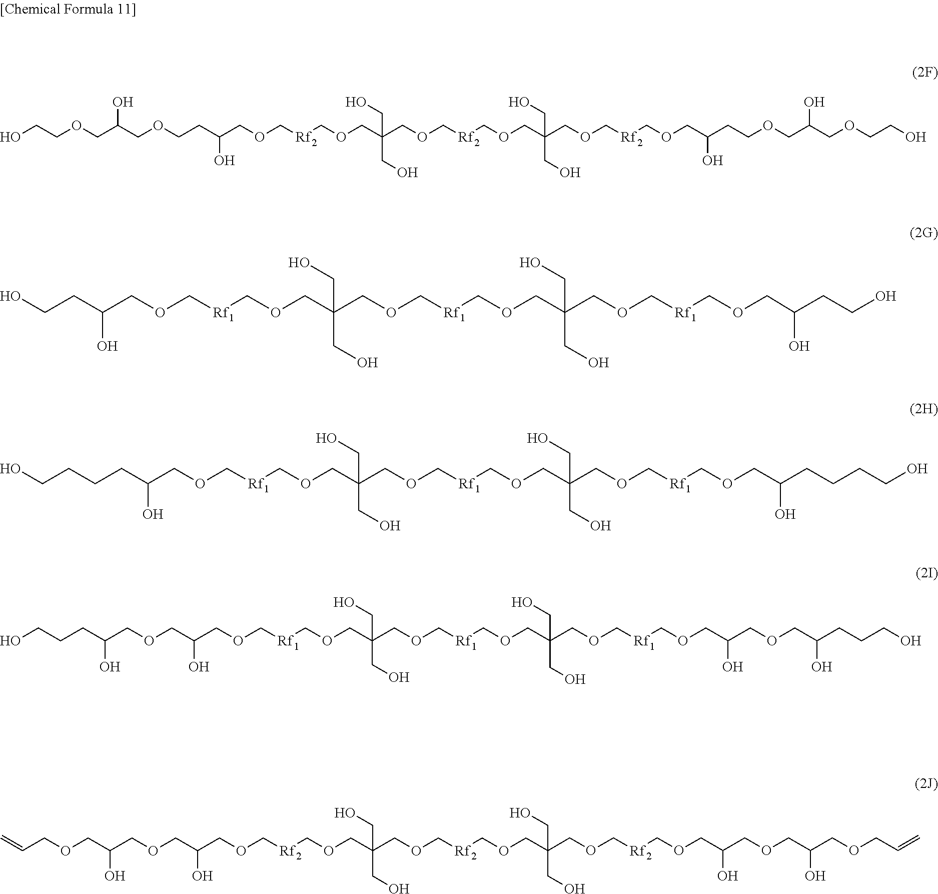

(In three Rf₂'s in Formula (2F), 1 represents the average degree of polymerization and represents 1 to 15. 1's in three Rf₂'s may be different from each other, or may be the same in part or in whole.)
(In three Rf₁'s in Formula (2G), j and k represent the average degrees of polymerization, j represents 1 to 20, and k represents 0 to 20. j and k in three Rf₁'s may be different from each other, or may be the same in part or in whole.)
(In three Rf₁'s in Formula (2H), j and k represent the average degrees of polymerization, j represents 1 to 20, and k represents 0 to 20. j and k in three Rf₁'s may be different from each other, or may be the same in part or in whole.)
(In three Rf₁'s in Formula (2I), j and k represent the average degrees of polymerization, j represents 1 to 20, and k represents 0 to 20. j and k in three Rf₁'s may be different from each other, or may be the same in part or in whole.)
(In three Rf₂'s in Formula (2J), 1 represents the average degree of polymerization and represents 1 to 15. 1's in three Rf₂'s may be different from each other, or may be the same in part or in whole.)

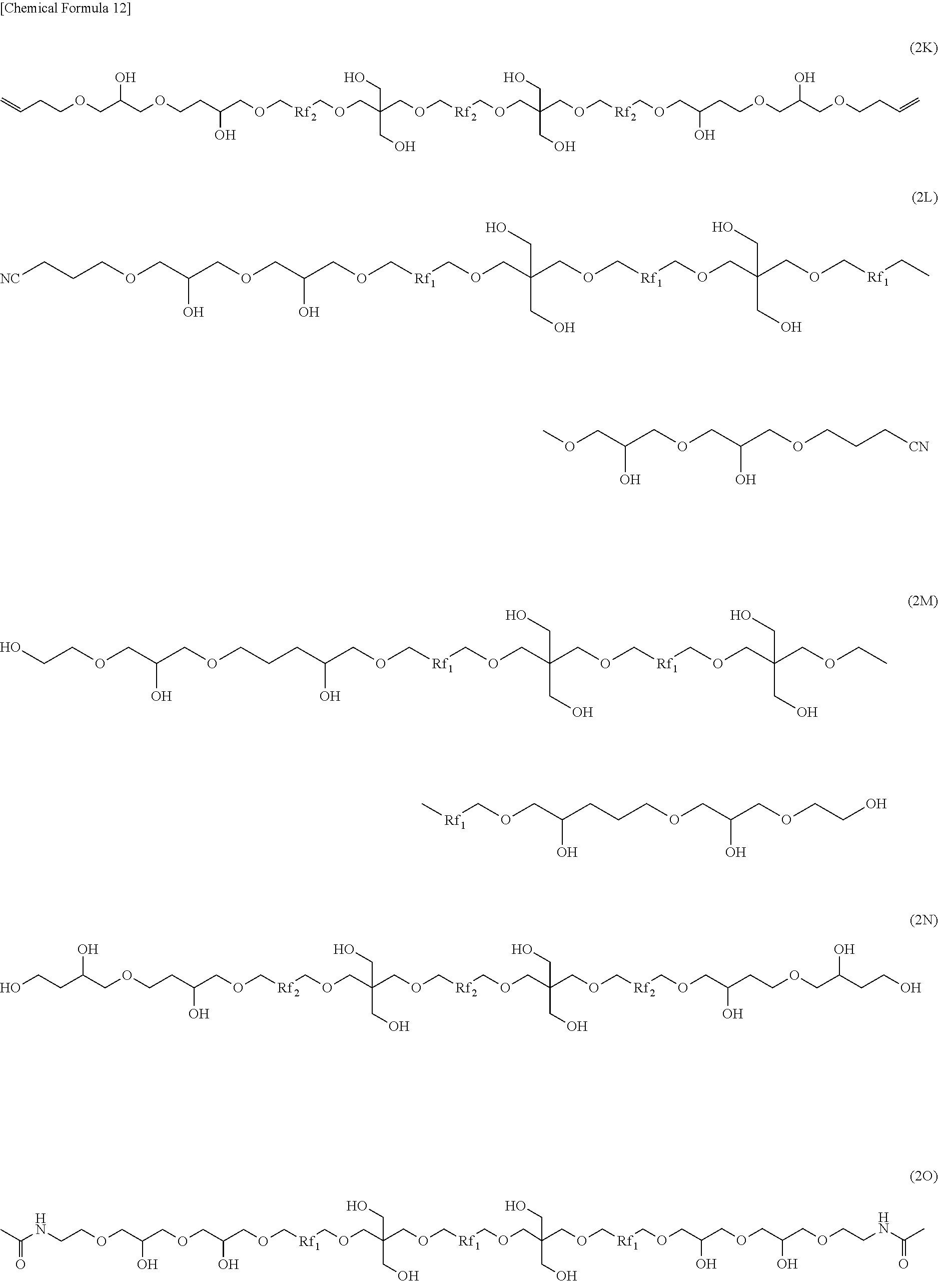

(In three Rf₂'s in Formula (2K), l represents the average degree of polymerization and represents 1 to 15. 1's in three Rf₂'s may be different from each other, or may be the same in part or in whole.)
(In three Rf₁'s in Formula (2L), j and k represent the average degrees of polymerization, j represents 1 to 20, and k represents 0 to 20. j and k in three Rf₁'s may be different from each other, or may be the same in part or in whole.)
(In three Rf₁'s in Formula (2M), j and k represent the average degrees of polymerization, j represents 1 to 20, and k represents 0 to 20. j and k in three Rf₁'s may be different from each other, or may be the same in part or in whole.)
(In three Rf₂'s in Formula (2N), 1 represents the average degree of polymerization and represents 1 to 15. 1's in three Rf₂'s may be different from each other, or may be the same in part or in whole.)
(In three Rf₁'s in Formula (20), j and k represent the average degrees of polymerization, j represents 1 to 20, and k represents 0 to 20. j and k in three Rf₁'s may be different from each other, or may be the same in part or in whole.)

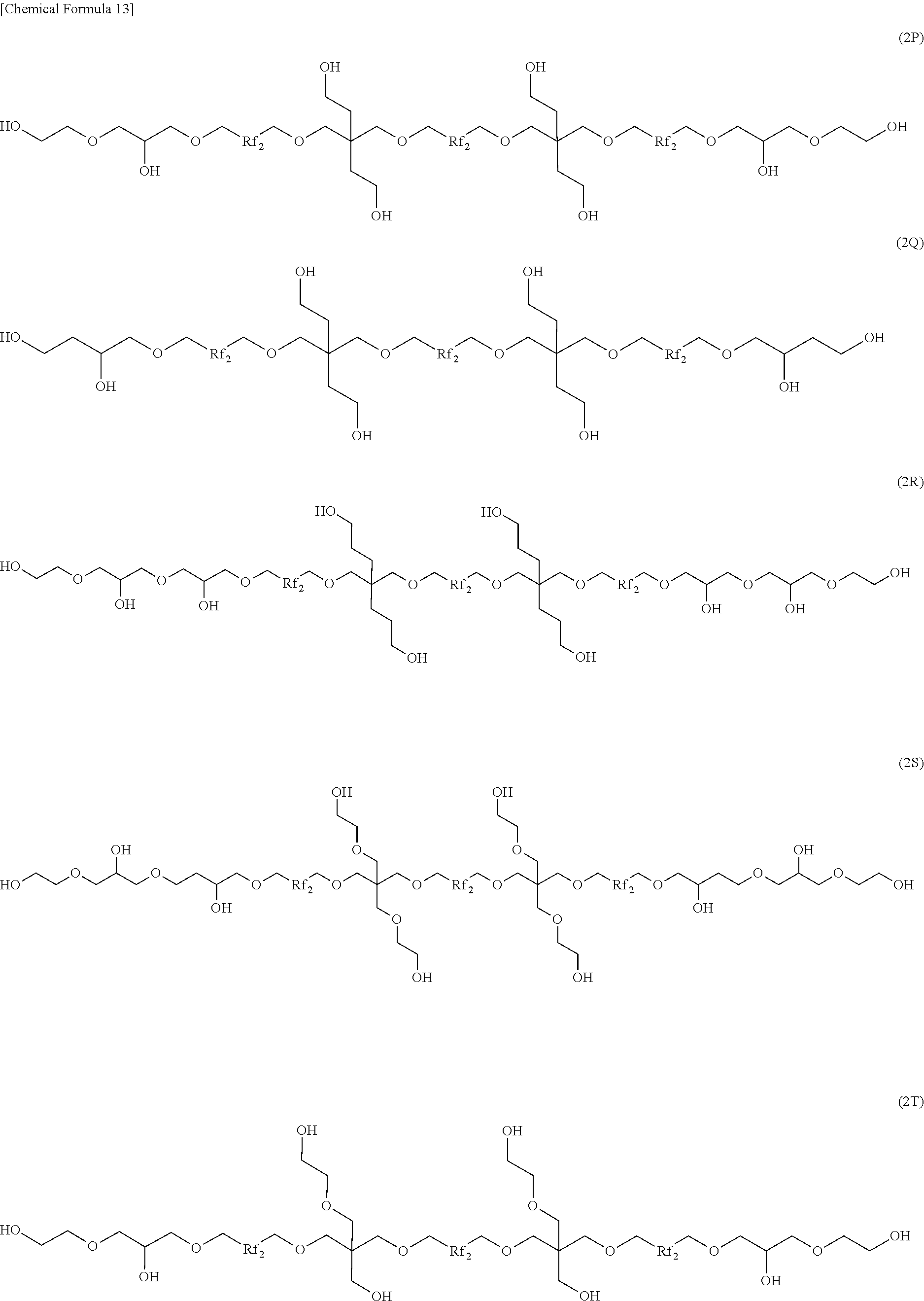

(In three Rf₂'s in Formula (2P), 1 represents the average degree of polymerization and represents 1 to 15. 1's in three Rf₂'s may be different from each other, or may be the same in part or in whole.)
(In three Rf₂'s in Formula (2Q), 1 represents the average degree of polymerization and represents 1 to 15. 1's in three Rf₂'s may be different from each other, or may be the same in part or in whole.)
(In three Rf₂'s in Formula (2R), 1 represents the average degree of polymerization and represents 1 to 15. 1's in three Rf₂'s may be different from each other, or may be the same in part or in whole.)
(In three Rf₂'s in Formula (2S), 1 represents the average degree of polymerization and represents 1 to 15. 1's in three Rf₂'s may be different from each other, or may be the same in part or in whole.)
(In three Rf₂'s in Formula (2T), 1 represents the average degree of polymerization and represents 1 to 15. 1's in three Rf₂'s may be different from each other, or may be the same in part or in whole.)

The number-average molecular weight (Mn) of the fluorine-containing ether compound of the present embodiment is preferably in a range of 500 to 10000, more preferably in a range of 500 to 5000, and particularly preferably in a range of 1000 to 3000. In a case where the number-average molecular weight is 500 or more, the lubricant containing the fluorine-containing ether compound of the present embodiment is less likely to transpiration, and it is possible to prevent the transpiration and transfer to the magnetic head of the lubricant. In addition, in a case where the number-average molecular weight is 10,000 or less, the viscosity of the fluorine-containing ether compound becomes appropriate, and a lubricating layer having a thin thickness can be easily formed by applying a lubricant including the fluorine-containing ether compound. In a case where the number-average molecular weight is 5,000 or less, the viscosity becomes easy to handle in the case of being applied to the lubricant, which is more preferable.

The number-average molecular weight (Mn) of the fluorine-containing ether compound is a value measured by ¹H-NMR and ¹⁹F-NMR using AVANCE III 400 manufactured by Bruker Biospin. Specifically, the number of repeating units of the PFPE chain is calculated from the integral value measured by ¹⁹F-NMR, and the number-average molecular weight is determined. In the measurement of nuclear magnetic resonance (NMR), the sample was diluted in a single solvent such as hexafluorobenzene, d-acetone, or d-tetrahydrofuran or a solvent mixture and was used for the measurement. As the reference for the ¹⁹F-NMR chemical shift, the peak of hexafluorobenzene was set to −164.7 ppm. As the reference of the ¹H-NMR chemical shift, the peak of acetone was set to 2.2 ppm.

In the fluorine-containing ether compound of the present embodiment, it is preferable that the polydispersity (weight-average molecular weight (Mw)/number-average molecular weight (Mn) ratio) is set to 1.3 or less by cutting off the molecular weight by an appropriate method.

In the present embodiment, a method for cutting off the molecular weight is not particularly limited, and for example, molecular weight cutting-off by a silica gel column chromatography method, a gel permeation chromatography (GPC) method, or the like, molecular weight cutting-off by a supercritical extraction method, or the like can be used.

### “Production Method”

A method for producing the fluorine-containing ether compound of the present embodiment is not particularly limited, and the fluorine-containing ether compound can be produced using a known production method in the related art. The fluorine-containing ether compound of the present embodiment can be produced by, for example, the following production method.

### <First Production Method>

In the case of producing a fluorine-containing ether compound in which z in Formula (1) is 1, R¹ and R⁵ are the same as each other, and two PFPE chains represented by R² are the same as each other, a production method shown in Formula (5) can be used.

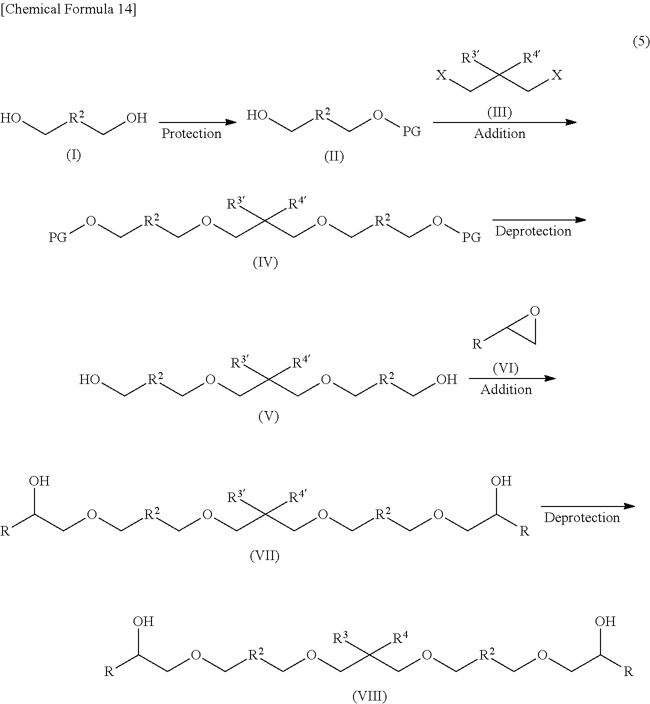

(In Formula (5), PG represents a protective group. X represents a (pseudo)halogen group. R^{3′} and R^{4′} are each a structure in which a protective group is bonded to hydroxy groups included in R³ and R⁴ in Formula (1), and represent groups having a carbon atom bonded to a tetrasubstituted carbon atom. R represents partial structures of the terminal groups represented by R¹ and R⁵ in Formula (1).)

### (First Reaction)

A fluorine-based compound (I) having hydroxymethyl groups (—CH₂OH) at both terminals of the PFPE chain corresponding to R² in Formula (1) is prepared. An appropriate protective group (PG) is introduced into the hydroxymethyl group at one terminal of the fluorine-based compound (I) to obtain a first intermediate compound (II).

### (Second Reaction)

Next, a compound (III) having a partial structure (—CR^{3′}R^{4′}—) corresponding to —CR³R⁴— and two (pseudo)halogen groups (X) is reacted with the hydroxymethyl group (—CH₂OH) which is not substituted with the protective group (PG) in the first intermediate compound (II) obtained by the first reaction. The reaction ratio between the first intermediate compound (II) and the compound (11I) is preferably about 2:1 (molar ratio). The protective groups (PG) at both terminals of a compound (IV) having a linking group including the obtained partial structure (—CR³R⁴—) corresponding to —CR^{3′}R^{4′}— and two perfluoropolyether chains (R²) are deprotected, whereby a second intermediate compound (V) is produced.

As the (pseudo)halogen group (X) in the compound (III) described above, for example, at least one selected from a chloro group, a bromo group, an iodo group, a p-toluenesulfonyloxy group, a methanesulfonyloxy group, a trifluoromethanesulfonyloxy group, a perfluoroalkylsulfonyloxy group, or a nitrobenzenesulfonyloxy group can be used.

### (Third Reaction)

Thereafter, an epoxy compound (VI) having a group (R) serving as R¹ (=R⁵) in Formula (1) is reacted with hydroxy groups at both terminals of the second intermediate compound (V). R in the epoxy compound (VI) has a structure corresponding to a part of the terminal group represented by R¹ or R⁵ in Formula (1). A third reaction is performed, whereby a third intermediate compound (VII) having terminal group corresponding to R¹ (=R⁵) at both terminals of a chain-like structure having a linking group including a partial structure (—CR^{3′}R^{4′}—) corresponding to —CR³R⁴— and two perfluoropolyether chains (R²) is obtained.

### (Fourth Reaction)

Finally, the protective group included in the third intermediate compound (VII) obtained by the third reaction is deprotected by an appropriate method. As a result, a fluorine-containing ether compound (VIII) is obtained.

The above-described steps make it possible to produce a fluorine-containing ether compound in which z in Formula (1) is 1, two terminal groups represented by R¹ and R⁵ are the same as each other, and two PFPE chains represented by R² are the same as each other.

In the case of producing the fluorine-containing ether compound of the present embodiment using the above-described first production method, examples of the compound (III) used in the second reaction include compounds represented by Formulae (A) to (S). The compound (III) can be produced using a known method. As the compound (III), a commercially available product may be purchased and used.

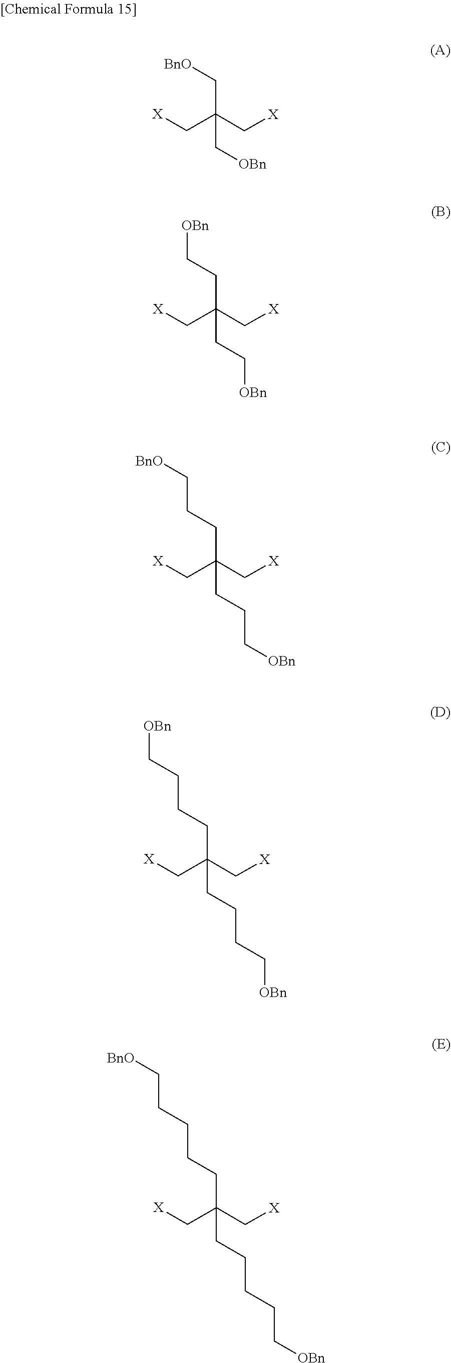

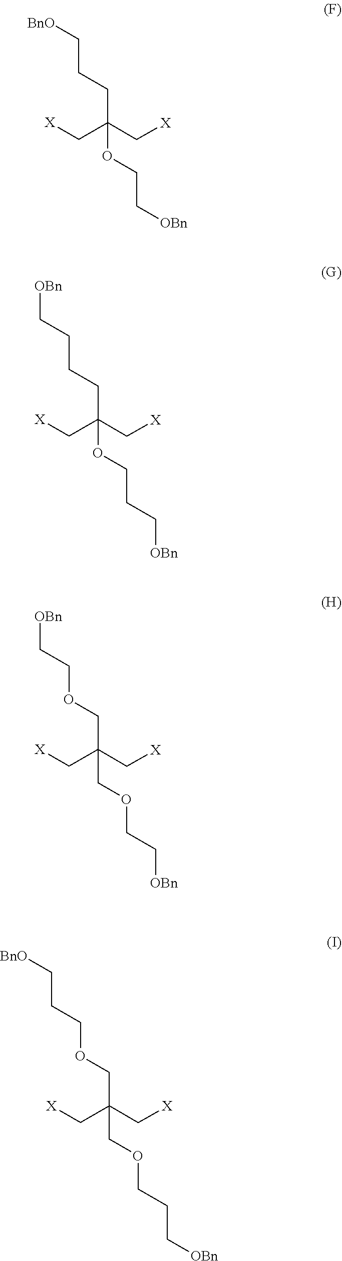

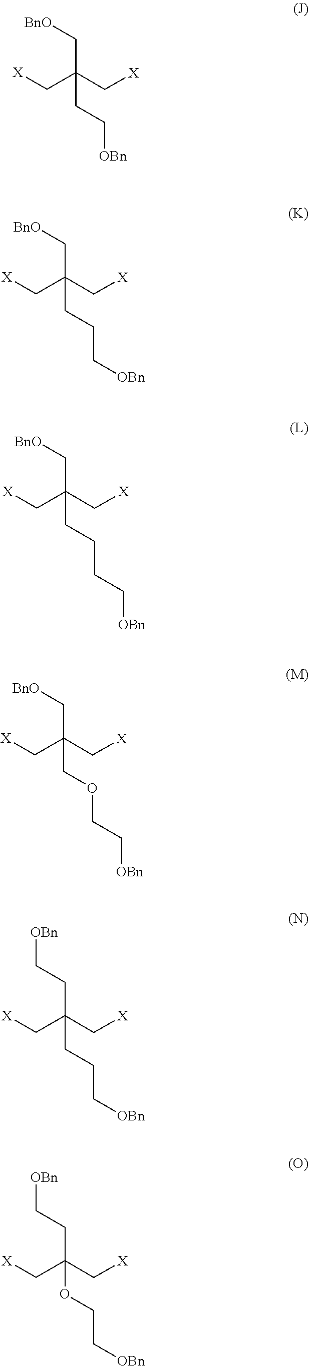

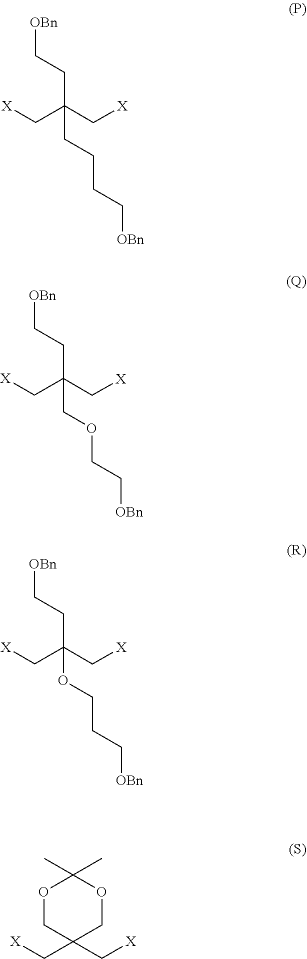

(In Formulae (A) to (S), X represents a (pseudo)halogen group, and two X's in the same molecule may be the same as or different from each other. In Formulae (A) to (R), Bn represents a benzyl group.)

### (Method for Producing Compound (III))

The compound (III) can be synthesized using, for example, a production method shown in Formula (6).

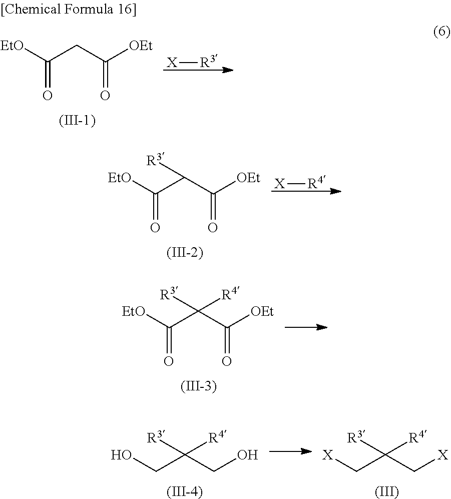

(In Formula (6), Et represents an ethyl group. X represents a (pseudo)halogen group. R^{3′} and R^{4′} are each a structure in which a protective group is bonded to hydroxy groups included in R³ and R⁴ in Formula (1), and represent groups having a carbon atom bonded to a tetrasubstituted carbon atom.)

First, a malonic acid ester (III-1) is alkylated using a (pseudo)halide (X—R^{3′}) of an organic group corresponding to R^{3′}, thereby obtaining an intermediate compound (III-2). Next, a (pseudo)halide (X—R^{4′}) of an organic group corresponding to R^{4′} is further reacted with the intermediate compound (III-2). As a result, an intermediate compound (III-3) including a tetrasubstituted carbon atom is obtained. Subsequently, two ester groups in the intermediate compound (III-3) are reduced to hydroxymethyl groups to obtain an intermediate compound (III-4). Thereafter, a hydroxy group of the obtained intermediate compound (III-4) is converted into an appropriate (pseudo)halogen group. The above-described steps make a compound (III) used in the second reaction obtained.

In the present embodiment, the organic group corresponding to R^{3′} is bonded to the malonic acid ester (III-1), and then the organic group corresponding to R^{4′} is bonded thereto to construct a tetrasubstituted carbon atom. However, a tetrasubstituted carbon atom may be constructed by bonding the organic group corresponding to R⁴′ to the malonic acid ester (III-1), and then bonding the organic group corresponding to R³′ thereto.

In the case of producing a fluorine-containing ether compound in which one of R³ or R⁴ in Formula (1) has an oxygen atom bonded to a tetrasubstituted carbon atom and the other has a carbon atom bonded to the tetrasubstituted carbon atom, using the above-described first production method, as the compound (III) used in the second reaction, for example, a compound synthesized using a production method shown in Formula (7) can be used.

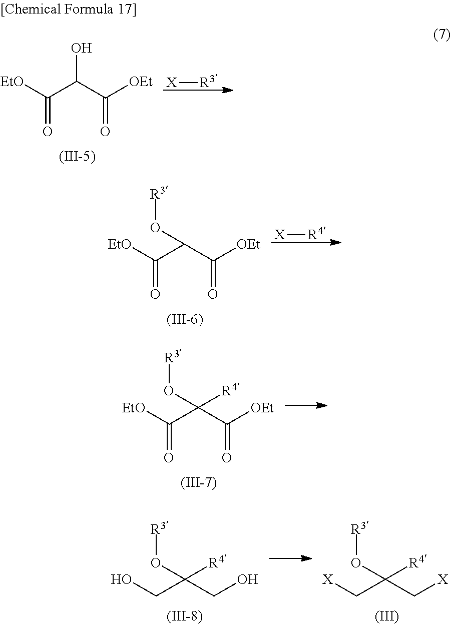

(In Formula (7), Et represents an ethyl group. X represents a (pseudo)halogen group. R^{3′} is a structure in which a protective group is bonded to a hydroxy group included in R³, and represents a group from which an oxygen atom being bonded to a tetrasubstituted carbon atom has been removed. R^{4′} is a structure in which a protective group is bonded to a hydroxy group included in R⁴, and represents a group having a carbon atom bonded to a tetrasubstituted carbon atom.)

First, a 2-hydroxymalonate ester (III-5) is alkylated using a (pseudo)halide (X—R^{3′}) of an organic group corresponding to R^{3′}, thereby obtaining an intermediate compound (III-6). Next, a (pseudo)halide (X—R^{4′}) of an organic group corresponding to R^{4′} is further reacted with the intermediate compound (III-6). As a result, an intermediate compound (III-7) including a tetrasubstituted carbon atom is obtained. Subsequently, two ester groups in the intermediate compound (III-7) are reduced to hydroxymethyl groups to obtain an intermediate compound (III-8). Thereafter, a hydroxy group of the obtained intermediate compound (III-8) is converted into an appropriate (pseudo)halogen group. The above-described steps make the compound (III) used in the second reaction obtained in the case of producing the fluorine-containing ether compound in which one of R³ and R⁴ in Formula (1) has an oxygen atom bonded to a tetrasubstituted carbon atom and the other has a carbon atom bonded to the tetrasubstituted carbon atom.

In the present embodiment, the organic group corresponding to R^{3′} is bonded to the 2-hydroxymalonate ester (III-5), and then the organic group corresponding to R^{4′} is bonded thereto to construct a tetrasubstituted carbon atom. However, a tetrasubstituted carbon atom may be constructed by bonding the organic group corresponding to R⁴′ to the 2-hydroxymalonate ester (III-5), and then bonding the organic group corresponding to R^{3′} thereto.

As the (pseudo)halides of the organic groups corresponding to R^{3′} and R^{4′} used in the production of the compound (III), for example, compounds represented by Formulae (III-9) to (III-13) can be used.

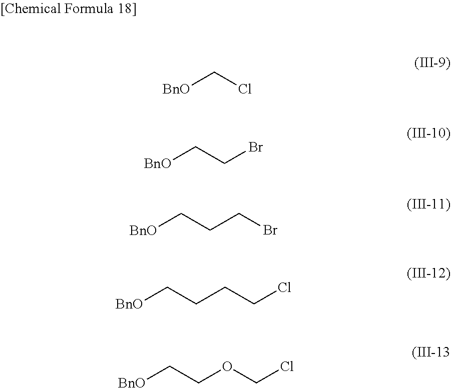 [SMILES: ClCOCc1ccccc1 ; BrCCOCc1ccccc1 ; BrCCCOCc1ccccc1 ; ClCCCCOCc1ccccc1 ; ClCOCCOCc1ccccc1]

(In Formulae (III-9) to (III-13), Bn represents a benzyl group.)

In the case of producing the fluorine-containing ether compound of the present embodiment using the above-described first production method, the epoxy compound (VI) having the group (R) as R¹ (=R⁵) in Formula (1) used in the third reaction can be produced using, for example, a method shown in Formula (8). That is, as shown in Formula (8), the compound can be produced using a method of reacting an alcohol having an R group representing a partial structure of the terminal group represented by R¹ or R⁵ in Formula (1) with a halogen compound having an epoxy group (in Formula (8), a bromine compound).

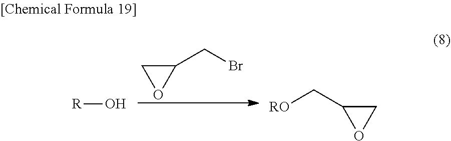

(In Formula (8), R represents the partial structures of the terminal groups represented by R¹ and R⁵ in Formula (1).)

The epoxy compound (VI) may be produced by the following method. That is, as shown in Formula (9), an alcohol having the R group representing a partial structure of the terminal group represented by R¹ or R⁵ in Formula (1) is subjected to an addition reaction with allyl glycidyl ether. Thereafter, an unsaturated bond site contained in the compound obtained by the addition reaction is oxidized by making m-chloroperbenzoic acid (mCPBA) act thereon. The above-described steps make it possible to obtain the epoxy compound (VI) is obtained.

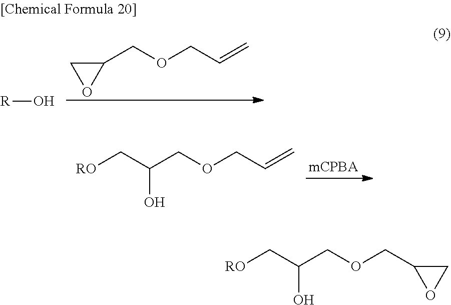

(In Formula (9), R represents the partial structures of the terminal groups represented by R¹ and R⁵ in Formula (1).)

The epoxy compound (VI) may be produced by the following method. That is, as shown in Formula (10), an alcohol having the R group representing a partial structure of a terminal group represented by R¹ or R⁵ in Formula (1) is reacted with a halogen compound having an alkenyl group (In Formula (10), a bromine compound). Thereafter, an unsaturated bond site contained in the obtained compound is oxidized by making m-chloroperbenzoic acid (mCPBA) act thereon. The above-described steps make it possible to obtain the epoxy compound (VI) is obtained.

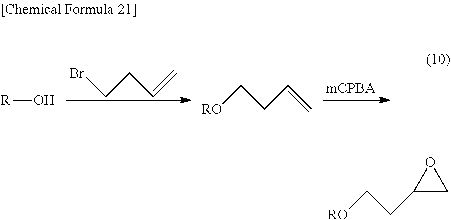

(In Formula (10), R represents the partial structures of the terminal groups represented by R¹ and R⁵ in Formula (1).)

As the epoxy compound (VI), a commercially available product may be purchased and used.

In the case of producing the fluorine-containing ether compound of the present embodiment using the above-described first production method, the epoxy compound (VI) may be reacted with the second intermediate compound (V) in the third reaction after a polar group contained in the R group representing the partial structures of the terminal groups represented by R¹ or R⁵ in Formula (1) is protected using an appropriate protective group.

As the first production method, the following method may be used instead of the method represented by Formula (5) described above.

### (First Reaction)

The epoxy compound (VI) is reacted with a hydroxymethyl group (—CH₂HO) at one terminal of the fluorine-based compound (I) in Formula (5) to obtain a first intermediate compound.

### (Second Reaction)

Next, the compound (III) is reacted with a hydroxymethyl group at one terminal of the first intermediate compound obtained by the first reaction. The reaction ratio between the first intermediate compound and the compound (III) is preferably about 2:1 (molar ratio). The protective group included in the obtained compound is deprotected by an appropriate method.

The above-described steps make it possible to produce a fluorine-containing ether compound in which z in Formula (1) is 1, two terminal groups represented by R¹ and R⁵ are the same as each other, and two PFPE chains represented by R² are the same as each other.

### <Second Production Method>

In the case of producing a fluorine-containing ether compound in which z in Formula (1) is 1 and any one or more of terminal groups represented by R¹ and R⁵ or two PFPE chains represented by R² are different, the following production method can be used.

### (First Reaction)

First, a fluorine-based compound having hydroxymethyl groups (—CH₂OH) at both terminals of the PFPE chain corresponding to R² on the R¹ side is prepared, and an epoxy compound having a partial structure corresponding to R¹ is reacted with one terminal hydroxy group thereof. This produces a first intermediate compound having a group corresponding to R¹ at one terminal of the PFPE chain corresponding to R² on the R¹ side.

### (Second Reaction)

Next, the first intermediate compound is reacted with the compound (III) in the first production method. The reaction ratio between the first intermediate compound and the compound (III) is preferably about 1:1 (molar ratio). As a result, a second intermediate compound which has a group corresponding to R¹ at one terminal of the PFPE chain corresponding to R² on the R¹ side and has a partial structure corresponding to —CR³R⁴— and a (pseudo)halogen group disposed in this order at the other terminal is produced.

### (Third Reaction)

Next, a third intermediate compound having a group corresponding to R⁵ at one terminal of the PFPE chain corresponding to R² on the R⁵ side is produced in the same manner as the first intermediate compound.

Thereafter, the second intermediate compound and the third intermediate compound are reacted with each other. As a result, a fourth intermediate compound having terminal groups corresponding to R¹ and R⁵ at both terminals of a chain-like structure having a linking group including a partial structure corresponding to —CR³R⁴— between two PFPE chains is obtained.

### (Fourth Reaction)

Finally, the protective group included in the obtained fourth intermediate compound is deprotected by an appropriate method.

A fluorine-containing ether compound in which z in Formula (1) is 1, and any one or more of the terminal groups represented by R¹ and R⁵, or two PFPE chains represented by R² are different can be produced by sequentially performing the above-described steps.

### <Third Production Method>

In the case of producing a fluorine-containing ether compound in which z in Formula (1) is 2, three PFPE chains represented by R² are the same as one another, groups represented by —CR³R⁴— included in two linking groups are the same as each other, and terminal groups represented by R¹ and R⁵ are the same as each other, or in the case of producing a fluorine-containing ether compound in which z in Formula (1) is 2, only the center R² among three PFPE chains represented by R² is different, groups represented by —CR³R⁴— included in two linking groups are the same as each other, and terminal groups represented by R¹ and R⁵ are the same as each other, the following production method can be used.

### (First Reaction)

A fluorine-based compound in which a hydroxymethyl group (—CH₂OH) is disposed at each of both terminals of the PFPE chain corresponding to R² in the center of the molecule in Formula (1) is prepared. Next, hydroxy groups of the hydroxymethyl groups disposed at both terminals of the fluorine-based compound are reacted with the compound (III) in the first production method. The reaction ratio between the fluorine-based compound and the compound (III) is preferably about 1:2 (molar ratio). As a result, a first intermediate compound in which a partial structure corresponding to —CR³R⁴— and a (pseudo)halogen group are disposed in this order at each of both terminals of the perfluoropolyether chain corresponding to R² in the center of the molecule in Formula (1) is obtained.

### (Second Reaction)

Next, a second intermediate compound in which an appropriate protective group is introduced into one terminal of the PFPE chain corresponding to R² (R² which is not positioned at the center of the molecule) adjacent to R¹ or R⁵ in Formula (1) is produced in the same manner as in the first reaction in the first production method described above.

### (Third Reaction)

Thereafter, the first intermediate compound and the second intermediate compound are mixed and reacted with each other. Thereafter, the protective groups at both terminals of a compound having two linking groups including the obtained partial structure corresponding to —CR³R⁴— and three PFPE chains are deprotected, whereby a third intermediate compound is produced. The reaction ratio between the first intermediate compound and the second intermediate compound is preferably about 1:2 (molar ratio).

### (Fourth Reaction)

Next, the epoxy compound (VI) in the first production method is reacted with the hydroxy groups at both terminals of the third intermediate compound obtained by the third reaction. A fourth intermediate compound having terminal groups corresponding to R¹ (=R⁵) at both terminals of a chain-like structure having two linking groups including a partial structure corresponding to —CR³R⁴— and three PFPE chains is obtained by performing a fourth reaction.

### (Fifth Reaction)

Finally, the protective group included in the fourth intermediate compound obtained by the fourth reaction is deprotected by an appropriate method. The above-described steps make it possible to produce a fluorine-containing ether compound in which z in Formula (1) is 2, three PFPE chains represented by R² are the same as one another, groups represented by —CR³R⁴— included in two linking groups are the same as each other, and terminal groups represented by R¹ and R⁵ are the same as each other, or a fluorine-containing ether compound in which z in Formula (1) is 2, only the center R² among three PFPE chains represented by R² is different, groups represented by —CR³R⁴— included in two linking groups are the same as each other, and terminal groups represented by R¹ and R⁵ are the same as each other.

In the above-described third production method, the second reaction is performed after the first reaction, but the first reaction may be performed after the second reaction.

In addition, as the third production method, the following method may be used instead of the second reaction to the fifth reaction described above.

### (Second Reaction)

A fluorine-based compound having hydroxymethyl groups (—CH₂OH) at both terminals of the PFPE chain corresponding to R² (R² which is not positioned at the center of the molecule) adjacent to R¹ or R⁵ in Formula (1) is prepared. The epoxy compound (VI) in the first production method is reacted with the hydroxymethyl group at one terminal of this fluorine-based compound to obtain a second intermediate compound.

### (Third Reaction)

Next, the first intermediate compound obtained by the first reaction is reacted with a hydroxymethyl group at one terminal of the second intermediate compound obtained by the second reaction. The reaction ratio between the first intermediate compound and the second intermediate compound is preferably about 1:2 (molar ratio). The protective group included in the compound obtained in this manner is deprotected by an appropriate method. The above-described steps make it possible to produce a fluorine-containing ether compound.

### <Fourth Production Method>

In the case of producing a fluorine-containing ether compound in which z in Formula (1) is 2, groups represented by —CR³R⁴— included in two linking groups are the same as each other, and terminal groups represented by R¹ and R⁵ and any one or more of three PFPE chains are different, the following production method can be used.

### (First Reaction)

First, a first intermediate compound having a group corresponding to R¹ at one terminal of the PFPE chain corresponding to R² on the R¹ side is produced in the same manner as in the first reaction of the above-described second production method.

### (Second Reaction)

A second intermediate compound having a group corresponding to R⁵ at one terminal of the PFPE chain corresponding to R² on the R⁵ side is produced in the same manner as in the first reaction of the fourth production method.

### (Third Reaction)

The first intermediate compound in the third production method is produced in the same manner as in the first reaction of the third production method described above and used as a third intermediate compound in the fourth production method.

### (Fourth Reaction and Fifth Reaction)

A fourth reaction of reacting the first intermediate compound with the third intermediate compound obtained by the third reaction and a fifth reaction of reacting the second intermediate compound with the fourth intermediate compound obtained by the fourth reaction are sequentially performed. As a result, a fifth intermediate compound having terminal groups corresponding to R¹ and R⁵ at both terminals of a chain-like structure having two linking groups including a partial structure corresponding to —CR³R⁴— and three PFPE chains is obtained.

### (Sixth Reaction)

Finally, the protective group included in the fifth intermediate compound obtained by the fifth reaction is deprotected by an appropriate method, whereby a fluorine-containing ether compound is produced.

The above-described steps make it possible to produce a fluorine-containing ether compound in which z in Formula (1) is 2, groups represented by —CR³R⁴— included in two linking groups are the same as each other, and terminal groups represented by R¹ and R⁵ and any one or more of three PFPE chains are different.

In the above-described fourth production method, the first reaction, the second reaction, and the third reaction are performed in this order, but the order of performing the first reaction to the third reaction is not particularly limited.

In addition, in the above-described fourth production method, the first intermediate compound is used in the fourth reaction, and the second intermediate compound is used in the fifth reaction, but the second intermediate compound may be used in the fourth reaction, and the first intermediate compound may be used in the fifth reaction.

### <Fifth Production Method>

In the case of producing a fluorine-containing ether compound in which z in Formula (1) is 2, groups represented by —CR³R⁴— included in two linking groups are different from each other, terminal groups represented by R¹ and R⁵ are the same as or different from each other, and three PFPE chains represented by R² are the same as one another or at least one is different, the following production method can be used.

### (First Reaction)

First, a first intermediate compound having a group corresponding to R¹ at one terminal of the PFPE chain corresponding to R² on the R¹ side is produced in the same manner as in the first reaction of the above-described second production method.

### (Second Reaction)

Next, a second intermediate compound which has a group corresponding to R at one terminal of the PFPE chain corresponding to R² on the R¹ side and has a partial structure corresponding to —CR³R⁴— on the R¹ side and a (pseudo)halogen group disposed in this order at the other terminal is produced in the same manner as in the second reaction of the above-described second production method.

### (Third Reaction)

A third intermediate compound having a group corresponding to R⁵ at one terminal of the PFPE chain corresponding to R² on the R⁵ side is produced in the same manner as in the first reaction of the fifth production method.

### (Fourth Reaction)

A fourth intermediate compound which has a group corresponding to R⁵ at one terminal of the PFPE chain corresponding to R² on the R⁵ side and a partial structure corresponding to —CR³R⁴— on the R⁵ side and a (pseudo)halogen group disposed in this order at the other terminal is produced in the same manner as in the second reaction of the fifth production method.

### (Fifth Reaction and Sixth Reaction)

A fluorine-based compound in which a hydroxymethyl group (—CH₂OH) is disposed at each of both terminals of the PFPE chain corresponding to R² in the center of the molecule in Formula (1) is prepared. Then, a fifth reaction of reacting the second intermediate compound with one terminal hydroxy group of the fluorine-based compound and a sixth reaction of reacting the fourth intermediate compound with a terminal hydroxy group of the obtained fifth intermediate compound are sequentially performed. As a result, a sixth intermediate compound having terminal groups corresponding to R¹ and R⁵ at both terminals of a chain-like structure having three PFPE chains and two linking groups including a partial structure corresponding to —CR³R⁴— disposed between adjacent PFPE chains is obtained.

### (Seventh Reaction)

Finally, the protective group included in the sixth intermediate compound obtained by the sixth reaction is deprotected by an appropriate method, whereby a fluorine-containing ether compound is produced.

The above-described steps make it possible to produce a fluorine-containing ether compound in which z in Formula (1) is 2, groups represented by —CR³R⁴— included in two linking groups are different from each other, terminal groups represented by R¹ and R⁵ are the same as or different from each other, and three PFPE chains represented by R² are the same as one another or at least one is different.

In the above-described fifth production method, the third reaction and the fourth reaction are performed after the first reaction and the second reaction are performed, but the first reaction and the second reaction may be performed after the third reaction and the fourth reaction are performed.

In addition, in the above-described fifth production method, the second intermediate compound is used in the fifth reaction, and the fourth intermediate compound is used in the sixth reaction, but the fourth intermediate compound may be used in the fifth reaction, and the second intermediate compound may be used in the sixth reaction.

### [Lubricant for Magnetic Recording Medium]

A lubricant for a magnetic recording medium of the present embodiment contains a fluorine-containing ether compound represented by Formula (1).

In the lubricant of the present embodiment, known materials used as materials for lubricants can be mixed and used as necessary as long as the characteristics are not impaired by the inclusion of the fluorine-containing ether compound represented by Formula (1).

Specific examples of the known materials include FOMBLIN (registered trademark) ZDIAC, FOMBLIN ZDEAL, and FOMBLIN AM-2001 (all of which are manufactured by Solvay Solexis), and Moresco A20H (manufactured by Moresco Corporation). The known material used by being mixed with the lubricant of the present embodiment preferably has a number-average molecular weight of 500 to 10000.

In a case where the lubricant of the present embodiment includes another material of the fluorine-containing ether compound represented by Formula (1), the content of the fluorine-containing ether compound represented by Formula (1) in the lubricant of the present embodiment is preferably 50% by mass or more, and more preferably 70% by mass or more. The content of the fluorine-containing ether compound represented by Formula (1) may be 80% by mass or more or 90% by mass or more.

Since the lubricant of the present embodiment includes the fluorine-containing ether compound represented by Formula (1), the lubricant has excellent adhesion to the protective layer, and can coat the surface of the protective layer at a high coating rate even in a case where the thickness is reduced, and thus can form a lubricating layer having good coatability. Therefore, with the lubricant of the present embodiment, even in a case where the thickness is small, the chemical substance resistance of the magnetic recording medium can be increased, and a lubricating layer having an excellent pickup suppression effect can be obtained.

### [Magnetic Recording Medium]

A magnetic recording medium of the present embodiment has at least a magnetic layer, a protective layer, and a lubricating layer sequentially provided on a substrate.

In the magnetic recording medium of the present embodiment, one or two or more underlayers can be provided between the substrate and the magnetic layer as necessary. In addition, an adhesion layer and/or a soft magnetic layer can be provided between the underlayer and the substrate.

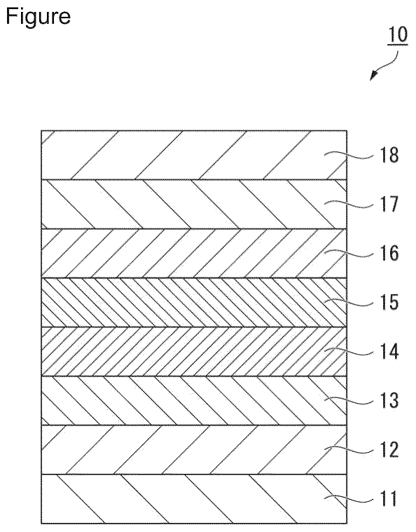

FIG. 1 is a schematic cross-sectional view showing one embodiment of a magnetic recording medium of the present invention.

A magnetic recording medium 10 of the present embodiment has a structure in which an adhesion layer 12, a soft magnetic layer 13, a first underlayer 14, a second underlayer 15, a magnetic layer 16, a protective layer 17, and a lubricating layer 18 are sequentially provided on a substrate 11.

“Substrate” As the substrate 11, for example, a non-magnetic substrate having a film made of NiP or a NiP alloy formed on a substrate made of a metal such as Al or an Al alloy or an alloy material can be used.

In addition, as the substrate 11, a non-magnetic substrate made of a non-metallic material such as glass, ceramics, silicon, silicon carbide, carbon, or a resin may be used, or a non-magnetic substrate having a film of NiP or a NiP alloy formed on a base made of these non-metallic materials may be used.

### “Adhesion Layer”

The adhesion layer 12 prevents the progress of the corrosion of the substrate 11, which occurs in a case where the substrate 11 and the soft magnetic layer 13 provided on the adhesion layer 12 are disposed in contact with each other.

The material of the adhesion layer 12 can be appropriately selected from, for example, Cr, a Cr alloy, Ti, a Ti alloy, CrTi, NiAl, and an AlRu alloy. The adhesion layer 12 can be formed by, for example, a sputtering method.

### “Soft Magnetic Layer”

The soft magnetic layer 13 preferably has a structure in which a first soft magnetic film, an interlayer made of a Ru film, and a second soft magnetic film are laminated in this order. That is, it is preferable that the soft magnetic layer 13 has a structure in which the soft magnetic films above and below the interlayer are anti-ferro-coupling (AFC) bonded by interposing the interlayer made of the Ru film between two soft magnetic films.

Examples of the material of the first soft magnetic film and the second soft magnetic film include a CoZrTa alloy and a CoFe alloy.

It is preferable that any one of Zr, Ta, or Nb is added to the CoFe alloy used for the first soft magnetic film and the second soft magnetic film. This makes the amorphization of the first soft magnetic film and the second soft magnetic film promoted. As a result, it becomes possible to improve the orientation of the first underlayer (seed layer) and to reduce the floating height of the magnetic head.

The soft magnetic layer 13 can be formed by, for example, a sputtering method.

### “First Underlayer”

The first underlayer 14 is a layer that controls the orientations and crystal sizes of the second underlayer 15 and the magnetic layer 16 provided thereon.

Examples of the first underlayer 14 include layers made of a Cr layer, a Ta layer, a Ru layer, a CrMo alloy layer, a CoW alloy layer, a CrW alloy layer, a CrV alloy layer, or a CrTi alloy layer.

The first underlayer 14 can be formed by, for example, a sputtering method.

### “Second Underlayer”

The second underlayer 15 is a layer that controls the orientation of the magnetic layer 16 to be good. The second underlayer 15 is preferably a layer made of Ru or a Ru alloy.

The second underlayer 15 may be a single layer or may be composed of a plurality of layers. In a case where the second underlayer 15 is made of a plurality of layers, all of the layers may be composed of the same material, or at least one layer may be composed of a different material.

The second underlayer 15 can be formed by, for example, a sputtering method.

### “Magnetic Layer”

The magnetic layer 16 is made of a magnetic film in which the magnetization easy axis is oriented in a direction perpendicular or horizontal to the substrate surface. The magnetic layer 16 is a layer containing Co and Pt. In order to improve the SNR characteristics, the magnetic layer 16 may be a layer containing an oxide, Cr, B, Cu, Ta, Zr, or the like.

Examples of the oxide contained in the magnetic layer 16 include SiO₂, SiO, Cr₂O₃, CoO, Ta₂O₃, and TiO₂.

The magnetic layer 16 may be composed of one layer, or may be composed of a plurality of magnetic layers made of materials having different compositions. For example, in a case where the magnetic layer 16 is made up of three layers of a first magnetic layer, a second magnetic layer, and a third magnetic layer, which are laminated in this order, the first magnetic layer is preferably a granular structure made of a material containing Co, Cr, and Pt, and further containing an oxide. As the oxide contained in the first magnetic layer, for example, an oxide of Cr, Si, Ta, Al, Ti, Mg, Co, or the like is preferably used. Among these, TiO₂, Cr₂O₃, SiO₂, and the like can be suitably used. In addition, the first magnetic layer is preferably made of a composite oxide obtained by adding two or more kinds of oxides. Among these, Cr₂O₃—SiO₂, Cr₂O₃—TiO₂, SiO₂—TiO₂, and the like can be suitably used. The first magnetic layer can contain one or more elements selected from B, Ta, Mo, Cu, Nd, W, Nb, Sm, Tb, Ru, and Re, in addition to Co, Cr, Pt, and the oxide.

The same material as that of the first magnetic layer can be used for the second magnetic layer. The second magnetic layer is preferably a granular structure.

The third magnetic layer is preferably a non-granular structure made of a material containing Co, Cr, and Pt and not containing an oxide. The third magnetic layer can contain one or more elements selected from B, Ta, Mo, Cu, Nd, W, Nb, Sm, Tb, Ru, Re, and Mn, in addition to Co, Cr, and Pt.

In a case where the magnetic layer 16 is formed of a plurality of magnetic layers, it is preferable that a non-magnetic layer is provided between the adjacent magnetic layers. In a case where the magnetic layer 16 is made up of three layers of a first magnetic layer, a second magnetic layer, and a third magnetic layer, it is preferable to provide non-magnetic layers between the first magnetic layer and the second magnetic layer and between the second magnetic layer and the third magnetic layer.

As the non-magnetic layer provided between the adjacent magnetic layers in the magnetic layer 16, for example, Ru, a Ru alloy, a CoCr alloy, or a CoCrX1 alloy (X1 represents one or two or more elements selected from Pt, Ta, Zr, Re, Ru, Cu, Nb, Ni, Mn, Ge, Si, O, N, W, Mo, Ti, V, or B) can be suitably used.

It is preferable to use an alloy material containing an oxide, a metal nitride, or a metal carbide in the non-magnetic layer provided between the adjacent magnetic layers in the magnetic layer 16. Specifically, as the oxide, for example, SiO₂, Al₂O₃, Ta₂O₅, Cr₂O₃, MgO, Y₂O₃, TiO₂, or the like can be used. As the metal nitride, for example, ALN, Si₃N₄, TaN, CrN, or the like can be used. As the metal carbide, for example, TaC, BC, SiC, and the like can be used.

The non-magnetic layer can be formed by, for example, a sputtering method.

In order to realize a higher recording density, the magnetic layer 16 is preferably a magnetic layer of perpendicular magnetic recording in which the magnetization easy axis is oriented in a direction perpendicular to the substrate surface. The magnetic layer 16 may be a magnetic layer of in-plane magnetic recording.

The magnetic layer 16 may be formed by any known method in the related art, such as a vapor deposition method, an ion beam sputtering method, or a magnetron sputtering method. The magnetic layer 16 is usually formed by a sputtering method.

### “Protective Layer”

The protective layer 17 protects the magnetic layer 16. The protective layer 17 may be composed of one layer or may be composed of a plurality of layers. Examples of the material of the protective layer 17 include carbon, carbon containing nitrogen, and silicon carbide. As the protective layer 17, a carbon-based protective layer can be preferably used, and an amorphous carbon protective layer is particularly preferable. It is preferable that the protective layer 17 is a carbon-based protective layer since the interaction with the polar group contained in the fluorine-containing ether compound in the lubricating layer 18 is further enhanced.

The adhesion force between the carbon-based protective layer and the lubricating layer 18 can be controlled by producing the carbon-based protective layer with hydrogenated carbon and/or nitrided carbon and adjusting the hydrogen content and/or the nitrogen content in the carbon-based protective layer. The hydrogen content in the carbon-based protective layer is preferably 3 atomic % to 20 atomic % in the case of being measured by hydrogen forward scattering (HFS). In addition, the nitrogen content in the carbon-based protective layer is preferably 4 atomic % to 15 atomic % in the case of being measured by X-ray photoelectron spectroscopy (XPS).

The hydrogen and/or nitrogen contained in the carbon-based protective layer does not need to be uniformly contained in the entire carbon-based protective layer. The carbon-based protective layer is suitably a composition gradient layer in which nitrogen is contained on the lubricating layer 18 side of the protective layer 17 and hydrogen is contained on the magnetic layer 16 side of the protective layer 17. In this case, the adhesion force between the magnetic layer 16 and the lubricating layer 18 and the carbon-based protective layer is further improved.

The film thickness of the protective layer 17 is preferably 1 nm to 7 nm. In a case where the film thickness of the protective layer 17 is 1 nm or more, the performance as the protective layer 17 can be sufficiently obtained. In a case where the film thickness of the protective layer 17 is 7 nm or less, it is preferable from the viewpoint of thinning the protective layer 17.

As a method for forming the protective layer 17, a sputtering method using a target material containing carbon, a chemical vapor deposition (CVD) method using a hydrocarbon raw material such as ethylene or toluene, an ion beam deposition (IBD) method, or the like can be used.

In a case where a carbon-based protective layer is formed as the protective layer 17, the film can be formed by, for example, a DC magnetron sputtering method. In particular, in a case where a carbon-based protective layer is formed as the protective layer 17, it is preferable to form an amorphous carbon protective layer by a plasma CVD method. The amorphous carbon protective layer formed by the plasma CVD method has a uniform surface and a small roughness.

### “Lubricating Layer”

The lubricating layer 18 prevents the contamination of the magnetic recording medium 10. In addition, the lubricating layer 18 reduces the frictional force of the magnetic head of a magnetic recording and reproducing device that slides on the magnetic recording medium 10, and improves the durability of the magnetic recording medium 10.

As shown in FIG. 1, the lubricating layer 18 is formed in contact with the protective layer 17. The lubricating layer 18 contains the above-described fluorine-containing ether compound.

In a case where the protective layer 17 disposed below the lubricating layer 18 is a carbon-based protective layer, particularly, the lubricating layer 18 is bonded to the protective layer 17 with a high bonding force. As a result, even in a case where the thickness of the lubricating layer 18 is small, it becomes easy to obtain the magnetic recording medium 10 in which the surface of the protective layer 17 is coated at a high coating rate, and the contamination of the surface of the magnetic recording medium 10 can be effectively prevented.

The average film thickness of the lubricating layer 18 is preferably 0.5 nm (5 Å) to 2.0 nm (20 Å) and more preferably 0.5 nm (5 Å) to 1.0 nm (10 Å). In a case where the average film thickness of the lubricating layer 18 is 0.5 nm or more, the lubricating layer 18 is formed with a uniform film thickness without being formed in an island shape or a mesh shape. Therefore, the surface of the protective layer 17 can be coated with the lubricating layer 18 at a high coating rate. In addition, when the average film thickness of the lubricating layer 18 is set to 2.0 nm or less, the lubricating layer 18 can be sufficiently thinned, and the floating height of the magnetic head can be sufficiently reduced.

In a case where the surface of the protective layer 17 is not coated with the lubricating layer 18 at a sufficiently high coating rate, an environmental substance adsorbed on the surface of the magnetic recording medium 10 passes through the gap of the lubricating layer 18 and invade the lower layer of the lubricating layer 18. The environmental substance that has intruded the lower layer of the lubricating layer 18 is adsorbed and bonded to the protective layer 17, and generates a contaminant. In addition, in the case of magnetic recording and reproducing, this contaminant (aggregation component) adheres (is transferred) to the magnetic head as smear, and damages the magnetic head or degrades the magnetic recording and reproducing characteristics of the magnetic recording and reproducing device.

Examples of the environmental substance that generates a contaminant include a siloxane compound (cyclic siloxane, linear siloxane), an ionic impurity, a hydrocarbon having a relatively high molecular weight, such as octacosane, and a plasticizer such as dioctyl phthalate. Examples of a metal ion contained in the ionic impurity include a sodium ion and a potassium ion. Examples of an inorganic ion contained in the ionic impurity include a chlorine ion, a bromine ion, a nitrate ion, a sulfate ion, and an ammonium ion. Examples of an organic ion contained in the ionic impurity include an oxalate ion and a formate ion.

### “Method of Forming Lubricating Layer”

Examples of a method for forming the lubricating layer 18 include a method of preparing a magnetic recording medium in the middle of manufacturing in which each layer up to the protective layer 17 has been formed on the substrate 11, applying a solution for forming the lubricating layer onto the protective layer 17, and drying the solution.

The solution for forming the lubricating layer is obtained by dispersing and dissolving the lubricant for a magnetic recording medium of the above-described embodiment in a solvent as necessary and adjusting the viscosity and the concentration to be suitable for the application method.

Examples of the solvent used in the solution for forming the lubricating layer include fluorine-based solvents such as Vertrel (registered trademark) XF (product name, manufactured by Mitsui DuPont Fluorochemicals Co., Ltd.) and/or ASAHIKLIN (registered trademark) AE-3000 (product name, manufactured by AGC Inc.).

The method for applying the solution for forming the lubricating layer is not particularly limited, and examples thereof include a spin coating method, a spray coating method, a paper coating method, and a dipping method.

In a case where the dipping method is used, for example, the following method can be used. First, the substrate 11 on which each layer up to the protective layer 17 has been formed is immersed in the solution for forming the lubricating layer, which has been put into an immersion tank of a dip coating device. Next, the substrate 11 is pulled up from the immersion tank at a predetermined speed. As a result, the solution for forming the lubricating layer is applied onto the surface of the protective layer 17 on the substrate 11.

The use of the dipping method makes it possible to uniformly apply the solution for forming the lubricating layer to the surface of the protective layer 17, and makes it possible to form the lubricating layer 18 on the protective layer 17 with a uniform film thickness.

In the present embodiment, it is preferable to perform a heat treatment on the substrate 11 on which the lubricating layer 18 has been formed. The heat treatment improves the adhesion between the lubricating layer 18 and the protective layer 17 and improves the adhesion force between the lubricating layer 18 and the protective layer 17.

The heat treatment temperature is preferably set to 100° C. to 180° C. In a case where the heat treatment temperature is 100° C. or higher, an effect of improving the adhesion between the lubricating layer 18 and the protective layer 17 can be sufficiently obtained. In addition, when the heat treatment temperature is set to 180° C. or lower, the thermal decomposition of the lubricating layer 18 can be prevented. The heat treatment time is preferably set to 10 to 120 minutes.

The magnetic recording medium 10 of the present embodiment has at least the magnetic layer 16, the protective layer 17, and the lubricating layer 18 sequentially provided on the substrate 11. In the magnetic recording medium 10 of the present embodiment, the lubricating layer 18 containing the above-described fluorine-containing ether compound is formed in contact with the protective layer 17. This lubricating layer 18 has excellent adhesion to the protective layer 17, has an appropriate surface energy, can coat the surface of the protective layer 17 at a high coating rate in a uniform coating state even in a case where the thickness is small, and has good coatability. Therefore, in the magnetic recording medium 10 of the present embodiment, an environmental substance that generates a contaminant such as an ionic impurity is prevented from intruding through the gap of the lubricating layer 18. In addition, the lubricating layer 18 in the magnetic recording medium 10 of the present embodiment is less likely to generate a foreign matter (smear) and can suppress pickup. Therefore, the magnetic recording medium 10 of the present embodiment has a small amount of a contaminant present on the surface, has excellent chemical substance resistance, and has good reliability and durability.

### EXAMPLES

Hereinafter, the present invention will be described in more detail with reference to Examples and Comparative Examples. The present invention is not limited to Examples described below.

### Example 1

A compound represented by Formula (TA) was obtained by the following method.

### (First Reaction)

20 g of a compound represented by
HOCH₂CF₂CF₂O(CF₂CF₂CF₂O)ᵢCF₂CF₂CH₂OH (1 representing the average degree of polymerization in the formula was 3.8) (number-average molecular weight: 909, molecular weight distribution: 1.1), 1.95 g of 3,4-dihydro-2H-pyran, and 44 mL of a mixed solution of ASAHIKLIN (registered trademark) AE-3000 (manufactured by AGC Inc.) as a fluorine-based solvent and dichloromethane (volume ratio: 1:1) were charged into a 300 mL eggplant flask under a nitrogen gas atmosphere, and the mixture was stirred at 0° C. until uniform, thereby obtaining a mixed solution. 0.084 g of p-toluenesulfonic acid monohydrate was added to this mixed solution, stirred at 0° C. for 30 minutes, and then stirred at room temperature for 2 hours to carry out a reaction.

The reaction product obtained after the reaction was cooled to 0° C., 50 mL of saturated aqueous sodium bicarbonate was added thereto, and the reaction was stopped. The obtained reaction solution was transferred to a separatory funnel and extracted three times with 100 mL of ethyl acetate. The organic layer was washed with saline and dewatered with anhydrous sodium sulfate. After filtering off a desiccant, the filtrate was thickened, and the residue was purified by silica gel column chromatography to obtain 10.8 g of a compound represented by Formula (11) as an intermediate compound (1A-1).

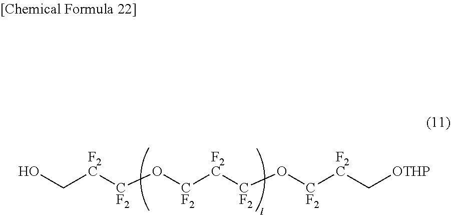

(In Formula (11), THP represents a tetrahydropyranyl group, and 1 representing the average degree of polymerization represents 3.8.)

### (Second Reaction)

10.8 g of a compound represented by Formula (11) as an intermediate compound (1A-1) (number-average molecular weight: 993, 10.9 mmol) 3.40 g of a compound (A1) represented by the following formula acquired by the following synthetic method (molecular weight: 624, 5.45 mmol), 0.36 g of potassium iodide (molecular weight: 166, 2.2 mmol), and 22 mL of N,N′-dimethylformamide (DMF) were charged into a 200 mL eggplant flask under a nitrogen gas atmosphere, and stirred at room temperature to obtain a mixed solution. 10.6 g of cesium carbonate (molecular weight: 325, 32.7 mmol) was added to this mixed solution, stirred and reacted at 70° C. for 5 hours.

The compound represented by Formula (A1) was synthesized by reacting benzyl chloromethyl ether with diethyl malonate, then, reducing the ester with lithium borohydride, and reacting the resulting two primary hydroxy groups with p-toluenesulfonyl chloride.

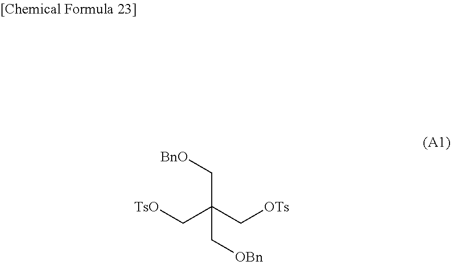 [SMILES: Cc1ccc(S(=O)(=O)OCC(COCc2ccccc2)(COCc2ccccc2)COS(=O)(=O)c2ccc(C)cc2)cc1]

(In Formula (A1), Ts represents a p-toluenesulfonyl group, and Bn represents a benzyl group.)

The reaction solution obtained after the reaction was returned to room temperature, 28 g of a 10% hydrogen chloride/methanol solution (hydrogen chloride-methanol reagent (5% to 10%) manufactured by Tokyo Chemical Industry Co., Ltd.) was added thereto, and the mixture was stirred at room temperature for 2 hours. The reaction solution was transferred little by little to a separatory funnel containing 100 mL of saline, and the mixture was extracted three times with 200 mL of ethyl acetate. The organic layer was washed with 100 mL of saline, 100 mL of saturated aqueous sodium bicarbonate, and 100 mL of saline in this order, and dewatered with anhydrous sodium sulfate. After filtering off a desiccant, the filtrate was thickened, and the residue was purified by silica gel column chromatography. The above-described steps made 8.0 g of a compound represented by Formula (12) obtained as an intermediate compound (1A-2).

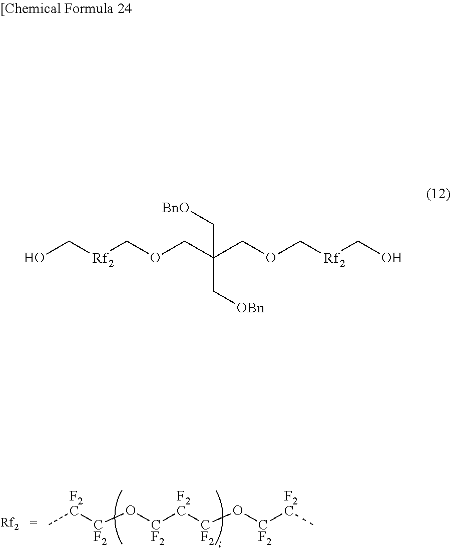

(In Formula (12), Bn represents a benzyl group, Rf₂ is represented by the above formula, and 1 representing the average degree of polymerization in Rf₂ represents 3.8.)

### (Third Reaction)

5.0 g of a compound represented by Formula (12) (number-average molecular weight: 2,102, 2.4 mmol) as an intermediate compound (1A-2), 1.4 g of a compound (Ep-1) represented by Formula (13) (molecular weight: 202, 7.1 mmol), and 22 mL of t-butanol were charged into a 200 mL eggplant flask under a nitrogen gas atmosphere, and the mixture was stirred at room temperature until uniform. 0.053 g of potassium tert-butoxide (molecular weight: 113, 0.48 mmol) was further added to this uniform liquid, and the mixture was stirred at 70° C. for 16 hours to carry out a reaction.

A compound (Ep-1) represented by Formula (13) was synthesized by a method of oxidizing a compound in which a hydroxy group of ethylene glycol monoallyl ether was protected using dihydropyran.

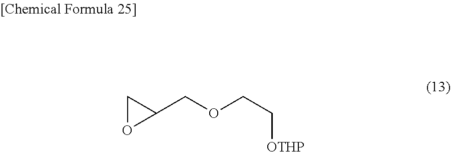

(In Formula (13), THP represents a tetrahydropyranyl group)

A reaction product obtained after the reaction was cooled to 25° C., transferred to a separatory funnel containing 100 mL of water, and extracted three times with 100 mL of ethyl acetate. The organic layer was washed with water and dewatered with anhydrous sodium sulfate. After filtering off a desiccant, the filtrate was thickened, and the residue was purified by silica gel column chromatography to obtain 4.5 g of a compound represented by Formula (14) as an intermediate compound (1A-3).

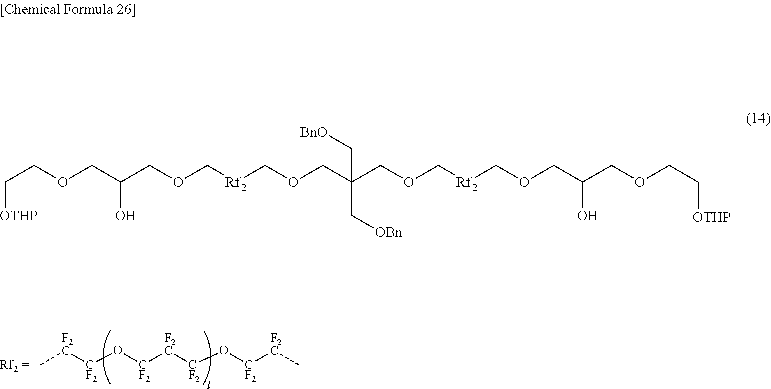

(In Formula (14), THP represents a tetrahydropyranyl group, Bn represents a benzyl group, Rf₂ is represented by the above formula, and 1 representing the average degree of polymerization in Rf₂ represents 3.8.)

### (Fourth Reaction)

4.5 g of a compound represented by Formula (14) (number-average molecular weight: 2,506, 1.8 mmol) as an intermediate compound (1A-3), 45 mL of methanol, and 4.5 mL of formic acid were charged into a 200 mL eggplant flask under a nitrogen gas atmosphere, and the mixture was stirred at room temperature until uniform. 0.45 g of palladium carbon (Pd/C) was further added to this uniform liquid, and the mixture was stirred at 70° C. for 5 hours to carry out a reaction.

The reaction solution obtained after the reaction was filtered to remove Pd/C, and the filtrate was thickened. After the thickening, the residue was purified by silica gel column chromatography to obtain 3.6 g of a compound (1 Å) represented by Formula (15) (number-average molecular weight: 2156, 1.7 mmol).

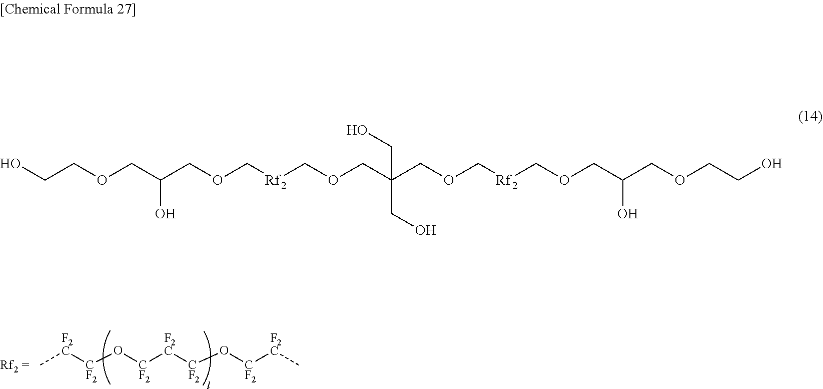

(In Formula (15), Rf₂ is represented by the above formula, and 1 representing the average degree of polymerization in Rf₂ represents 3.8.)

The obtained compound (TA) was subjected to ¹H-NMR and ¹⁹F-NMR measurements, and the structure was identified from the following results.

¹H-NMR (CD₃COCD₃): δ [ppm]=3.39 to 4.34 (40H)

¹⁹F-NMR (CD₃COCD₃): δ[ppm]=−84.0 to −83.0 (30.4F), −86.4 (8F), −124.3 (8F), −130.0 to −129.0 (15.2F)

### Example 2

### (Third Reaction)

The operation up to the third reaction was performed in the same manner as in Example 1 except that in the third reaction in Example 1 described above, 1.5 g (molecular weight: 216, 7.1 mmol) of a compound (Ep-2) represented by Formula (16) was used instead of the compound (Ep-1) represented by Formula (13), and 4.6 g of an intermediate compound (1B-3) represented by Formula (17) was obtained.

The compound (Ep-2) represented by Formula (16) was synthesized by protecting one hydroxy group of 1,3-propanediol with a tetrahydropyranyl (THP) group and reacting the other hydroxy group with epibromohydrin.

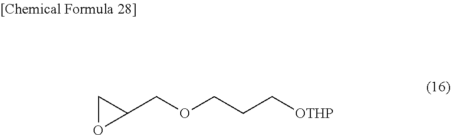

(In Formula (16), THP represents a tetrahydropyranyl group)

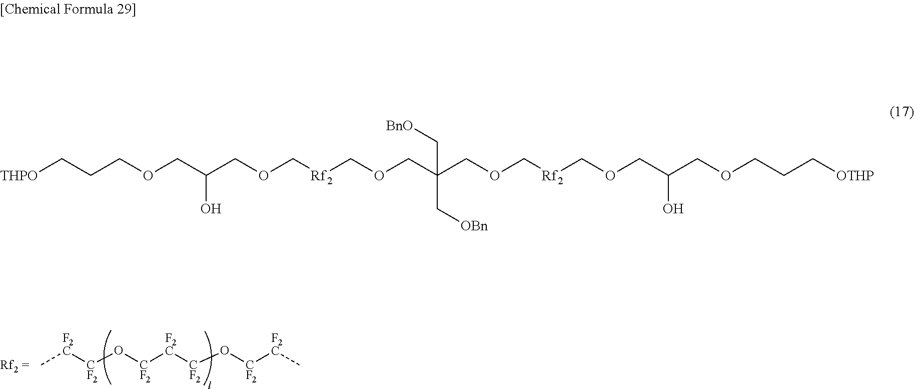

(In Formula (17), THP represents a tetrahydropyranyl group, Bn represents a benzyl group, Rf₂ is represented by the above formula, and 1 representing the average degree of polymerization in Rf₂ represents 3.8.)

### (Fourth Reaction)

4.5 g of a compound represented by Formula (17) (number-average molecular weight: 2,534, 1.8 mmol) as an intermediate compound (1B-3), 45 mL of methanol, and 4.5 mL of formic acid were charged into a 200 mL eggplant flask under a nitrogen gas atmosphere, and the mixture was stirred at room temperature until uniform. 0.45 g of palladium carbon (Pd/C) was further added to this uniform liquid, and the mixture was stirred at 70° C. for 5 hours to carry out a reaction.

The reaction solution obtained after the reaction was filtered to remove Pd/C, and the filtrate was thickened. After the thickening, the residue was purified by silica gel column chromatography to obtain 3.6 g of a compound (1B) represented by Formula (18) (number-average molecular weight: 2184, 1.7 mmol).

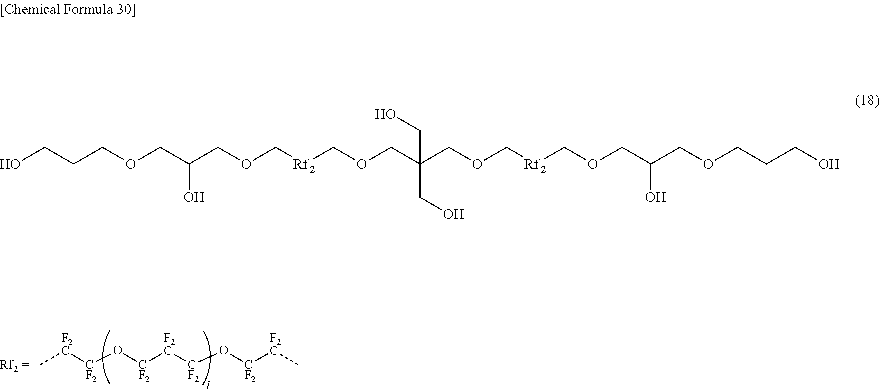

(In Formula (18), Rf₂ is represented by the above formula, and 1 representing the average degree of polymerization in Rf₂ represents 3.8.)

The obtained compound (1B) was subjected to ¹H-NMR and ¹⁹F-NMR measurements, and the structure was identified from the following results.

¹H-NMR (CD₃COCD₃): δ [ppm]=1.64 to 1.83 (4H), 3.36 to 4.34 (40H)

¹⁹F-NMR (CD₃COCD₃): δ [ppm]=−84.1 to −83.0 (30.4F), −86.2 (8F), −124.4 (8F), −130.0 to −129.1 (15.2F)

### Example 3

### (Third Reaction)

The operation up to the third reaction was performed in the same manner as in Example 1 except that in the third reaction in Example 1 described above, 2.3 g (molecular weight: 320, 7.1 mmol) of a compound (Ep-3) represented by Formula (20) was used instead of the compound (Ep-1) represented by Formula (13), and 4.4 g of an intermediate compound (1C-3) represented by Formula (19) was obtained.

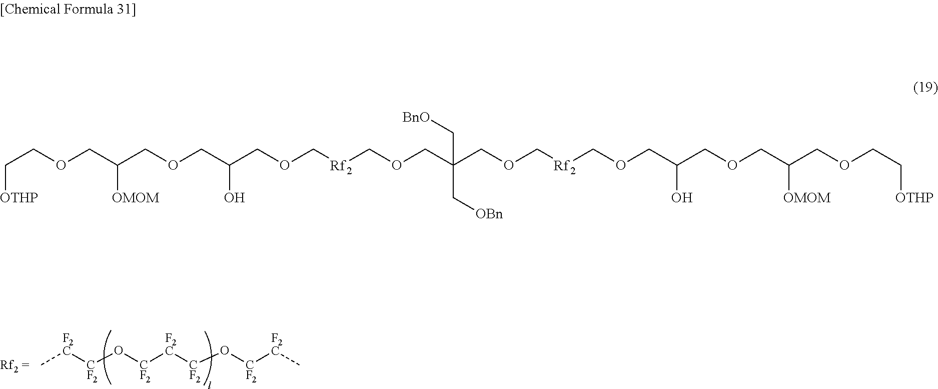

(In Formula (19), THP represents a tetrahydropyranyl group, Bn represents a benzyl group, and MOM represents a methoxymethyl group. Rf₂ is represented by the above formula, and 1 representing the average degree of polymerization in Rf₂ represents 3.8.)

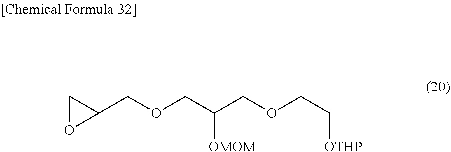

(In Formula (20), THP represents a tetrahydropyranyl group, and MOM represents a methoxymethyl group.)

A compound (Ep-3) represented by Formula (20) was synthesized by the following method.

A tert-butyldimethylsilyl (TBS) group was introduced as a protective group into a primary hydroxy group of 3-allyloxy-1,2-propanediol, and a methoxymethyl (MOM) group was introduced as a protective group into a secondary hydroxy group of the obtained compound. The TBS group was removed from the obtained compound, and 2-bromoethoxytetrahydropyran was reacted with the generated primary hydroxy group. The double bond of the obtained compound was oxidized. The above-described steps made a compound (Ep-3) represented by Formula (20) obtained.

### (Fourth Reaction)

4.4 g of a compound represented by Formula (19) (number-average molecular weight: 2,743, 1.6 mmol) as an intermediate compound (1C-3), 44 mL of methanol, and 4.4 mL of formic acid were charged into a 200 mL eggplant flask under a nitrogen gas atmosphere, and the mixture was stirred at room temperature until uniform. 0.44 g of palladium carbon (Pd/C) was further added to this uniform liquid, and the mixture was stirred at 70° C. for 5 hours to carry out a reaction.

The reaction solution obtained after the reaction was filtered to remove Pd/C, and the filtrate was thickened. After the thickening, the residue was purified by silica gel column chromatography to obtain 3.4 g of a compound (IC) represented by Formula (21) (number-average molecular weight: 2305, 1.5 mmol).

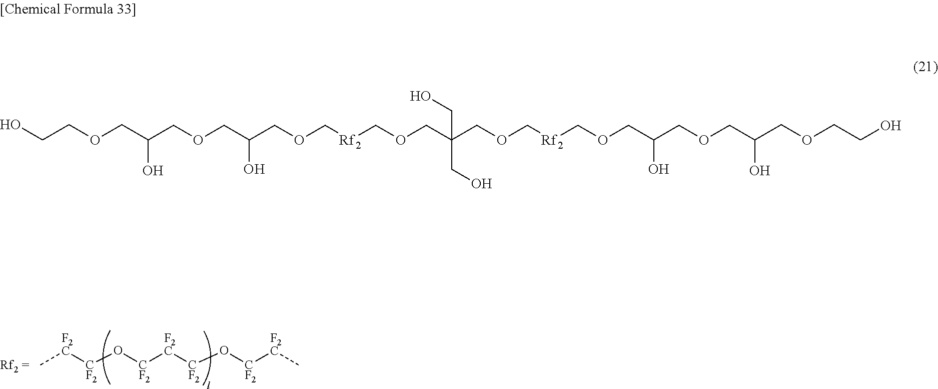

(In Formula (21), Rf₂ is represented by the above formula, and 1 representing the average degree of polymerization in Rf₂ represents 3.8.)

The obtained compound (1C) was subjected to ¹H-NMR and ¹⁹F-NMR measurements, and the structure was identified from the following results.

¹H-NMR (CD₃COCD₃): δ [ppm]=3.34 to 4.37 (52H)

¹⁹F-NMR (CD₃COCD₃): δ [ppm]=−83.9 to −83.1 (30.4F), −86.4 (8F), −124.2 (8F), −130.2 to −129.0 (15.2F)

### Example 4

### (Third Reaction)

The operation up to the third reaction was performed in the same manner as in Example 1 except that in the third reaction in Example 1 described above, 2.4 g (molecular weight: 334, 7.1 mmol) of a compound (Ep-4) represented by Formula (23) was used instead of the compound (Ep-1) represented by Formula (13), and 4.6 g of an intermediate compound (1D-3) represented by Formula (22) was obtained.

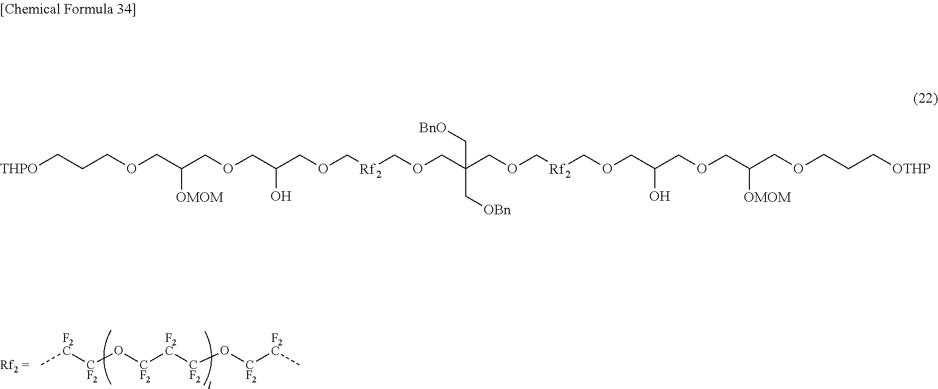

(In Formula (22), Bn represents a benzyl group, THP represents a tetrahydropyranyl group, and MOM represents a methoxymethyl group. Rf₂ is represented by the above formula, and 1 representing the average degree of polymerization in Rf₂ represents 3.8.)

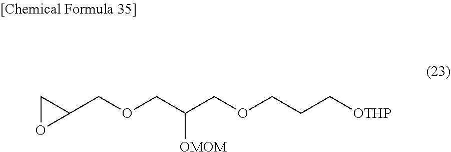

(In Formula (23), THP represents a tetrahydropyranyl group, and MOM represents a methoxymethyl group.)

A compound (Ep-4) represented by Formula (23) was synthesized by the following method.

A tert-butyldimethylsilyl (TBS) group was introduced as a protective group into a primary hydroxy group of 3-allyloxy-1,2-propanediol, and a methoxymethyl (MOM) group was introduced as a protective group into a secondary hydroxy group of the obtained compound. The TBS group was removed from the obtained compound, and 2-(chloropropoxy)tetrahydro-2H-pyran was reacted with the generated primary hydroxy group. The double bond of the obtained compound was oxidized. The above-described steps made a compound (Ep-4) represented by Formula (23) obtained.

### (Fourth Reaction)

4.4 g of a compound represented by Formula (22) (number-average molecular weight: 2,771, 1.6 mmol) as an intermediate compound (1D-3), 44 mL of methanol, and 4.4 mL of formic acid were charged into a 200 mL eggplant flask under a nitrogen gas atmosphere, and the mixture was stirred at room temperature until uniform. 0.44 g of palladium carbon (Pd/C) was further added to this uniform liquid, and the mixture was stirred at 70° C. for 5 hours to carry out a reaction.

The reaction solution obtained after the reaction was filtered to remove Pd/C, and the filtrate was thickened. After the thickening, the residue was purified by silica gel column chromatography to obtain 3.1 g of a compound (1D) represented by Formula (24) (number-average molecular weight: 2333, 1.3 mmol).

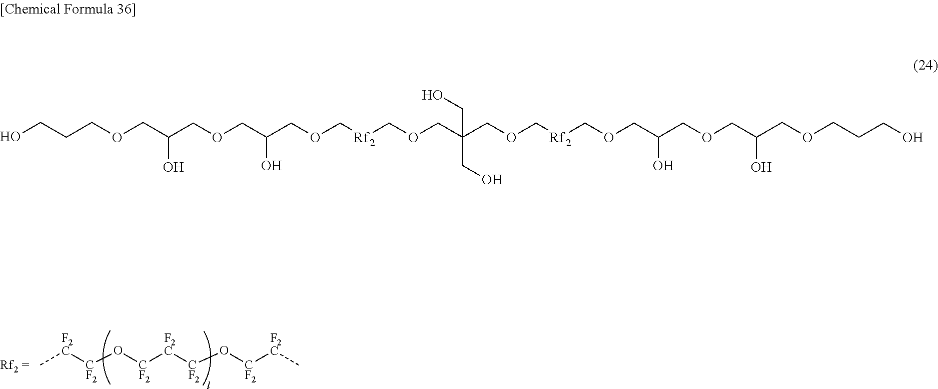

(In Formula (24), Rf₂ is represented by the above formula, and 1 representing the average degree of polymerization in Rf₂ represents 3.8.)

The obtained compound (1D) was subjected to ¹H-NMR and ¹⁹F-NMR measurements, and the structure was identified from the following results.

¹H-NMR (CD₃COCD₃): δ [ppm]=1.63 to 1.83 (4H), 3.37 to 4.36 (52H)

¹⁹F-NMR (CD₃COCD₃): δ [ppm]=−83.9 to −83.1 (30.4F), −86.4 (8F), −124.2 (8F), −130.2 to −128.9 (15.2F)

### Example 5

### (Third Reaction)

The operation up to the third reaction was performed in the same manner as in Example 1 except that in the third reaction in Example 1 described above, 1.5 g (molecular weight: 216, 7.1 mmol) of a compound (Ep-5) represented by Formula (26) was used instead of the compound (Ep-1) represented by Formula (13), and 4.4 g of an intermediate compound (1E-3) represented by Formula (25) was obtained.

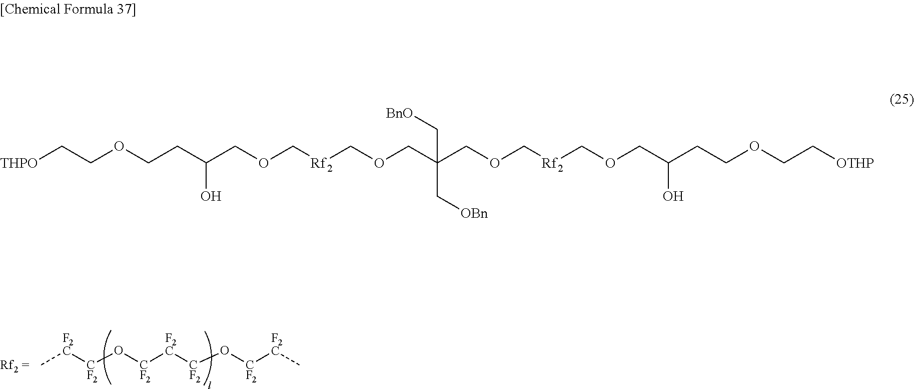

(In Formula (25), Bn represents a benzyl group, and THP represents a tetrahydropyranyl group. Rf₂ is represented by the above formula, and 1 representing the average degree of polymerization in Rf₂ represents 3.8.)

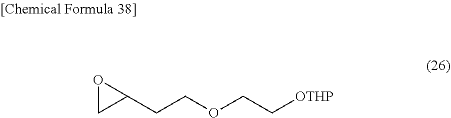

(In Formula (26), THP represents a tetrahydropyranyl group)

A compound (Ep-5) represented by Formula (26) was synthesized by oxidizing a double bond of a compound obtained by reacting 3-buten-1-ol with 2-bromoethoxytetrahydropyran.

### (Fourth Reaction)

4.4 g of a compound represented by Formula (25) (number-average molecular weight: 2,534, 1.7 mmol) as an intermediate compound (TE-3), 44 mL of methanol, and 4.4 mL of formic acid were charged into a 200 mL eggplant flask under a nitrogen gas atmosphere, and the mixture was stirred at room temperature until uniform. 0.44 g of palladium carbon (Pd/C) was further added to this uniform liquid, and the mixture was stirred at 70° C. for 5 hours to carry out a reaction.

The reaction solution obtained after the reaction was filtered to remove Pd/C, and the filtrate was thickened. After the thickening, the residue was purified by silica gel column chromatography to obtain 3.3 g of a compound (1E) represented by Formula (27) (number-average molecular weight: 2184, 1.6 mmol).

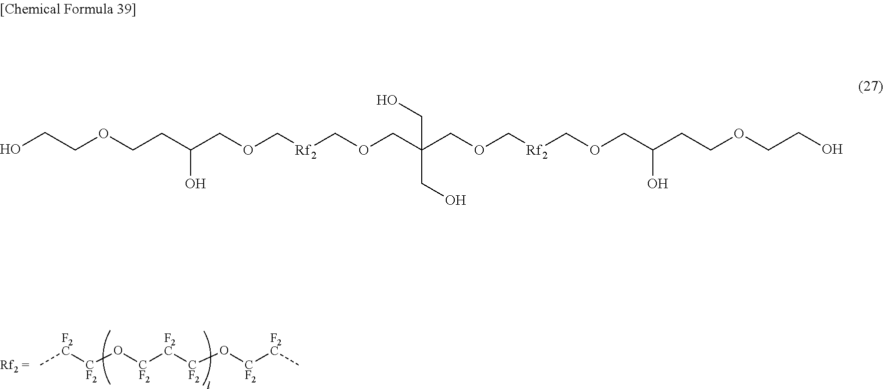

(In Formula (27), Rf₂ is represented by the above formula, and 1 representing the average degree of polymerization in Rf₂ represents 3.8.)

The obtained compound (1E) was subjected to ¹H-NMR and ¹⁹F-NMR measurements, and the structure was identified from the following results.

¹H-NMR (CD₃COCD₃): δ [ppm]=1.65 to 1.79 (4H), 3.41 to 4.33 (40H)

¹⁹F-NMR (CD₃COCD₃): a[ppm]=−84.0 to −83.0 (30.4F), −86.4 (8F), −124.3 (8F), −130.0 to −129.0 (15.2F)

### Example 6

### (Third Reaction)

The operation up to the third reaction was performed in the same manner as in Example 1 except that in the third reaction in Example 1 described above, 2.4 g (molecular weight: 334, 7.1 mmol) of a compound (Ep-6) represented by Formula (29) was used instead of the compound (Ep-1) represented by Formula (13), and 4.6 g of an intermediate compound (1F-3) represented by Formula (28) was obtained.

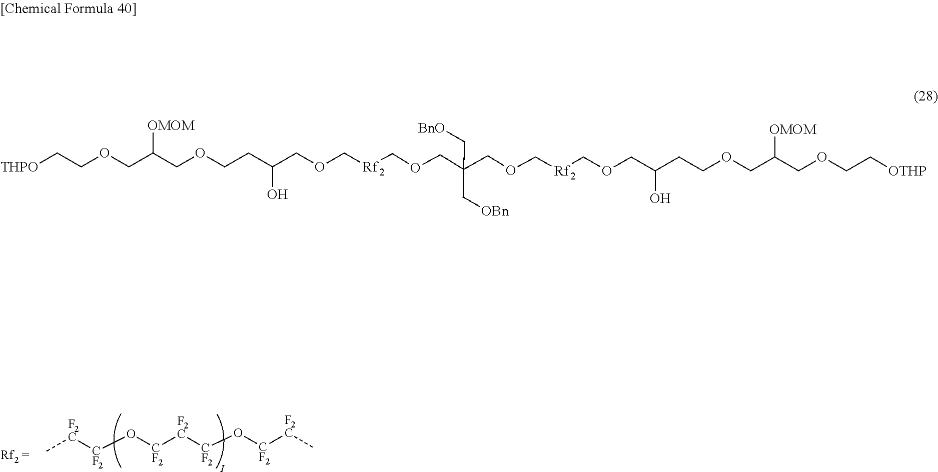

(In Formula (28), Bn represents a benzyl group, THP represents a tetrahydropyranyl group, and MOM represents a methoxymethyl group. Rf₂ is represented by the above formula, and 1 representing the average degree of polymerization in Rf₂ represents 3.8.)

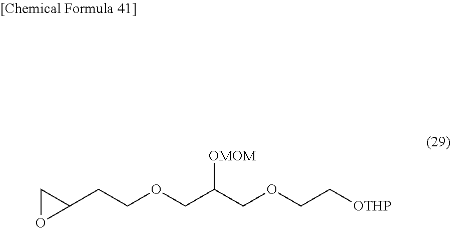

(In Formula (29), THP represents a tetrahydropyranyl group, and MOM represents a methoxymethyl group.)

A compound (Ep-6) represented by Formula (29) was synthesized by the following method.

A hydroxy group of ethylene glycol monoallyl ether was protected using dihydropyran, and the double bond of the obtained compound was oxidized. An epoxy group of the compound obtained by oxidizing a double bond was reacted with a hydroxy group of 3-butene-1-ol. The secondary hydroxy group of the obtained compound was protected with a methoxymethyl (MOM) group, and the double bond of the obtained compound was oxidized. The above-described steps made a compound (Ep-6) represented by Formula (29) obtained.

### (Fourth Reaction)

4.6 g of a compound represented by Formula (28) (number-average molecular weight: 2,771, 1.7 mmol) as an intermediate compound (1F-3), 46 mL of methanol, and 4.6 mL of formic acid were charged into a 200 mL eggplant flask under a nitrogen gas atmosphere, and the mixture was stirred at room temperature until uniform. 0.46 g of palladium carbon (Pd/C) was further added to this uniform liquid, and the mixture was stirred at 70° C. for 5 hours to carry out a reaction.

The reaction solution obtained after the reaction was filtered to remove Pd/C, and the filtrate was thickened. After the thickening, the residue was purified by silica gel column chromatography to obtain 3.6 g of a compound (1F) represented by Formula (30) (number-average molecular weight: 2333, 1.6 mmol).

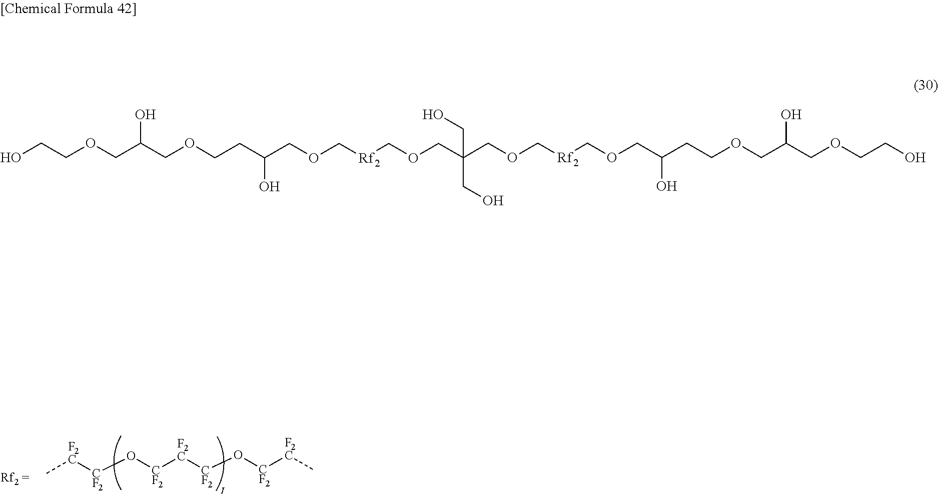

(In Formula (30), Rf₂ is represented by the above formula, and 1 representing the average degree of polymerization in Rf₂ represents 3.8.)

The obtained compound (1F) was subjected to ¹H-NMR and ¹⁹F-NMR measurements, and the structure was identified from the following results.

¹H-NMR (CD₃COCD₃): δ [ppm]=1.64 to 1.80 (4H), 3.40 to 4.33 (52H)

¹⁹F-NMR (CD₃COCD₃): δ [ppm]=−84.1 to −83.0 (30.4F), −86.4 (8F), −124.4 (8F), −130.1 to −128.9 (15.2F)

### Example 7

A compound represented by Formula (1G) was obtained by the following method.

### (First Reaction)

20 g of a compound represented by

HOCH₂CF₂O(CF₂CF₂O)ⱼ(CF₂O)ₖCF₂CH₂OH (j representing the average degree of polymerization in the formula was 4.0, and k representing the average degree of polymerization was 4.0) (number-average molecular weight: 906, molecular weight distribution: 1.1), 1.95 g of 3,4-dihydro-2H-pyran, and 44 mL of a mixed solution of ASAHIKLIN (registered trademark) AE-3000 (manufactured by AGC Inc.) as a fluorine-based solvent and dichloromethane (volume ratio: 1:1) were charged into a 300 mL eggplant flask under a nitrogen gas atmosphere, and the mixture was stirred at 0° C. until uniform, thereby obtaining a mixed solution. 0.084 g of p-toluenesulfonic acid monohydrate was added to this mixed solution, stirred at 0° C. for 30 minutes, and then stirred at room temperature for 2 hours to carry out a reaction.

The reaction product obtained after the reaction was cooled to 0° C., 50 mL of saturated aqueous sodium bicarbonate was added thereto, and the reaction was stopped. The obtained reaction solution was transferred to a separatory funnel and extracted three times with 100 mL of ethyl acetate. The organic layer was washed with saline and dewatered with anhydrous sodium sulfate. After filtering off a desiccant, the filtrate was thickened, and the residue was purified by silica gel column chromatography to obtain 10.5 g of a compound represented by Formula (31) as an intermediate compound (1G-1).

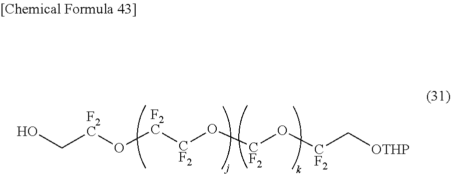

(In Formula (31), THP represents a tetrahydropyranyl group, j representing the average degree of polymerization is 4.0, and k representing the average degree of polymerization is 4.0.)

### (Second Reaction)

10.5 g of a compound represented by Formula (31) as an intermediate compound (1G-1) (number-average molecular weight: 990, 10.6 mmol), 3.4 g of a compound represented by Formula (Al) (molecular weight: 624, 5.45 mmol), 0.36 g of potassium iodide (molecular weight: 166, 2.2 mmol), and 22 mL of N,N′-dimethylformamide (DMF) were charged into a 200 mL eggplant flask under a nitrogen gas atmosphere, and stirred at room temperature to obtain a mixed solution. 10.64 g of cesium carbonate (molecular weight: 325, 32.7 mmol) was added to this mixed solution, stirred and reacted at 70° C. for 5 hours.

The reaction solution obtained after the reaction was returned to room temperature, 28 g of a 10% hydrogen chloride/methanol solution (hydrogen chloride-methanol reagent (5% to 10%) manufactured by Tokyo Chemical Industry Co., Ltd.) was added thereto, and the mixture was stirred at room temperature for 2 hours. The reaction solution was transferred little by little to a separatory funnel containing 100 mL of saline, and the mixture was extracted three times with 200 mL of ethyl acetate. The organic layer was washed with 100 mL of saline, 100 mL of saturated aqueous sodium bicarbonate, and 100 mL of saline in this order, and dewatered with anhydrous sodium sulfate. After filtering off a desiccant, the filtrate was thickened, and the residue was purified by silica gel column chromatography. The above-described steps made 7.9 g of a compound represented by Formula (32) obtained as an intermediate compound (1G-2).

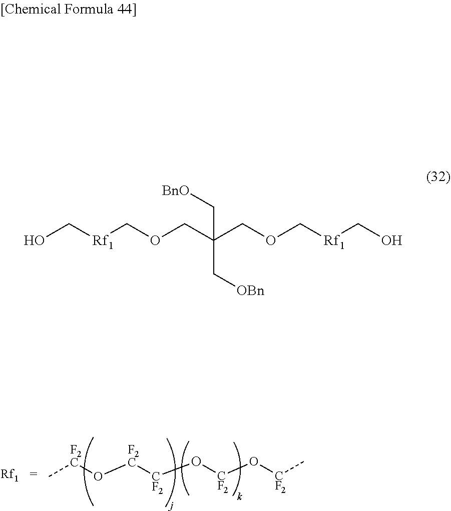

(In Formula (32), Bn represents a benzyl group, Rf₁ is represented by the above formula, j representing the average degree of polymerization in Rf₁ is 4.0, and k representing the average degree of polymerization is 4.0.)

### (Third Reaction)

5.0 g of a compound represented by Formula (32) (number-average molecular weight: 2,096, 2.4 mmol) as an intermediate compound (1G-2), 1.2 g of a compound (Ep-7) represented by Formula (33) (molecular weight: 172, 7.2 mmol), and 22 mL of t-butanol were charged into a 200 mL eggplant flask under a nitrogen gas atmosphere, and the mixture was stirred at room temperature until uniform. 0.054 g of potassium tert-butoxide (molecular weight: 112, 0.54 mmol) was further added to this uniform liquid, and the mixture was stirred at 70° C. for 16 hours to carry out a reaction.

A compound (Ep-7) represented by Formula (33) was synthesized by a method of introducing a tetrahydropyranyl (THP) group into a primary hydroxy group of 3-butene-1-ol and oxidizing a double bond of the obtained compound.

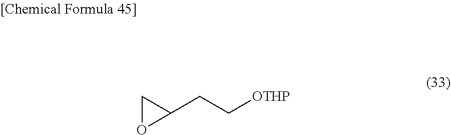

(In Formula (33), THP represents a tetrahydropyranyl group)

A reaction product obtained after the reaction was cooled to 25° C., transferred to a separatory funnel containing 100 mL of water, and extracted three times with 100 mL of ethyl acetate. The organic layer was washed with water and dewatered with anhydrous sodium sulfate. After filtering off a desiccant, the filtrate was thickened, and the residue was purified by silica gel column chromatography to obtain 4.1 g of a compound represented by Formula (34) as an intermediate compound (1G-3).

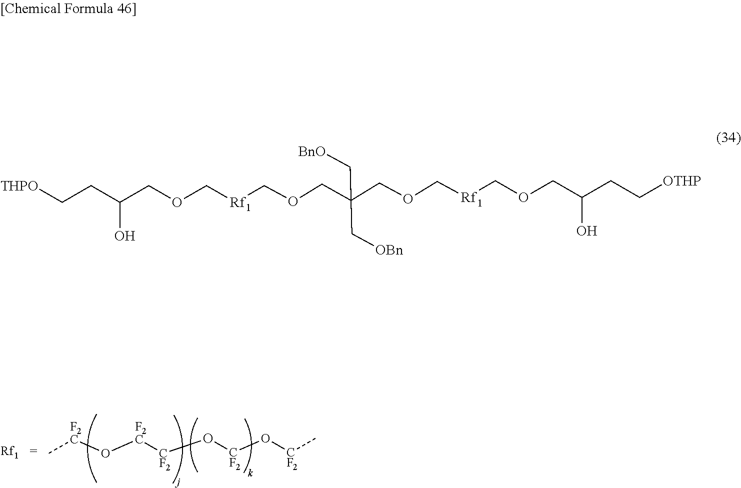

(In Formula (34), THP represents a tetrahydropyranyl group, and Bn represents a benzyl group. Rf₁ is represented by the above formula, j representing the average degree of polymerization in Rf₁ is 4.0, and k representing the average degree of polymerization is 4.0.)

### (Fourth Reaction)

4.1 g of a compound represented by Formula (34) (number-average molecular weight: 2,440, 1.7 mmol) as an intermediate compound (1G-3), 41 mL of methanol, and 4.1 mL of formic acid were charged into a 200 mL eggplant flask under a nitrogen gas atmosphere, and the mixture was stirred at room temperature until uniform. 0.41 g of palladium carbon (Pd/C) was further added to this uniform liquid, and the mixture was stirred at 70° C. for 5 hours to carry out a reaction.

The reaction solution obtained after the reaction was filtered to remove Pd/C, and the filtrate was thickened. After the thickening, the residue was purified by silica gel column chromatography to obtain 3.1 g of a compound (IG) represented by Formula (35) (number-average molecular weight: 2091, 1.6 mmol).

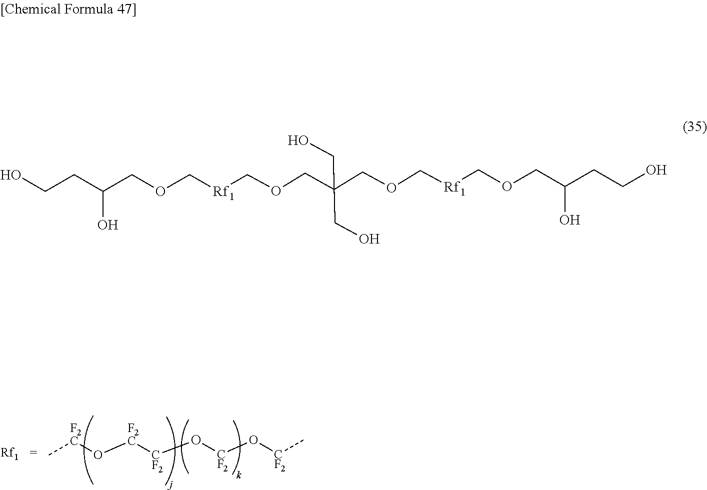

(In Formula (35), Rf₁ is represented by the above formula, j representing the average degree of polymerization in Rf₁ is 4.0, and k representing the average degree of polymerization is 4.0.)

The obtained compound (1G) was subjected to ¹H-NMR and ¹⁹F-NMR measurements, and the structure was identified from the following results.

¹H-NMR (CD₃COCD₃): δ [ppm]=1.66 to 1.79 (4H), 3.42 to 4.34 (32H)

¹⁹F-NMR (CD₃COCD₃): δ [ppm]=−55.6 to −50.6 (16F), −77.7 (4F), −80.3 (4F), −91.0 to −88.5 (32F)

### Example 8

### (Third Reaction)

The operation up to the third reaction was performed in the same manner as in Example 7 except that in the third reaction in Example 7 described above, 1.4 g (molecular weight: 200, 7.2 mmol) of a compound (Ep-8) represented by Formula (37) was used instead of the compound (Ep-7) represented by Formula (33), and 4.2 g of an intermediate compound (1H-3) represented by Formula (36) was obtained.

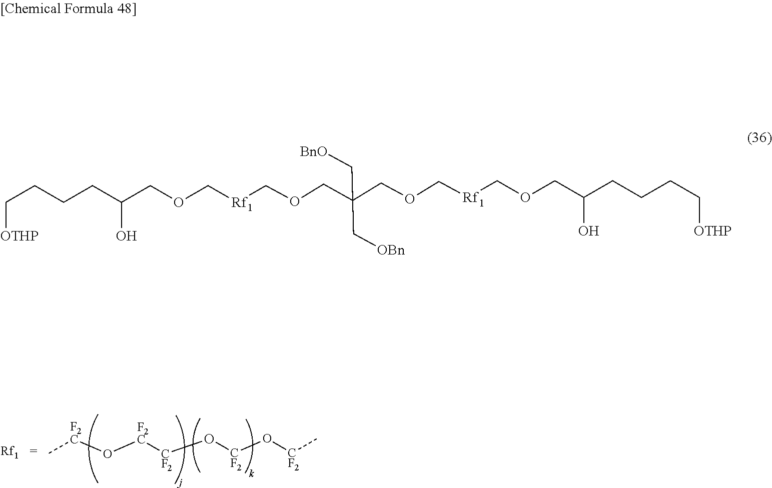

(In Formula (36), THP represents a tetrahydropyranyl group, and Bn represents a benzyl group. Rf₁ is represented by the above formula, j representing the average degree of polymerization in Rf₁ is 4.0, and k representing the average degree of polymerization is 4.0.)

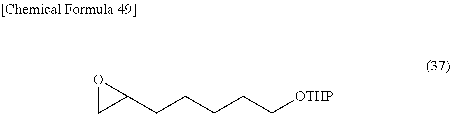

(In Formula (37), THP represents a tetrahydropyranyl group)

A compound (Ep-8) represented by Formula (37) was produced by the following method. That is, the compound was synthesized by a method of introducing a tetrahydropyranyl (THP) group into a primary hydroxy group of 5-hexen-1-ol and oxidizing a double bond of the obtained compound.

### (Fourth Reaction)

4.2 g of a compound represented by Formula (36) (number-average molecular weight: 2,497, 1.7 mmol) as an intermediate compound (1H-3), 42 mL of methanol, and 4.2 mL of formic acid were charged into a 200 mL eggplant flask under a nitrogen gas atmosphere, and the mixture was stirred at room temperature until uniform. 0.42 g of palladium carbon (Pd/C) was further added to this uniform liquid, and the mixture was stirred at 70° C. for 5 hours to carry out a reaction.

The reaction solution obtained after the reaction was filtered to remove Pd/C, and the filtrate was thickened. After the thickening, the residue was purified by silica gel column chromatography to obtain 3.2 g of a compound (1H) represented by Formula (38) (number-average molecular weight: 2147, 1.6 mmol).

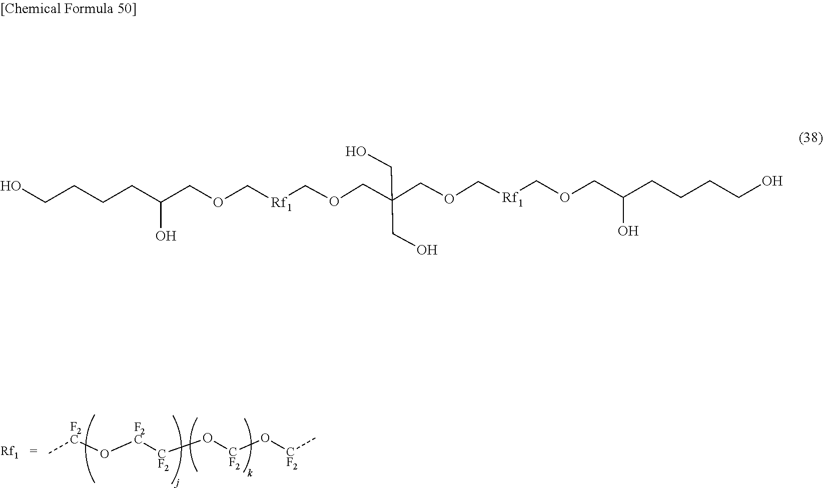

(In Formula (38), Rf₁ is represented by the above formula, j representing the average degree of polymerization in Rf₁ is 4.0, and k representing the average degree of polymerization is 4.0.)

The obtained compound (1H) was subjected to ¹H-NMR and ¹⁹F-NMR measurements, and the structure was identified from the following results.

¹H-NMR (CD₃COCD₃): δ [ppm]=1.35 to 1.83 (12H), 3.36 to 4.32 (32H)

¹⁹F-NMR (CD₃COCD₃): δ [ppm]=−55.5 to −50.6 (16F), −77.6 (4F), −80.3 (4F), −91.1 to −88.4 (32F)

### Example 9

A compound represented by Formula (11) was obtained by the following method.

### (First Reaction)

20 g of a compound represented by HOCH₂CF₂O(CF₂CF₂O)ⱼ(CF₂O)ₖCF₂CH₂OH (j representing the average degree of polymerization in the formula was 6.3, and k representing the average degree of polymerization was 0) (number-average molecular weight: 909, molecular weight distribution: 1.1), 1.94 g of 3,4-dihydro-2H-pyran, and 44 mL of a mixed solution of ASAHIKLIN (registered trademark) AE-3000 (manufactured by AGC Inc.) as a fluorine-based solvent and dichloromethane (volume ratio: 1:1) were charged into a 300 mL eggplant flask under a nitrogen gas atmosphere, and the mixture was stirred at 0° C. until uniform, thereby obtaining a mixed solution. 0.084 g of p-toluenesulfonic acid monohydrate was added to this mixed solution, stirred at 0° C. for 30 minutes, and then stirred at room temperature for 2 hours to carry out a reaction.

The reaction product obtained after the reaction was cooled to 0° C., 50 mL of saturated aqueous sodium bicarbonate was added thereto, and the reaction was stopped. The obtained reaction solution was transferred to a separatory funnel and extracted three times with 100 mL of ethyl acetate. The organic layer was washed with saline and dewatered with anhydrous sodium sulfate. After filtering off a desiccant, the filtrate was thickened, and the residue was purified by silica gel column chromatography to obtain 10.0 g of a compound represented by Formula (39) as an intermediate compound (11-1).

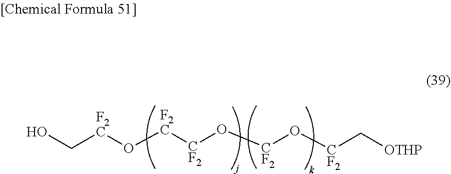

(In Formula (39), THP represents a tetrahydropyranyl group, j representing the average degree of polymerization is 6.3, and k representing the average degree of polymerization is 0.)

### (Second Reaction)

10.0 g of a compound represented by Formula (39) as an intermediate compound (11-1) (number-average molecular weight: 992, 10.1 mmol), 3.16 g of a compound represented by Formula (Al) (molecular weight: 624, 5.0 mmol), 0.33 g of potassium iodide (molecular weight: 166, 2.0 mmol), and 20 mL of N,N′-dimethylformamide (DMF) were charged into a 200 mL eggplant flask under a nitrogen gas atmosphere, and stirred at room temperature to obtain a mixed solution. 9.83 g of cesium carbonate (molecular weight: 325, 30.2 mmol) was added to this mixed solution, stirred and reacted at 70° C. for 5 hours.

The reaction solution obtained after the reaction was returned to room temperature, 26 g of a 10% hydrogen chloride/methanol solution (hydrogen chloride-methanol reagent (5% to 10%) manufactured by Tokyo Chemical Industry Co., Ltd.) was added thereto, and the mixture was stirred at room temperature for 2 hours. The reaction solution was transferred little by little to a separatory funnel containing 100 mL of saline, and the mixture was extracted three times with 200 mL of ethyl acetate. The organic layer was washed with 100 mL of saline, 100 mL of saturated aqueous sodium bicarbonate, and 100 mL of saline in this order, and dewatered with anhydrous sodium sulfate. After filtering off a desiccant, the filtrate was thickened, and the residue was purified by silica gel column chromatography. The above-described steps made 7.4 g of a compound represented by Formula (40) obtained as an intermediate compound (11-2).

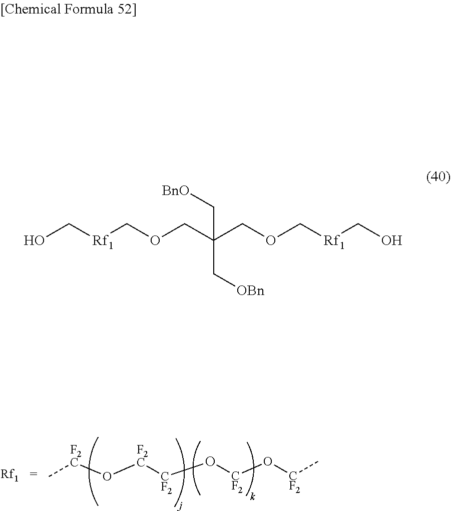

(In Formula (40), Bn represents a benzyl group, Rf₁ is represented by the above formula, j representing the average degree of polymerization in Rf₁ is 6.3, and k representing the average degree of polymerization is 0.)

### (Third Reaction)

5.0 g of a compound represented by Formula (40) (number-average molecular weight: 2,101, 2.4 mmol) as an intermediate compound (11-2), 2.2 g of a compound (Ep-9) represented by Formula (41) (molecular weight: 304, 7.1 mmol), and 22 mL of t-butanol were charged into a 200 mL eggplant flask under a nitrogen gas atmosphere, and the mixture was stirred at room temperature until uniform. 0.053 g of potassium tert-butoxide (molecular weight: 112, 0.48 mmol) was further added to this uniform liquid, and the mixture was stirred at 70° C. for 16 hours to carry out a reaction.

A compound (Ep-9) represented by Formula (41) was synthesized by the following method.

A tetrahydropyranyl (THP) group was introduced into a primary hydroxy group of 4-penten-1-ol, and the double bond of the obtained compound was oxidized. The compound obtained by oxidizing a double bond was reacted with allyl alcohol. The secondary hydroxy group of the obtained compound was protected with a methoxymethyl (MOM) group, and the double bond of the obtained compound was oxidized. The above-described steps made a compound (Ep-9) represented by Formula (41) obtained.

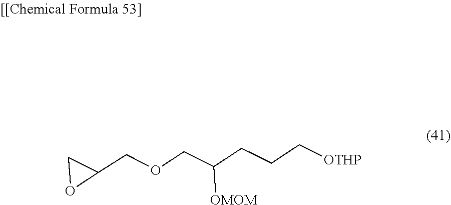

(In Formula (41), THP represents a tetrahydropyranyl group, and MOM represents a methoxymethyl group.)

A reaction product obtained after the reaction was cooled to 25° C., transferred to a separatory funnel containing 100 mL of water, and extracted three times with 100 mL of ethyl acetate. The organic layer was washed with water and dewatered with anhydrous sodium sulfate. After filtering off a desiccant, the filtrate was thickened, and the residue was purified by silica gel column chromatography to obtain 4.6 g of a compound represented by Formula (42) as an intermediate compound (11-3).

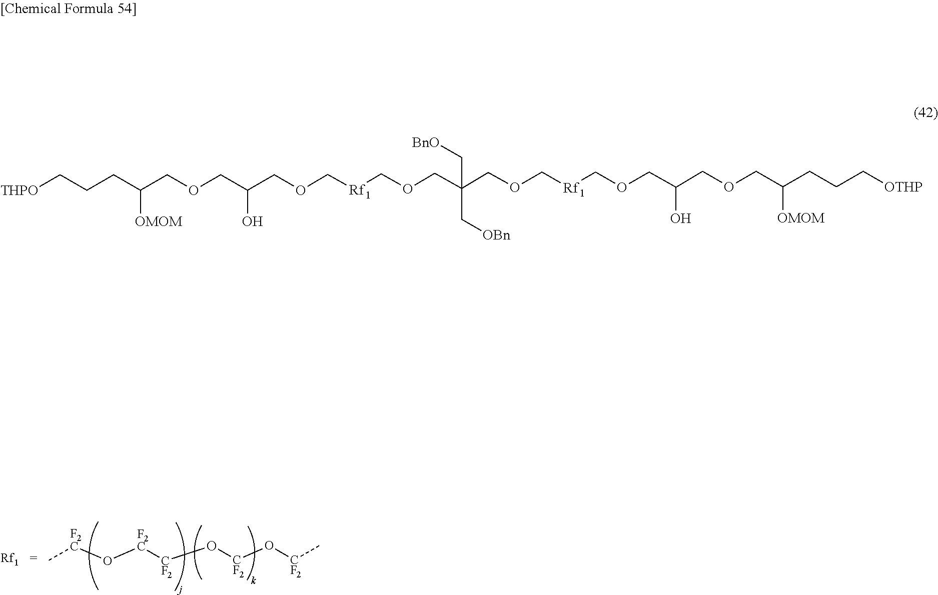

(In Formula (42), THP represents a tetrahydropyranyl group, MOM represents a methoxymethyl group, and Bn represents a benzyl group. Rf₁ is represented by the above formula, j representing the average degree of polymerization in Rf₁ is 6.3, and k representing the average degree of polymerization is 0.)

### (Fourth Reaction)

4.6 g of a compound represented by Formula (42) (number-average molecular weight: 2,710, 1.7 mmol) as an intermediate compound (11-3), 46 mL of methanol, and 4.6 mL of formic acid were charged into a 200 mL eggplant flask under a nitrogen gas atmosphere, and the mixture was stirred at room temperature until uniform. 0.46 g of palladium carbon (Pd/C) was further added to this uniform liquid, and the mixture was stirred at 70° C. for 5 hours to carry out a reaction.

The reaction solution obtained after the reaction was filtered to remove Pd/C, and the filtrate was thickened. After the thickening, the residue was purified by silica gel column chromatography to obtain 3.5 g of a compound (11) represented by Formula (43) (number-average molecular weight: 2272, 1.5 mmol).

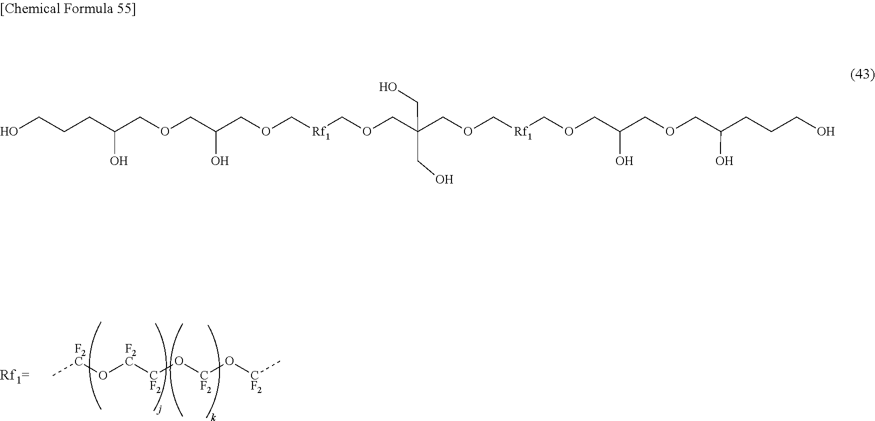

(In Formula (43), Rf₁ is represented by the above formula, j representing the average degree of polymerization in Rf₁ is 6.3, and k representing the average degree of polymerization is 0.)

The obtained compound (11) was subjected to ¹H-NMR and ¹⁹F-NMR measurements, and the structure was identified from the following results.

¹H-NMR (CD₃COCD₃): δ [ppm]=1.34 to 1.67 (8H), 3.39 to 4.34 (44H)

¹⁹F-NMR (CD₃COCD₃): δ[ppm]=−78.6 (4F), −81.3 (4F), −90.0 to −88.5 (50.4F)

### Example 10

### (First Reaction)

10 g of a compound represented by HOCH₂CF₂CF₂O (CF₂CF₂CF₂O)₁CF₂CF₂CH₂OH (1 in the formula represents an average degree of polymerization of 3.8) (number-average molecular weight: 909, molecular weight distribution: 1.1), 2.4 g of a compound (Ep-10) represented by Formula (44) (molecular weight: 272, 8.8 mmol), and 10 mL of t-butanol were charged into a 200 mL eggplant flask under a nitrogen gas atmosphere, and the mixture was stirred at room temperature until uniform. 0.247 g of potassium tert-butoxide (molecular weight: 112, 2.2 mmol) was further added to this uniform liquid, and the mixture was stirred at 70° C. for 16 hours to carry out a reaction.

A compound (Ep-10) represented by Formula (44) was synthesized by the following method.

1,3-diallyloxy-2-propanol was reacted with 3,4-dihydro-2H-pyran. One side double bond of the obtained compound was oxidized using m-chloroperbenzoic acid. The above-described steps made a compound (Ep-10) represented by Formula (44) obtained.

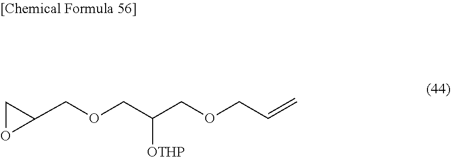

(In Formula (44), THP represents a tetrahydropyranyl group)

A reaction product obtained after the reaction was cooled to 25° C., transferred to a separatory funnel containing 100 mL of water, and extracted three times with 100 mL of ethyl acetate. The organic layer was washed with water and dewatered with anhydrous sodium sulfate. After filtering off a desiccant, the filtrate was thickened, and the residue was purified by silica gel column chromatography to obtain 5.5 g of a compound represented by Formula (45) as an intermediate compound (1I-1).

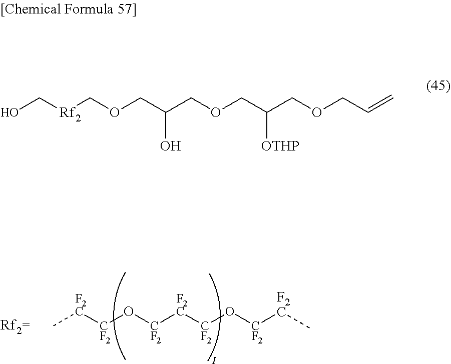

(In Formula (45), THP represents a tetrahydropyranyl group, Rf₂ is represented by the above formula, and 1 representing the average degree of polymerization in Rf₂ represents 3.8.)

(Second reaction) 5.5 g of a compound represented by Formula (45) as an intermediate compound (1J-1) (number-average molecular weight: 1181, 4.7 mmol), 0.70 g of a compound represented by Formula (S1) (molecular weight: 302, 2.3 mmol), 0.16 g of potassium iodide (molecular weight: 166, 0.9 mmol), and 7 mL of N,N′-dimethylformamide (DMF) were charged into a 200 mL eggplant flask under a nitrogen gas atmosphere, and stirred at room temperature to obtain a mixed solution. 4.5 g of cesium carbonate (molecular weight: 325, 14.0 mmol) was added to this mixed solution, stirred and reacted at 70° C. for 5 hours.

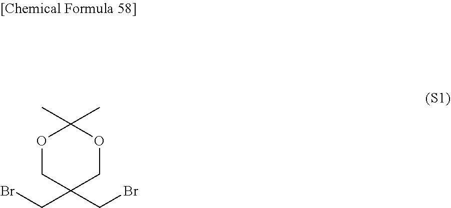

The reaction solution obtained after the reaction was returned to room temperature, 24 g of a 10% hydrogen chloride/methanol solution (hydrogen chloride-methanol reagent (5% to 10%) manufactured by Tokyo Chemical Industry Co., Ltd.) was added thereto, and the mixture was stirred at room temperature for 2 hours. The reaction solution was transferred little by little to a separatory funnel containing 100 mL of saline, and the mixture was extracted three times with 200 mL of ethyl acetate. The organic layer was washed with 100 mL of saline, 100 mL of saturated aqueous sodium bicarbonate, and 100 mL of saline in this order, and dewatered with anhydrous sodium sulfate. After filtering off a desiccant, the filtrate was thickened, and the residue was purified by silica gel column chromatography. The above-described steps made 3.2 g (number-average molecular weight: 2297, 1.4 mmol) of a compound (1J) represented by Formula (46) obtained.

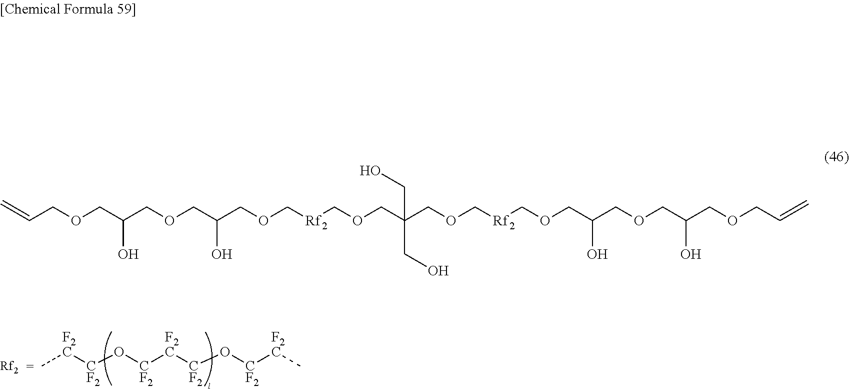

(In Formula (46), Rf₂ is represented by the above formula, and 1 representing the average degree of polymerization in Rf₂ represents 3.8.)

The obtained compound (1J) was subjected to ¹H-NMR and ¹⁹F-NMR measurements, and the structure was identified from the following results.

¹H-NMR (CD₃COCD₃): δ f[ppm]=3.39 to 4.34 (46H), 5.14 to 5.22 (2H), 5.26 to 5.35 (2H), 5.87 to 5.91 (2H)

¹⁹F-NMR (CD₃COCD₃): δ [ppm]=−84.0 to −83.0 (30.4F), −86.4 (8F), −124.3 (8F), −130.0 to −129.0 (15.2F)

### Example 11

### (First Reaction)

10 g of a compound represented by HOCH₂CF₂CF₂O (CF₂CF₂CF₂O)ᵢCF₂CF₂CH₂OH (1 in the formula represents an average degree of polymerization of 3.8) (number-average molecular weight: 909, molecular weight distribution: 1.1), 2.6 g of a compound (Ep-11) represented by Formula (47) (molecular weight: 300, 8.8 mmol), and 10 mL of t-butanol were charged into a 200 mL eggplant flask under a nitrogen gas atmosphere, and the mixture was stirred at room temperature until uniform. 0.247 g of potassium tert-butoxide (molecular weight: 112, 2.2 mmol) was further added to this uniform liquid, and the mixture was stirred at 70° C. for 16 hours to carry out a reaction.

A compound (Ep-11) represented by Formula (47) was synthesized by the following method.

2 equivalents of 3-butene-1-ol was reacted with 1 equivalent of epichlorohydrin. The obtained compound was reacted with 3,4-dihydro-2H-pyran to protect the secondary hydroxy group of the compound with a tetrahydropyranyl (THP) group. One side double bond of the obtained compound was oxidized using m-chloroperbenzoic acid. The above-described steps made a compound (Ep-11) represented by Formula (47) obtained.

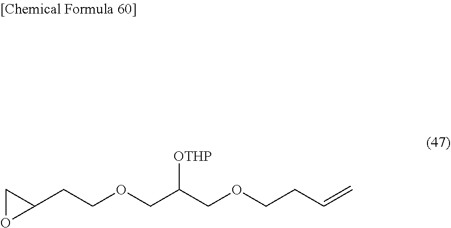

(In Formula (47), THP represents a tetrahydropyranyl group)

A reaction product obtained after the reaction was cooled to 25° C., transferred to a separatory funnel containing 100 mL of water, and extracted three times with 100 mL of ethyl acetate. The organic layer was washed with water and dewatered with anhydrous sodium sulfate. After filtering off a desiccant, the filtrate was thickened, and the residue was purified by silica gel column chromatography to obtain 5.6 g of a compound represented by Formula (48) as an intermediate compound (1K-1).

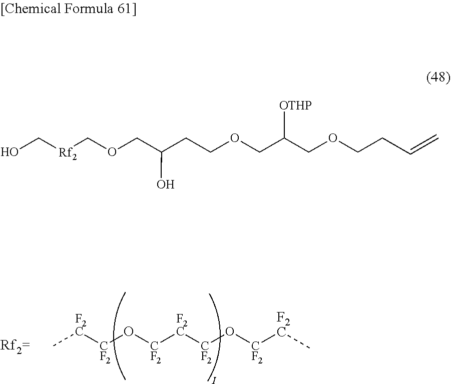

(In Formula (48), THP represents a tetrahydropyranyl group, Rf₂ is represented by the above formula, and 1 representing the average degree of polymerization in Rf₂ represents 3.8.)

### (Second Reaction)

5.6 g of a compound represented by Formula (48) as an intermediate compound (1K-1) (number-average molecular weight: 1209, 4.6 mmol), 0.70 g of a compound represented by Formula (Si) (molecular weight: 302, 2.3 mmol), 0.15 g of potassium iodide (molecular weight: 166, 0.9 mmol), and 7 mL of N,N′-dimethylformamide (DMF) were charged into a 200 mL eggplant flask under a nitrogen gas atmosphere, and stirred at room temperature to obtain a mixed solution. 4.5 g of cesium carbonate (molecular weight: 325, 13.9 mmol) was added to this mixed solution, stirred and reacted at 70° C. for 5 hours.

The reaction solution obtained after the reaction was returned to room temperature, 24 g of a 10% hydrogen chloride/methanol solution (hydrogen chloride-methanol reagent (5% to 10%) manufactured by Tokyo Chemical Industry Co., Ltd.) was added thereto, and the mixture was stirred at room temperature for 2 hours. The reaction solution was transferred little by little to a separatory funnel containing 100 mL of saline, and the mixture was extracted three times with 200 mL of ethyl acetate. The organic layer was washed with 100 mL of saline, 100 mL of saturated aqueous sodium bicarbonate, and 100 mL of saline in this order, and dewatered with anhydrous sodium sulfate. After filtering off a desiccant, the filtrate was thickened, and the residue was purified by silica gel column chromatography. The above-described steps made 2.8 g (number-average molecular weight: 2,353, 1.2 mmol) of a compound (1K) represented by Formula (49) obtained.

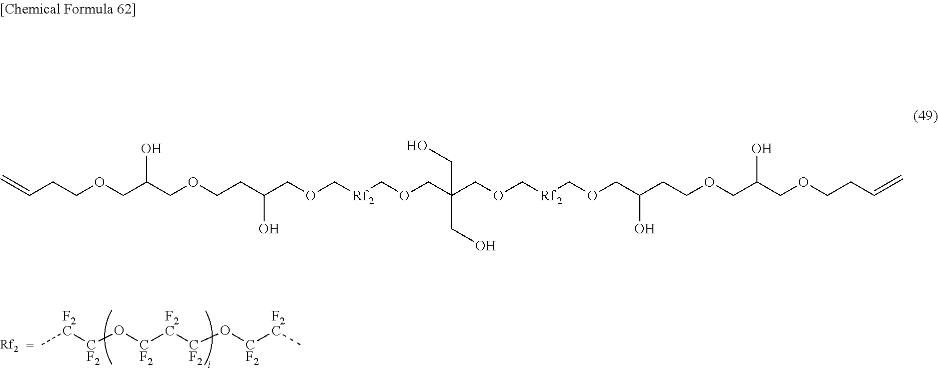

(In Formula (49), Rf₂ is represented by the above formula, and 1 representing the average degree of polymerization in Rf₂ represents 3.8.)

The obtained compound (1K) was subjected to ¹H-NMR and ¹⁹F-NMR measurements, and the structure was identified from the following results. ¹H-NMR (CD₃COCD₃): δ [ppm]=1.66 to 1.81 (4H), 2.33 to 2.43 (4H), 3.39 to 4.34 (46H), 5.14 to 5.22 (2H), 5.26 to 5.35 (2H), 5.87 to 5.91 (2H) ¹⁹F-NMR (CD₃COCD₃): δ[ppm]=−84.0 to −83.0 (30.4F), −86.4 (8F), −124.3 (8F), −130.0 to −129.0 (15.2F)

### Example 12

### (Third Reaction)

The operation up to the third reaction was performed in the same manner as in Example 9 except that in the third reaction in Example 9 described above, 2.1 g (molecular weight: 299, 7.1 mmol) of a compound (Ep-12) represented by Formula (50) was used instead of the compound (Ep-9) represented by Formula (41), and 4.4 g of an intermediate compound (1L-3) represented by Formula (51) was obtained.

A compound (Ep-12) represented by Formula (50) was synthesized by the following method.

A reactant obtained by reacting cyanopropanol with epibromohydrin was hydrolyzed. The primary hydroxy group of the obtained compound was protected with a tert-butyldimethylsilyl group, and then the secondary hydroxy group was protected with a tetrahydropyranyl group. The tert-butyldimethylsilyl group was deprotected from the compound in which the secondary hydroxy group was protected, and the compound was reacted with epibromohydrin. The above-described steps made a compound (Ep-12) represented by Formula (50) obtained.

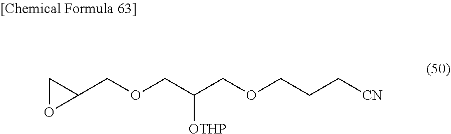

(In Formula (50), THP represents a tetrahydropyranyl group)

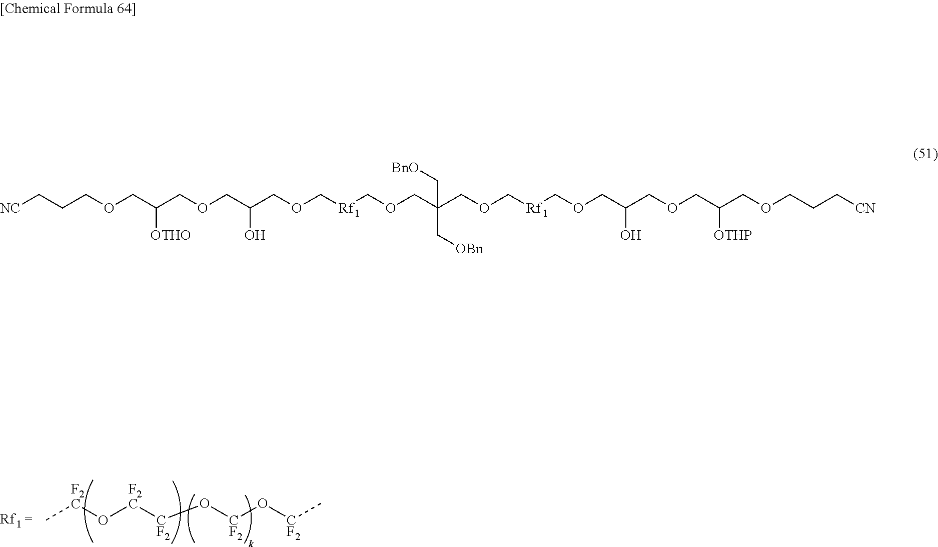

(In Formula (51), THP represents a tetrahydropyranyl group, and Bn represents a benzyl group. Rf₁ is represented by the above formula, j representing the average degree of polymerization in Rf₁ is 6.3, and k representing the average degree of polymerization is 0.)

### (Fourth Reaction)

4.4 g of a compound represented by Formula (51) (number-average molecular weight: 2,700, 1.6 mmol) as an intermediate compound (1L-3), 44 mL of methanol, and 4.4 mL of formic acid were charged into a 200 mL eggplant flask under a nitrogen gas atmosphere, and the mixture was stirred at room temperature until uniform. 0.44 g of palladium carbon (Pd/C) was further added to this uniform liquid, and the mixture was stirred at 70° C. for 5 hours to carry out a reaction.

The reaction solution obtained after the reaction was filtered to remove Pd/C, and the filtrate was thickened. After the thickening, the residue was purified by silica gel column chromatography to obtain 3.4 g of a compound (1L) represented by Formula (52) (number-average molecular weight: 2350, 1.5 mmol).

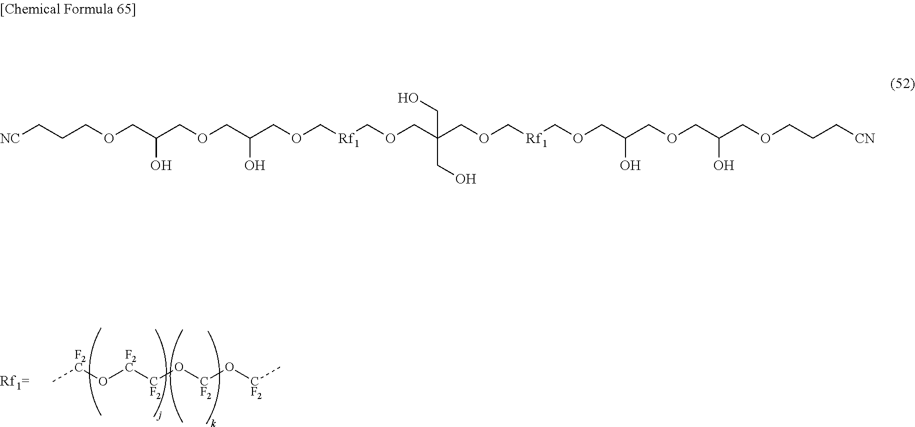

(In Formula (52), Rf₁ is represented by the above formula, j representing the average degree of polymerization in Rf₁ is 6.3, and k representing the average degree of polymerization is 0.)

The obtained compound (1L) was subjected to ¹H-NMR and ¹⁹F-NMR measurements, and the structure was identified from the following results.

¹H-NMR (CD₃COCD₃): δ [ppm]=1.13 to 1.25 (4H), 2.01 to 2.12 (4H), 3.39 to 4.36 (46H) ¹⁹F-NMR (CD₃COCD₃): δ [ppm]=−78.5 (4F), −81.3 (4F), −90.1 to −88.7 (50.4F)

### Example 13

### (Third Reaction)

The operation up to the third reaction was performed in the same manner as in Example 7 except that in the third reaction in Example 7 described above, 2.8 g (molecular weight: 389, 7.2 mmol) of a compound (Ep-13) represented by Formula (54) was used instead of the compound (Ep-7) represented by Formula (33), and 4.6 g of an intermediate compound (1M-3) represented by Formula (53) was obtained.

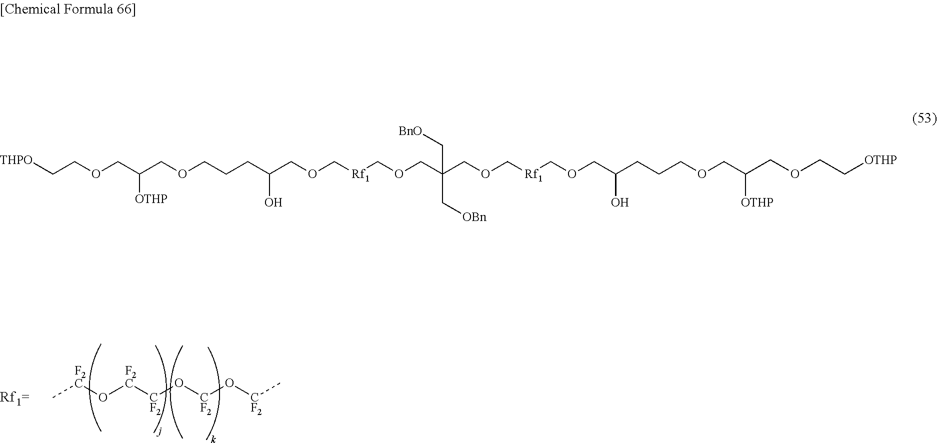

(In Formula (53), THP represents a tetrahydropyranyl group, and Bn represents a benzyl group. Rf₁ is represented by the above formula, j representing the average degree of polymerization in Rf₁ is 4.0, and k representing the average degree of polymerization is 4.0.)

A compound (Ep-13) represented by Formula (54) was synthesized by the following method.

A hydroxy group of ethylene glycol monoallyl ether was protected using dihydropyran, and the double bond of the obtained compound was oxidized. An epoxy group of the compound obtained by oxidizing a double bond was reacted with a hydroxy group of 4-penten-1-ol. The secondary hydroxy group of the obtained compound was protected with a THP group, and the double bond of the obtained compound was oxidized. The above-described steps made a compound (Ep-13) represented by Formula (54) obtained.

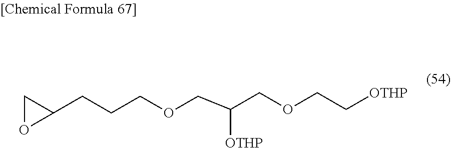

(In Formula (54), THP represents a tetrahydropyranyl group)

### (Fourth Reaction)

4.6 g of a compound represented by Formula (53) (number-average molecular weight: 2,873, 1.6 mmol) as an intermediate compound (1M-3), 46 mL of methanol, and 4.6 mL of formic acid were charged into a 200 mL eggplant flask under a nitrogen gas atmosphere, and the mixture was stirred at room temperature until uniform. 0.46 g of palladium carbon (Pd/C) was further added to this uniform liquid, and the mixture was stirred at 70° C. for 5 hours to carry out a reaction.

The reaction solution obtained after the reaction was filtered to remove Pd/C, and the filtrate was thickened. After the thickening, the residue was purified by silica gel column chromatography to obtain 3.4 g of a compound (1M) represented by Formula (55) (number-average molecular weight: 2355, 1.5 mmol).

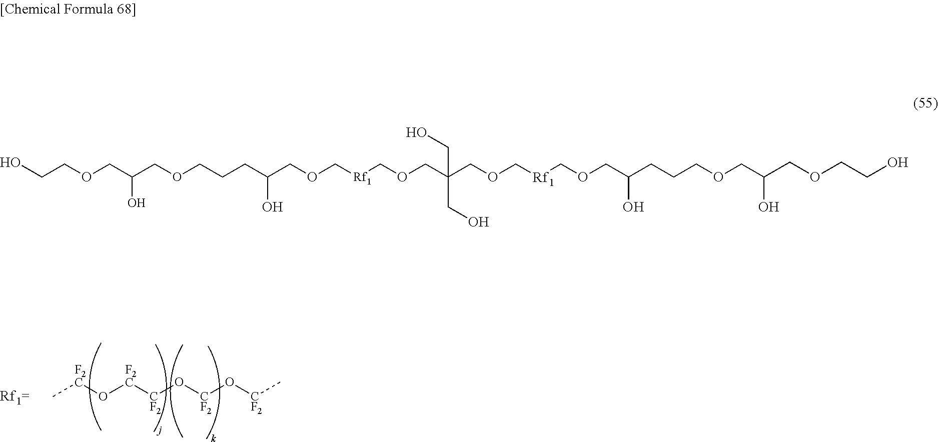

(In Formula (55), Rf₁ is represented by the above formula, j representing the average degree of polymerization in Rf₁ is 4.0, and k representing the average degree of polymerization is 4.0.)

The obtained compound (1M) was subjected to ¹H-NMR and 19F-NMR measurements, and the structure was identified from the following results. ¹H-NMR (CD₃COCD₃): δ [ppm]=1.60 to 1.83 (8H), 3.36 to 4.35 (52H) ¹⁹F-NMR (CD₃COCD₃): δ [ppm]=−55.6 to −50.7 (16F), −77.7 (4F), −80.3 (4F), −90.9 to −88.6 (32F)

### Example 14

### (Third Reaction)

The operation up to the third reaction was performed in the same manner as in Example 1 except that in the third reaction in Example 1 described above, 1.9 g (molecular weight: 264, 7.1 mmol) of a compound (Ep-14) represented by Formula (56) was used instead of the compound (Ep-1) represented by Formula (13), and 4.4 g of an intermediate compound (1N-3) represented by Formula (57) was obtained.

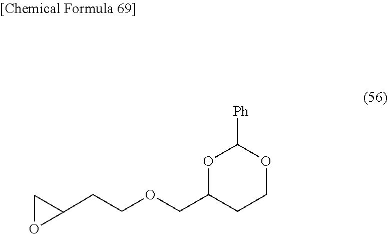 [SMILES: c1ccc(C2OCCC(COCCC3CO3)O2)cc1]

(In Formula (56), Ph represents a phenyl group.)

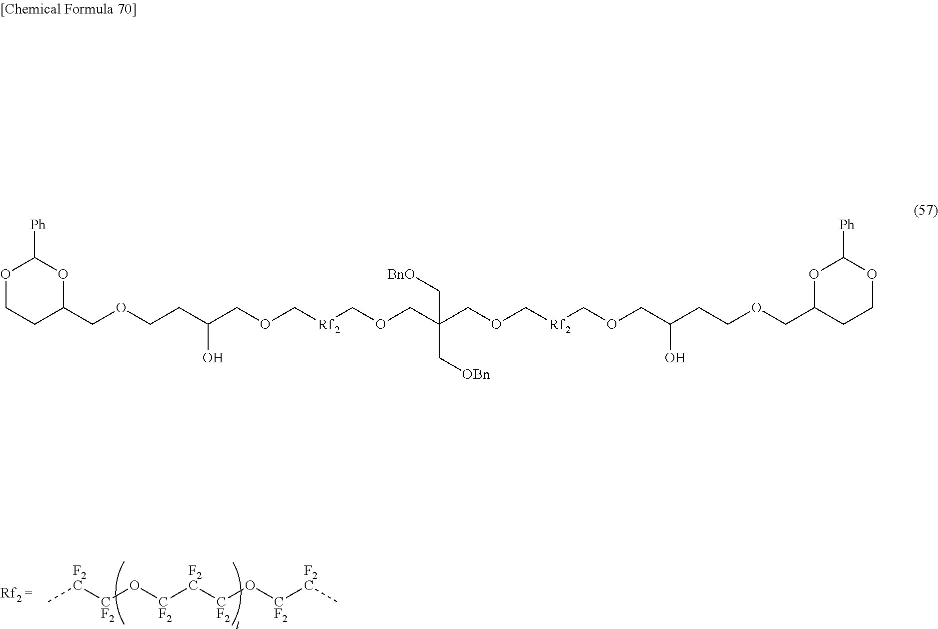

(In Formula (57), Ph represents a phenyl group, and Bn represents a benzyl group. Rf₂ is represented by the above formula, and 1 representing the average degree of polymerization in Rf₂ represents 3.8.)

A compound (Ep-14) represented by Formula (56) was synthesized by the following method.

1,2,4-butanetriol was reacted with benzaldehyde dimethyl acetal. As a result, a compound in which a hydroxy group bonded to the carbon at the 2-position and the carbon at the 4-position of 1,2,4-butanetriol was protected was synthesized. This compound was reacted with 2-bromoethyloxirane. The above-described steps made a compound (Ep-14) represented by Formula (56) obtained.

### (Fourth Reaction)

4.4 g of a compound represented by Formula (57) (number-average molecular weight: 2,630, 1.8 mmol) as an intermediate compound (1N-3), 44 mL of methanol, and 4.4 mL of formic acid were charged into a 200 mL eggplant flask under a nitrogen gas atmosphere, and the mixture was stirred at room temperature until uniform. 0.44 g of palladium carbon (Pd/C) was further added to this uniform liquid, and the mixture was stirred at 70° C. for 5 hours to carry out a reaction.

The reaction solution obtained after the reaction was filtered to remove Pd/C, and the filtrate was thickened. After the thickening, the residue was purified by silica gel column chromatography to obtain 3.5 g of a compound (1N) represented by Formula (58) (number-average molecular weight: 2273, 1.5 mmol).

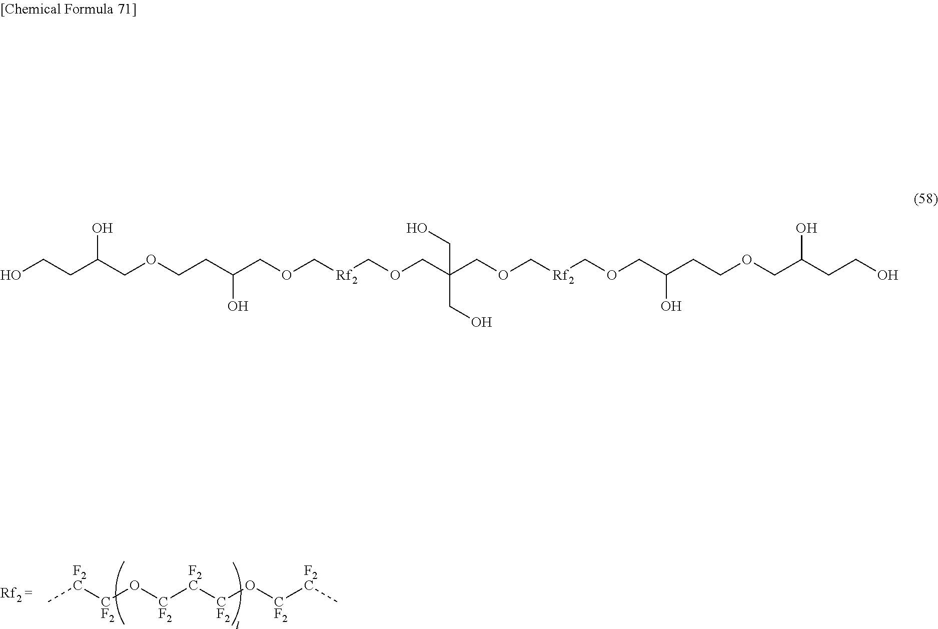

(In Formula (58), Rf₂ is represented by the above formula, and 1 representing the average degree of polymerization in Rf₂ represents 3.8.)

The obtained compound (1N) was subjected to ¹H-NMR and ¹⁹F-NMR measurements, and the structure was identified from the following results.

¹H-NMR (CD₃COCD₃): δ [ppm]=1.63 to 1.88 (8H), 3.29 to 4.44 (44H) ¹⁹F-NMR (CD₃COCD₃): δ [ppm]=−84.0 to −83.2 (30.4F), −86.3 (8F), −124.3 (8F), −130.1 to −129.0 (15.2F)

### Example 15

### (Third Reaction)

The operation up to the third reaction was performed in the same manner as in Example 9 except that in the third reaction in Example 9 described above, 2.3 g (molecular weight: 317, 7.1 mmol) of a compound (Ep-15) represented by Formula (59) was used instead of the compound (Ep-9) represented by Formula (41), and 4.5 g of an intermediate compound (10-3) represented by Formula (60) was obtained.

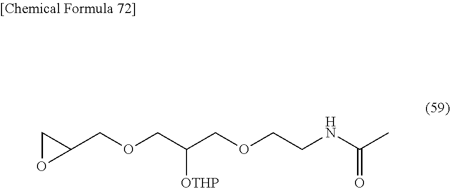

(In Formula (59), THP represents a tetrahydropyranyl group)

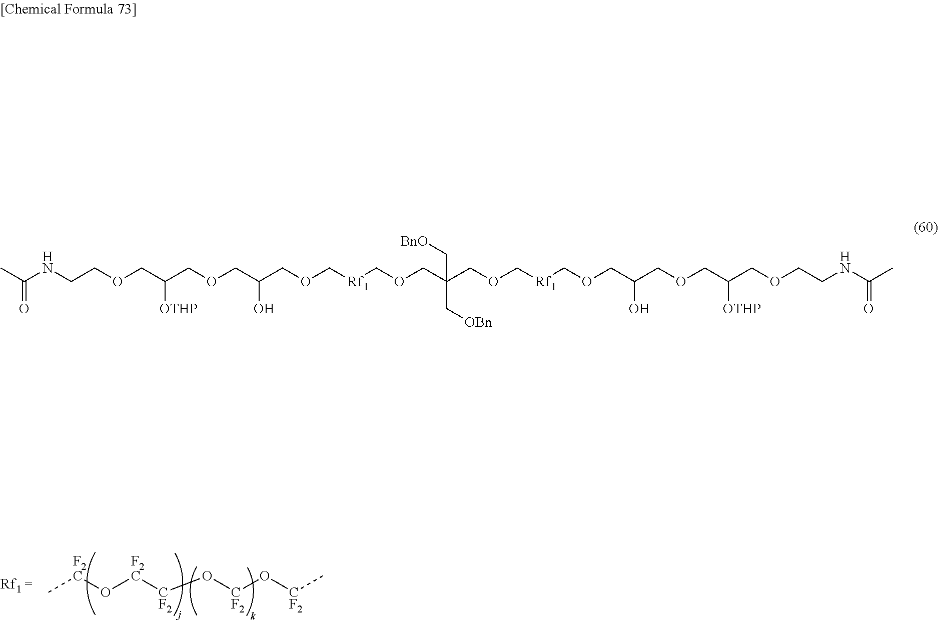

(In Formula (60), THP represents a tetrahydropyranyl group, and Bn represents a benzyl group. Rf₁ is represented by the above formula, j representing the average degree of polymerization in Rf₁ is 6.3, and k representing the average degree of polymerization is 0.)

A compound (Ep-15) represented by Formula (59) was synthesized by the following method.

2-acetamidoethanol and allyl glycidyl ether were reacted with each other to obtain a compound. Next, the secondary hydroxy group of the obtained compound was protected with a tetrahydropyranyl (THP) group. The terminal double bond of the obtained compound was oxidized in dichloromethane using metachloroperbenzoic acid. The above-described steps made a compound (Ep-15) represented by Formula (59) obtained.

### (Fourth Reaction)

4.5 g of a compound represented by Formula (60) (number-average molecular weight: 2,736, 1.7 mmol) as an intermediate compound (10-3), 45 mL of methanol, and 4.5 mL of formic acid were charged into a 200 mL eggplant flask under a nitrogen gas atmosphere, and the mixture was stirred at room temperature until uniform. 0.45 g of palladium carbon (Pd/C) was further added to this uniform liquid, and the mixture was stirred at 70° C. for 5 hours to carry out a reaction.

The reaction solution obtained after the reaction was filtered to remove Pd/C, and the filtrate was thickened. After the thickening, the residue was purified by silica gel column chromatography to obtain 3.6 g of a compound (10) represented by Formula (61) (number-average molecular weight: 2386, 1.5 mmol).

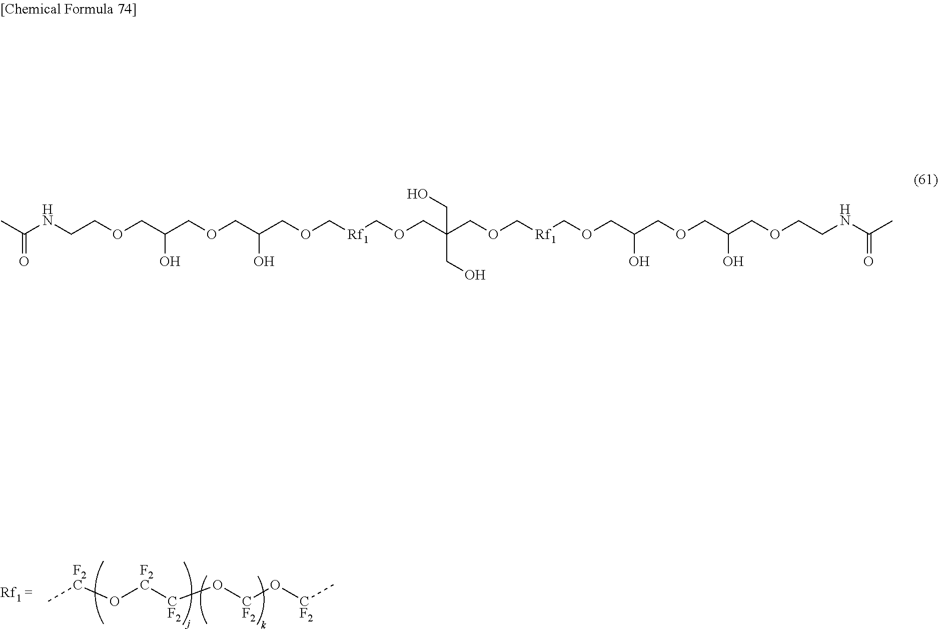

(In Formula (61), Rf₁ is represented by the above formula, j representing the average degree of polymerization in Rf₁ is 6.3, and k representing the average degree of polymerization is 0.)

The obtained compound (10) was subjected to ¹H-NMR and ¹⁹F-NMR measurements, and the structure was identified from the following results.

¹H-NMR (CD₃COCD₃): δ [ppm]=1.78 to 1.92 (6H), 3.30 to 4.40 (50H), 7.24 to 7.43 (2H)

¹⁹F-NMR (CD₃COCD₃): δ [ppm]=−78.5 (4F), −81.3 (4F), −90.0 to −88.4 (50.4F)

### Example 16

### (Second Reaction)

The operation up to the second reaction was performed in the same manner as in Example 1 except that in the second reaction in Example 1 described above, 3.3 g (molecular weight: 652, 5.0 mmol) of a compound represented by Formula (B1) was used instead of the compound represented by Formula (Al), and 7.0 g of an intermediate compound (1P-2) represented by Formula (62) was obtained.

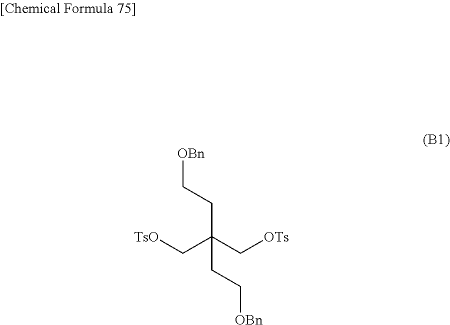 [SMILES: Cc1ccc(S(=O)(=O)OCC(CCOCc2ccccc2)(CCOCc2ccccc2)COS(=O)(=O)c2ccc(C)cc2)cc1]

(In Formula (B1), Ts represents a p-toluenesulfonyl group, and Bn represents a benzyl group.)

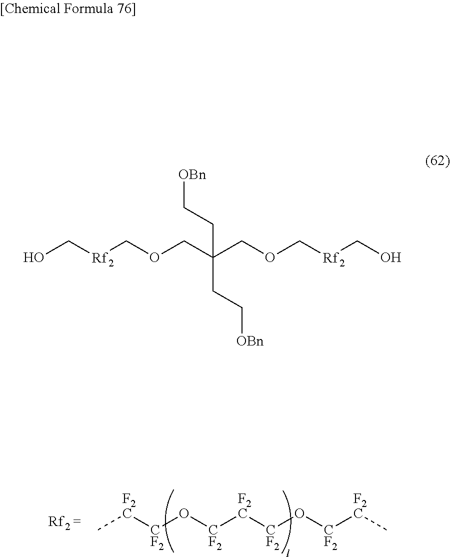

(In Formula (62), Bn represents a benzyl group, Rf₂ is represented by the above formula, and 1 representing the average degree of polymerization in Rf₂ represents 3.8.)

The compound represented by Formula (B1) was synthesized by reacting benzyl chloroethyl ether with diethyl malonate, then, reducing the ester with lithium borohydride, and reacting the resulting two primary hydroxy groups with p-toluenesulfonyl chloride.

### (Third Reaction)

5.0 g of a compound represented by Formula (62) (number-average molecular weight: 2,130, 2.4 mmol) as an intermediate compound (1P-2), 1.4 g of a compound (Ep-1) represented by Formula (13) (molecular weight: 202, 7.1 mmol), and 22 mL of t-butanol were charged into a 200 mL eggplant flask under a nitrogen gas atmosphere, and the mixture was stirred at room temperature until uniform. 0.053 g of potassium tert-butoxide (molecular weight: 112, 0.47 mmol) was further added to this uniform liquid, and the mixture was stirred at 70° C. for 16 hours to carry out a reaction.

A reaction product obtained after the reaction was cooled to 25° C., transferred to a separatory funnel containing 100 mL of water, and extracted three times with 100 mL of ethyl acetate. The organic layer was washed with water and dewatered with anhydrous sodium sulfate. After filtering off a desiccant, the filtrate was thickened, and the residue was purified by silica gel column chromatography to obtain 4.0 g of a compound represented by Formula (63) as an intermediate compound (1P-3).

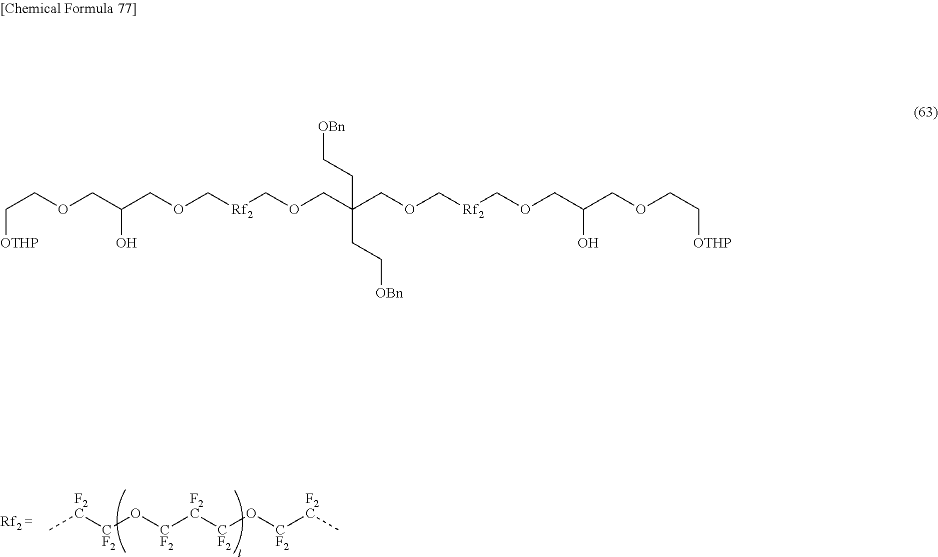

(In Formula (63), THP represents a tetrahydropyranyl group, and Bn represents a benzyl group. Rf₂ is represented by the above formula, and 1 representing the average degree of polymerization in Rf₂ represents 3.8.)

### (Fourth Reaction)

4.0 g of a compound represented by Formula (63) (number-average molecular weight: 2,534, 1.6 mmol) as an intermediate compound (1P-3), 40 mL of methanol, and 4.0 mL of formic acid were charged into a 200 mL eggplant flask under a nitrogen gas atmosphere, and the mixture was stirred at room temperature until uniform. 0.40 g of palladium carbon (Pd/C) was further added to this uniform liquid, and the mixture was stirred at 70° C. for 5 hours to carry out a reaction.

The reaction solution obtained after the reaction was filtered to remove Pd/C, and the filtrate was thickened. After the thickening, the residue was purified by silica gel column chromatography to obtain 3.1 g of a compound (1P) represented by Formula (64) (number-average molecular weight: 2184, 1.4 mmol).

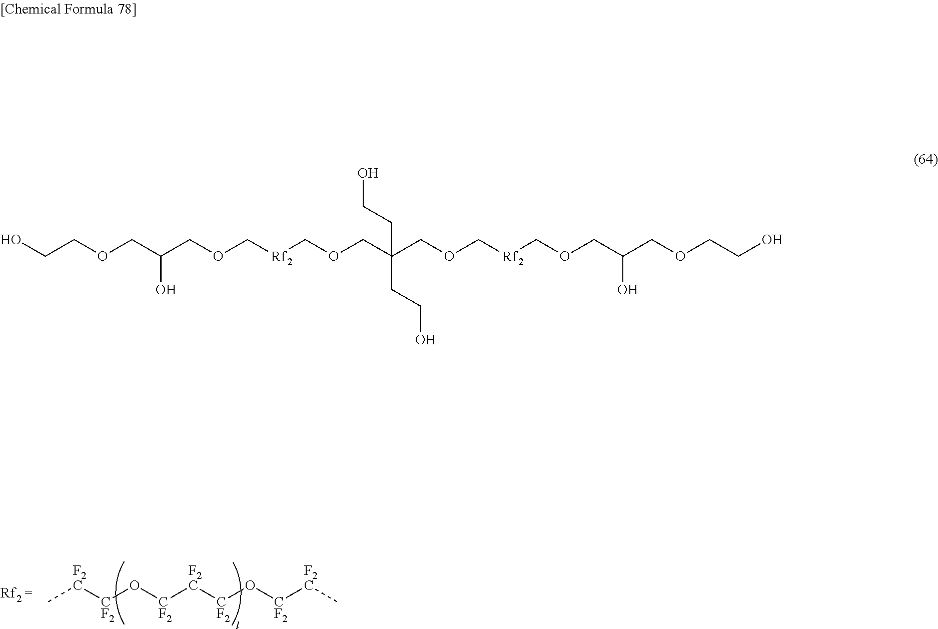

(In Formula (64), Rf₂ is represented by the above formula, and 1 representing the average degree of polymerization in Rf₂ represents 3.8.)

The obtained compound (1P) was subjected to ¹H-NMR and ¹⁹F-NMR measurements, and the structure was identified from the following results.

¹H-NMR (CD₃COCD₃): δ [ppm]=1.63 to 1.84 (4H), 3.39 to 4.34 (40H)

¹⁹F-NMR (CD₃COCD₃): δ[ppm]=−84.0 to −83.0 (30.4F), −86.4 (8F), −124.3 (8F), −130.0 to −129.0 (15.2F)

### Example 17

### (Third Reaction)

The operation up to the third reaction was performed in the same manner as in Example 16 except that in the third reaction in Example 16 described above, 1.2 g (molecular weight: 172, 7.1 mmol) of a compound (Ep-7) represented by Formula (33) was used instead of the compound (Ep-1) represented by Formula (13), and 3.9 g of an intermediate compound (1Q-3) represented by Formula (65) was obtained.

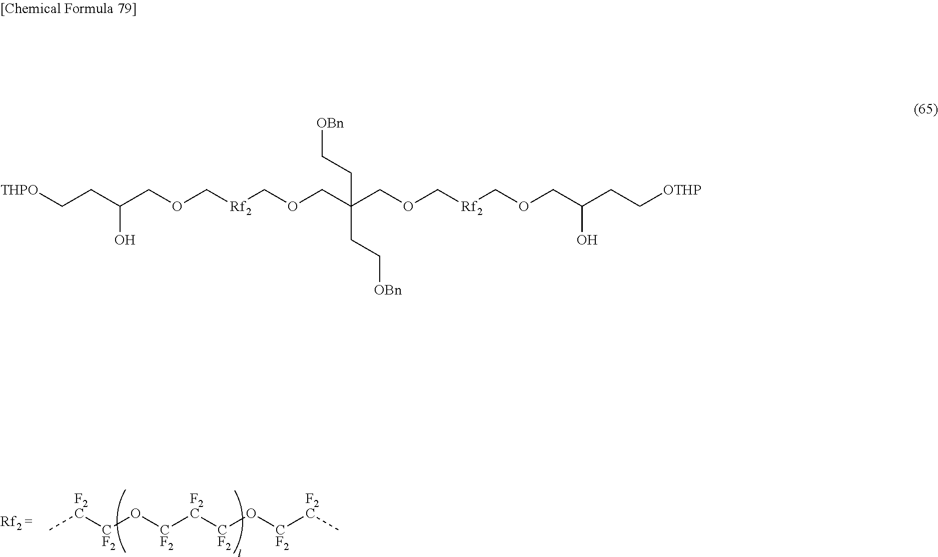

(In Formula (65), THP represents a tetrahydropyranyl group, and Bn represents a benzyl group. Rf₂ is represented by the above formula, and 1 representing the average degree of polymerization in Rf₂ represents 3.8.)

### (Fourth Reaction)

3.9 g of a compound represented by Formula (65) (number-average molecular weight: 2,474, 1.6 mmol) as an intermediate compound (1Q-3), 39 mL of methanol, and 3.9 mL of formic acid were charged into a 200 mL eggplant flask under a nitrogen gas atmosphere, and the mixture was stirred at room temperature until uniform. 0.39 g of palladium carbon (Pd/C) was further added to this uniform liquid, and the mixture was stirred at 70° C. for 5 hours to carry out a reaction.

The reaction solution obtained after the reaction was filtered to remove Pd/C, and the filtrate was thickened. After the thickening, the residue was purified by silica gel column chromatography to obtain 3.0 g of a compound (1Q) represented by Formula (66) (number-average molecular weight: 2124, 1.4 mmol).

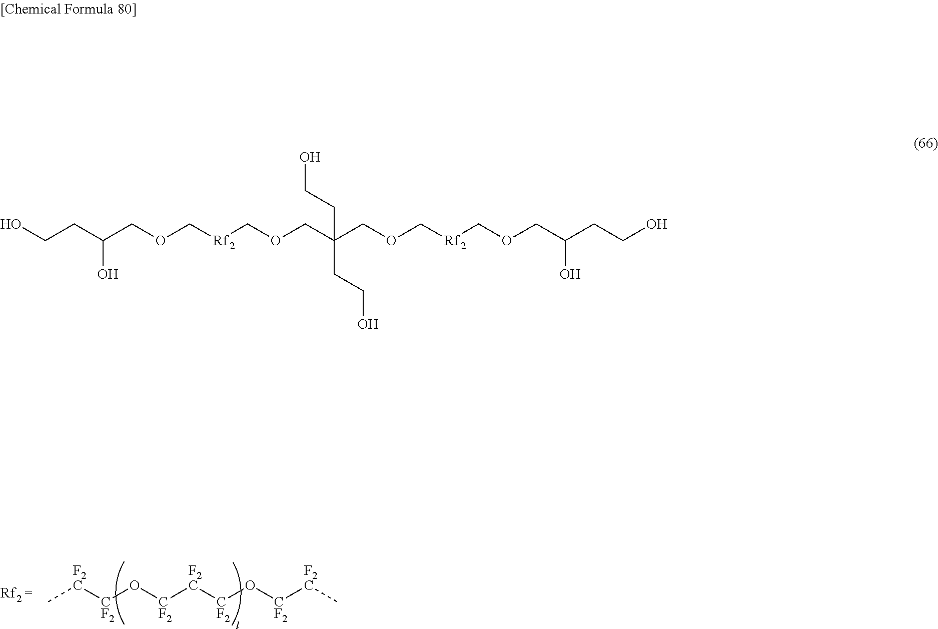

(In Formula (66), Rf₂ is represented by the above formula, and 1 representing the average degree of polymerization in Rf₂ represents 3.8.)

The obtained compound (1Q) was subjected to ¹H-NMR and ¹⁹F-NMR measurements, and the structure was identified from the following results.

¹H-NMR (CD₃COCD₃): δ [ppm]=1.61 to 1.85 (8H), 3.43 to 4.32 (32H)

¹⁹F-NMR (CD₃COCD₃): δ [ppm]=−55.6 to −50.6 (16F), −77.8 (4F), −80.3 (4F), −91.0 to −88.4 (32F)

### Example 18

### (Second Reaction)

The operation up to the second reaction was performed in the same manner as in Example 1 except that in the second reaction in Example 1 described above, 3.4 g (molecular weight: 680, 5.0 mmol) of a compound represented by Formula (C1) was used instead of the compound represented by Formula (A1), and 7.3 g of an intermediate compound (1R-2) represented by Formula (67) was obtained.

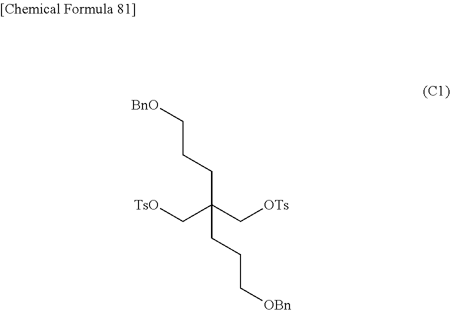 [SMILES: Cc1ccc(S(=O)(=O)OCC(CCCOCc2ccccc2)(CCCOCc2ccccc2)COS(=O)(=O)c2ccc(C)cc2)cc1]

(In Formula (C1), Ts represents a p-toluenesulfonyl group, and Bn represents a benzyl group.)

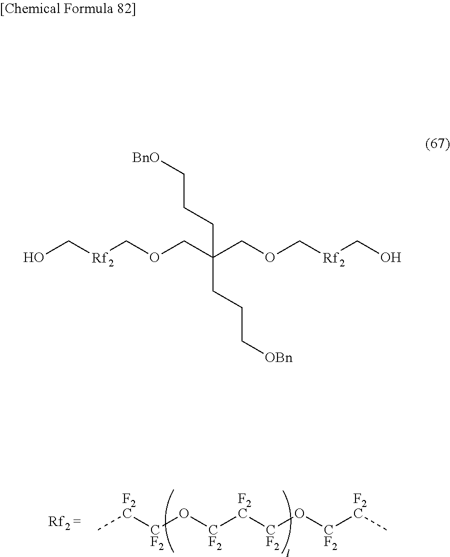

(In Formula (67), Bn represents a benzyl group, Rf₂ is represented by the above formula, and 1 representing the average degree of polymerization in Rf₂ represents 3.8.)

The compound represented by Formula (C1) was synthesized by reacting benzylchloropropyl ether with diethyl malonate, reducing the ester with lithium borohydride, and reacting the resulting two primary hydroxy groups with p-toluenesulfonyl chloride.

### (Third Reaction)

5.0 g of a compound represented by Formula (67) (number-average molecular weight: 2,158, 2.2 mmol) as an intermediate compound (1R-2), 2.1 g of a compound (Ep-3) represented by Formula (20) (molecular weight: 320, 6.5 mmol), and 16 mL of t-butanol were charged into a 200 mL eggplant flask under a nitrogen gas atmosphere, and the mixture was stirred at room temperature until uniform. 0.048 g of potassium tert-butoxide (molecular weight: 112, 0.43 mmol) was further added to this uniform liquid, and the mixture was stirred at 70° C. for 16 hours to carry out a reaction.

A reaction product obtained after the reaction was cooled to 25° C., transferred to a separatory funnel containing 100 mL of water, and extracted three times with 100 mL of ethyl acetate. The organic layer was washed with water and dewatered with anhydrous sodium sulfate. After filtering off a desiccant, the filtrate was thickened, and the residue was purified by silica gel column chromatography to obtain 4.2 g of a compound represented by Formula (68) as an intermediate compound (1R-3).

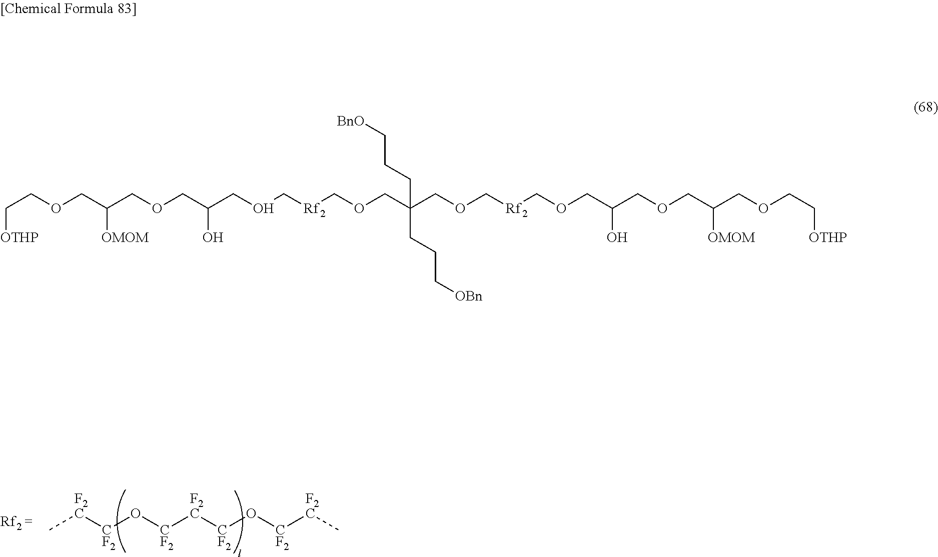

(In Formula (68), THP represents a tetrahydropyranyl group, Bn represents a benzyl group, and MOM represents a methoxymethyl group. Rf₂ is represented by the above formula, and 1 representing the average degree of polymerization in Rf₂ represents 3.8.)

### (Fourth Reaction)

4.2 g of a compound represented by Formula (68) (number-average molecular weight: 2,799, 2.2 mmol) as an intermediate compound (1R-3), 42 mL of methanol, and 4.2 mL of formic acid were charged into a 200 mL eggplant flask under a nitrogen gas atmosphere, and the mixture was stirred at room temperature until uniform. 0.42 g of palladium carbon (Pd/C) was further added to this uniform liquid, and the mixture was stirred at 70° C. for 5 hours to carry out a reaction.

The reaction solution obtained after the reaction was filtered to remove Pd/C, and the filtrate was thickened. After the thickening, the residue was purified by silica gel column chromatography to obtain 3.3 g of a compound (1R) represented by Formula (69) (number-average molecular weight: 2361, 1.3 mmol).

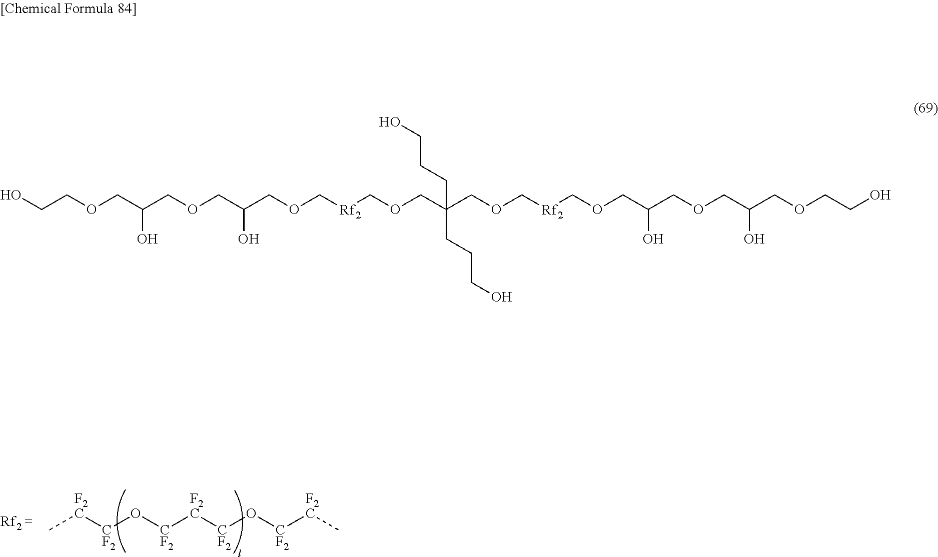

(In Formula (69), Rf₂ is represented by the above formula, and 1 representing the average degree of polymerization in Rf₂ represents 3.8.)

The obtained compound (1R) was subjected to ¹H-NMR and ¹⁹F-NMR measurements, and the structure was identified from the following results.

¹H-NMR (CD₃COCD₃): δ [ppm]=1.52 to 1.81 (8H), 3.35 to 4.38 (52H)

¹⁹F-NMR (CD₃COCD₃): S[ppm]=−84.1 to −83.0 (30.4F), −86.4 (8F), −124.3 (8F), −130.2 to −129.1 (15.2F)

### Example 19

### (Second Reaction)

The operation up to the second reaction was performed in the same manner as in Example 1 except that in the second reaction in Example 1 described above, 3.6 g (molecular weight: 712, 5.0 mmol) of a compound represented by Formula (H1) was used instead of the compound represented by Formula (A1), and 7.5 g of an intermediate compound (1S-2) represented by Formula (70) was obtained.

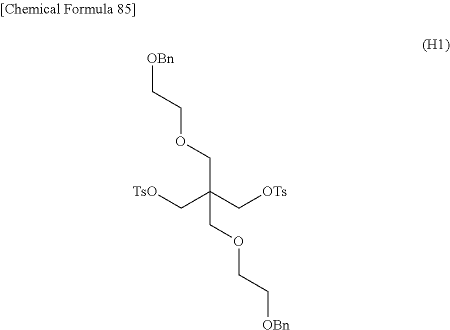 [SMILES: Cc1ccc(S(=O)(=O)OCC(COCCOCc2ccccc2)(COCCOCc2ccccc2)COS(=O)(=O)c2ccc(C)cc2)cc1]

(In Formula (H1), Ts represents a p-toluenesulfonyl group, and Bn represents a benzyl group.)

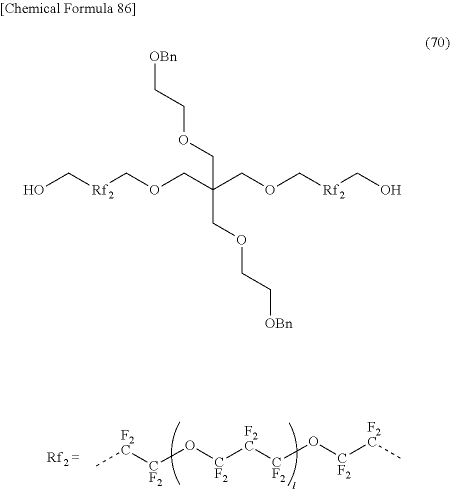

(In Formula (70), Bn represents a benzyl group, Rf₂ is represented by the above formula, and 1 representing the average degree of polymerization in Rf₂ represents 3.8.)

The compound represented by Formula (H1) was synthesized by reacting 2-(chloromethoxy)ethoxymethylbenzene with diethyl malonate, then, reducing the ester with lithium borohydride, and reacting the resulting two primary hydroxy groups with p-toluenesulfonyl chloride.

### (Third Reaction)

5.0 g of a compound represented by Formula (70) (number-average molecular weight: 2,190, 2.1 mmol) as an intermediate compound (1S-2), 2.1 g of a compound (Ep-6) represented by Formula (29) (molecular weight: 334, 6.4 mmol), and 16 mL of t-butanol were charged into a 200 mL eggplant flask under a nitrogen gas atmosphere, and the mixture was stirred at room temperature until uniform. 0.048 g of potassium tert-butoxide (molecular weight: 112, 0.43 mmol) was further added to this uniform liquid, and the mixture was stirred at 70° C. for 16 hours to carry out a reaction.

A reaction product obtained after the reaction was cooled to 25° C., transferred to a separatory funnel containing 100 mL of water, and extracted three times with 100 mL of ethyl acetate. The organic layer was washed with water and dewatered with anhydrous sodium sulfate. After filtering off a desiccant, the filtrate was thickened, and the residue was purified by silica gel column chromatography to obtain 4.4 g of a compound represented by Formula (71) as an intermediate compound (1S-3).

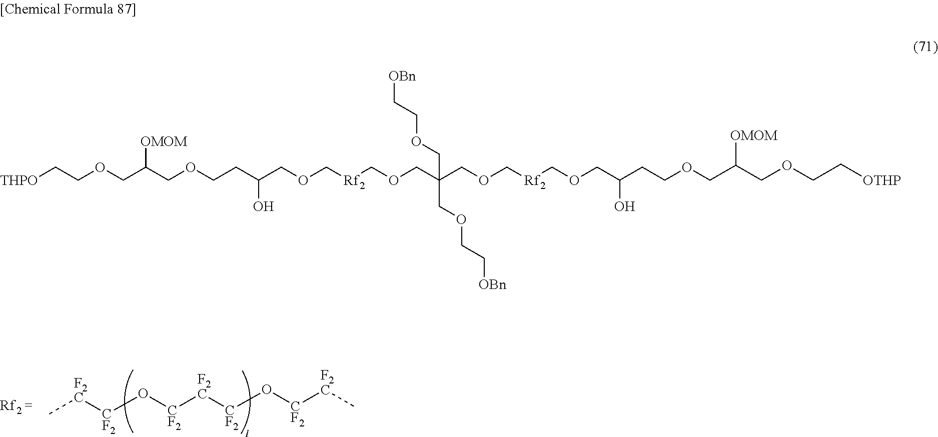

(In Formula (71), THP represents a tetrahydropyranyl group, Bn represents a benzyl group, and MOM represents a methoxymethyl group. Rf₂ is represented by the above formula, and 1 representing the average degree of polymerization in Rf₂ represents 3.8.)

### (Fourth Reaction)

4.4 g of a compound represented by Formula (71) (number-average molecular weight: 2,859, 1.4 mmol) as an intermediate compound (1S-3), 42 mL of methanol, and 4.2 mL of formic acid were charged into a 200 mL eggplant flask under a nitrogen gas atmosphere, and the mixture was stirred at room temperature until uniform. 0.42 g of palladium carbon (Pd/C) was further added to this uniform liquid, and the mixture was stirred at 70° C. for 5 hours to carry out a reaction.

The reaction solution obtained after the reaction was filtered to remove Pd/C, and the filtrate was thickened. After the thickening, the residue was purified by silica gel column chromatography to obtain 3.2 g of a compound (1S) represented by Formula (72) (number-average molecular weight: 2435, 1.2 mmol).

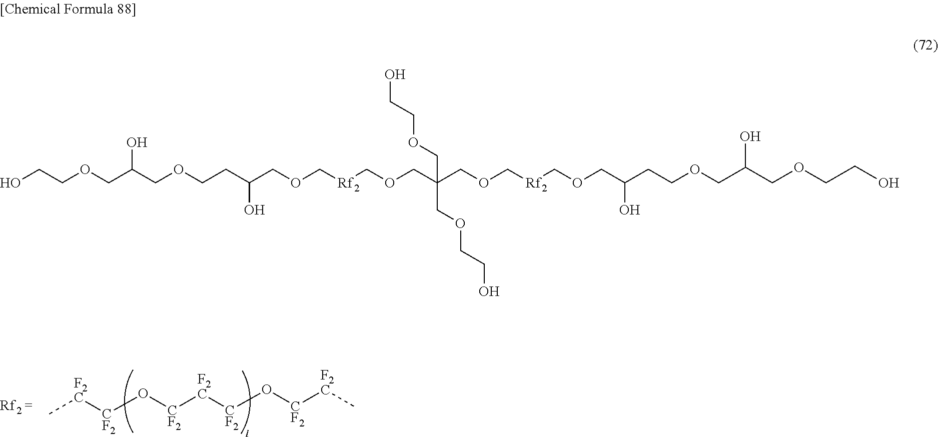

(In Formula (72), Rf₂ is represented by the above formula, and 1 representing the average degree of polymerization in Rf₂ represents 3.8.)

The obtained compound (1S) was subjected to ¹H-NMR and ¹⁹F-NMR measurements, and the structure was identified from the following results.

¹H-NMR (CD₃COCD₃): δ [ppm]=1.64 to 1.80 (4H), 3.41 to 4.32 (60H)

¹⁹F-NMR (CD₃COCD₃): δ [ppm]=−55.6 to −50.6 (16F), −77.7 (4F), −80.3 (4F), −91.0 to −88.5 (32F)

### Example 20

(Second Reaction) The operation up to the second reaction was performed in the same manner as in Example 1 except that in the second reaction in Example 1 described above, 3.4 g (molecular weight: 668, 5.0 mmol) of a compound represented by Formula (M1) was used instead of the compound represented by Formula (Al), and 7.4 g of an intermediate compound (1T-2) represented by Formula (73) was obtained.

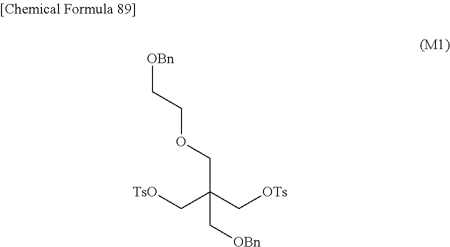 [SMILES: Cc1ccc(S(=O)(=O)OCC(COCCOCc2ccccc2)(COCc2ccccc2)COS(=O)(=O)c2ccc(C)cc2)cc1]

(In Formula (M1), Ts represents a p-toluenesulfonyl group, and Bn represents a benzyl group.)

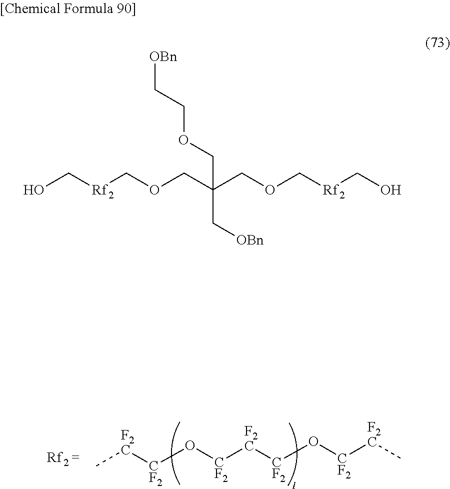

(In Formula (73), Bn represents a benzyl group, Rf₂ is represented by the above formula, and 1 representing the average degree of polymerization in Rf₂ represents 3.8.)

The compound represented by Formula (M1) was synthesized by sequentially reacting 2-(chloromethoxy)ethoxymethylbenzene and benzyl chloromethyl ether with diethyl malonate, then, reducing the ester with lithium borohydride, and reacting the resulting two primary hydroxy groups with p-toluenesulfonyl chloride.

### (Third Reaction)

5.0 g of a compound represented by Formula (73) (number-average molecular weight: 2,146, 2.1 mmol) as an intermediate compound (1T-2), 1.3 g of a compound (Ep-1) represented by Formula (13) (molecular weight: 202, 6.4 mmol), and 16 mL of t-butanol were charged into a 200 mL eggplant flask under a nitrogen gas atmosphere, and the mixture was stirred at room temperature until uniform. 0.048 g of potassium tert-butoxide (molecular weight: 112, 0.43 mmol) was further added to this uniform liquid, and the mixture was stirred at 70° C. for 16 hours to carry out a reaction.

A reaction product obtained after the reaction was cooled to 25° C., transferred to a separatory funnel containing 100 mL of water, and extracted three times with 100 mL of ethyl acetate. The organic layer was washed with water and dewatered with anhydrous sodium sulfate. After filtering off a desiccant, the filtrate was thickened, and the residue was purified by silica gel column chromatography to obtain 3.9 g of a compound represented by Formula (74) as an intermediate compound (1T-3).

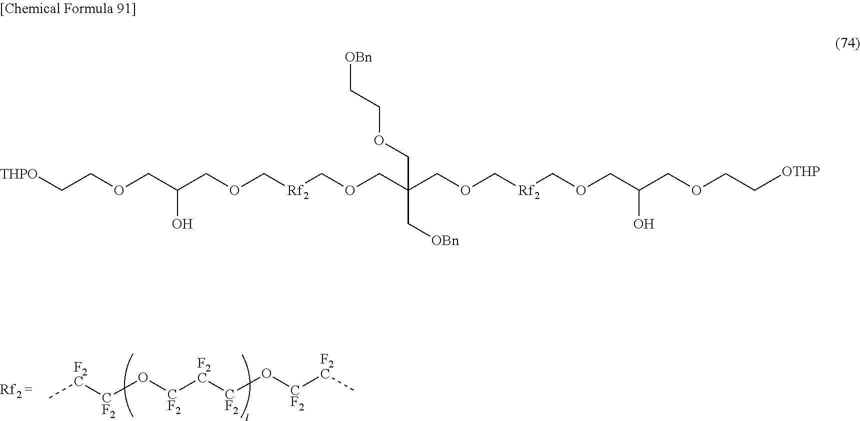

(In Formula (74), THP represents a tetrahydropyranyl group, and Bn represents a benzyl group. Rf₂ is represented by the above formula, and 1 representing the average degree of polymerization in Rf₂ represents 3.8.)

### (Fourth Reaction)

3.9 g of a compound represented by Formula (74) (number-average molecular weight: 2,550, 1.4 mmol) as an intermediate compound (1T-3), 39 mL of methanol, and 3.9 mL of formic acid were charged into a 200 mL eggplant flask under a nitrogen gas atmosphere, and the mixture was stirred at room temperature until uniform. 0.39 g of palladium carbon (Pd/C) was further added to this uniform liquid, and the mixture was stirred at 70° C. for 5 hours to carry out a reaction.

The reaction solution obtained after the reaction was filtered to remove Pd/C, and the filtrate was thickened. After the thickening, the residue was purified by silica gel column chromatography to obtain 3.0 g of a compound (1T) represented by Formula (75) (number-average molecular weight: 2200, 1.3 mmol).

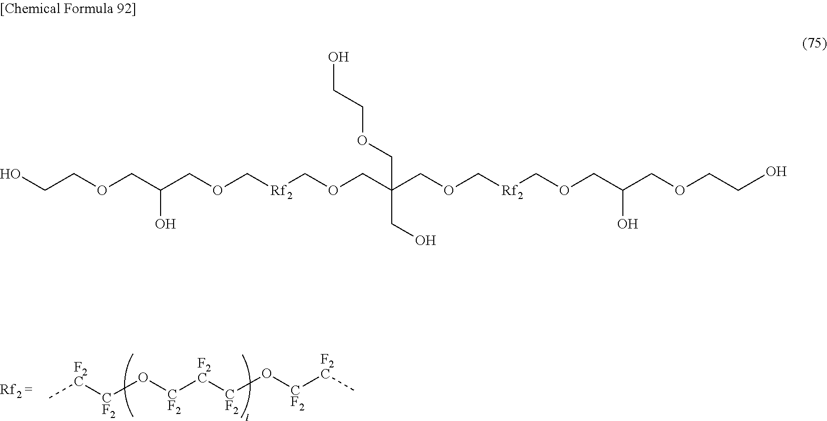

(In Formula (75), Rf₂ is represented by the above formula, and 1 representing the average degree of polymerization in Rf₂ represents 3.8.)

The obtained compound (1T) was subjected to ¹H-NMR and ¹⁹F-NMR measurements, and the structure was identified from the following results.

¹H-NMR (CD₃COCD₃): δ [ppm]=3.37 to 4.37 (44H)

¹⁹F-NMR (CD₃COCD₃): δ[ppm]=−84.0 to −83.0 (30.4F), −86.4 (8F), −124.3 (8F), −130.0 to −129.0 (15.2F)

### Example 21

### (First Reaction)

10 g of a compound represented by

HOCH₂CF₂CF₂O(CF₂CF₂CF₂O)ᵢCF₂CF₂CH₂OH (1 representing the average degree of polymerization in the formula was 2.0) (number-average molecular weight: 610, molecular weight distribution: 1.1), 25.6 g of a compound represented by Formula (A1) (molecular weight: 624, 41.0 mmol), 1.1 g of potassium iodide (molecular weight: 166, 6.6 mmol), and 33 mL of N,N′-dimethylformamide (DMF) were charged into a 300 mL eggplant flask under a nitrogen gas atmosphere, and stirred at room temperature to obtain a mixed solution. 21.3 g of cesium carbonate (molecular weight: 325, 65.6 mmol) was added to this mixed solution, stirred and reacted at 70° C. for 10 hours.

The reaction product obtained after the reaction was cooled to 0° C., 100 mL of water was added thereto, and the reaction was stopped. The obtained reaction solution was transferred to a separatory funnel and extracted three times with 100 mL of ethyl acetate. The organic layer was washed with saline and dewatered with anhydrous sodium sulfate. After filtering off a desiccant, the filtrate was thickened, and the residue was purified by silica gel column chromatography to obtain 11.0 g of a compound represented by Formula (76) as an intermediate compound (2A-1).

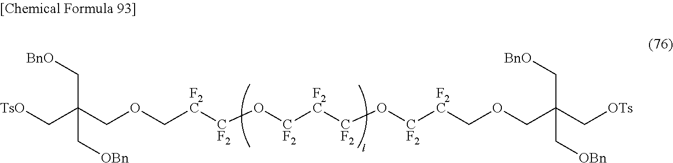

(In Formula (76), Ts represents a p-toluenesulfonyl group, Bn represents a benzyl group, and 1 representing the average degree of polymerization represents 2.0.)

### (Second Reaction)

20 g of a compound represented by

HOCH₂CF₂CF₂O(CF₂CF₂CF₂O)ᵢCF₂CF₂CH₂OH (1 representing the average degree of polymerization in the formula was 2.0) (number-average molecular weight: 610, molecular weight distribution: 1.1), 2.9 g of 3,4-dihydro-2H-pyran, and 66 mL of a mixed solution of ASAHIKLIN (registered trademark) AE-3000 (manufactured by AGC Inc.) as a fluorine-based solvent and dichloromethane (volume ratio: 1:1) were charged into a 300 mL eggplant flask under a nitrogen gas atmosphere, and the mixture was stirred at 0° C. until uniform, thereby obtaining a mixed solution. 0.13 g of p-toluenesulfonic acid monohydrate was added to this mixed solution, stirred at 0° C. for 30 minutes, and then stirred at room temperature for 2 hours to carry out a reaction.

The reaction product obtained after the reaction was cooled to 0° C., 50 mL of saturated aqueous sodium bicarbonate was added thereto, and the reaction was stopped. The obtained reaction solution was transferred to a separatory funnel and extracted three times with 100 mL of ethyl acetate. The organic layer was washed with saline and dewatered with anhydrous sodium sulfate. After filtering off a desiccant, the filtrate was thickened, and the residue was purified by silica gel column chromatography to obtain 10.0 g of a compound represented by Formula (77) as an intermediate compound (2A-2).

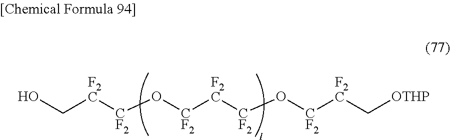

(In Formula (77), THP represents a tetrahydropyranyl group, and 1 representing the average degree of polymerization represents 2.0.)

### (Third Reaction)

7.0 g of a compound represented by Formula (76) as an intermediate compound (2A-1) (number-average molecular weight: 1517, 4.6 mmol), 7.0 g of a compound represented by Formula (77) as an intermediate compound (2A-2) (number-average molecular weight: 694, 10.2 mmol), 0.31 g of potassium iodide (molecular weight: 166, 1.9 mmol), and 10 mL of N,N′-dimethylformamide (DMF) were charged into a 200 mL eggplant flask under a nitrogen gas atmosphere, and stirred at room temperature to obtain a mixed solution. 6.0 g of cesium carbonate (molecular weight: 325, 18.5 mmol) was added to this mixed solution, stirred and reacted at 70° C. for 10 hours.

The reaction solution obtained after the reaction was returned to room temperature, 12 g of a 10% hydrogen chloride/methanol solution (hydrogen chloride-methanol reagent (5% to 10%) manufactured by Tokyo Chemical Industry Co., Ltd.) was added thereto, and the mixture was stirred at room temperature for 2 hours. The reaction solution was transferred little by little to a separatory funnel containing 100 mL of saline, and the mixture was extracted three times with 200 mL of ethyl acetate. The organic layer was washed with 100 mL of saline, 100 mL of saturated aqueous sodium bicarbonate, and 100 mL of saline in this order, and dewatered with anhydrous sodium sulfate. After filtering off a desiccant, the filtrate was thickened, and the residue was purified by silica gel column chromatography. The above-described steps made 5.0 g of a compound represented by Formula (78) obtained as an intermediate compound (2A-3).

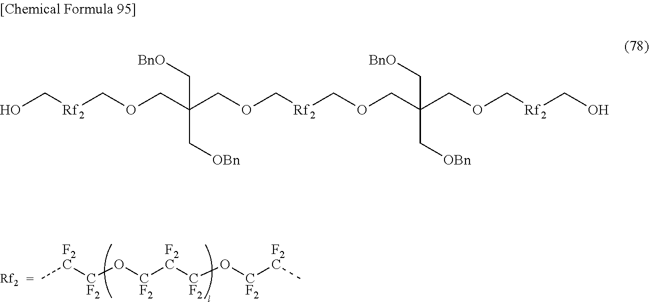

(In Formula (78), Bn represents a benzyl group, Rf₂ is represented by the above formula, and 1 representing the average degree of polymerization in Rf₂ represents 2.0.)

### (Fourth Reaction)

5.0 g of a compound represented by Formula (78) (number-average molecular weight: 2,392, 2.3 mmol) as an intermediate compound (2A-3), 1.0 g of a compound (Ep-1) represented by Formula (13) (molecular weight: 202, 4.9 mmol), and 2.1 mL of t-butanol were charged into a 200 mL eggplant flask under a nitrogen gas atmosphere, and the mixture was stirred at room temperature until uniform. 0.076 g of potassium tert-butoxide (molecular weight: 113, 0.67 mmol) was further added to this uniform liquid, and the mixture was stirred at 70° C. for 16 hours to carry out a reaction.

A reaction product obtained after the reaction was cooled to 25° C., transferred to a separatory funnel containing 100 mL of water, and extracted three times with 100 mL of ethyl acetate. The organic layer was washed with water and dewatered with anhydrous sodium sulfate. After filtering off a desiccant, the filtrate was thickened, and the residue was purified by silica gel column chromatography to obtain 3.5 g of a compound represented by Formula (79) as an intermediate compound (2A-4).

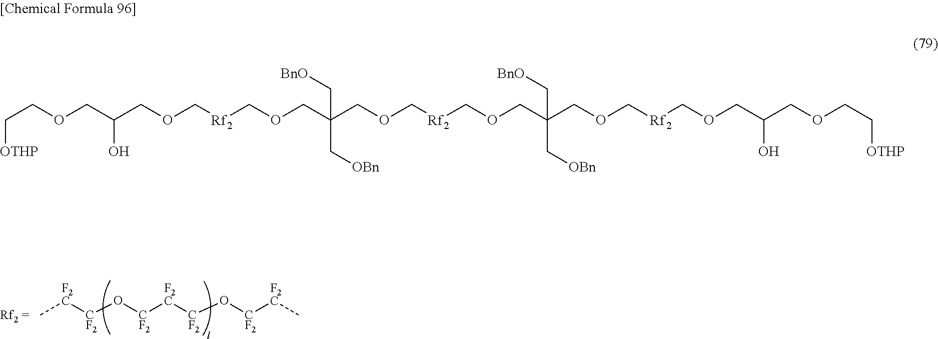

(In Formula (79), THP represents a tetrahydropyranyl group, and Bn represents a benzyl group. Rf₂ is represented by the above formula, and 1 representing the average degree of polymerization in Rf₂ represents 2.0.)

### (Fifth Reaction)

3.5 g of a compound represented by Formula (79) (number-average molecular weight: 2,797, 1.3 mmol) as an intermediate compound (2A-4), 35 mL of methanol, and 3.5 mL of formic acid were charged into a 200 mL eggplant flask under a nitrogen gas atmosphere, and the mixture was stirred at room temperature until uniform. 0.35 g of palladium carbon (Pd/C) was further added to this uniform liquid, and the mixture was stirred at 70° C. for 8 hours to carry out a reaction.

The reaction solution obtained after the reaction was filtered to remove Pd/C, and the filtrate was thickened. After the thickening, the residue was purified by silica gel column chromatography to obtain 2.6 g of a compound (2 Å) represented by Formula (80) (number-average molecular weight: 2271, 1.2 mmol).

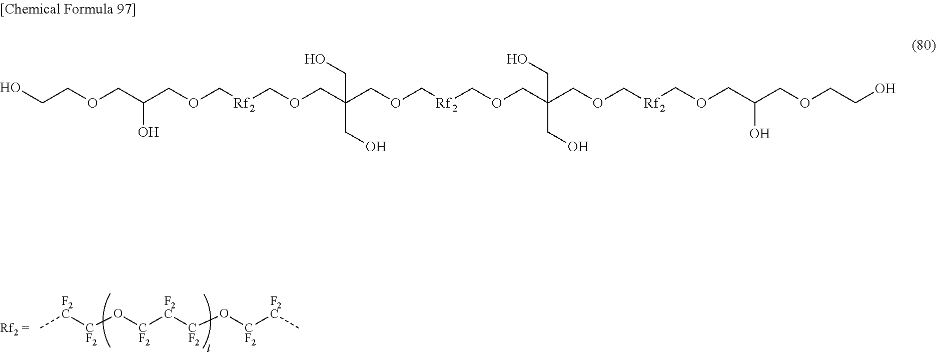

(In Formula (80), Rf₂ is represented by the above formula, and 1 representing the average degree of polymerization in Rf₂ represents 2.0.)

The obtained compound (2 Å) was subjected to ¹H-NMR and ¹⁹F-NMR measurements, and the structure was identified from the following results. ¹H-NMR (CD₃COCD₃): δ [ppm]=3.37 to 4.35 (54H) ¹⁹F-NMR (CD₃COCD₃): δ [ppm]=−84.2 to −83.0 (24F), −86.4 (12F), −124.3 (12F), −130.0 to −128.9 (12F)

### Example 22

### (Fourth Reaction)

The operation up to the fourth reaction was performed in the same manner as in Example 21 except that in the fourth reaction in Example 21 described above, 1.4 g (molecular weight: 216, 4.9 mmol) of a compound (Ep-2) represented by Formula (16) was used instead of the compound (Ep-1) represented by Formula (13), and 3.9 g of an intermediate compound (2B-4) represented by Formula (81) was obtained.

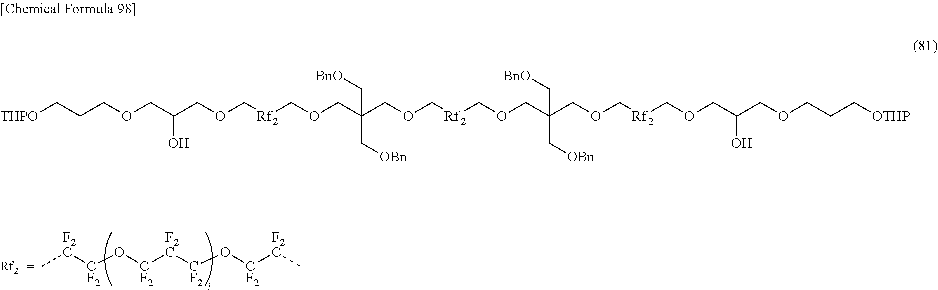

(In Formula (81), THP represents a tetrahydropyranyl group, and Bn represents a benzyl group. Rf₂ is represented by the above formula, and 1 representing the average degree of polymerization in Rf₂ represents 2.0.)

### (Fifth Reaction)

3.9 g of a compound represented by Formula (81) (number-average molecular weight: 2,948, 1.4 mmol) as an intermediate compound (2B-4), 39 mL of methanol, and 3.9 mL of formic acid were charged into a 200 mL eggplant flask under a nitrogen gas atmosphere, and the mixture was stirred at room temperature until uniform. 0.39 g of palladium carbon (Pd/C) was further added to this uniform liquid, and the mixture was stirred at 70° C. for 8 hours to carry out a reaction.

The reaction solution obtained after the reaction was filtered to remove Pd/C, and the filtrate was thickened. After the thickening, the residue was purified by silica gel column chromatography to obtain 3.0 g of a compound (2B) represented by Formula (82) (number-average molecular weight: 2299, 1.2 mmol).

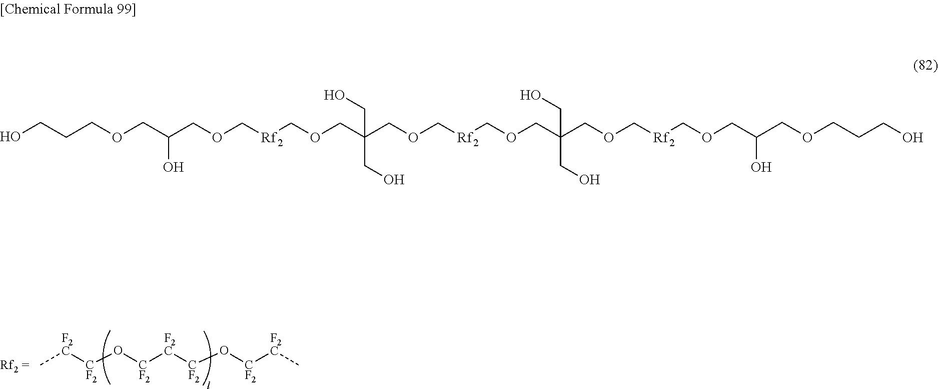

(In Formula (82), Rf₂ is represented by the above formula, and 1 representing the average degree of polymerization in Rf₂ represents 2.0.)

The obtained compound (2B) was subjected to ¹H-NMR and ¹⁹F-NMR measurements, and the structure was identified from the following results. ¹H-NMR (CD₃COCD₃): δ [ppm]=1.63 to 1.82 (4H), 3.38 to 4.38 (54H) ¹⁹F-NMR (CD₃COCD₃): δ [ppm]=−84.0 to −83.0 (24F), −86.4 (12F), −124.3 (12F), −130.0 to −129.0 (12F)

### Example 23

### (Fourth Reaction)

The operation up to the fourth reaction was performed in the same manner as in Example 21 except that in the fourth reaction in Example 21 described above, 1.6 g (molecular weight: 320, 4.9 mmol) of a compound (Ep-3) represented by Formula (20) was used instead of the compound (Ep-1) represented by Formula (13), and 3.7 g of an intermediate compound (2C-4) represented by Formula (83) was obtained.

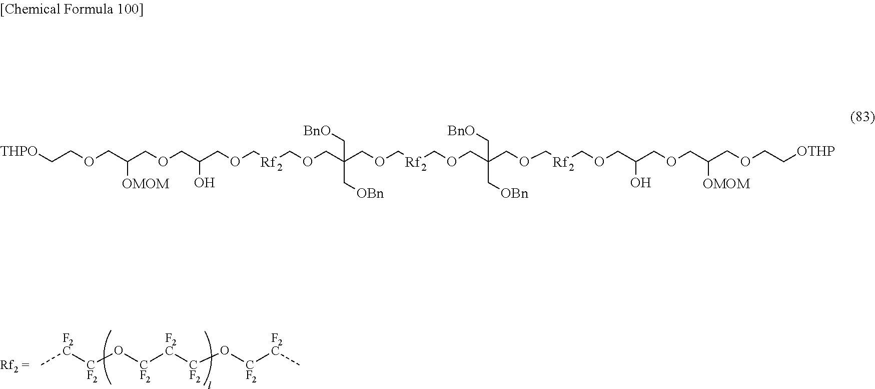

(In Formula (83), THP represents a tetrahydropyranyl group, Bn represents a benzyl group, and MOM represents a methoxymethyl group. Rf₂ is represented by the above formula, and 1 representing the average degree of polymerization in Rf₂ represents 2.0.)

### (Fifth Reaction)

3.8 g of a compound represented by Formula (83) (number-average molecular weight: 3,032, 1.3 mmol) as an intermediate compound (2C-4), 38 mL of methanol, and 3.8 mL of formic acid were charged into a 200 mL eggplant flask under a nitrogen gas atmosphere, and the mixture was stirred at room temperature until uniform. 0.38 g of palladium carbon (Pd/C) was further added to this uniform liquid, and the mixture was stirred at 70° C. for 8 hours to carry out a reaction.

The reaction solution obtained after the reaction was filtered to remove Pd/C, and the filtrate was thickened. After the thickening, the residue was purified by silica gel column chromatography to obtain 2.6 g of a compound (2C) represented by Formula (84) (number-average molecular weight: 2419, 1.2 mmol).

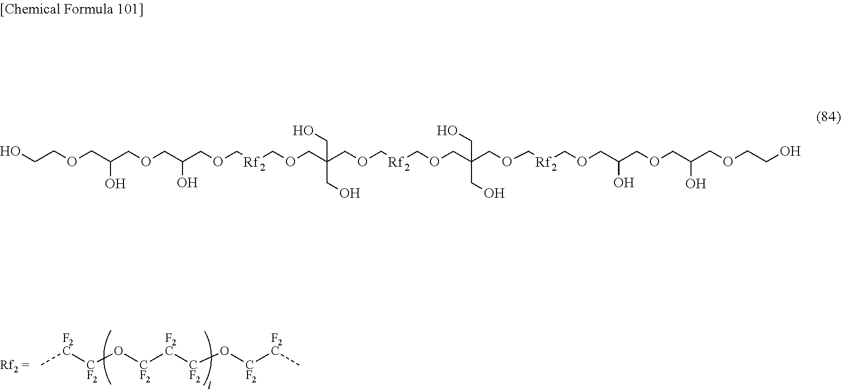

(In Formula (84), Rf₂ is represented by the above formula, and 1 representing the average degree of polymerization in Rf₂ represents 2.0.)

The obtained compound (2C) was subjected to ¹H-NMR and ¹⁹F-NMR measurements, and the structure was identified from the following results.

¹H-NMR (CD₃COCD₃): δ [ppm]=3.36 to 4.39 (66H)

¹⁹F-NMR (CD₃COCD₃): δ [ppm]=−84.2 to −83.0 (24F), −86.4 (12F), −124.2 (12F), −130.2 to −129.1 (12F)

### Example 24

### (Fourth Reaction)

The operation up to the fourth reaction was performed in the same manner as in Example 21 except that in the fourth reaction in Example 21 described above, 1.7 g (molecular weight: 334, 4.9 mmol) of a compound (Ep-4) represented by Formula (23) was used instead of the compound (Ep-1) represented by Formula (13), and 3.8 g of an intermediate compound (2D-4) represented by Formula (85) was obtained.

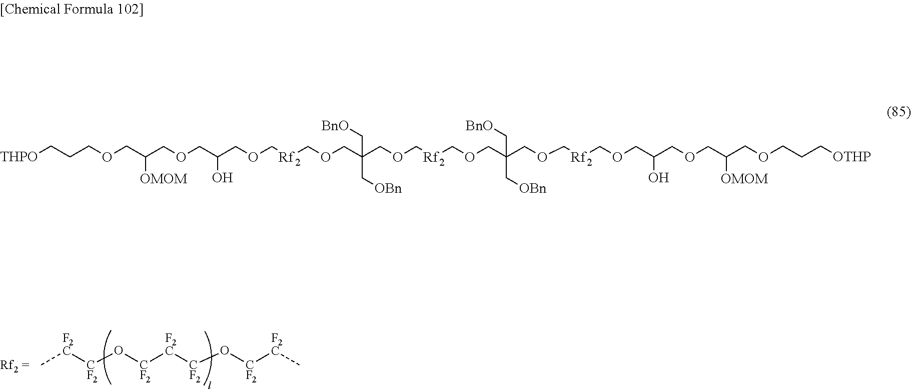

(In Formula (85), THP represents a tetrahydropyranyl group, Bn represents a benzyl group, and MOM represents a methoxymethyl group. Rf₂ is represented by the above formula, and 1 representing the average degree of polymerization in Rf₂ represents 2.0.)

### (Fifth Reaction)

3.7 g of a compound represented by Formula (85) (number-average molecular weight: 3,062, 1.3 mmol) as an intermediate compound (2D-4), 37 mL of methanol, and 3.7 mL of formic acid were charged into a 200 mL eggplant flask under a nitrogen gas atmosphere, and the mixture was stirred at room temperature until uniform. 0.37 g of palladium carbon (Pd/C) was further added to this uniform liquid, and the mixture was stirred at 70° C. for 8 hours to carry out a reaction.

The reaction solution obtained after the reaction was filtered to remove Pd/C, and the filtrate was thickened. After the thickening, the residue was purified by silica gel column chromatography to obtain 2.6 g of a compound (2D) represented by Formula (86) (number-average molecular weight: 2447, 1.2 mmol).

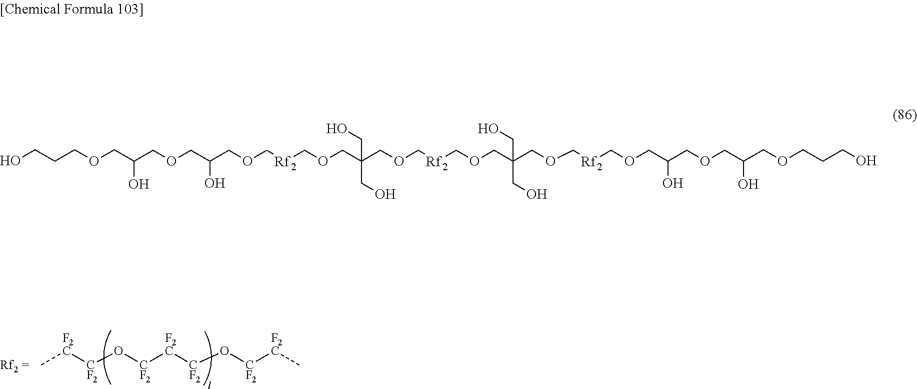

(In Formula (86), Rf₂ is represented by the above formula, and 1 representing the average degree of polymerization in Rf₂ represents 2.0.)

The obtained compound (2D) was subjected to ¹H-NMR and ¹⁹F-NMR measurements, and the structure was identified from the following results.

¹H-NMR (CD₃COCD₃): δ [ppm]=1.63 to 1.85 (4H), 3.36 to 4.35 (66H)

¹⁹F-NMR (CD₃COCD₃): δ [ppm]=−84.0 to −83.0 (24F), −86.4 (12F), −124.3 (12F), −130.0 to −129.0 (12F)

### Example 25

### (Fourth Reaction)

The operation up to the fourth reaction was performed in the same manner as in Example 21 except that in the fourth reaction in Example 21 described above, 1.4 g (molecular weight: 216, 4.9 mmol) of a compound (Ep-5) represented by Formula (26) was used instead of the compound (Ep-1) represented by Formula (13), and 3.8 g of an intermediate compound (2E-4) represented by Formula (87) was obtained.

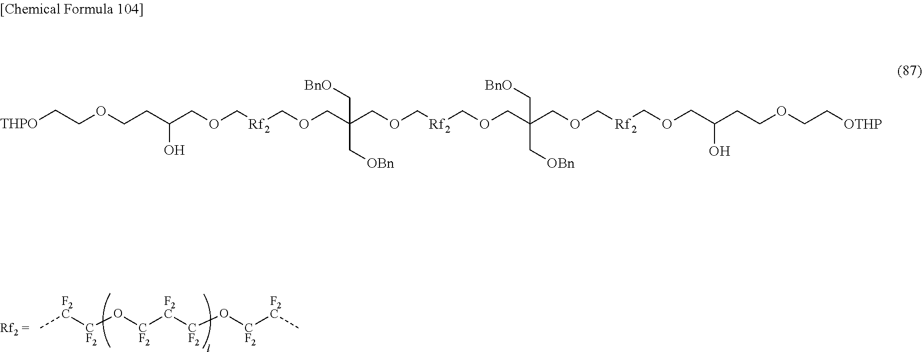

(In Formula (87), THP represents a tetrahydropyranyl group, and Bn represents a benzyl group. Rf₂ is represented by the above formula, and 1 representing the average degree of polymerization in Rf₂ represents 2.0.)

### (Fifth Reaction)

3.8 g of a compound represented by Formula (87) (number-average molecular weight: 2,925, 1.4 mmol) as an intermediate compound (2E-4), 39 mL of methanol, and 3.8 mL of formic acid were charged into a 200 mL eggplant flask under a nitrogen gas atmosphere, and the mixture was stirred at room temperature until uniform. 0.38 g of palladium carbon (Pd/C) was further added to this uniform liquid, and the mixture was stirred at 70° C. for 8 hours to carry out a reaction.

The reaction solution obtained after the reaction was filtered to remove Pd/C, and the filtrate was thickened. After the thickening, the residue was purified by silica gel column chromatography to obtain 2.7 g of a compound (2E) represented by Formula (88) (number-average molecular weight: 2299, 1.2 mmol).

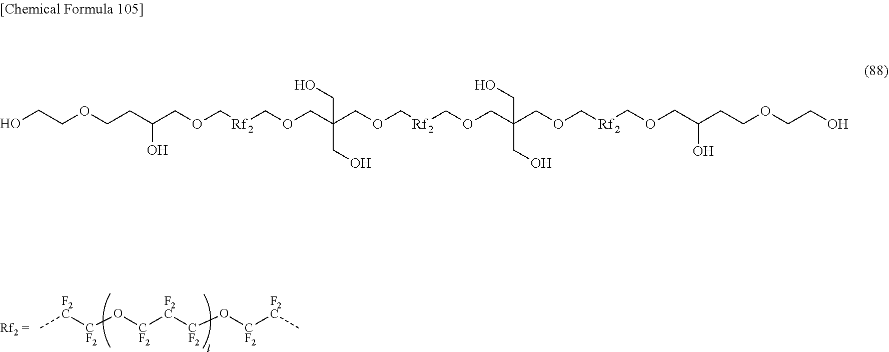

(In Formula (88), Rf₂ is represented by the above formula, and 1 representing the average degree of polymerization in Rf₂ represents 2.0.)

The obtained compound (2E) was subjected to ¹H-NMR and ¹⁹F-NMR measurements, and the structure was identified from the following results.

¹H-NMR (CD₃COCD₃): δ [ppm]=1.65 to 1.79 (4H), 3.41 to 4.33 (54H)

¹⁹F-NMR (CD₃COCD₃): δ [ppm]=−84.0 to −83.0 (24F), −86.4 (12F), −124.3 (12F), −130.0 to −129.0 (12F)

### Example 26

### (Fourth Reaction)

The operation up to the fourth reaction was performed in the same manner as in Example 21 except that in the fourth reaction in Example 21 described above, 1.4 g (molecular weight: 334, 4.9 mmol) of a compound (Ep-6) represented by Formula (29) was used instead of the compound (Ep-1) represented by Formula (13), and 3.6 g of an intermediate compound (2F-4) represented by Formula (89) was obtained.

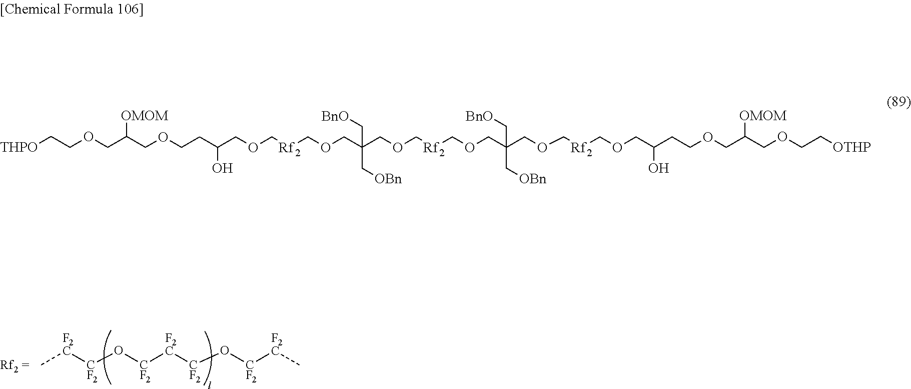

(In Formula (89), THP represents a tetrahydropyranyl group, Bn represents a benzyl group, and MOM represents a methoxymethyl group. Rf₂ is represented by the above formula, and 1 representing the average degree of polymerization in Rf₂ represents 2.0.)

### (Fifth Reaction)

3.8 g of a compound represented by Formula (89) (number-average molecular weight: 2,925, 1.4 mmol) as an intermediate compound (2F-4), 39 mL of methanol, and 3.8 mL of formic acid were charged into a 200 mL eggplant flask under a nitrogen gas atmosphere, and the mixture was stirred at room temperature until uniform. 0.38 g of palladium carbon (Pd/C) was further added to this uniform liquid, and the mixture was stirred at 70° C. for 8 hours to carry out a reaction.

The reaction solution obtained after the reaction was filtered to remove Pd/C, and the filtrate was thickened. After the thickening, the residue was purified by silica gel column chromatography to obtain 2.7 g of a compound (2F) represented by Formula (90) (number-average molecular weight: 2447, 1.2 mmol).

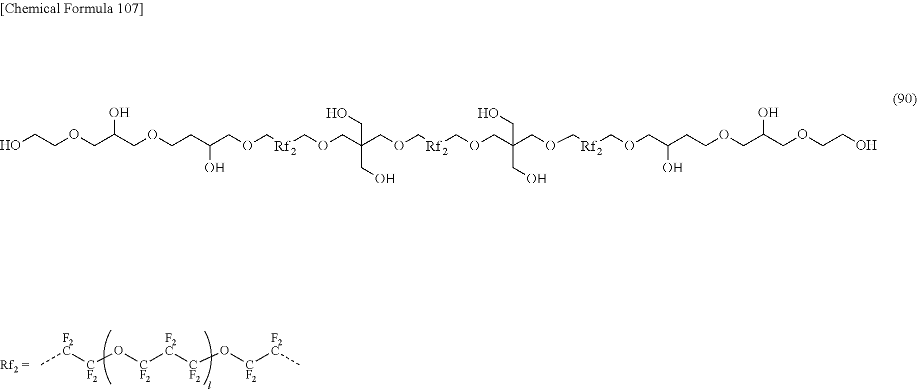

(In Formula (90), Rf₂ is represented by the above formula, and 1 representing the average degree of polymerization in Rf₂ represents 2.0.)

The obtained compound (2F) was subjected to ¹H-NMR and ¹⁹F-NMR measurements, and the structure was identified from the following results.

¹H-NMR (CD₃COCD₃): δ [ppm]=1.64 to 1.80 (4H), 3.43 to 4.32 (66H)

¹⁹F-NMR (CD₃COCD₃): δ [ppm]=−84.0 to −83.0 (24F), −86.4 (12F), −124.3 (12F), −130.0 to −129.0 (12F)

### Example 27

### (First Reaction)

10 g of a compound represented by

HOCH₂CF₂O(CF₂CF₂O)ⱼ(CF₂O)ₖCF₂CH₂OH (j representing the average degree of polymerization in the formula was 2.4, and k representing the average degree of polymerization in the formula was 2.4) (number-average molecular weight: 614, molecular weight distribution: 1.1), 25.4 g of a compound represented by Formula (A1) (molecular weight: 624, 40.7 mmol), 1.1 g of potassium iodide (molecular weight: 166, 6.5 mmol), and 33 mL of N,N′-dimethylformamide (DMF) were charged into a 300 mL eggplant flask under a nitrogen gas atmosphere, and stirred at room temperature to obtain a mixed solution. 21.2 g of cesium carbonate (molecular weight: 325, 65.1 mmol) was added to this mixed solution, stirred and reacted at 70° C. for 10 hours.

The reaction product obtained after the reaction was cooled to 0° C., 100 mL of water was added thereto, and the reaction was stopped. The obtained reaction solution was transferred to a separatory funnel and extracted three times with 100 mL of ethyl acetate. The organic layer was washed with saline and dewatered with anhydrous sodium sulfate. After filtering off a desiccant, the filtrate was thickened, and the residue was purified by silica gel column chromatography to obtain 10.5 g of a compound represented by Formula (91) as an intermediate compound (2G-1).

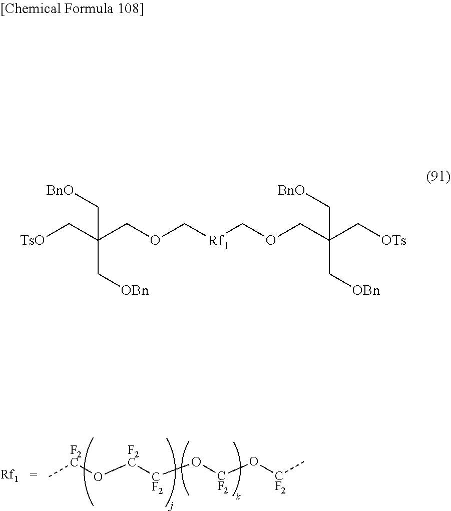

(In Formula (91), Ts represents a p-toluenesulfonyl group, and Bn represents a benzyl group. Rf₁ is represented by the above formula, j representing the average degree of polymerization in Rf₁ is 2.4, and k representing the average degree of polymerization is 2.4.)

### (Second Reaction)

20 g of a compound represented by

HOCH₂CF₂O(CF₂CF₂O)ⱼ(CF₂O)ₖCF₂CH₂OH (j representing the average degree of polymerization in the formula was 2.4, and k representing the average degree of polymerization was 2.4) (number-average molecular weight: 614, molecular weight distribution: 1.1), 2.9 g of 3,4-dihydro-2H-pyran, and 66 mL of a mixed solution of ASAHIKLIN (registered trademark) AE-3000 (manufactured by AGC Inc.) as a fluorine-based solvent and dichloromethane (volume ratio: 1:1) were charged into a 300 mL eggplant flask under a nitrogen gas atmosphere, and the mixture was stirred at 0° C. until uniform, thereby obtaining a mixed solution. 0.12 g of p-toluenesulfonic acid monohydrate was added to this mixed solution, stirred at 0° C. for 30 minutes, and then stirred at room temperature for 2 hours to carry out a reaction.

The reaction product obtained after the reaction was cooled to 0° C., 50 mL of saturated aqueous sodium bicarbonate was added thereto, and the reaction was stopped. The obtained reaction solution was transferred to a separatory funnel and extracted three times with 100 mL of ethyl acetate. The organic layer was washed with saline and dewatered with anhydrous sodium sulfate. After filtering off a desiccant, the filtrate was thickened, and the residue was purified by silica gel column chromatography to obtain 10.0 g of a compound represented by Formula (92) as an intermediate compound (2G-2).

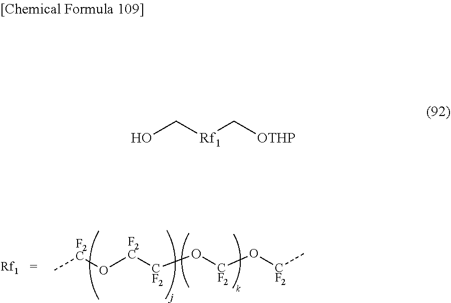

(In Formula (92), THP represents a tetrahydropyranyl group, Rf₁ is represented by the above formula, j representing the average degree of polymerization in Rf₁ is 2.4, and k representing the average degree of polymerization is 2.4.)

### (Third Reaction)

7.0 g of a compound represented by Formula (91) as an intermediate compound (2G-1) (number-average molecular weight: 1522, 4.6 mmol), 7.1 g of a compound represented by Formula (92) as an intermediate compound (2G-2) (number-average molecular weight: 699, 10.1 mmol), 0.31 g of potassium iodide (molecular weight: 166, 1.9 mmol), and 10 mL of N,N′-dimethylformamide (DMF) were charged into a 200 mL eggplant flask under a nitrogen gas atmosphere, and stirred at room temperature to obtain a mixed solution. 6.0 g of cesium carbonate (molecular weight: 325, 18.4 mmol) was added to this mixed solution, stirred and reacted at 70° C. for 10 hours.

The reaction solution obtained after the reaction was returned to room temperature, 12 g of a 10% hydrogen chloride/methanol solution (hydrogen chloride-methanol reagent (5% to 10%) manufactured by Tokyo Chemical Industry Co., Ltd.) was added thereto, and the mixture was stirred at room temperature for 2 hours. The reaction solution was transferred little by little to a separatory funnel containing 100 mL of saline, and the mixture was extracted three times with 200 mL of ethyl acetate. The organic layer was washed with 100 mL of saline, 100 mL of saturated aqueous sodium bicarbonate, and 100 mL of saline in this order, and dewatered with anhydrous sodium sulfate. After filtering off a desiccant, the filtrate was thickened, and the residue was purified by silica gel column chromatography. The above-described steps made 5.2 g of a compound represented by Formula (93) obtained as an intermediate compound (2G-3).

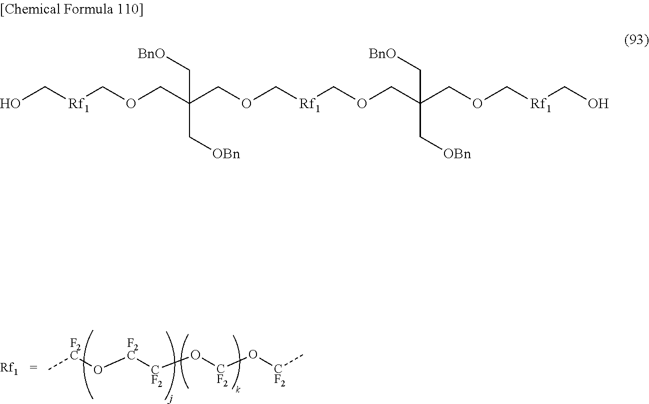

(In Formula (93), Bn represents a benzyl group, Rf₁ is represented by the above formula, j representing the average degree of polymerization in Rf₁ is 2.4, and k representing the average degree of polymerization is 2.4.)

### (Fourth Reaction)

5.0 g of a compound represented by Formula (93) (number-average molecular weight: 2,407, 2.2 mmol) as an intermediate compound (2G-3), 0.8 g of a compound (Ep-7) represented by Formula (33) (molecular weight: 172, 4.9 mmol), and 2.1 mL of t-butanol were charged into a 200 mL eggplant flask under a nitrogen gas atmosphere, and the mixture was stirred at room temperature until uniform. 0.076 g of potassium tert-butoxide (molecular weight: 113, 0.67 mmol) was further added to this uniform liquid, and the mixture was stirred at 70° C. for 16 hours to carry out a reaction.

A reaction product obtained after the reaction was cooled to 25° C., transferred to a separatory funnel containing 100 mL of water, and extracted three times with 100 mL of ethyl acetate. The organic layer was washed with water and dewatered with anhydrous sodium sulfate. After filtering off a desiccant, the filtrate was thickened, and the residue was purified by silica gel column chromatography to obtain 3.6 g of a compound represented by Formula (94) as an intermediate compound (2G-4).

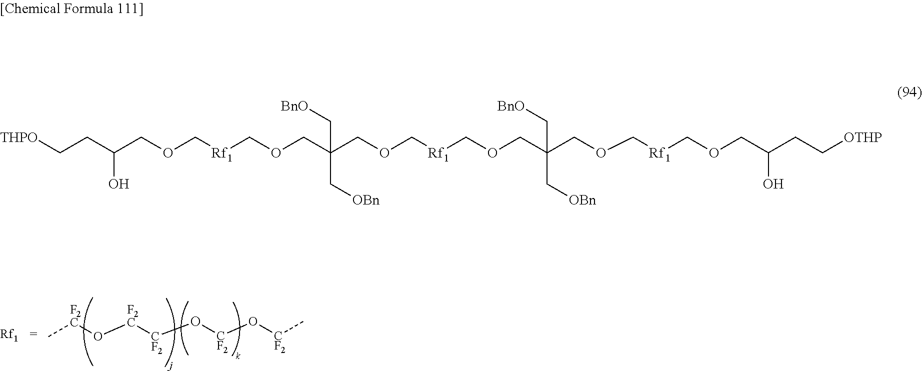

(In Formula (94), THP represents a tetrahydropyranyl group, Bn represents a benzyl group, Rf₁ is represented by the above formula, j representing the average degree of polymerization in Rf₁ is 2.4, and k representing the average degree of polymerization is 2.4.)

### (Fifth Reaction)

3.6 g of a compound represented by Formula (94) (number-average molecular weight: 2,752, 1.4 mmol) as an intermediate compound (2G-4), 36 mL of methanol, and 3.6 mL of formic acid were charged into a 200 mL eggplant flask under a nitrogen gas atmosphere, and the mixture was stirred at room temperature until uniform. 0.36 g of palladium carbon (Pd/C) was further added to this uniform liquid, and the mixture was stirred at 70° C. for 8 hours to carry out a reaction.

The reaction solution obtained after the reaction was filtered to remove Pd/C, and the filtrate was thickened. After the thickening, the residue was purified by silica gel column chromatography to obtain 2.5 g of a compound (2G) represented by Formula (95) (number-average molecular weight: 2225, 1.2 mmol).

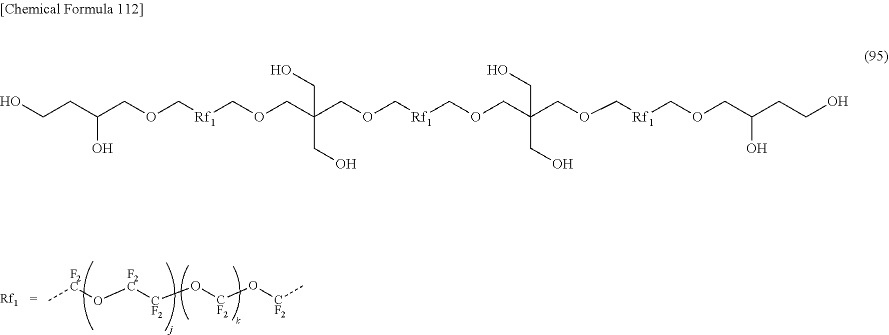

(In Formula (95), Rf₁ is represented by the above formula, j representing the average degree of polymerization in Rf₁ is 2.4, and k representing the average degree of polymerization is 2.4.)

The obtained compound (2G) was subjected to ¹H-NMR and ¹⁹F-NMR measurements, and the structure was identified from the following results.

¹H-NMR (CD₃COCD₃): δ [ppm]=1.64 to 1.81 (4H), 3.44 to 4.31 (46H)

¹⁹F-NMR (CD₃COCD₃): δ [ppm]=−55.6 to −50.6 (14.4F), −77.7 (6F), −80.3 (6F), −91.0 to −88.5 (28.8F)

### Example 28

### (Fourth Reaction)

The operation up to the fourth reaction was performed in the same manner as in Example 27 except that in the fourth reaction in Example 27 described above, 1.0 g (molecular weight: 200, 4.9 mmol) of a compound (Ep-8) represented by Formula (37) described above was used instead of the compound (Ep-7) represented by Formula (33), and 3.7 g of an intermediate compound (2H-4) represented by Formula (96) was obtained.

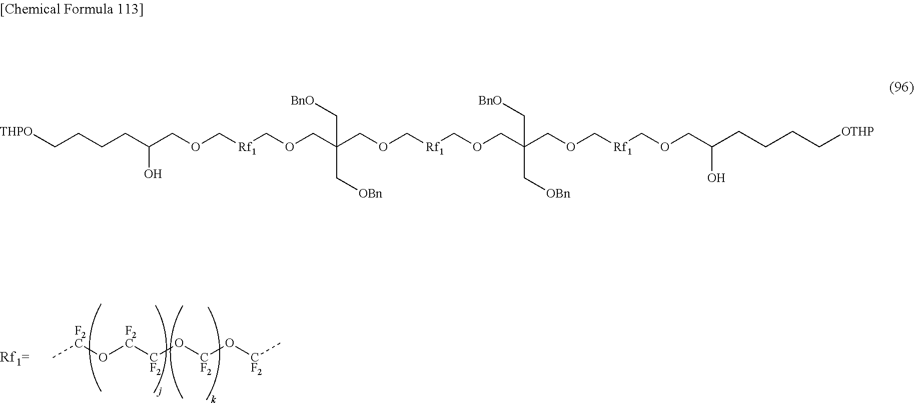

(In Formula (96), THP represents a tetrahydropyranyl group, and Bn represents a benzyl group. Rf₁ is represented by the above formula, j representing the average degree of polymerization in Rf₁ is 2.4, and k representing the average degree of polymerization is 2.4.)

### (Fifth Reaction)

3.7 g of a compound represented by Formula (96) (number-average molecular weight: 2,807, 1.4 mmol) as an intermediate compound (2H-4), 37 mL of methanol, and 3.7 mL of formic acid were charged into a 200 mL eggplant flask under a nitrogen gas atmosphere, and the mixture was stirred at room temperature until uniform. 0.37 g of palladium carbon (Pd/C) was further added to this uniform liquid, and the mixture was stirred at 70° C. for 8 hours to carry out a reaction.

The reaction solution obtained after the reaction was filtered to remove Pd/C, and the filtrate was thickened. After the thickening, the residue was purified by silica gel column chromatography to obtain 2.6 g of a compound (2H) represented by Formula (97) (number-average molecular weight: 2281, 1.2 mmol).

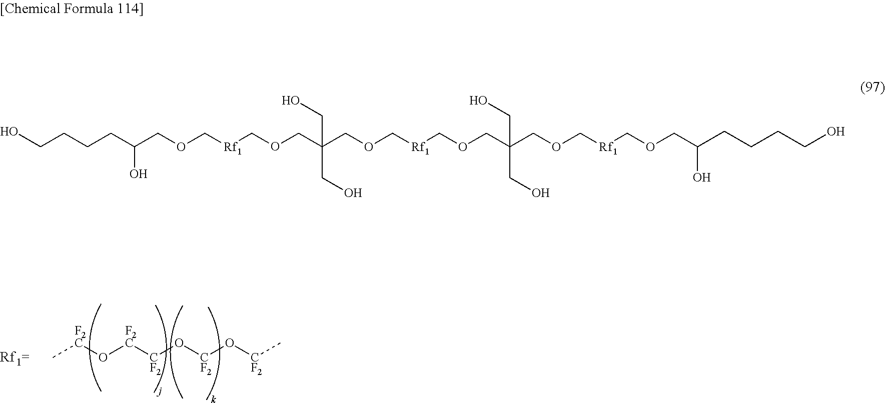

(In Formula (97), Rf₁ is represented by the above formula, j representing the average degree of polymerization in Rf₁ is 2.4, and k representing the average degree of polymerization is 2.4.)

The obtained compound (2H) was subjected to ¹H-NMR and ¹⁹F-NMR measurements, and the structure was identified from the following results.

¹H-NMR (CD₃COCD₃): δ [ppm]=1.32 to 1.80 (12H), 3.39 to 4.35 (46H)

¹⁹F-NMR (CD₃COCD₃): δ [ppm]=−55.7 to −50.5 (14.4F), −77.7 (6F), −80.3 (6F), −91.1 to −88.4 (28.8F)

### Example 29

### (First Reaction)

10 g of a compound represented by

HOCH₂CF₂O(CF₂CF₂O)ⱼ(CF₂O)ₖCF₂CH₂OH (j representing the average degree of polymerization in the formula was 3.8, and k representing the average degree of polymerization in the formula was 0) (number-average molecular weight: 618, molecular weight distribution: 1.1), 25.2 g of a compound represented by Formula (A1) (molecular weight: 624, 40.4 mmol), 1.1 g of potassium iodide (molecular weight: 166, 6.5 mmol), and 33 mL of N,N′-dimethylformamide (DMF) were charged into a 300 mL eggplant flask under a nitrogen gas atmosphere, and stirred at room temperature to obtain a mixed solution. 21.0 g of cesium carbonate (molecular weight: 325, 64.6 mmol) was added to this mixed solution, stirred and reacted at 70° C. for 10 hours.

The reaction product obtained after the reaction was cooled to 0° C., 100 mL of water was added thereto, and the reaction was stopped. The obtained reaction solution was transferred to a separatory funnel and extracted three times with 100 mL of ethyl acetate. The organic layer was washed with saline and dewatered with anhydrous sodium sulfate. After filtering off a desiccant, the filtrate was thickened, and the residue was purified by silica gel column chromatography to obtain 10.0 g of a compound represented by Formula (98) as an intermediate compound (2I-1).

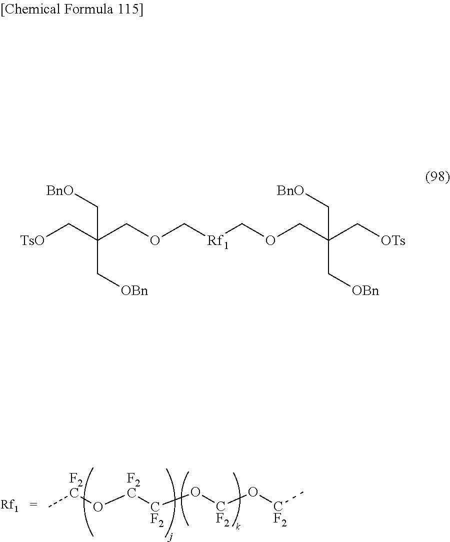

(In Formula (98), Ts represents a p-toluenesulfonyl group, and Bn represents a benzyl group. Rf₁ is represented by the above formula, j representing the average degree of polymerization in Rf₁ is 3.8, and k representing the average degree of polymerization is 0.)

### (Second Reaction)

20 g of a compound represented by

HOCH₂CF₂O(CF₂CF₂O)ⱼ(CF₂O)ₖCF₂CH₂OH (j representing the average degree of polymerization in the formula was 3.8, and k representing the average degree of polymerization was 0) (number-average molecular weight: 618, molecular weight distribution: 1.1), 2.9 g of 3,4-dihydro-2H-pyran, and 66 mL of a mixed solution of ASAHIKLIN (registered trademark) AE-3000 (manufactured by AGC Inc.) as a fluorine-based solvent and dichloromethane (volume ratio: 1:1) were charged into a 300 mL eggplant flask under a nitrogen gas atmosphere, and the mixture was stirred at 0° C. until uniform, thereby obtaining a mixed solution. 0.12 g of p-toluenesulfonic acid monohydrate was added to this mixed solution, stirred at 0° C. for 30 minutes, and then stirred at room temperature for 2 hours to carry out a reaction.

The reaction product obtained after the reaction was cooled to 0° C., 50 mL of saturated aqueous sodium bicarbonate was added thereto, and the reaction was stopped. The obtained reaction solution was transferred to a separatory funnel and extracted three times with 100 mL of ethyl acetate. The organic layer was washed with saline and dewatered with anhydrous sodium sulfate. After filtering off a desiccant, the filtrate was thickened, and the residue was purified by silica gel column chromatography to obtain 9.9 g of a compound represented by Formula (99) as an intermediate compound (21-2).

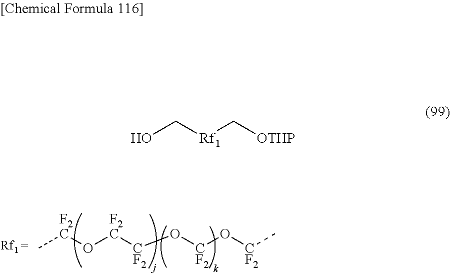

(In Formula (99). T HP represents a tetrahydropvranyl group. Rf₁ is represented by the above formula, j representing the average degree of polymerization in Rf₁ is 3.8, and k representing the average degree of polymerization is 0.)

### (Third Reaction)

7.1 g of a compound represented by Formula (98) as an intermediate compound (21-1) (number-average molecular weight: 1525, 4.6 mmol), 7.0 g of a compound represented by Formula (99) as an intermediate compound (21-2) (number-average molecular weight: 702, 10.1 mmol), 0.31 g of potassium iodide (molecular weight: 166, 1.8 mmol), and 10 mL of N,N′-dimethylformamide (DMF) were charged into a 200 mL eggplant flask under a nitrogen gas atmosphere, and stirred at room temperature to obtain a mixed solution. 6.0 g of cesium carbonate (molecular weight: 325, 18.4 mmol) was added to this mixed solution, stirred and reacted at 70° C. for 10 hours.

The reaction solution obtained after the reaction was returned to room temperature, 12 g of a 10% hydrogen chloride/methanol solution (hydrogen chloride-methanol reagent (5% to 10%) manufactured by Tokyo Chemical Industry Co., Ltd.) was added thereto, and the mixture was stirred at room temperature for 2 hours. The reaction solution was transferred little by little to a separatory funnel containing 100 mL of saline, and the mixture was extracted three times with 200 mL of ethyl acetate. The organic layer was washed with 100 mL of saline, 100 mL of saturated aqueous sodium bicarbonate, and 100 mL of saline in this order, and dewatered with anhydrous sodium sulfate. After filtering off a desiccant, the filtrate was thickened, and the residue was purified by silica gel column chromatography. The above-described steps made 5.0 g of a compound represented by Formula (100) obtained as an intermediate compound (21-3).

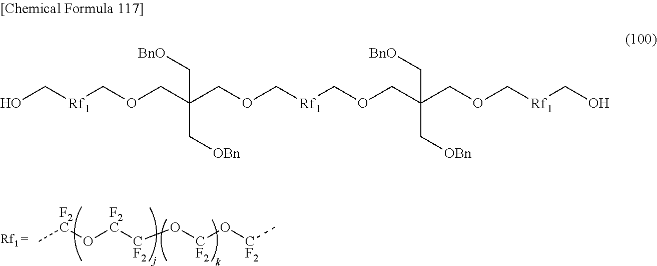

(In Formula (100), Bn represents a benzyl group, Rf₁ is represented by the above formula, j representing the average degree of polymerization in Rf₁ is 3.8, and k representing the average degree of polymerization is 0.)

### (Fourth Reaction)

5.0 g of a compound represented by Formula (100) (number-average molecular weight: 2,418, 2.2 mmol) as an intermediate compound (2I-3), 1.5 g of a compound (Ep-9) represented by Formula (41) (molecular weight: 172, 4.9 mmol), and 2.1 mL of t-butanol were charged into a 200 mL eggplant flask under a nitrogen gas atmosphere, and the mixture was stirred at room temperature until uniform. 0.076 g of potassium tert-butoxide (molecular weight: 113, 0.67 mmol) was further added to this uniform liquid, and the mixture was stirred at 70° C. for 16 hours to carry out a reaction.

A reaction product obtained after the reaction was cooled to 25° C., transferred to a separatory funnel containing 100 mL of water, and extracted three times with 100 mL of ethyl acetate. The organic layer was washed with water and dewatered with anhydrous sodium sulfate. After filtering off a desiccant, the filtrate was thickened, and the residue was purified by silica gel column chromatography to obtain 3.5 g of a compound represented by Formula (101) as an intermediate compound (2I-4).

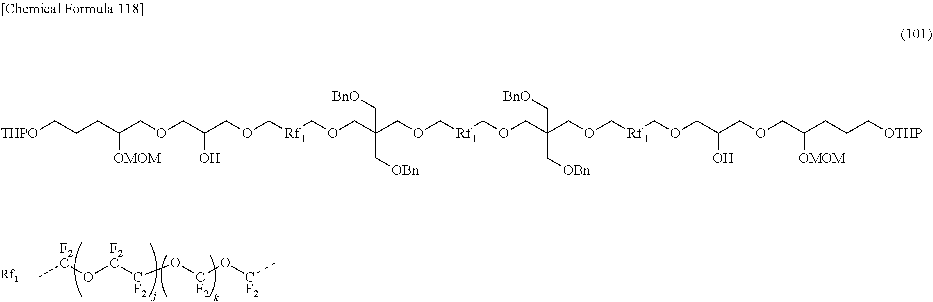

(In Formula (101), THP represents a tetrahydropyranyl group, Bn represents a benzyl group, and MOM represents a methoxymethyl group. Rf₁ is represented by the above formula, j representing the average degree of polymerization in Rf₁ is 3.8, and k representing the average degree of polymerization is 0.)

### (Fifth Reaction)

3.5 g of a compound represented by Formula (101) (number-average molecular weight: 3,027, 1.2 mmol) as an intermediate compound (21-4), 35 mL of methanol, and 3.5 mL of formic acid were charged into a 200 mL eggplant flask under a nitrogen gas atmosphere, and the mixture was stirred at room temperature until uniform. 0.35 g of palladium carbon (Pd/C) was further added to this uniform liquid, and the mixture was stirred at 70° C. for 8 hours to carry out a reaction.

The reaction solution obtained after the reaction was filtered to remove Pd/C, and the filtrate was thickened. After the thickening, the residue was purified by silica gel column chromatography to obtain 2.4 g of a compound (21) represented by Formula (102) (number-average molecular weight: 2413, 1.1 mmol).

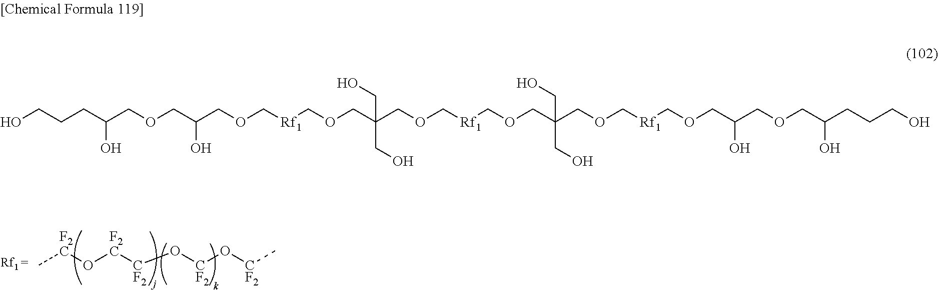

(In Formula (102), Rf₁ is represented by the above formula, j representing the average degree of polymerization in Rf₁ is 3.8, and k representing the average degree of polymerization is 0.)

The obtained compound (21) was subjected to ¹H-NMR and ¹⁹F-NMR measurements, and the structure was identified from the following results.

¹H-NMR (CD₃COCD₃): δ [ppm]=1.32 to 1.70 (8H), 3.41 to 4.30 (58H)

¹⁹F-NMR (CD₃COCD₃): δ [ppm]=−78.6 (6F), −81.3 (6F), −90.0 to −88.5 (45.6F)

### Example 30

### (First Reaction)

10 g of a compound represented by

HOCH₂CF₂CF₂O(CF₂CF₂CF₂O)ᵢCF₂CF₂CH₂OH (1 representing the average degree of polymerization in the formula was 2.0) (number-average molecular weight: 610, molecular weight distribution: 1.1), 12.4 g of a compound represented by Formula (S1) (molecular weight: 302, 41.0 mmol), 1.1 g of potassium iodide (molecular weight: 166, 6.6 mmol), and 33 mL of N,N′-dimethylformamide (DMF) were charged into a 300 mL eggplant flask under a nitrogen gas atmosphere, and stirred at room temperature to obtain a mixed solution. 21.3 g of cesium carbonate (molecular weight: 325, 65.6 mmol) was added to this mixed solution, stirred and reacted at 70° C. for 10 hours.

The reaction product obtained after the reaction was cooled to 0° C., 100 mL of water was added thereto, and the reaction was stopped. The obtained reaction solution was transferred to a separatory funnel and extracted three times with 100 mL of ethyl acetate. The organic layer was washed with saline and dewatered with anhydrous sodium sulfate. After filtering off a desiccant, the filtrate was thickened, and the residue was purified by silica gel column chromatography to obtain 10.0 g of a compound represented by Formula (103) as an intermediate compound (2J-1).

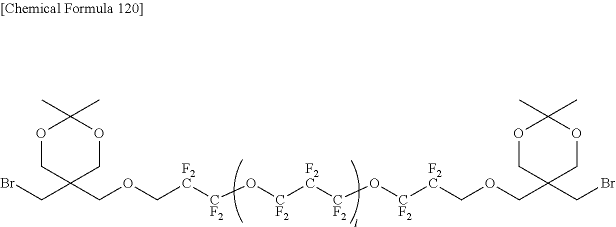

(In Formula (103), 1 representing the average degree of polymerization represents 2.0.)

### (Second Reaction)

20 g of a compound represented by HOCH₂CF₂CF₂O (CF₂CF₂CF₂O)ₗCF₂CF₂CH₂OH (1 in the formula represents an average degree of polymerization of 2.0) (number-average molecular weight: 610, molecular weight distribution: 1.1), 7.1 g of a compound (Ep-10) represented by Formula (44) (molecular weight: 272, 26.2 mmol), and 31 mL of t-butanol were charged into a 200 mL eggplant flask under a nitrogen gas atmosphere, and the mixture was stirred at room temperature until uniform. 0.736 g of potassium tert-butoxide (molecular weight: 113, 6.6 mmol) was further added to this uniform liquid, and the mixture was stirred at 70° C. for 16 hours to carry out a reaction.

A reaction product obtained after the reaction was cooled to 25° C., transferred to a separatory funnel containing 100 mL of water, and extracted three times with 100 mL of ethyl acetate. The organic layer was washed with water and dewatered with anhydrous sodium sulfate. After filtering off a desiccant, the filtrate was thickened, and the residue was purified by silica gel column chromatography to obtain 9.7 g of a compound represented by Formula (104) as an intermediate compound (2J-2).

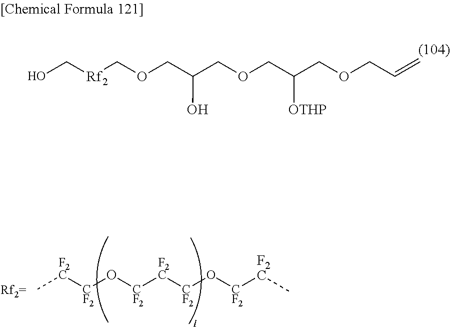

(In Formula (104), THP represents a tetrahydropyranyl group, Rf₂ is represented by the above formula, and 1 representing the average degree of polymerization in Rf₂ represents 2.0.)

### (Third Reaction)

5.0 g of a compound represented by Formula (103) as an intermediate compound (2J-1) (number-average molecular weight: 1052, 4.8 mmol), 9.2 g of a compound represented by Formula (104) as an intermediate compound (2J-2) (number-average molecular weight: 882, 10.5 mmol), 0.32 g of potassium iodide (molecular weight: 166, 1.9 mmol), and 10 mL of N,N′-dimethylformamide (DMF) were charged into a 200 mL eggplant flask under a nitrogen gas atmosphere, and stirred at room temperature to obtain a mixed solution. 6.2 g of cesium carbonate (molecular weight: 325, 19.0 mmol) was added to this mixed solution, stirred and reacted at 70° C. for 10 hours.

The reaction solution obtained after the reaction was returned to room temperature, 25 g of a 10% hydrogen chloride/methanol solution (hydrogen chloride-methanol reagent (5% to 10%) manufactured by Tokyo Chemical Industry Co., Ltd.) was added thereto, and the mixture was stirred at room temperature for 2 hours. The reaction solution was transferred little by little to a separatory funnel containing 100 mL of saline, and the mixture was extracted three times with 200 mL of ethyl acetate. The organic layer was washed with 100 mL of saline, 100 mL of saturated aqueous sodium bicarbonate, and 100 mL of saline in this order, and dewatered with anhydrous sodium sulfate. After filtering off a desiccant, the filtrate was thickened, and the residue was purified by silica gel column chromatography. The above-described steps made 3.2 g (number-average molecular weight: 2411, 1.2 mmol) of a compound (2J) represented by Formula (105) obtained.

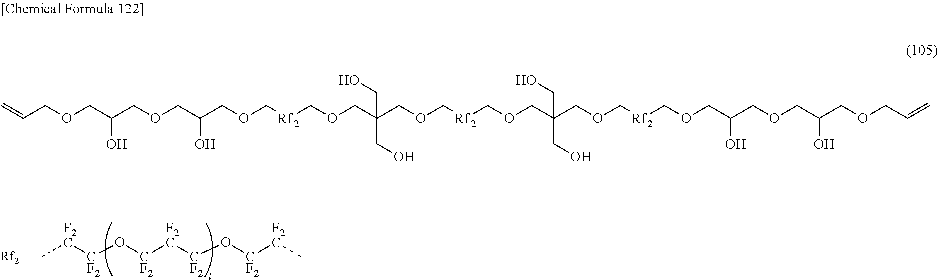

(In Formula (105), Rf₂ is represented by the above formula, and 1 representing the average degree of polymerization in Rf₂ represents 2.0.)

The obtained compound (2J) was subjected to ¹H-NMR and ¹⁹F-NMR measurements, and the structure was identified from the following results.

¹H-NMR (CD₃COCD₃): δ [ppm]=3.39 to 4.34 (60H), 5.14 to 5.22 (2H), 5.26 to 5.35 (2H), 5.87 to 5.91 (2H)

¹⁹F-NMR (CD₃COCD₃): δ [ppm]=−84.0 to −83.0 (24F), −86.4 (12F), −124.3 (12F), −130.0 to −129.0 (12F)

### Example 31

### (First Reaction)

The operation was performed in the same manner as in the first reaction in Example 30 described above, thereby obtaining an intermediate compound (2J-1) represented by Formula (103).

### (Second Reaction)

20 g of a compound represented by HOCH₂CF₂CF₂O (CF₂CF₂CF₂O)ᵢCF₂CF₂CH₂OH (1 in the formula represents an average degree of polymerization of 2.0) (number-average molecular weight: 610, molecular weight distribution: 1.1), 7.9 g of a compound (Ep-11) represented by Formula (47) (molecular weight: 300, 26.2 mmol), and 31 mL of t-butanol were charged into a 200 mL eggplant flask under a nitrogen gas atmosphere, and the mixture was stirred at room temperature until uniform. 0.736 g of potassium tert-butoxide (molecular weight: 113, 6.6 mmol) was further added to this uniform liquid, and the mixture was stirred at 70° C. for 16 hours to carry out a reaction.

A reaction product obtained after the reaction was cooled to 25° C., transferred to a separatory funnel containing 100 mL of water, and extracted three times with 100 mL of ethyl acetate. The organic layer was washed with water and dewatered with anhydrous sodium sulfate. After filtering off a desiccant, the filtrate was thickened, and the residue was purified by silica gel column chromatography to obtain 9.8 g of a compound represented by Formula (106) as an intermediate compound (2K-2).

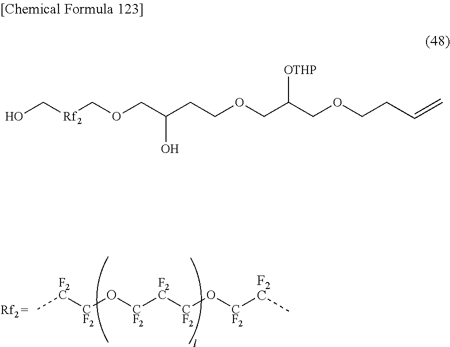

(In Formula (106), THP represents a tetrahydropyranyl group, Rf₂ is represented by the above formula, and 1 representing the average degree of polymerization in Rf₂ represents 2.0.)

### (Third Reaction)

5.0 g of a compound represented by Formula (103) as an intermediate compound (2J-1) (number-average molecular weight: 1052, 4.8 mmol), 9.5 g of a compound represented by Formula (106) as an intermediate compound (2K-2) (number-average molecular weight: 910, 10.5 mmol), 0.32 g of potassium iodide (molecular weight: 166, 1.9 mmol), and 10 mL of N,N′-dimethylformamide (DMF) were charged into a 200 mL eggplant flask under a nitrogen gas atmosphere, and stirred at room temperature to obtain a mixed solution. 6.2 g of cesium carbonate (molecular weight: 325, 19.0 mmol) was added to this mixed solution, stirred and reacted at 70° C. for 10 hours.

The reaction solution obtained after the reaction was returned to room temperature, 25 g of a 10% hydrogen chloride/methanol solution (hydrogen chloride-methanol reagent (5% to 10%) manufactured by Tokyo Chemical Industry Co., Ltd.) was added thereto, and the mixture was stirred at room temperature for 2 hours. The reaction solution was transferred little by little to a separatory funnel containing 100 mL of saline, and the mixture was extracted three times with 200 mL of ethyl acetate. The organic layer was washed with 100 mL of saline, 100 mL of saturated aqueous sodium bicarbonate, and 100 mL of saline in this order, and dewatered with anhydrous sodium sulfate. After filtering off a desiccant, the filtrate was thickened, and the residue was purified by silica gel column chromatography. The above-described steps made 3.2 g (number-average molecular weight: 2467, 1.2 mmol) of a compound (2K) represented by Formula (107) obtained.

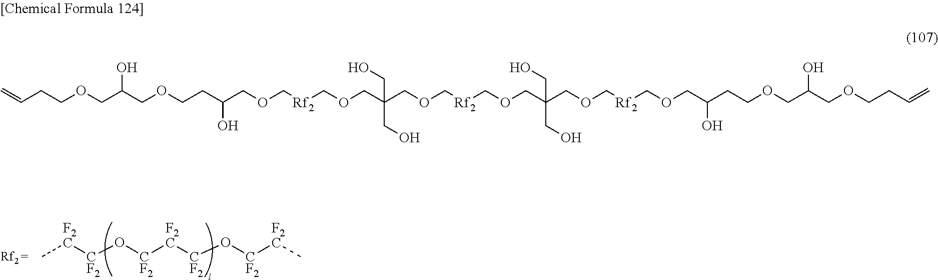

(In Formula (107), Rf₂ is represented by the above formula, and 1 representing the average degree of polymerization in Rf₂ represents 2.0.)

The obtained compound (2K) was subjected to ¹H-NMR and ¹⁹F-NMR measurements, and the structure was identified from the following results.

¹H-NMR (CD₃COCD₃): δ [ppm]=1.66 to 1.81 (4H), 2.33 to 2.43 (4H), 3.39 to 4.34 (60H), 5.14 to 5.22 (2H), 5.26 to 5.35 (2H), 5.87 to 5.91 (2H)

¹⁹F-NMR (CD₃COCD₃): δ [ppm]=−84.0 to −83.0 (24F), −86.4 (12F), −124.3 (12F), −130.0 to −129.0 (12F)

### Example 32

### (Fourth Reaction)

The operation up to the fourth reaction was performed in the same manner as in Example 29 except that in the fourth reaction in Example 29 described above, 1.5 g (molecular weight: 299, 4.9 mmol) of a compound (Ep-12) represented by Formula (50) described above was used instead of the compound (Ep-9) represented by Formula (41), and 3.6 g of an intermediate compound (2L-4) represented by Formula (108) was obtained.

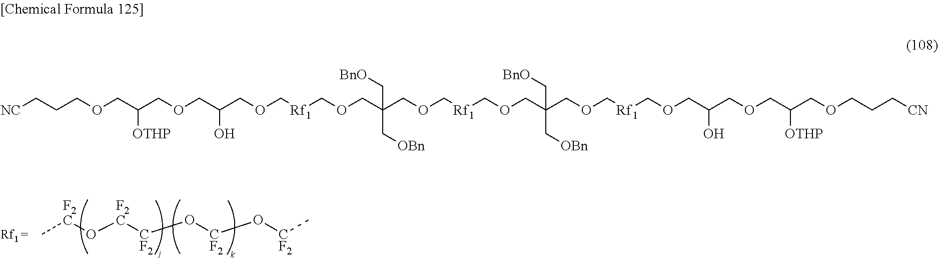

(In Formula (108), THP represents a tetrahydropyranyl group, and Bn represents a benzyl group. Rf₁ is represented by the above formula, j representing the average degree of polymerization in Rf₁ is 3.8, and k representing the average degree of polymerization is 0.)

### (Fifth Reaction)

3.6 g of a compound represented by Formula (108) (number-average molecular weight: 3,017, 1.3 mmol) as an intermediate compound (2L-4), 36 mL of methanol, and 3.6 mL of formic acid were charged into a 200 mL eggplant flask under a nitrogen gas atmosphere, and the mixture was stirred at room temperature until uniform. 0.36 g of palladium carbon (Pd/C) was further added to this uniform liquid, and the mixture was stirred at 70° C. for 8 hours to carry out a reaction.

The reaction solution obtained after the reaction was filtered to remove Pd/C, and the filtrate was thickened. After the thickening, the residue was purified by silica gel column chromatography to obtain 2.5 g of a compound (2L) represented by Formula (109) (number-average molecular weight: 2491, 1.1 mmol).

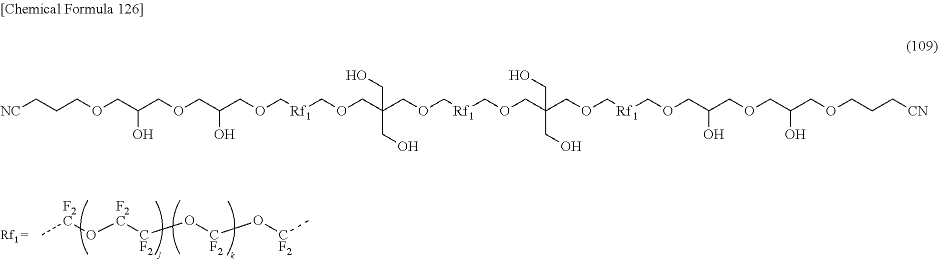

(In Formula (109), Rf₁ is represented by the above formula, j representing the average degree of polymerization in Rf₁ is 3.8, and k representing the average degree of polymerization is 0.)

The obtained compound (2L) was subjected to ¹H-NMR and ¹⁹F-NMR measurements, and the structure was identified from the following results. ¹H-NMR (CD₃COCD₃): δ [ppm]=1.13 to 1.27 (4H), 2.00 to 2.10 (4H), 3.37 to 4.36 (60H) ¹⁹F-NMR (CD₃COCD₃): δ [ppm]=−78.6 (6F), −81.3 (6F), −90.0 to −88.5 (45.6F)

### Example 33

### (Fourth Reaction)

The operation up to the fourth reaction was performed in the same manner as in Example 27 except that in the fourth reaction in Example 27 described above, 1.9 g (molecular weight: 388, 4.9 mmol) of a compound (Ep-13) represented by Formula (54) described above was used instead of the compound (Ep-7) represented by Formula (33), and 3.8 g of an intermediate compound (2M-4) represented by Formula (110) was obtained.

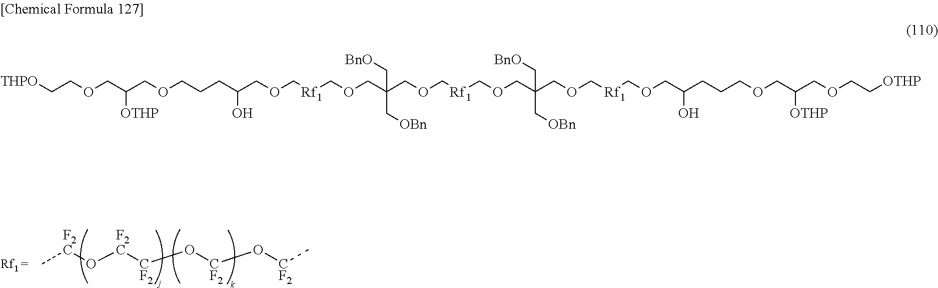

(In Formula (110), THP represents a tetrahydropyranyl group, and Bn represents a benzyl group. Rf₁ is represented by the above formula, j representing the average degree of polymerization in Rf₁ is 2.4, and k representing the average degree of polymerization is 2.4.)

### (Fifth Reaction)

3.8 g of a compound represented by Formula (110) (number-average molecular weight: 3,184, 1.3 mmol) as an intermediate compound (2M-4), 38 mL of methanol, and 3.8 mL of formic acid were charged into a 200 mL eggplant flask under a nitrogen gas atmosphere, and the mixture was stirred at room temperature until uniform. 0.38 g of palladium carbon (Pd/C) was further added to this uniform liquid, and the mixture was stirred at 70° C. for 8 hours to carry out a reaction.

The reaction solution obtained after the reaction was filtered to remove Pd/C, and the filtrate was thickened. After the thickening, the residue was purified by silica gel column chromatography to obtain 2.7 g of a compound (2M) represented by Formula (III) (number-average molecular weight: 2490, 1.2 mmol).

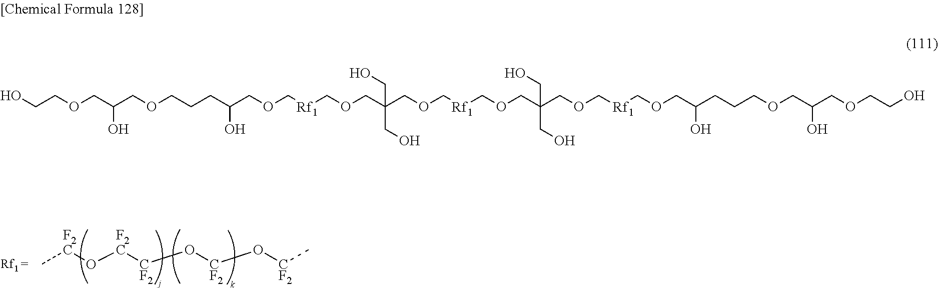

(In Formula (III), Rf₁ is represented by the above formula, j representing the average degree of polymerization in Rf₁ is 2.4, and k representing the average degree of polymerization is 2.4.)

The obtained compound (2M) was subjected to ¹H-NMR and ¹⁹F-NMR measurements, and the structure was identified from the following results.

¹H-NMR (CD₃COCD₃): δ [ppm]=1.64 to 1.81 (8H), 3.37 to 4.36 (62H)

¹⁹F-NMR (CD₃COCD₃): δ [ppm]=−55.8 to −50.5 (14.4F), −77.7 (6F), −80.3 (6F), −91.0 to −88.3 (28.8F)

### Example 34

### (Fourth Reaction)

The operation up to the fourth reaction was performed in the same manner as in Example 21 except that in the fourth reaction in Example 21 described above, 1.3 g (molecular weight: 264, 4.9 mmol) of a compound (Ep-14) represented by Formula (56) described above was used instead of the compound (Ep-1) represented by Formula (13), and 3.6 g of an intermediate compound (2N-4) represented by Formula (112) was obtained.

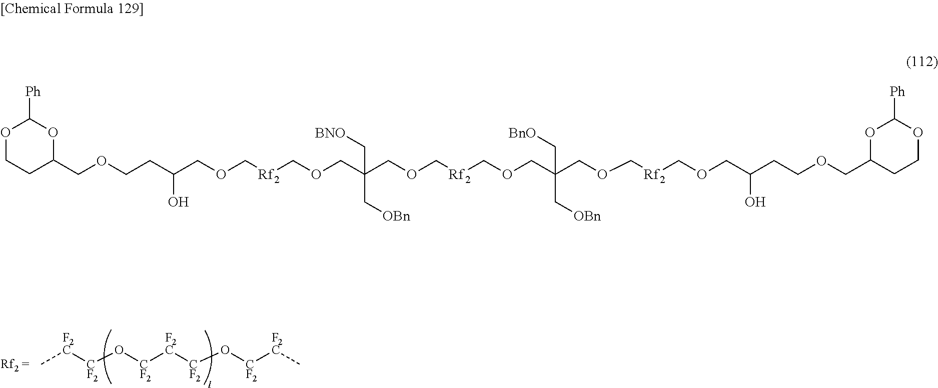

(In Formula (112), Ph represents a phenyl group, and Bn represents a benzyl group. Rf₂ is represented by the above formula, and 1 representing the average degree of polymerization in Rf₂ represents 2.0.)

### (Fifth Reaction)

3.6 g of a compound represented by Formula (112) (number-average molecular weight: 2,921, 1.3 mmol) as an intermediate compound (2N-4), 36 mL of methanol, and 3.6 mL of formic acid were charged into a 200 mL eggplant flask under a nitrogen gas atmosphere, and the mixture was stirred at room temperature until uniform. 0.36 g of palladium carbon (Pd/C) was further added to this uniform liquid, and the mixture was stirred at 70° C. for 8 hours to carry out a reaction.

The reaction solution obtained after the reaction was filtered to remove Pd/C, and the filtrate was thickened. After the thickening, the residue was purified by silica gel column chromatography to obtain 2.6 g of a compound (2N) represented by Formula (113) (number-average molecular weight: 2387, 1.2 mmol).

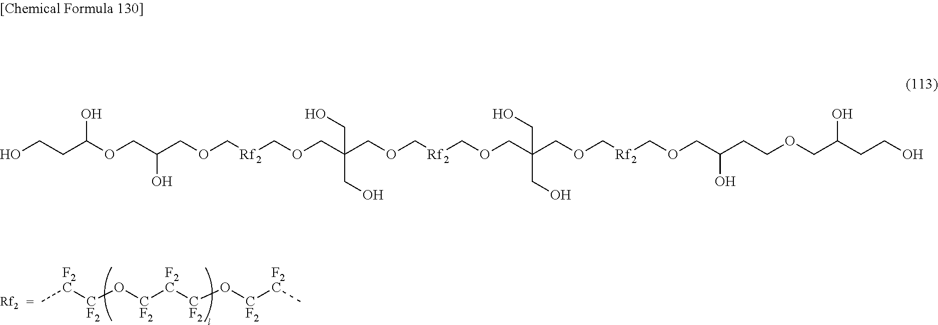

(In Formula (113), Rf₂ is represented by the above formula, and 1 representing the average degree of polymerization in Rf₂ represents 2.0.)

The obtained compound (2N) was subjected to ¹H-NMR and ¹⁹F-NMR measurements, and the structure was identified from the following results.

¹H-NMR (CD₃COCD₃): δ [ppm]=1.63 to 1.87 (8H), 3.30 to 4.45 (58H)

¹⁹F-NMR (CD₃COCD₃): δ [ppm]=−84.0 to −83.0 (24F), −86.4 (12F), −124.3 (12F), −130.0 to −129.0 (12F)

### Example 35

### (Fourth Reaction)

The operation up to the fourth reaction was performed in the same manner as in Example 29 except that in the fourth reaction in Example 29 described above, 1.6 g (molecular weight: 317, 4.9 mmol) of a compound (Ep-15) represented by Formula (59) described above was used instead of the compound (Ep-9) represented by Formula (41), and 3.7 g of an intermediate compound (20-4) represented by Formula (114) was obtained.

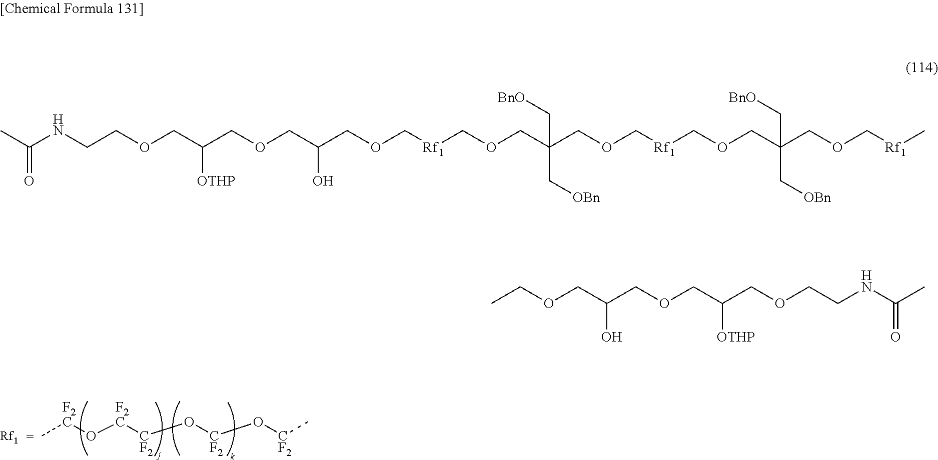

(In Formula (114), THP represents a tetrahydropyranyl group, Bn represents a benzyl group, Rf₁ is represented by the above formula, j representing the average degree of polymerization in Rf₁ is 3.8, and k representing the average degree of polymerization is 0.)

### (Fifth Reaction)

3.7 g of a compound represented by Formula (114) (number-average molecular weight: 3,053, 1.3 mmol) as an intermediate compound (20-4), 37 mL of methanol, and 3.7 mL of formic acid were charged into a 200 mL eggplant flask under a nitrogen gas atmosphere, and the mixture was stirred at room temperature until uniform. 0.37 g of palladium carbon (Pd/C) was further added to this uniform liquid, and the mixture was stirred at 70° C. for 8 hours to carry out a reaction.

The reaction solution obtained after the reaction was filtered to remove Pd/C, and the filtrate was thickened. After the thickening, the residue was purified by silica gel column chromatography to obtain 2.6 g of a compound (20) represented by Formula (115) (number-average molecular weight: 2527, 1.1 mmol).

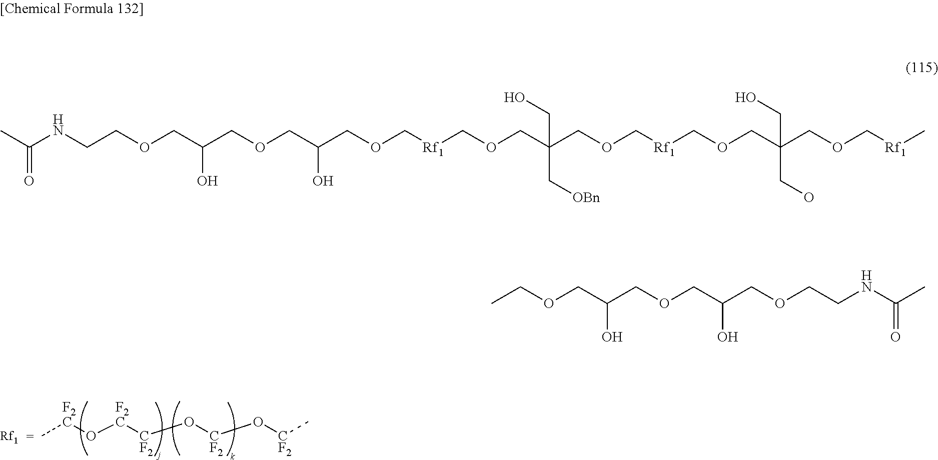

(In Formula (115), Rf₁ is represented by the above formula, j representing the average degree of polymerization in Rf₁ is 3.8, and k representing the average degree of polymerization is 0.)

The obtained compound (20) was subjected to ¹H-NMR and ¹⁹F-NMR measurements, and the structure was identified from the following results.

¹H-NMR (CD₃COCD₃): δ [ppm]=1.75 to 1.93 (6H), 3.30 to 4.40 (64H), 7.25 to 7.41 (2H)

¹⁹F-NMR (CD₃COCD₃): δ [ppm]=−78.6 (6F), −81.3 (6F), −90.0 to −88.5 (45.6F)

### Example 36

### (First Reaction)

10 g of a compound represented by

HOCH₂CF₂CF₂O(CF₂CF₂CF₂O)ₗCF₂CF₂CH₂OH (1 representing the average degree of polymerization in the formula was 2.0) (number-average molecular weight: 610, molecular weight distribution: 1.1), 26.8 g of a compound represented by Formula (B1) (molecular weight: 652, 41.0 mmol), 1.1 g of potassium iodide (molecular weight: 166, 6.6 mmol), and 33 mL of N,N′-dimethylformamide (DMF) were charged into a 300 mL eggplant flask under a nitrogen gas atmosphere, and stirred at room temperature to obtain a mixed solution. 21.3 g of cesium carbonate (molecular weight: 325, 65.6 mmol) was added to this mixed solution, stirred and reacted at 70° C. for 10 hours.

The reaction product obtained after the reaction was cooled to 0° C., 100 mL of water was added thereto, and the reaction was stopped. The obtained reaction solution was transferred to a separatory funnel and extracted three times with 100 mL of ethyl acetate. The organic layer was washed with saline and dewatered with anhydrous sodium sulfate. After filtering off a desiccant, the filtrate was thickened, and the residue was purified by silica gel column chromatography to obtain 11.5 g of a compound represented by Formula (116) as an intermediate compound (2P-1).

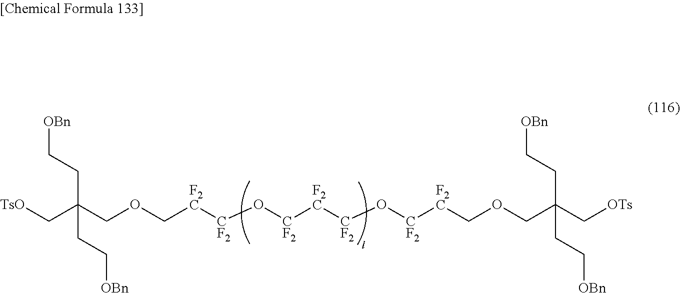

(In Formula (116), Ts represents a p-toluenesulfonyl group, Bn represents a benzyl group, and 1 representing the average degree of polymerization represents 2.0.)

### (Second Reaction)

The operation was performed in the same manner as in the second reaction in Example 21 described above, thereby obtaining an intermediate compound (2A-2) represented by Formula (77).

### (Third Reaction)

7.0 g (number-average molecular weight: 1,571, 4.5 mmol) of a compound represented by Formula (116) as an intermediate compound (2P-1), 6.8 g (number-average molecular weight: 694, 9.8 mmol) of a compound represented by Formula (77) as an intermediate compound (2A-2), 0.30 g of potassium iodide (molecular weight: 166, 1.8 mmol), and 9 mL of N,N′-dimethylformamide (DMF) were charged into a 200 mL eggplant flask under a nitrogen gas atmosphere, and the mixture was stirred at room until uniform. 5.8 g of cesium carbonate (molecular weight: 325, 17.8 mmol) was added to this uniform liquid, stirred and reacted at 70° C. for 10 hours.

The reaction solution obtained after the reaction was returned to room temperature, 12 g of a 10% hydrogen chloride/methanol solution (hydrogen chloride-methanol reagent (5% to 10%) manufactured by Tokyo Chemical Industry Co., Ltd.) was added thereto, and the mixture was stirred at room temperature for 2 hours. The reaction solution was transferred little by little to a separatory funnel containing 100 mL of saline, and the mixture was extracted three times with 200 mL of ethyl acetate. The organic layer was washed with 100 mL of saline, 100 mL of saturated aqueous sodium bicarbonate, and 100 mL of saline in this order, and dewatered with anhydrous sodium sulfate. After filtering off a desiccant, the filtrate was thickened, and the residue was purified by silica gel column chromatography. The above-described steps made 5.0 g of a compound represented by Formula (117) obtained as an intermediate compound (2P-3).

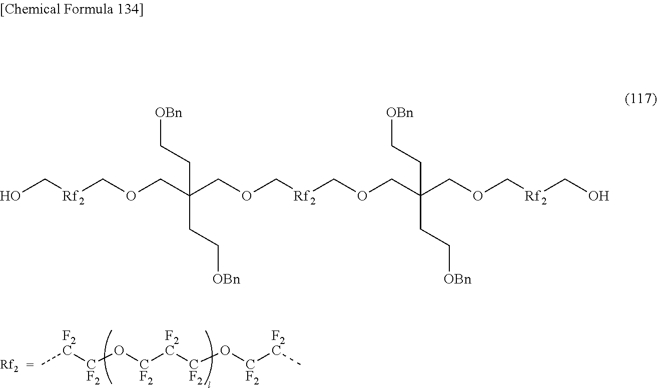

(In Formula (117), Bn represents a benzyl group, Rf₂ is represented by the above formula, and 1 representing the average degree of polymerization in Rf₂ represents 2.0.)

### (Fourth Reaction)

5.0 g of a compound represented by Formula (117) (number-average molecular weight: 2,446, 2.2 mmol) as an intermediate compound (2P-3), 0.98 g of a compound (Ep-1) represented by Formula (13) (molecular weight: 202, 4.8 mmol), and 2.1 mL of t-butanol were charged into a 200 mL eggplant flask under a nitrogen gas atmosphere, and the mixture was stirred at room temperature until uniform. 0.074 g of potassium tert-butoxide (molecular weight: 113, 0.66 mmol) was further added to this uniform liquid, and the mixture was stirred at 70° C. for 16 hours to carry out a reaction.

A reaction product obtained after the reaction was cooled to 25° C., transferred to a separatory funnel containing 100 mL of water, and extracted three times with 100 mL of ethyl acetate. The organic layer was washed with water and dewatered with anhydrous sodium sulfate. After filtering off a desiccant, the filtrate was thickened, and the residue was purified by silica gel column chromatography to obtain 3.6 g of a compound represented by Formula (118) as an intermediate compound (2P-4).

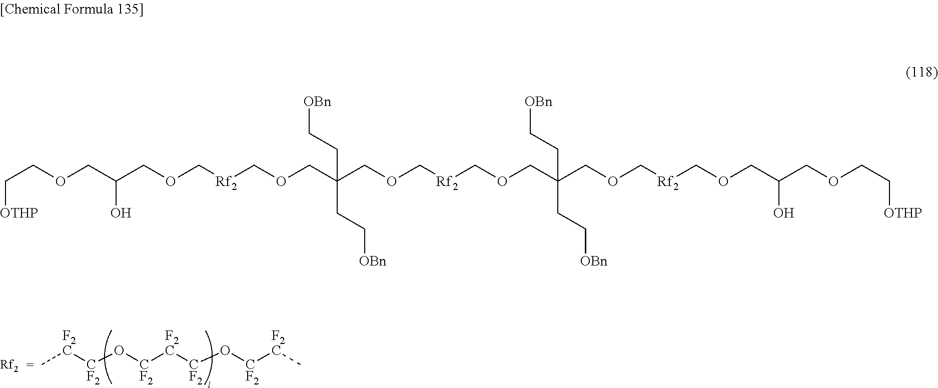

(In Formula (118), THP represents a tetrahydropyranyl group, and Bn represents a benzyl group. Rf₂ is represented by the above formula, and 1 representing the average degree of polymerization in Rf₂ represents 2.0.)

### (Fifth Reaction)

3.6 g of a compound represented by Formula (118) (number-average molecular weight: 2,851, 1.3 mmol) as an intermediate compound (2P-4), 36 mL of methanol, and 3.6 mL of formic acid were charged into a 200 mL eggplant flask under a nitrogen gas atmosphere, and the mixture was stirred at room temperature until uniform. 0.36 g of palladium carbon (Pd/C) was further added to this uniform liquid, and the mixture was stirred at 70° C. for 8 hours to carry out a reaction.

The reaction solution obtained after the reaction was filtered to remove Pd/C, and the filtrate was thickened. After the thickening, the residue was purified by silica gel column chromatography to obtain 2.5 g of a compound (2P) represented by Formula (119) (number-average molecular weight: 2327, 1.2 mmol).

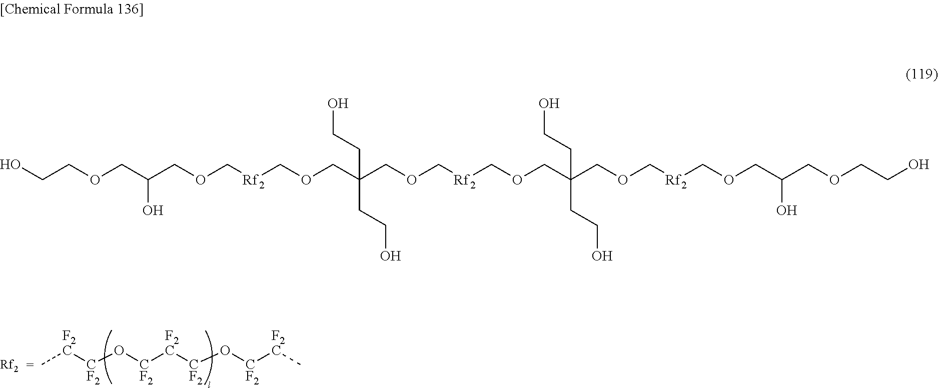

(In Formula (119), Rf₂ is represented by the above formula, and 1 representing the average degree of polymerization in Rf₂ represents 2.0.)

The obtained compound (2P) was subjected to ¹H-NMR and ¹⁹F-NMR measurements, and the structure was identified from the following results.

¹H-NMR (CD₃COCD₃): δ [ppm]=1.60 to 1.87 (8H), 3.37 to 4.37 (54H)

¹⁹F-NMR (CD₃COCD₃): δ [ppm]=−84.0 to −83.0 (24F), −86.4 (12F), −124.3 (12F), −130.0 to −129.0 (12F)

### Example 37

### (Fourth Reaction)

The operation up to the fourth reaction was performed in the same manner as in Example 36 except that in the fourth reaction in Example 36 described above, 0.8 g (molecular weight: 172, 4.8 mmol) of a compound (Ep-7) represented by Formula (33) described above was used instead of the compound (Ep-1) represented by Formula (13), and 3.5 g of an intermediate compound (2Q-4) represented by Formula (120) was obtained.

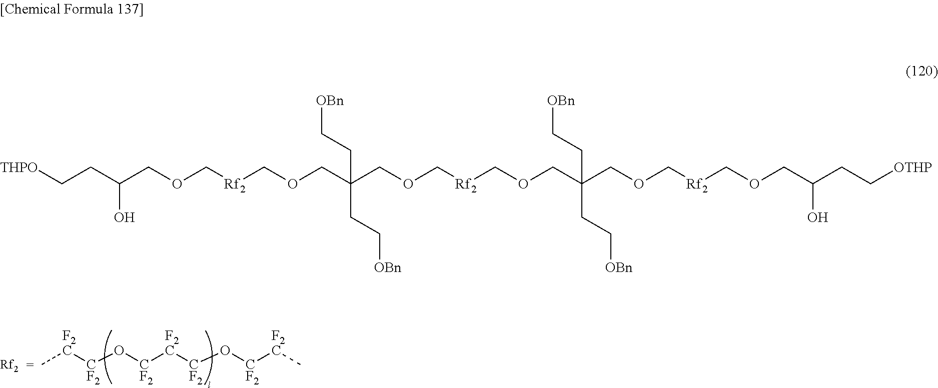

(In Formula (120), THP represents a tetrahydropyranyl group, Bn represents a benzyl group, Rf₂ is represented by the above formula, and 1 representing the average degree of polymerization in Rf₂ represents 2.0.)

### (Fifth Reaction)

3.5 g of a compound represented by Formula (120) (number-average molecular weight: 2,791, 1.3 mmol) as an intermediate compound (2Q-4), 35 mL of methanol, and 3.5 mL of formic acid were charged into a 200 mL eggplant flask under a nitrogen gas atmosphere, and the mixture was stirred at room temperature until uniform. 0.35 g of palladium carbon (Pd/C) was further added to this uniform liquid, and the mixture was stirred at 70° C. for 8 hours to carry out a reaction.

The reaction solution obtained after the reaction was filtered to remove Pd/C, and the filtrate was thickened. After the thickening, the residue was purified by silica gel column chromatography to obtain 2.5 g of a compound (2Q) represented by Formula (121) (number-average molecular weight: 2267, 1.2 mmol).

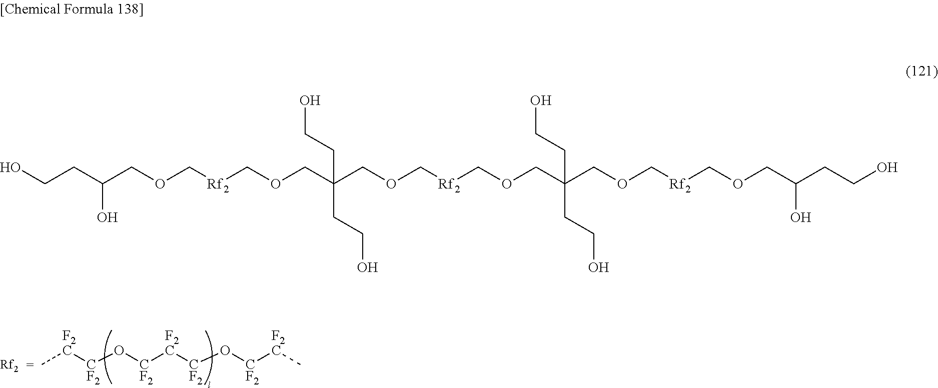

(In Formula (121), Rf₂ is represented by the above formula, and 1 representing the average degree of polymerization in Rf₂ represents 2.0.)

The obtained compound (2Q) was subjected to ¹H-NMR and ¹⁹F-NMR measurements, and the structure was identified from the following results.

¹H-NMR (CD₃COCD₃): δ [ppm]=1.60 to 1.86 (12H), 3.37 to 4.37 (46H) ¹⁹F-NMR (CD₃COCD₃): δ [ppm]=−84.0 to −83.0 (24F), −86.4 (12F), −124.3 (12F), −130.0 to −129.0 (12F)

### Example 38

### (First Reaction)

10 g of a compound represented by

HOCH₂CF₂CF₂O(CF₂CF₂CF₂O)ₗCF₂CF₂CH₂OH (1 representing the average degree of polymerization in the formula was 2.0) (number-average molecular weight: 610, molecular weight distribution: 1.1), 27.9 g of a compound represented by Formula (C1) (molecular weight: 680, 41.0 mmol), 1.1 g of potassium iodide (molecular weight: 166, 6.6 mmol), and 33 mL of N,N′-dimethylformamide (DMF) were charged into a 300 mL eggplant flask under a nitrogen gas atmosphere, and stirred at room temperature to obtain a mixed solution. 21.3 g of cesium carbonate (molecular weight: 325, 65.6 mmol) was added to this mixed solution, stirred and reacted at 70° C. for 10 hours.

The reaction product obtained after the reaction was cooled to 0° C., 100 mL of water was added thereto, and the reaction was stopped. The obtained reaction solution was transferred to a separatory funnel and extracted three times with 100 mL of ethyl acetate. The organic layer was washed with saline and dewatered with anhydrous sodium sulfate. After filtering off a desiccant, the filtrate was thickened, and the residue was purified by silica gel column chromatography to obtain 11.0 g of a compound represented by Formula (122) as an intermediate compound (2R-1).

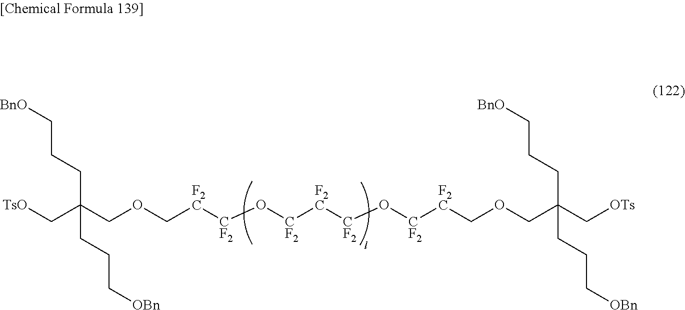

(In Formula (122), Ts represents a p-toluenesulfonyl group, Bn represents a benzyl group, and 1 representing the average degree of polymerization represents 2.0.)

### (Second Reaction)

The operation was performed in the same manner as in the second reaction in Example 21 described above, thereby obtaining an intermediate compound (2A-2) represented by Formula (77).

### (Third Reaction)

7.0 g of a compound represented by Formula (122) as an intermediate compound (2R-1) (number-average molecular weight: 1627, 4.3 mmol), 6.6 g of a compound represented by Formula (77) as an intermediate compound (2A-2) (number-average molecular weight: 694, 9.5 mmol), 0.29 g of potassium iodide (molecular weight: 166, 1.7 mmol), and 9 mL of N,N′-dimethylformamide (DMF) were charged into a 200 mL eggplant flask under a nitrogen gas atmosphere, and stirred at room temperature to obtain a mixed solution. 5.6 g of cesium carbonate (molecular weight: 325, 17.2 mmol) was added to this mixed solution, stirred and reacted at 70° C. for 10 hours.

The reaction solution obtained after the reaction was returned to room temperature, 12 g of a 10% hydrogen chloride/methanol solution (hydrogen chloride-methanol reagent (5% to 10%) manufactured by Tokyo Chemical Industry Co., Ltd.) was added thereto, and the mixture was stirred at room temperature for 2 hours. The reaction solution was transferred little by little to a separatory funnel containing 100 mL of saline, and the mixture was extracted three times with 200 mL of ethyl acetate. The organic layer was washed with 100 mL of saline, 100 mL of saturated aqueous sodium bicarbonate, and 100 mL of saline in this order, and dewatered with anhydrous sodium sulfate. After filtering off a desiccant, the filtrate was thickened, and the residue was purified by silica gel column chromatography. The above-described steps made 5.0 g of a compound represented by Formula (123) obtained as an intermediate compound (2R-3).

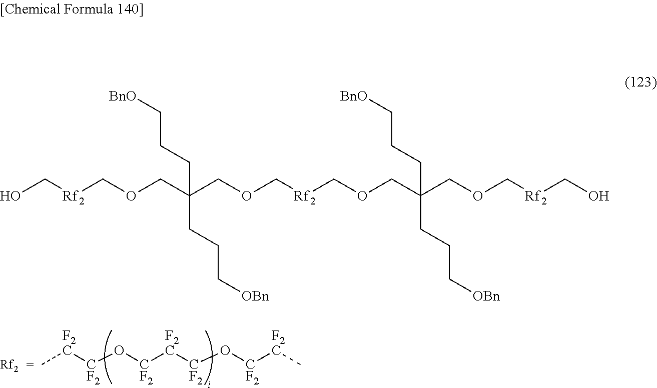

(In Formula (123), Bn represents a benzyl group, Rf₂ is represented by the above formula, and 1 representing the average degree of polymerization in Rf₂ represents 2.0.)

### (Fourth Reaction)

5.0 g of a compound represented by Formula (123) (number-average molecular weight: 2,502, 2.1 mmol) as an intermediate compound (2R-3), 1.5 g of a compound (Ep-3) represented by Formula (20) (molecular weight: 320, 4.7 mmol), and 2.0 mL of t-butanol were charged into a 200 mL eggplant flask under a nitrogen gas atmosphere, and the mixture was stirred at room temperature until uniform. 0.072 g of potassium tert-butoxide (molecular weight: 113, 0.64 mmol) was further added to this uniform liquid, and the mixture was stirred at 70° C. for 16 hours to carry out a reaction.

A reaction product obtained after the reaction was cooled to 25° C., transferred to a separatory funnel containing 100 mL of water, and extracted three times with 100 mL of ethyl acetate. The organic layer was washed with water and dewatered with anhydrous sodium sulfate. After filtering off a desiccant, the filtrate was thickened, and the residue was purified by silica gel column chromatography to obtain 3.7 g of a compound represented by Formula (124) as an intermediate compound (2R-4).

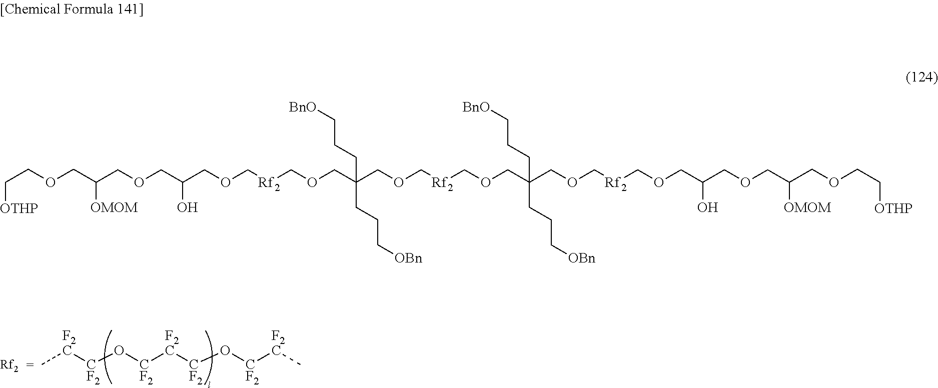

(In Formula (124), THP represents a tetrahydropyranyl group, Bn represents a benzyl group, and MOM represents a methoxymethyl group. Rf₂ is represented by the above formula, and 1 representing the average degree of polymerization in Rf₂ represents 2.0.)

### (Fifth Reaction)

3.7 g of a compound represented by Formula (124) (number-average molecular weight: 3,143, 1.2 mmol) as an intermediate compound (2R-4), 37 mL of methanol, and 3.7 mL of formic acid were charged into a 200 mL eggplant flask under a nitrogen gas atmosphere, and the mixture was stirred at room temperature until uniform. 0.37 g of palladium carbon (Pd/C) was further added to this uniform liquid, and the mixture was stirred at 70° C. for 8 hours to carry out a reaction.

The reaction solution obtained after the reaction was filtered to remove Pd/C, and the filtrate was thickened. After the thickening, the residue was purified by silica gel column chromatography to obtain 2.6 g of a compound (2R) represented by Formula (125) (number-average molecular weight: 2531, 1.1 mmol).

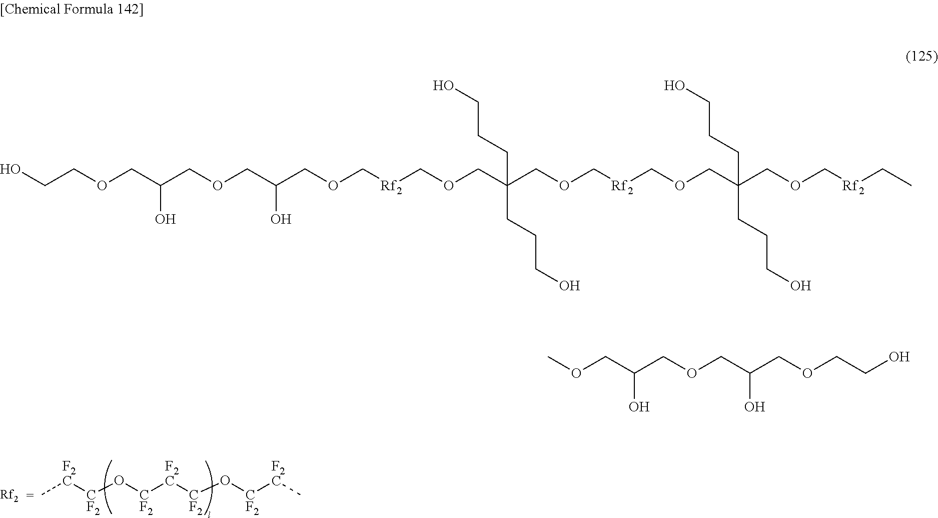

(In Formula (125), Rf₂ is represented by the above formula, and 1 representing the average degree of polymerization in Rf₂ represents 2.0.)

The obtained compound (2R) was subjected to ¹H-NMR and ¹⁹F-NMR measurements, and the structure was identified from the following results.

¹H-NMR (CD₃COCD₃): δ [ppm]=1.60 to 1.90 (16H), 3.35 to 4.39 (66H)

¹⁹F-NMR (CD₃COCD₃): δ [ppm]=−84.0 to −83.0 (24F), −86.4 (12F), −124.3 (12F), −130.0 to −129.0 (12F)

### Example 39

### (First Reaction)

10 g of a compound represented by

HOCH₂CF₂CF₂O(CF₂CF₂CF₂O)ᵢCF₂CF₂CH₂OH (1 representing the average degree of polymerization in the formula was 2.0) (number-average molecular weight: 610, molecular weight distribution: 1.1), 29.2 g of a compound represented by Formula (H1) (molecular weight: 712, 41.0 mmol), 1.1 g of potassium iodide (molecular weight: 166, 6.6 mmol), and 33 mL of N,N′-dimethylformamide (DMF) were charged into a 300 mL eggplant flask under a nitrogen gas atmosphere, and stirred at room temperature to obtain a mixed solution. 21.3 g of cesium carbonate (molecular weight: 325, 65.6 mmol) was added to this mixed solution, stirred and reacted at 70° C. for 10 hours.

The reaction product obtained after the reaction was cooled to 0° C., 100 mL of water was added thereto, and the reaction was stopped. The obtained reaction solution was transferred to a separatory funnel and extracted three times with 100 mL of ethyl acetate. The organic layer was washed with saline and dewatered with anhydrous sodium sulfate. After filtering off a desiccant, the filtrate was thickened, and the residue was purified by silica gel column chromatography to obtain 12.0 g of a compound represented by Formula (126) as an intermediate compound (2S-1).

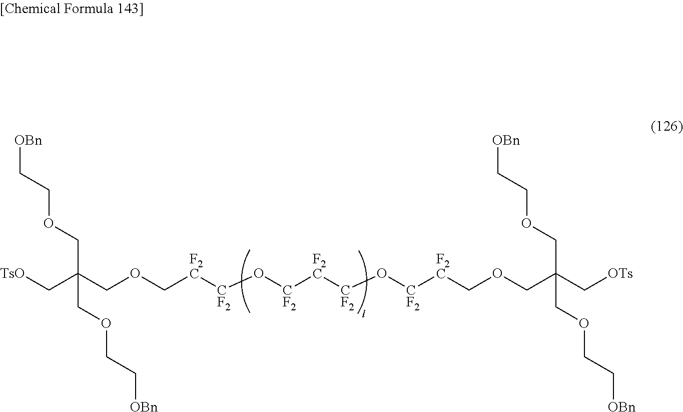

(In Formula (126), Ts represents a p-toluenesulfonyl group, Bn represents a benzyl group, and 1 representing the average degree of polymerization represents 2.0.)

### (Second Reaction)

The operation was performed in the same manner as in the second reaction in Example 21 described above, thereby obtaining an intermediate compound (2A-2) represented by Formula (77).

### (Third Reaction)

7.0 g of a compound represented by Formula (126) as an intermediate compound (2S-1) (number-average molecular weight: 1691, 4.1 mmol), 6.3 g of a compound represented by Formula (77) as an intermediate compound (2A-2) (number-average molecular weight: 694, 9.1 mmol), 0.28 g of potassium iodide (molecular weight: 166, 1.7 mmol), and 9 mL of N,N′-dimethylformamide (DMF) were charged into a 200 mL eggplant flask under a nitrogen gas atmosphere, and stirred at room temperature to obtain a mixed solution. 5.4 g of cesium carbonate (molecular weight: 325, 16.6 mmol) was added to this mixed solution, stirred and reacted at 70° C. for 10 hours.

The reaction solution obtained after the reaction was returned to room temperature, 12 g of a 10% hydrogen chloride/methanol solution (hydrogen chloride-methanol reagent (5% to 10%) manufactured by Tokyo Chemical Industry Co., Ltd.) was added thereto, and the mixture was stirred at room temperature for 2 hours. The reaction solution was transferred little by little to a separatory funnel containing 100 mL of saline, and the mixture was extracted three times with 200 mL of ethyl acetate. The organic layer was washed with 100 mL of saline, 100 mL of saturated aqueous sodium bicarbonate, and 100 mL of saline in this order, and dewatered with anhydrous sodium sulfate. After filtering off a desiccant, the filtrate was thickened, and the residue was purified by silica gel column chromatography. The above-described steps made 5.2 g of a compound represented by Formula (127) obtained as an intermediate compound (2S-3).

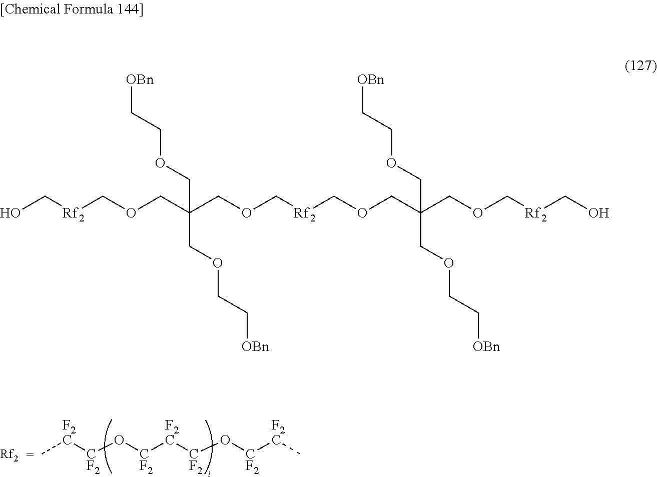

(In Formula (127), Bn represents a benzyl group, Rf₂ is represented by the above formula, and 1 representing the average degree of polymerization in Rf₂ represents 2.0.)

### (Fourth Reaction)

5.0 g of a compound represented by Formula (127) (number-average molecular weight: 2,472, 2.1 mmol) as an intermediate compound (2S-3), 1.5 g of a compound (Ep-6) represented by Formula (29) (molecular weight: 320, 4.7 mmol), and 2.0 mL of t-butanol were charged into a 200 mL eggplant flask under a nitrogen gas atmosphere, and the mixture was stirred at room temperature until uniform. 0.072 g of potassium tert-butoxide (molecular weight: 113, 0.64 mmol) was further added to this uniform liquid, and the mixture was stirred at 70° C. for 16 hours to carry out a reaction.

A reaction product obtained after the reaction was cooled to 25° C., transferred to a separatory funnel containing 100 mL of water, and extracted three times with 100 mL of ethyl acetate. The organic layer was washed with water and dewatered with anhydrous sodium sulfate. After filtering off a desiccant, the filtrate was thickened, and the residue was purified by silica gel column chromatography to obtain 3.7 g of a compound represented by Formula (128) as an intermediate compound (2S-4).

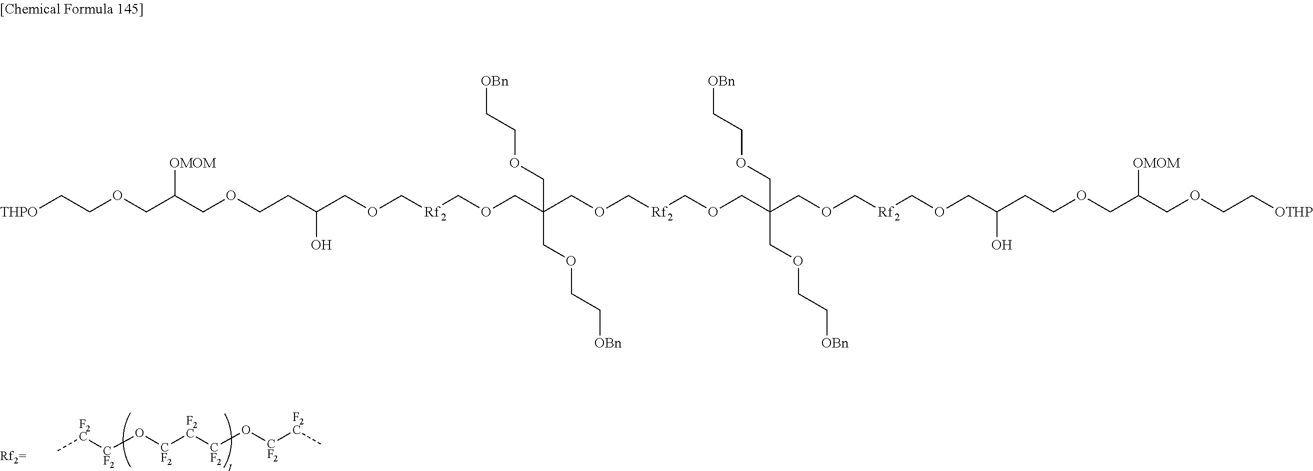

(In Formula (128), THP represents a tetrahydropyranyl group, Bn represents a benzyl group, and MOM represents a methoxymethyl group. Rf₂ is represented by the above formula, and 1 representing the average degree of polymerization in Rf₂ represents 2.0.)

### (Fifth Reaction)

3.7 g of a compound represented by Formula (128) (number-average molecular weight: 3,067, 1.2 mmol) as an intermediate compound (2S-4), 37 mL of methanol, and 3.7 mL of formic acid were charged into a 200 mL eggplant flask under a nitrogen gas atmosphere, and the mixture was stirred at room temperature until uniform. 0.37 g of palladium carbon (Pd/C) was further added to this uniform liquid, and the mixture was stirred at 70° C. for 8 hours to carry out a reaction.

The reaction solution obtained after the reaction was filtered to remove Pd/C, and the filtrate was thickened. After the thickening, the residue was purified by silica gel column chromatography to obtain 2.5 g of a compound (2S) represented by Formula (129) (number-average molecular weight: 2637, 1.0 mmol).

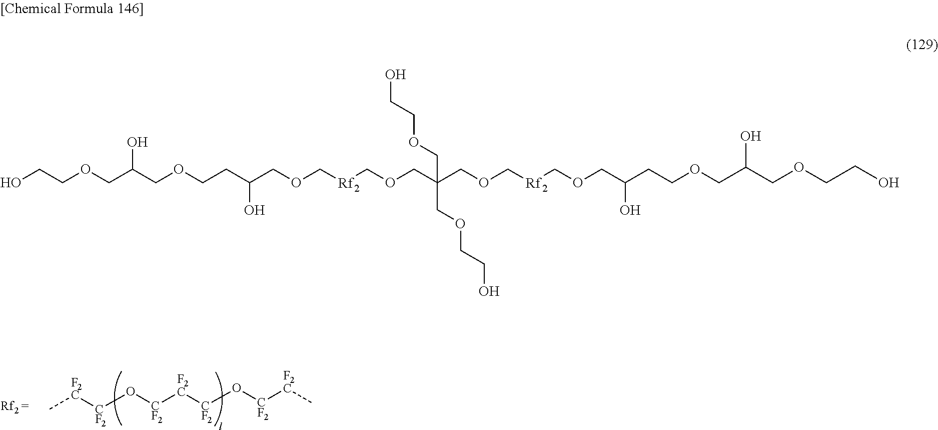

(In Formula (129), Rf₂ is represented by the above formula, and 1 representing the average degree of polymerization in Rf₂ represents 2.0.)

The obtained compound (2S) was subjected to ¹H-NMR and ¹⁹F-NMR measurements, and the structure was identified from the following results.

¹H-NMR (CD₃COCD₃): δ [ppm]=1.61 to 1.83 (20H), 3.38 to 4.40 (66H)

¹⁹F-NMR (CD₃COCD₃): δ [ppm]=−84.0 to −83.0 (24F), −86.4 (12F), −124.3 (12F), −130.0 to −129.0 (12F)

### Example 40

### (First Reaction)

10 g of a compound represented by

HOCH₂CF₂CF₂O(CF₂CF₂CF₂O)ᵢCF₂CF₂CH₂OH (1 representing the average degree of polymerization in the formula was 2.0) (number-average molecular weight: 610, molecular weight distribution: 1.1), 27.4 g of a compound represented by Formula (M1) (molecular weight: 668, 41.0 mmol), 1.1 g of potassium iodide (molecular weight: 166, 6.6 mmol), and 33 mL of N,N′-dimethylformamide (DMF) were charged into a 300 mL eggplant flask under a nitrogen gas atmosphere, and stirred at room temperature to obtain a mixed solution. 21.3 g of cesium carbonate (molecular weight: 325, 65.6 mmol) was added to this mixed solution, stirred and reacted at 70° C. for 10 hours.

The reaction product obtained after the reaction was cooled to 0° C., 100 mL of water was added thereto, and the reaction was stopped. The obtained reaction solution was transferred to a separatory funnel and extracted three times with 100 mL of ethyl acetate. The organic layer was washed with saline and dewatered with anhydrous sodium sulfate. After filtering off a desiccant, the filtrate was thickened, and the residue was purified by silica gel column chromatography to obtain 10.5 g of a compound represented by Formula (130) as an intermediate compound (2T-1).

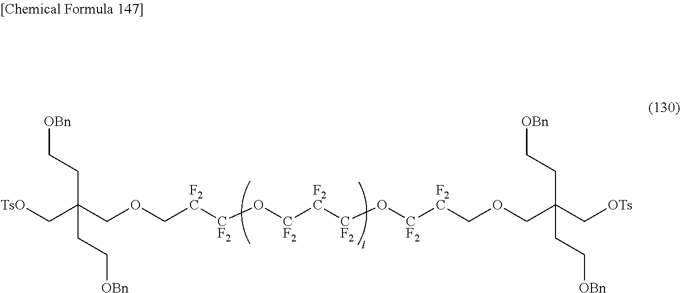

(In Formula (130), Ts represents a p-toluenesulfonyl group, Bn represents a benzyl group, and 1 representing the average degree of polymerization represents 2.0.)

### (Second Reaction)

The operation was performed in the same manner as in the second reaction in Example 21 described above, thereby obtaining an intermediate compound (2A-2) represented by Formula (77).

### (Third Reaction)

7.0 g of a compound represented by Formula (130) as an intermediate compound (2T-1) (number-average molecular weight: 1603, 4.4 mmol), 6.7 g of a compound represented by Formula (77) as an intermediate compound (2A-2) (number-average molecular weight: 694, 9.6 mmol), 0.29 g of potassium iodide (molecular weight: 166, 1.8 mmol), and 9 mL of N,N′-dimethylformamide (DMF) were charged into a 200 mL eggplant flask under a nitrogen gas atmosphere, and stirred at room temperature to obtain a mixed solution. 5.7 g of cesium carbonate (molecular weight: 325, 17.5 mmol) was added to this mixed solution, stirred and reacted at 70° C. for 10 hours.

The reaction solution obtained after the reaction was returned to room temperature, 12 g of a 10% hydrogen chloride/methanol solution (hydrogen chloride-methanol reagent (5% to 10%) manufactured by Tokyo Chemical Industry Co., Ltd.) was added thereto, and the mixture was stirred at room temperature for 2 hours. The reaction solution was transferred little by little to a separatory funnel containing 100 mL of saline, and the mixture was extracted three times with 200 mL of ethyl acetate. The organic layer was washed with 100 mL of saline, 100 mL of saturated aqueous sodium bicarbonate, and 100 mL of saline in this order, and dewatered with anhydrous sodium sulfate. After filtering off a desiccant, the filtrate was thickened, and the residue was purified by silica gel column chromatography. The above-described steps made 5.1 g of a compound represented by Formula (131) obtained as an intermediate compound (2T-3).

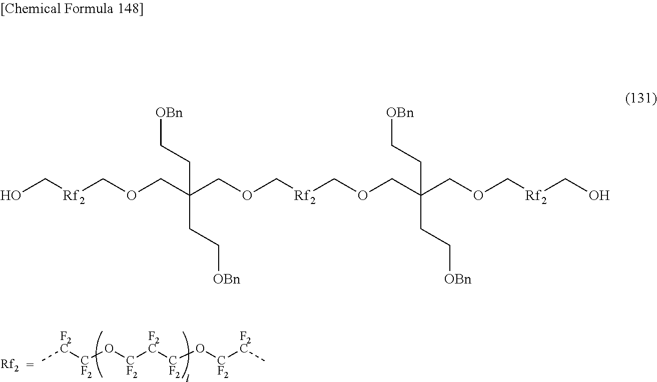

(In Formula (131), Bn represents a benzyl group, Rf₂ is represented by the above formula, and 1 representing the average degree of polymerization in Rf₂ represents 2.0.)

### (Fourth Reaction)

5.0 g of a compound represented by Formula (131) (number-average molecular weight: 2,478, 2.2 mmol) as an intermediate compound (2T-3), 1.0 g of a compound (Ep-1) represented by Formula (13) (molecular weight: 202, 4.8 mmol), and 2.0 mL of t-butanol were charged into a 200 mL eggplant flask under a nitrogen gas atmosphere, and the mixture was stirred at room temperature until uniform. 0.073 g of potassium tert-butoxide (molecular weight: 113, 0.65 mmol) was further added to this uniform liquid, and the mixture was stirred at 70° C. for 16 hours to carry out a reaction.

A reaction product obtained after the reaction was cooled to 25° C., transferred to a separatory funnel containing 100 mL of water, and extracted three times with 100 mL of ethyl acetate. The organic layer was washed with water and dewatered with anhydrous sodium sulfate. After filtering off a desiccant, the filtrate was thickened, and the residue was purified by silica gel column chromatography to obtain 3.6 g of a compound represented by Formula (132) as an intermediate compound (2T-4).

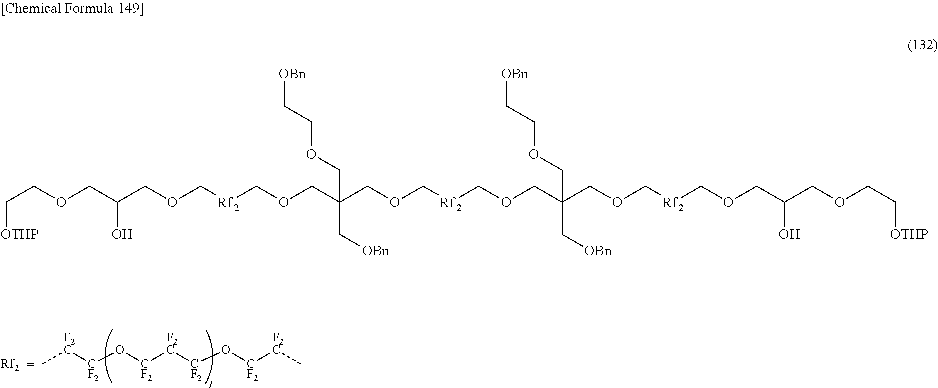

(In Formula (132), THP represents a tetrahydropyranyl group, Bn represents a benzyl group, Rf₂ is represented by the above formula, and 1 representing the average degree of polymerization in Rf₂ represents 2.0.)

### (Fifth Reaction)

3.6 g of a compound represented by Formula (132) (number-average molecular weight: 2,883, 1.3 mmol) as an intermediate compound (2T-4), 36 mL of methanol, and 3.6 mL of formic acid were charged into a 200 mL eggplant flask under a nitrogen gas atmosphere, and the mixture was stirred at room temperature until uniform. 0.36 g of palladium carbon (Pd/C) was further added to this uniform liquid, and the mixture was stirred at 70° C. for 8 hours to carry out a reaction.

The reaction solution obtained after the reaction was filtered to remove Pd/C, and the filtrate was thickened. After the thickening, the residue was purified by silica gel column chromatography to obtain 2.6 g of a compound (2T) represented by Formula (133) (number-average molecular weight: 2359, 1.2 mmol).

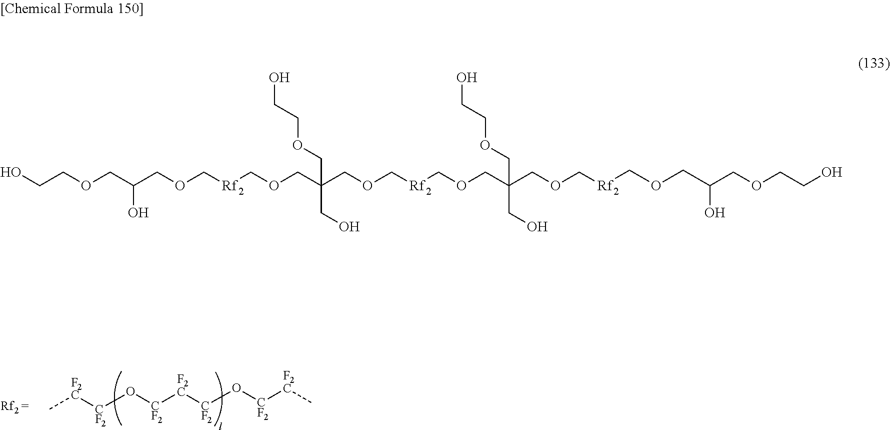

(In Formula (133), Rf₂ is represented by the above formula, and 1 representing the average degree of polymerization in Rf₂ represents 2.0.)

The obtained compound (2T) was subjected to ¹H-NMR and ¹⁹F-NMR measurements, and the structure was identified from the following results.

¹H-NMR (CD₃COCD₃): δ [ppm]=3.35 to 4.40 (62H)

¹⁹F-NMR (CD₃COCD₃): δ [ppm]=−84.0 to −83.0 (24F), −86.4 (12F), −124.3 (12F), −130.0 to −129.0 (12F)

The values of z in a case where each of the compounds (1 Å) to (1T) and (2 Å) to (2T) of Examples 1 to 40, which were obtained as described above, was applied to Formula (1) and the structures of R¹, R², R³, R⁴, and R⁵ are shown in Tables 1 to 5.

TABLE 1

| | z | R¹ | R² | R³ | R⁴ | R⁵ | Compound |
|---|---|---|---|---|---|---|---|
| Example 1 | 1 | 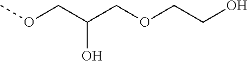 | Rf₂ l = 3.8 | (2-1) a = 1 | (2-1) a = 1 | Same as R¹ | (1A) |
| Example 2 | 1 | 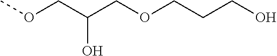 | Rf₂ l = 3.8 | (2-1) a = 1 | (2-1) a = 1 | Same as R¹ | (1B) |
| Example 3 | 1 | 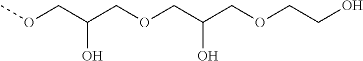 | Rf₂ l = 3.8 | (2-1) a = 1 | (2-1) a = 1 | Same as R¹ | (1C) |
| Example 4 | 1 | 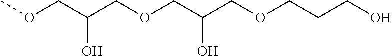 | Rf₂ l = 3.8 | (2-1) a = 1 | (2-1) a = 1 | Same as R¹ | (1D) |
| Example 5 | 1 | 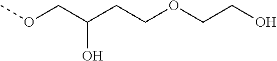 | Rf₂ l = 3.8 | (2-1) a = 1 a = 1 | (2-1) a = 1 a = 1 | Same as R¹ | (1E) |
| Example 6 | 1 | 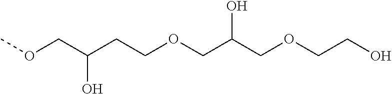 | Rf₂ l = 3.8 | (2-1) a = 1 | (2-1) a =1 | Same as R¹ | (1F) |
| Example 7 | 1 | 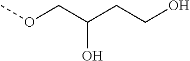 | Rf₁ j = 4.0 k = 4.0 | (2-1) a = 1 | (2-1) a = 1 | Same as R¹ | (1G) |
| Example 8 | 1 | 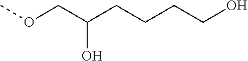 | Rf₁ j = 4.0 k = 4.0 | (2-1) a = 1 | (2-1) a = 1 | Same as R¹ | (1H) |

TABLE 2

| | z | R¹ | R² | R³ | R⁴ | R⁵ | Compound |
|---|---|---|---|---|---|---|---|
| Example 9 | 1 | 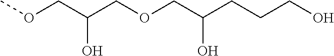 | Rf₁ j = 6.3 k = 0 | (2-1) a = 1 | (2-1) a = 1 | Same as R¹ | (1I) |
| Example 10 | 1 | 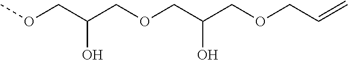 | Rf₂ l = 3.8 | (2-1) a = 1 | (2-1) a = 1 | Same as R¹ | (1J) |
| Example 11 | 1 | 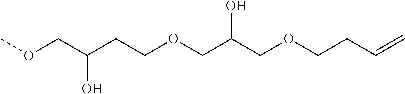 | Rf₂ l = 3.8 | (2-1) a = 1 | (2-1) a = 1 | Same as R¹ | (1K) |
| Example 12 | 1 | 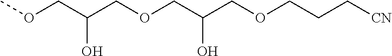 | Rf₁ j = 6.3 k = 0 | (2-1) a = 1 | (2-1) a = 1 | Same as R¹ | (1L) |
| Example 13 | 1 | 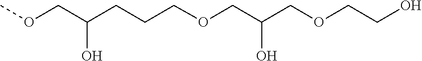 | Rf₁ j = 4.0 k = 4.0 | (2-1) a = 1 | (2-1) a = 1 | Same as R¹ | (1M) |
| Example 14 | 1 | 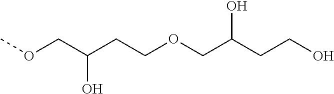 | Rf₂ l = 3.8 | (2-1) a = 1 | (2-1) a = 1 | Same as R¹ | (1N) |
| Example 15 | 1 | 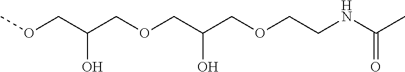 | Rf? j = 6.3 k = 0 | (2-1) a = 1 | (2-1) a = 1 | Same as R¹ | (1O) |
| Example 16 | 1 | 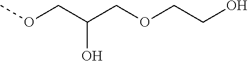 | Rf₂ l = 3.8 | (2-1) a = 1 a = 2 | (2-1) a = 1 a = 2 | Same as R¹ | (1P) |

TABLE 3

| | z | R¹ | R² | R³ | R⁴ | R⁵ | Compound |
|---|---|---|---|---|---|---|---|
| Example 17 | 1 | 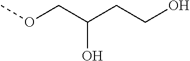 | Rf₂ l = 3.8 | (2-1) a = 2 | (2-1) a = 2 | Same as R¹ | (1Q) |
| Example 18 | 1 | 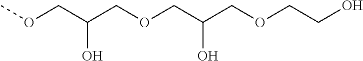 | Rf₂ l = 3.8 | (2-1) a = 3 | (2-1) a = 3 | Same as R¹ | (1R) |
| Example 19 | 1 | 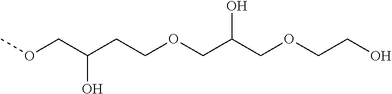 | Rf₂ l = 3.8 | (2-3) c = 1 d = 2 | (2-3) c = 1 d = 2 | Same as R¹ | (1S) |
| Example 20 | 1 | 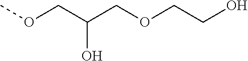 | Rf₂ l = 3.8 | (2-1) a = 1 | (2-3) c = 1 d = 2 | Same as R¹ | (1T) |
| Example 21 | 2 | 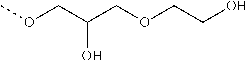 | Rf₂ l = 2.0 | (2-1) a = 1 | (2-1) a = 1 | Same as R¹ | (2A) |
| Example 22 | 2 | 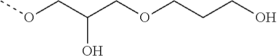 | Rf₂ l = 2.0 | (2-1) a = 1 | (2-1) a = 1 | Same as R¹ | (2B) |
| Example 23 | 2 | 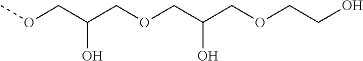 | Rf₂ l = 2.0 | (2-1) a = 1 | (2-1) a = 1 | Same as R¹ | (2C) |
| Example 24 | 2 | 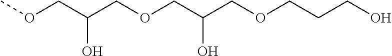 | Rf₂ l = 2.0 | (2-1) a = 1 | (2-1) a = 1 | Same as R¹ | (2D) |

TABLE 4

| | z | R¹ | R² | R³ | R⁴ | R⁵ | Compound |
|---|---|---|---|---|---|---|---|
| Example 25 | 2 | 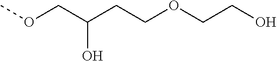 | Rf₂ l = 2.0 | (2-1) a = 1 | (2-1) a = 1 | Same as R¹ | (2E) |
| Example 26 | 2 | 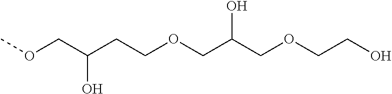 | Rf₂ l = 2.0 | (2-1) a = 1 | (2-1) a = 1 | Same as R¹ | (2F) |
| Example 27 | 2 | 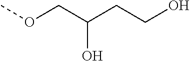 | Rf₁ j = 2.4 k = 2.4 | (2-1) a = 1 | (2-1) a = 1 | Same as R¹ | (2G) |
| Example 28 | 2 | 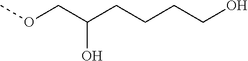 | Rf₁ j = 2.4 k = 2.4 | (2-1) a = 1 | (2-1) a = 1 | Same as R¹ | (2H) |
| Example 29 | 2 | 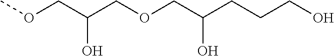 | Rf₁ j = 3.8 k = 0 | (2-1) a = 1 | (2-1) a = 1 | Same as R¹ | (2I) |
| Example 30 | 2 | 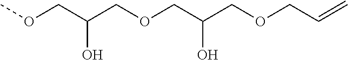 | Rf₂ l = 2.0 | (2-1) a = 1 | (2-1) a = 1 | Same as R¹ | (2J) |
| Example 31 | 2 | 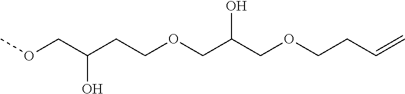 | Rf₂ l = 2.0 | (2-1) a = 1 | (2-1) a = 1 | Same as R¹ | (2K) |
| Example 32 | 2 | 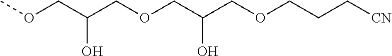 | Rf₁ j = 3.8 k = 0 | (2-1) a = 1 | (2-1) a = 1 | Same as R¹ | (2L) |

TABLE 5

| | z | R¹ | R² | R³ | R⁴ | R⁵ | Compound |
|---|---|---|---|---|---|---|---|
| Example 33 | 2 | 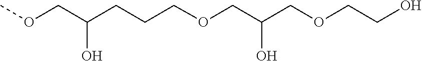 | Rf₁ j = 2.4 k = 2.4 | (2-1) a = 1 | (2-1) a = 1 | Same as R¹ | (2M) |
| Example 34 | 2 | 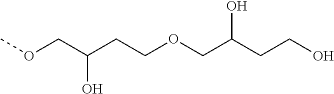 | 2Rf₂ l = 2.0 | (2-1) a = 1 | (2-1) a = 1 | Same as R¹ | (2N) |
| Example 35 | 2 | 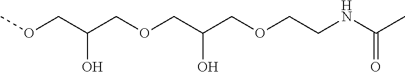 | Rf₁ j = 3.8 k = 0 | (2-1) a = 1 | (2-1) a = 1 | Same as R¹ | (20) |
| Example 36 | 2 | 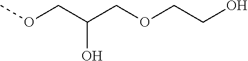 | Rf₂ l = 2.0 | (2-1) a = 2 | (2-1) a = 2 | Same as R¹ | (2P) |
| Example 37 | 2 | 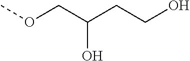 | Rf₂ l = 2.0 | (2-1) a = 2 | (2-1) a = 2 | Same as R¹ | (2Q) |
| Example 38 | 2 | 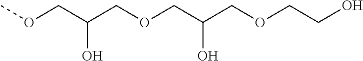 | Rf₂ l = 2.0 | (2-1) a = 3 | (2-1) a = 3 | Same as R¹ | (2R) |
| Example 39 | 2 | 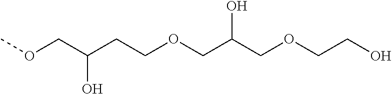 | Rf₂ l = 2.0 | (2-3) c = 1 d= 2 | (2-3) c = 1 d = 2 | Same as R¹ | (2S) |
| Example 40 | 2 | 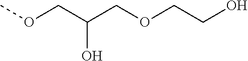 | Rf₂ l = 2.0 | (2-1) a = 1 | (2-3) c = 1 d = 2 | Same as R¹ | (2T) |

### Comparative Example 1

A compound (4 Å) represented by Formula (134) was synthesized by the method described in Patent Document 1.

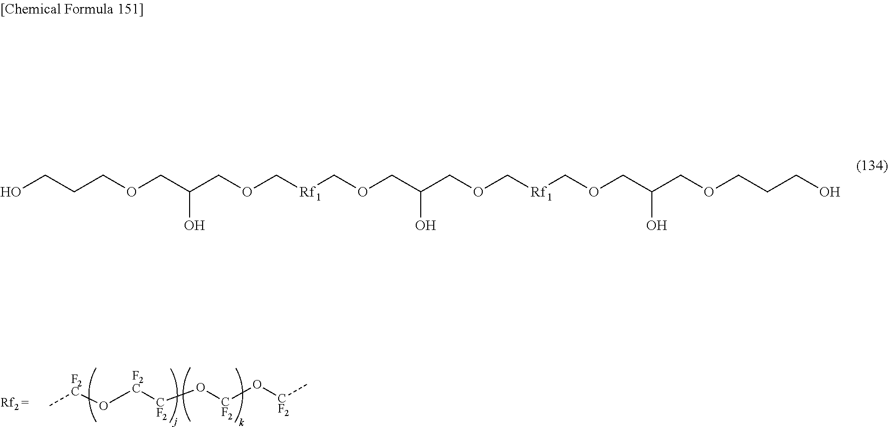

(In Formula (134), Rf₁ is represented by the above formula, j indicating the average degree of polymerization in Rf₁ represents 4.0, and k indicating the average degree of polymerization represents 4.0.)

### Comparative Example 2

A compound (4B) represented by Formula (135) was synthesized by the method described in Patent Document 4.

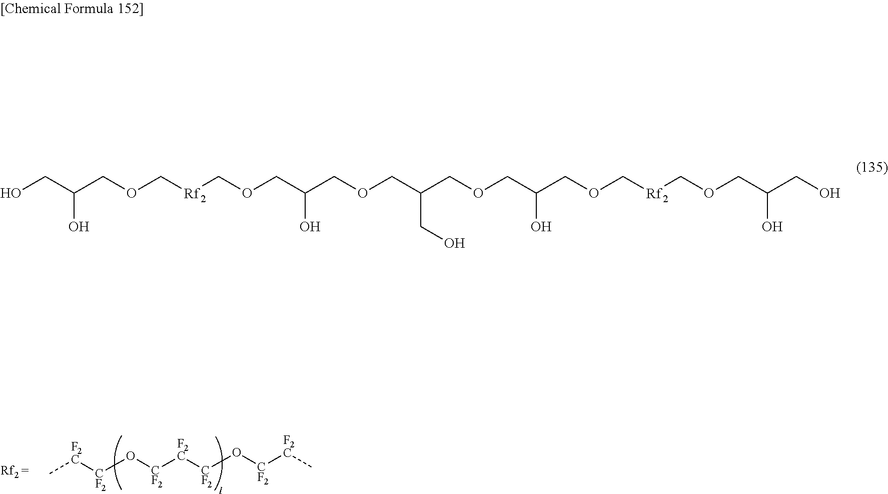

(In Formula (135), Rf₂ is represented by the above formula, and 1 representing the average degree of polymerization in Rf₂ represents 3.8.)

### Comparative Example 3

A compound (4C) represented by Formula (136) was synthesized by the method described in Patent Document 3.

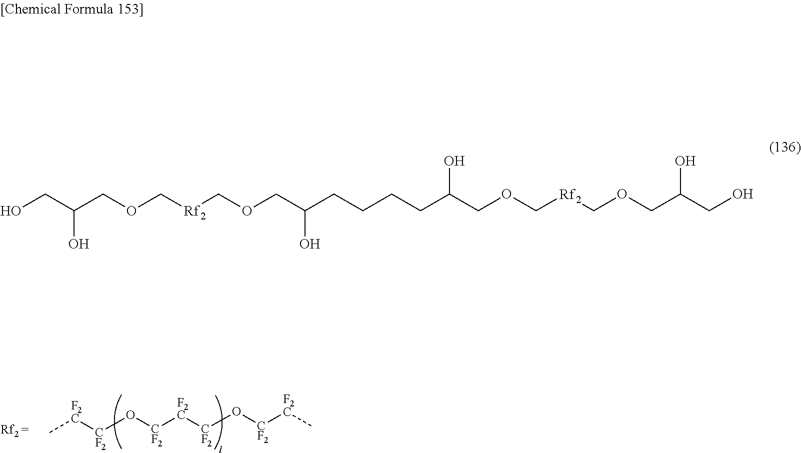

(In Formula (136), Rf₂ is represented by the above formula, and 1 representing the average degree of polymerization in Rf₂ represents 3.8.)

### Comparative Example 4

A compound (4D) represented by Formula (137) was synthesized by the method described in Patent Document 4.

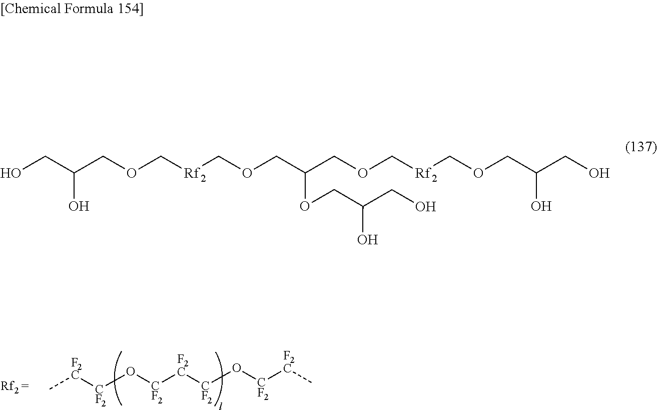

(In Formula (137), Rf₂ is represented by the above formula, and 1 representing the average degree of polymerization in Rf₂ represents 3.8.)

### Comparative Example 5

A compound (4E) represented by Formula (138) was synthesized by the method described in Patent Document 4.

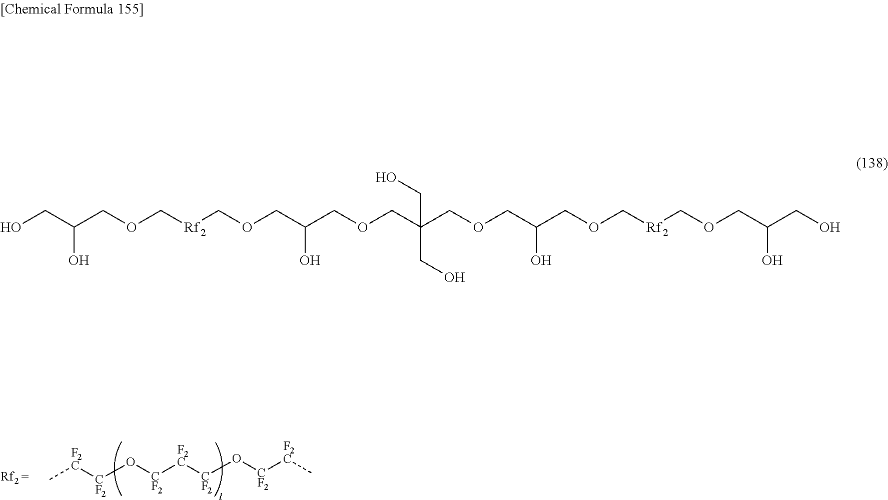

(In Formula (138), Rf₂ is represented by the above formula, and 1 representing the average degree of polymerization in Rf₂ represents 3.8.)

### Comparative Example 6

A compound (4F) represented by Formula (139) was synthesized by the method described in Patent Document 6.

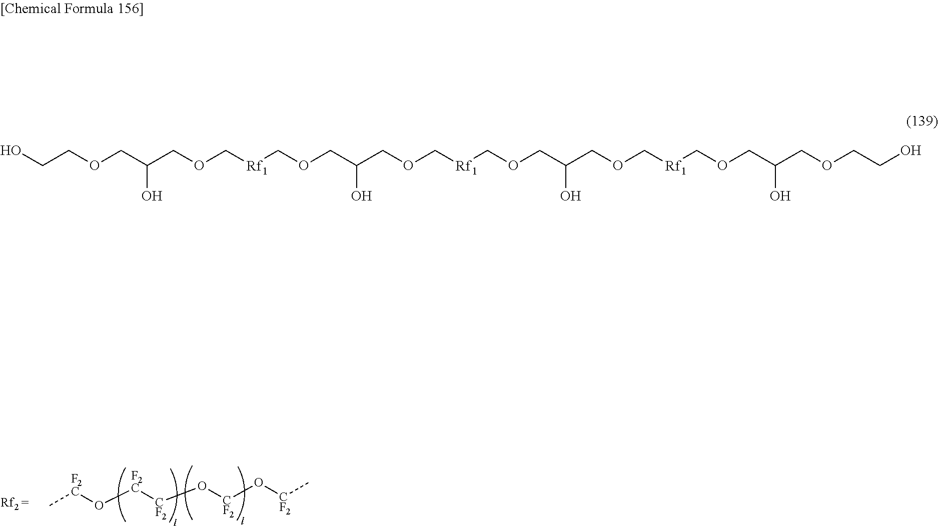

(In Formula (139), in the center Rf₁, j representing the average degree of polymerization represents 3.8, and k representing the average degree of polymerization represents 0. In two terminals of Rf₁, j representing the average degree of polymerization is 2.4, and k representing the average degree of polymerization is 2.4.)

### Comparative Example 7

A compound (4G) represented by Formula (140) was synthesized by the method described in Patent Document 5.

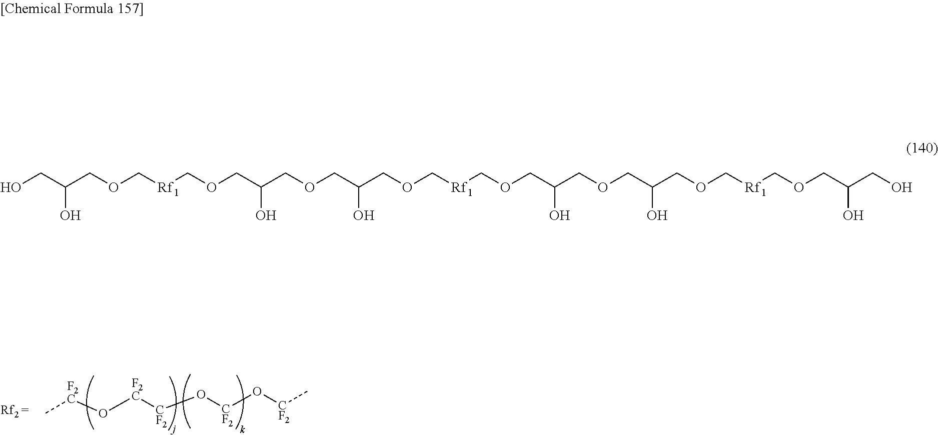

(In Formula (140), Rf₁ is represented by the above formula, j indicating the average degree of polymerization in Rf₁ represents 3.8, and k indicating the average degree of polymerization represents 0.)

The number-average molecular weights (Mn) of the compounds of Examples 1 to 40 and Comparative Examples 1 to 7 obtained as described above were determined from the measurement results of the above-described ¹H-NMR and ¹⁹F-NMR. The results are shown in Table 6 and Table 7. It is presumed that there is a variation of about 1 to 5 in the value of the average molecular weight of the synthesized compound due to the molecular weight distribution of the fluoropolyether used as a raw material of the compound, a difference in operation In the case of synthesizing the compound, and the like.

TABLE 6

| | | Number-average | Film | Pickup | Chemical |
|---|---|---|---|---|---|
| | | molecular weight | thickness | characteristics | substance |
| | Compound | (Mn) | (Å) | test | resistance test |
| Example 1 | (1A) | 2156 | 9.5 | A | A |
| Example 2 | (1B) | 2184 | 9.5 | A | A |
| Example 3 | (1C) | 2305 | 9.4 | A | B |
| Example 4 | (1D) | 2333 | 9.5 | A | B |
| Example 5 | (1E) | 2184 | 9.4 | A | A |
| Example 6 | (1F) | 2333 | 9.4 | A | B |
| Example 7 | (1G) | 2091 | 9.6 | A | A |
| Example 8 | (1H) | 2147 | 9.4 | A | A |
| Example 9 | (1I) | 2272 | 9.4 | A | B |
| Example 10 | (1J) | 2297 | 9.5 | B | A |
| Example 11 | (1K) | 2353 | 9.3 | B | A |
| Example 12 | (1L) | 2350 | 9.6 | B | A |
| Example 13 | (1M) | 2355 | 9.5 | A | B |
| Example 14 | (1N) | 2273 | 9.5 | A | B |
| Example 15 | (1O) | 2386 | 9.5 | A | B |
| Example 16 | (1P) | 2184 | 9.5 | A | A |
| Example 17 | (1Q) | 2124 | 9.4 | A | A |
| Example 18 | (1R) | 2361 | 9.5 | A | B |
| Example 19 | (1S) | 2435 | 9.4 | A | B |
| Example 20 | (1T) | 2200 | 9.4 | B | A |
| Example 21 | (2A) | 2271 | 9.6 | A | A |
| Example 22 | (2B) | 2299 | 9.6 | A | A |
| Example 23 | (2C) | 2419 | 9.5 | A | B |
| Example 24 | (2D) | 2447 | 9.5 | A | B |
| Example 25 | (2E) | 2299 | 9.4 | A | A |
| Example 26 | (2F) | 2447 | 9.6 | A | B |
| Example 27 | (2G) | 2225 | 9.5 | A | A |
| Example 28 | (2H) | 2281 | 9.5 | A | A |
| Example 29 | (2I) | 2413 | 9.5 | A | B |
| Example 30 | (2J) | 2411 | 9.4 | B | A |
| Example 31 | (2K) | 2467 | 9.5 | B | A |
| Example 32 | (2L) | 2491 | 9.5 | B | A |
| Example 33 | (2M) | 2490 | 9.5 | A | B |
| Example 34 | (2N) | 2387 | 9.6 | A | B |
| Example 35 | (2O) | 2527 | 9.6 | A | B |
| Example 36 | (2P) | 2327 | 9.6 | A | A |
| Example 37 | (2Q) | 2267 | 9.5 | A | A |
| Example 38 | (2R) | 2531 | 9.5 | A | B |
| Example 39 | (2S) | 2637 | 9.4 | A | B |
| Example 40 | (2T) | 2359 | 9.5 | B | A |

TABLE 7

| | | Number-average | Film | Pickup | Chemical |
|---|---|---|---|---|---|
| | | molecular weight | thickness | characteristics | substance |
| | Compound | (Mn) | (Å) | test | resistance test |
| Comparative | (4A) | 2133 | 9.4 | D | D |
| Example 1 | | | | | |
| Comparative | (4B) | 2184 | 9.4 | C | C |
| Example 2 | | | | | |
| Comparative | (4C) | 2108 | 9.5 | D | D |
| Example 3 | | | | | |
| Comparative | (4D) | 2096 | 9.6 | C | D |
| Example 4 | | | | | |
| Comparative | (4E) | 2214 | 9.4 | C | C |
| Example 5 | | | | | |
| Comparative | (4F) | 2225 | 9.5 | D | D |
| Example 6 | | | | | |
| Comparative | (4G) | 2107 | 9.6 | C | C |
| Example 7 | | | | | |

Next, solutions for forming a lubricating layer were prepared using the compounds obtained in Examples 1 to 40 and Comparative Examples 1 to 7 by a method described below. In addition, lubricating layers for a magnetic recording medium were formed using the obtained solutions for forming a lubricating layer by the following method, and magnetic recording media of Examples 1 to 40 and Comparative Examples 1 to 7 were obtained.

### “Solution for Forming Lubricating Layer”

Each of the fluorine-containing ether compounds obtained in Examples 1 to 40 and Comparative Examples 1 to 7 was dissolved in Vertrel (registered trademark) XF (trade name, manufactured by Mitsui DuPont Fluorochemicals Co., Ltd.), which is a fluorine-based solvent, and diluted with Vertrel XF so that the film thickness In the case of being applied onto the protective layer was 9 A to 10 A, thereby obtaining a solution for forming a lubricating layer.

### “Magnetic Recording Medium”

A magnetic recording medium in which an adhesion layer, a soft magnetic layer, a first underlayer, a second underlayer, a magnetic layer, and a protective layer were sequentially provided on a substrate having a diameter of 65 mm was prepared. The protective layer was formed of carbon.

The solutions for forming a lubricating layer of Examples 1 to 40 and Comparative Examples 1 to 7 were each applied onto the protective layer of the magnetic recording medium in which the individual layers up to the protective layer had been formed by a dipping method. The dipping method was performed under conditions of an immersion rate of 10 mm/see, an immersion time of 30 sec, and a lifting rate of 1.2 mm/sec.

Thereafter, the magnetic recording medium coated with the solution for forming a lubricating layer was placed in a thermostatic chamber at 120° C. and heated for 10 minutes to remove the solvent in the solution for forming a lubricating layer, thereby forming a lubricating layer on the protective layer, and obtaining a magnetic recording medium.

### (Film Thickness Measurement)

For the lubricating layers of the magnetic recording media of Examples 1 to 40 and Comparative Examples 1 to 7 obtained as described above, the peak heights in C—F vibration expansion and contraction were measured using FT-IR (trade name: Nicolet iS50, manufactured by Thermo Fisher Scientific Inc.). Next, the film thicknesses of the lubricating layers were calculated from the measured values of the peak heights in the C—F vibration and expansion and contraction of the lubricating layer, using a correlation expression obtained by the method described below.

### (Method for Calculating Correlation Expression)

A disk in which an adhesion layer, a soft magnetic layer, a first underlayer, a second underlayer, a magnetic layer, and a protective layer were sequentially provided on a substrate having a diameter of 65 mm was prepared. A lubricating layer was formed on the protective layer of the disk at each of film thicknesses of 6 to 20 Å (in increments of 2 Å).

Thereafter, for each disk on which the lubricating layer was formed, the increase in film thickness from the disk surface on which the lubricating layer was not formed was measured using an ellipsometer, and the measured value was defined as the film thickness of the lubricating layer. In addition, for each disk on which the lubricating layer was formed, the peak height in the C—F vibration and expansion and contraction was measured using FT-IR.

Then, a correlation expression between the peak height obtained by FT-IR and the film thickness of the lubricating layer obtained using the ellipsometer was obtained.

Next, the pickup characteristic test and the chemical substance resistance test were performed and evaluated for the magnetic recording media of Examples 1 to 40 and Comparative Examples 1 to 7 by the methods shown below. The results are shown in Table 6 and Table 7.

### (Pickup Characteristic Test)

The magnetic recording medium and a magnetic head were mounted on a spin stand, and the magnetic recording medium was rotated under normal temperature and under reduced pressure (about 250 torr) to cause the magnetic head to float at a constant point for 10 minutes. Thereafter, a surface of the magnetic head, which was opposite to the magnetic recording medium, was analyzed by using an electron spectroscopy for chemical analysis (ESCA) analyzer. The intensity (signal intensity (a.u.)) of the fluorine-derived peak obtained by the analysis using the ESCA analysis device indicates the adhesion amount of a lubricant to the magnetic head. The pickup characteristics were evaluated using the obtained signal intensity according to the evaluation standards shown below.

### (Evaluation Standard for Pickup Characteristics)

A (excellent): signal intensity of 160 or less (very small adhesion amount)
B (good): signal intensity of 161 to 300 (small adhesion amount)
C (acceptable): signal intensity of 301 to 1000 (large adhesion amount)
D (unacceptable): signal intensity of 1001 or more (very large adhesion amount)

### (Chemical Substance Resistance Test)

The degree of contamination of the magnetic recording medium with an environmental substance that generates a contaminant in a high temperature environment was evaluated by the following method. Si ions were used as the environmental substance. The degree of contamination of the magnetic recording medium with the contaminant generated by the environmental substance was evaluated by measuring the Si adsorption amount as the amount of the contaminant.

Specifically, the magnetic recording medium to be evaluated was held for 240 hours in a high temperature environment of a temperature of 85° C. and a humidity of 0% in the presence of the siloxane-based Si rubber. Next, the Si adsorption amount present on the surface of the magnetic recording medium was analyzed and measured by secondary ion mass spectrometry (SIMS), and the degree of contamination with Si ions was evaluated as the Si adsorption amount. The evaluation of the Si adsorption amount was evaluated using the numerical value in a case where the result of the Si adsorption amount of Comparative Example 1 was set to 1.00, based on the following evaluation standard. The results are shown in Table 6 and Table 7.

### (Evaluation Standard for Chemical Substance Resistance)

A (excellent): less than 0.70
B (good): 0.70 or more and less than 0.80
C (acceptable): 0.80 or more and less than 1.00
D (unacceptable): 1.00 or more

As shown in Table 6, in the magnetic recording media of Examples 1 to 40, the evaluations of the pickup characteristics test and the chemical substance resistance test were all A or B, and at least one of the evaluations was A. From this, it was possible to confirm that the pickup suppression effects of the magnetic recording media of Examples 1 to 40 were high and the chemical substance resistance of the magnetic recording media were high.

This is presumed to be because the compounds represented by (TA) to (1T) and (2 Å) to (2T) forming the lubricating layers of the magnetic recording media of Examples 1 to 40 were less likely to generate a polar group that did not bond to the functional group (active point) present on the protective layer. In other words, this is presumed to be because the polar groups in R¹ and R⁵ which were terminal groups included in the compounds represented by (1 Å) to (1T) and (2 Å) to (2T), and the primary hydroxy groups of R³ and R⁴ bonded to the tetrasubstituted carbon atom included in the linking group closely adhered to the protective layer at a high probability. As a result, the adhesion of the lubricating layer to the protective layer was improved, the adhesion of the lubricating layer to the magnetic head was suppressed, and the entrapment of a contaminant caused by a polar group that had not closely adhered to the protective layer included in the lubricating layer was suppressed. Therefore, it is presumed that excellent chemical substance resistance and an excellent pickup suppression effect could be obtained.

In addition, in the magnetic recording media of Examples 1 to 9, 13 to 19, 21 to 29, and 33 to 39, the evaluations of the pickup characteristics test were A, and in particular, the pickup suppression effects were excellent. This is presumed to be because the magnetic recording media of the above-described examples had a lubricating layer formed of a compound in which R¹ and R⁵ were any of those represented by Formulae (4-1) to (4-3), X¹ was a hydroxy group or a group having an amide bond, and the organic groups represented by R³ and R⁴ were the same as each other, and thus the adhesion of the lubricating layer to the protective layer was good and the lubricating layer was less likely to float from the protective layer.

In addition, the magnetic recording media of Examples 1, 2, 5, 7, 8, 10 to 12, 16, 17, 20 to 22, 25, 27, 28, 30 to 32, 36, 37, and 40 were evaluated as A in the chemical substance resistance test, which was good.

The magnetic recording media of Examples 10, 11, 30, and 31 were formed of a compound in which R¹ and R⁵ were Formula (4-1) or (4-2) and X¹ was an alkenyl group. Therefore, in the magnetic recording media of Examples 10, 11, 30, and 31, it is presumed that the 7t-7t interaction between the alkenyl group in the compound forming the lubricating layer and the protective layer improved the adhesion to the protective layer and improves the chemical substance resistance.

On the other hand, as shown in Table 7, in Comparative Examples 1 to 7 having the lubricating layer formed of any of the compounds (4 Å) to (4G), the evaluations of the pickup characteristics test and the chemical substance resistance test were all C or D, which were inferior to those of Examples 1 to 40.

This is presumed to be because, in Comparative Examples 1 to 7, the lubricating layer was formed using a fluorine-containing ether compound that did not satisfy Expression (1).

Specifically, the magnetic recording media of Comparative Examples 1 and 6 each had a lubricating layer formed of the compounds (4 Å) and (4F). In the compounds (4 Å) and (4F), the linking group disposed between two or three perfluoropolyether chains had one secondary hydroxy group. Therefore, in Comparative Examples 1 and 6, it is presumed that the adhesion between the secondary hydroxy group in the linking group and the protective layer was not sufficient, and the pickup characteristics and the chemical substance resistance deteriorated.

The magnetic recording media of Comparative Examples 3 and 7 each had a lubricating layer formed of the compounds (4C) and (4G). In the compounds (4C) and (4G), the linking group disposed between two or three perfluoropolyether chains had two secondary hydroxy groups. Therefore, in Comparative Examples 3 and 7, it is presumed that the adhesion between the secondary hydroxy groups in the linking group and the protective layer was not sufficient, and the pickup characteristics and the chemical substance resistance deteriorated.

The magnetic recording media of Comparative Examples 2, 4, and 5 each had a lubricating layer formed of the compounds (4B), (4D), and (4E). In the compounds (4B), (4D), and (4E), the linking group disposed between two perfluoropolyether chains had both a secondary hydroxy group and a primary hydroxy group. Between the hydroxy groups included in the linking group, the primary hydroxy group having high adsorption ability could be bonded to the protective layer, but the secondary hydroxy group having low mobility had low adsorption ability, and thus the adhesion to the protective layer was not sufficient. As a result, in Comparative Examples 2, 4, and 5, it is presumed that the pickup characteristics deteriorated, and the entrapment of a contaminant caused by a hydroxy group that had not closely adhered to the protective layer was caused, and thus the chemical substance resistance deteriorated.

### INDUSTRIAL APPLICABILITY

The use of the lubricant for a magnetic recording medium including the fluorine-containing ether compound of the present invention makes it possible to form a lubricating layer having excellent chemical substance resistance and an excellent pickup suppression effect even in a case where the thickness is small.

### REFERENCE SIGNS LIST

10 Magnetic recording medium
11 Substrate
12 Adhesion layer
13 Soft magnetic layer
14 First underlayer
15 Second underlayer
16 Magnetic layer
17 Protective layer
18 Lubricating layer

1. A fluorine-containing ether compound represented by Formula (1),
R¹—CH₂—R²—(CH₂—OCH₂—CR³R⁴—CH₂O—CH₂—R²)_{z}—CH₂—R⁵ (1)
(in Formula (1), z is 1 or 2, R² is a perfluoropolyether chain, (z+1) R²'s may be the same in part or in whole, or may be different from each other, R¹ and R⁵ may be the same as or different from each other and are terminal groups having 1 to 50 carbon atoms and having at least one polar group, R³ and R⁴ may be the same as or different from each other and are organic groups which do not include a secondary hydroxy group and a tertiary hydroxy group, include one primary hydroxy group, and have 1 to 5 carbon atoms bonded to a tetrasubstituted carbon atom, the organic group may include an ether oxygen atom between carbon atoms, a bonding terminal to the tetrasubstituted carbon atom may be the ether oxygen atom, and in a case where z is 2, two —CR³R⁴-'s may be the same as or different from each other).

2. The fluorine-containing ether compound according to claim 1, wherein R³ and R⁴ in Formula (1) are each independently any of groups represented by Formulae (2-1) to (2-3), 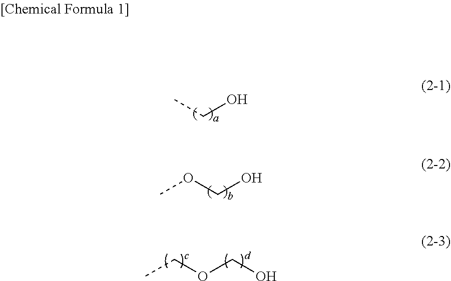
(in Formula (2-1), a is an integer of 1 to 5),
(in Formula (2-2), b is an integer of 1 to 5.), and
(in Formula (2-3), c and d are each independently an integer of 1 to 4, and c+d<5).

3. The fluorine-containing ether compound according to claim 1, wherein the numbers of polar groups contained in R¹ and R⁵ in Formula (1) are each any of 1 to 3.

4. The fluorine-containing ether compound according to claim 1, wherein the polar groups contained in R¹ and R⁵ in Formula (1) are at least one selected from the group consisting of a hydroxy group, an amino group, a carboxy group, a formyl group, a carbonyl group, a sulfo group, a cyano group, and a group having an amide bond.

5. The fluorine-containing ether compound according to claim 1, wherein R¹ and R⁵ in Formula (1) each independently have at least one hydroxy group as the polar group.

6. The fluorine-containing ether compound according to claim 1, wherein R¹ and R⁵ in Formula (1) are each independently any of terminal groups represented by Formulae (4-1) to (4-3), 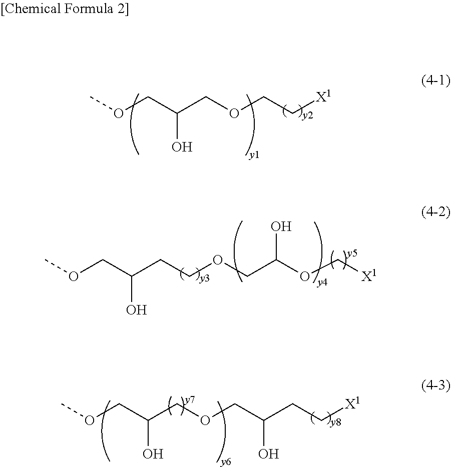
(in Formula (4-1), y1 is 1 or 2, y2 is an integer of 0 to 3, X¹ is an aromatic hydrocarbon group, an unsaturated heterocyclic group, an alkenyl group, an alkynyl group, or a polar group, in a case where y1 is 1, X¹ is a polar group, in a case where X¹ is an aromatic hydrocarbon group or an unsaturated heterocyclic group, an atom constituting a ring structure in X¹ is bonded to a methylene group adjacent to X¹, and in a case where X¹ is an alkenyl group or an alkynyl group, a carbon atom constituting an unsaturated bond in X¹ is bonded to a methylene group adjacent to XV),
(in Formula (4-2), y3 is an integer of 1 to 3, y4 is 0 or 1, y5 is an integer of 0 to 3, X¹ is an aromatic hydrocarbon group, an unsaturated heterocyclic group, an alkenyl group, an alkynyl group, or a polar group, in a case where y4 is 0, X¹ is a polar group, in a case where X¹ is an aromatic hydrocarbon group or an unsaturated heterocyclic group, an atom constituting a ring structure in X¹ is bonded to a methylene group adjacent to X¹, and in a case where X¹ is an alkenyl group or an alkynyl group, a carbon atom constituting an unsaturated bond in X¹ is bonded to a methylene group adjacent to X¹),
(in Formula (4-3), y6 is 0 or 1, y7 is an integer of 1 to 3, y8 is an integer of 1 to 3, X¹ is an aromatic hydrocarbon group, an unsaturated heterocyclic group, an alkenyl group, an alkynyl group, or a polar group, in a case where y6 is 0, X¹ is a polar group, in a case where X¹ is an aromatic hydrocarbon group or an unsaturated heterocyclic group, an atom constituting a ring structure in X¹ is bonded to a methylene group adjacent to X¹, and in a case where X¹ is an alkenyl group or an alkynyl group, a carbon atom constituting an unsaturated bond in X¹ is bonded to a methylene group adjacent to X¹).

7. The fluorine-containing ether compound according to claim 1, wherein (z+1) R²'s in Formula (1) are each independently a perfluoropolyether chain represented by Formula (3),
—(CF₂)_{w1}—O—(CF₂O)_{w2}—(CF₂CF₂O)_{w3}—(CF₂CF₂CF₂O)_{w4}—(CF₂CF₂CF₂CF₂O)_{w5}—(CF₂)_{w6}— (3)
(in Formula (3), w2, w3, w4, and w5 represent average degrees of polymerization and each independently represent 0 to 20, where, all of w2, w3, w4, and w5 are not zero at the same time, w1 and w6 are average values representing the number of CF₂'s and each independently represent 1 to 3, and an order of arrangement of (CF₂O), (CF₂CF₂O), (CF₂CF₂CF₂O), and (CF₂CF₂CF₂CF₂O), which are repeating units in Formula (3), is not particularly limited).

8. The fluorine-containing ether compound according to claim 1, wherein (z+1) R²'s in Formula (1) are each independently any one selected from perfluoropolyether chains represented by Formulae (3-1) to (3-4),
—CF₂—(OCF₂CF₂)ₙ—(OCF₂)ₒ—OCF₂— (3-1)
(in Formula (3-1), n and o represent average degrees of polymerization, n represents 1 to 20, and o represents 0 to 20),
—CF₂CF₂—(OCF₂CF₂CF₂)ₚ—OCF₂CF₂— (3-2)
(in Formula (3-2), p represents an average degree of polymerization, and represents 1 to 15),
CF₂CF₂CF₂—(OCF₂CF₂CF₂CF₂)_{q}—OCF₂CF₂CF₂— (3-3)
(in Formula (3-3), q represents an average degree of polymerization, and represents 1 to 10),
—(CF₂)_{w7}—O—(CF₂CF₂CF₂O)_{w8}—(CF₂CF₂O)_{W9}—(CF₂)_{w10}— (3-4)
(in Formula (3-4), w8 and w9 represent average degrees of polymerization, and each independently represent 1 to 20, and w7 and w10 are average values representing the number of CF₂'s and each independently represent 1 to 2).

9. The fluorine-containing ether compound according to claim 1, wherein in Formula (1), all of z R³'s and R⁴'s in —CR³R⁴— are the same as each other.

10. The fluorine-containing ether compound according to claim 1, wherein all (z+1) R²'s in Formula (1) are the same as each other.

11. The fluorine-containing ether compound according to claim 1, wherein R¹ and R⁵ in Formula (1) are the same as each other.

12. The fluorine-containing ether compound according to claim 1, wherein a number-average molecular weight is within a range of 500 to 10000.

13. A lubricant for a magnetic recording medium, comprising:
the fluorine-containing ether compound according to claim 1.

14. A magnetic recording medium comprising at least in order, on a substrate:
a magnetic layer;
a protective layer; and
a lubricating layer,
wherein the lubricating layer includes the fluorine-containing ether compound according to claim 1.

15. The magnetic recording medium according to claim 14,
wherein an average film thickness of the lubricating layer is 0.5 nm to 2.0 nm.
